(19) United States
(12) Patent Application Publication
(10) Pub. No.: US 2025/0361544 A1
(43) Pub. Date: Nov. 27, 2025

(54) TYPE III-D CRISPR-CAS SYSTEM AND USES THEREOF

(71) Applicants: BOARD OF REGENTS, THE UNIVERSITY OF TEXAS SYSTEM, Austin, TX (US); OTAGO INNOVATION LIMITED, Dunedin (NZ)

(72) Inventors: David M. Taylor, Austin, TX (US); Evan Schwartz, Austin, TX (US); Jack P.K. Bravo, Austin, TX (US); Robert David Fagerlund, Dunedin (NZ); Peter C. Fineran, Dunedin (NZ); Leah Smith, Dunedin (NZ); David Mayo-Munoz, Dunedin (NZ)

(21) Appl. No.: 18/873,888

(22) PCT Filed: Jun. 13, 2023
(86) PCT No.: PCT/NZ2023/050059

§ 371 Date: Dec. 11, 2024

(30) Foreign Application Priority Data
Jun. 13, 2022 (AU) 2022901608

(51) Int. Cl. C12Q 1/6813; C12N 9/22

(52) U.S. Cl. CPC C12Q 1/6813; C12N 9/224; C07K 2319/00

(57) ABSTRACT

The present invention is concerned with novel CRISPR-Cas systems which are configured to detect the presence of a target nucleic acid in a sample through activation of secondary nucleases which bind and cleave a nucleic acid probe modified with a (e.g.) fluorophore/quencher moieties, where a change in the property of the probe (e.g. modified fluorescence) reflects the presence of the target nucleic acid in a sample to be tested.

### CROSS REFERENCE TO RELATED APPLICATIONS

This application is a U.S. national stage of International Patent Application No. PCT/NZ2023/050059, filed Jun. 13, 2023, which claims the benefit of Australian Patent Application No. 2022901608, filed Jun. 13, 2022, the entire contents of each of which are fully incorporated herein by reference.

### STATEMENT OF GOVERNMENT SUPPORT

This invention was made with government support under Grant no. R35 GM138348 awarded by the National Institutes of Health. The government has certain rights in the invention.

### INCORPORATION BY REFERENCE OF SEQUENCE LISTING

The application is accompanied by a sequence listing in electronic format. The sequence listing is provided as a file entitled “70816_SubSeqListing.xml”; Size: 201,705 bytes; created: Dec. 26, 2024. The information in the electronic format of the sequence listing is incorporated by reference in its entirety.

### FIELD OF THE INVENTION

The present invention relates to methods of modifying or detecting single stranded nucleic acids in a targeted manner using Type III-D CRISPR-Cas systems, and to modified Type III-D CRISPR-Cas systems.

### BACKGROUND OF THE INVENTION

CRISPR-Cas (Clustered regularly interspaced short palindromic repeats-CRISPR associated proteins) are heritable prokaryotic adaptive immune mechanisms that provide cellular defence against mobile genetic elements such as phages and plasmids. CRISPR-Cas can be broken down into different types, types I-VI, each determined by ‘signature’ proteins. The mechanism of CRISPR-Cas can be broken down into three stages: 1. Adaptation 2. Expression and Processing 3. Interference. Interference involves a ribonucleoprotein effector complex that surveys invading nucleic acids. Upon recognition of a foreign complementary sequence, crRNA facilitates binding and the foreign sequence is degraded by Cas proteins.

Type I (50%) and Type III (25%) are the most abundant CRISPR-Cas systems. They are genetically diverse and have different methods of interference, yet a similar complex architecture made up of multiple subunits. Type I systems typically contain Cas5, Cas7, Cas6 and a Cas8 proteins, and upon specific binding to target DNA via PAM-mediated recognition, a Cas3 helicase-nuclease is then recruited to degrade DNA. Usually, two small subunits are present or none. In contrast, Type III systems contain the Cas10 subunit instead of Cas8, do not contain Cas3, have no PAM-mediated target recognition, they have the intrinsic ability to specifically bind and cleave RNA by virtue of Cas7, non-specifically cleave single stranded (ss) DNA via Cas10, and the ability to produce secondary messenger molecules (cyclic oligoadenylates) via Cas10. Accessory proteins are activated upon binding these cyclic oligoadenylates and can function to cleave RNA, DNA or proteins (dependent on the particular accessory protein).

Cyanobacteria represent an ancient and diverse phylum with key roles in marine, fresh water and terrestrial ecosystems, including global nitrogen and carbon cycling. They can be responsible for harmful toxic blooms and in biotechnology they are being developed as solar-powered biofactories. Cyanobacteria are under constant threat of phage infection and one mechanism used to counter these is the CRISPR-Cas defence system. Understanding CRISPR-Cas systems in cyanobacteria has attracted significant interest as such systems may have novel biotechnological applications. It has been found that cyanobacteria harbour a novel subtype of CRISPR-Cas system; the subtype III-Dv system. This system is of significant interest as it has an unusual series of Cas7 subunit fusions which effect single stranded nucleic acid cleavage, and bioinformatic studies suggest it appears to be an evolutionary intermediate between typical multiple subunit Type III-A or III-B and recently discovered single subunit Type III-E CRISPR systems.

In the race for survival between bacteria and bacteriophages, CRISPR-Cas systems evolved to provide adaptive immunity for bacteria (Barrangou et al., 2007). CRISPR-Cas effector complexes target foreign mobile genetic elements through sequence-specific hybridization with the crRNA guide and Cas nucleases (Brouns et al., 2008). Interference by Type III CRISPR-Cas effectors target nascent RNA transcripts with a 6-nt cleavage periodicity (Staals et al., 2013; Tamulaitis et al., 2014). Upon binding of an RNA target, Type III systems may initiate ssDNA cleavage using the HD domain of Cas10. Furthermore, RNA target binding induces cyclic oligoadenylate (cOA) production by the palm domain of Cas10 (Jia, Jones, et al., 2019; Kazlauskiene et al., 2017; Niewoehner et al., 2017; Sofos et al., 2020). Cyclic oligoadenylates are allosteric activators of accessory nucleases (often containing CARF domains), such as Csm6 (Kazlauskiene et al., 2017; Makarova, Timinskas, et al., 2020; Niewoehner et al., 2017), which provide the host a second line of defence.

Recently, two studies characterized Type III-E CRISPR-Cas systems (Özcan et al., 2021; van Beljouw et al., 2021b). The Type III-E effector is composed of a single polypeptide made up of multiple Cas7 subunit domain fusions, including one domain split by a large insertion. Interestingly, this complex lacks Cas10 and Cas5, but still contains a Cas11 domain (see FIG. 6). These studies demonstrated RNase activity by two of the four Cas7 domains.

A potential evolutionary intermediate between the multi-subunit Type III-A/B systems and the single-subunit Type III-E system is the Type III-D system (Makarova et al., 2020). The III-D systems are marked by the presence of csx10 (a specific variant of cas5), and often have a csx19 gene, of which the function remains unknown.

Analysis of the CRISPR-Cas systems in Synechocystis sp. PCC6803 (hereafter, Synechocystis) revealed another III-D variant, III-Dv (Matthias et al., 2014; Scholz et al., 2013). As noted above, the Type III-Dv system appears to also be an evolutionary intermediate between multi-subunit and single effector CRISPR-Cas complexes. It contains Cas10, Csx19, a Cas7-Cas7 fusion, a Cas7-Cas5-Cas11 fusion, Cas7 with an insertion, and crRNA. Unlike the Type III-D2 intermediate, the Type III-Dv system contains the unusual fusion for the cas7-cas5-cas11 genes, csx19 and fusion of just two cas7 genes (Makarova et al. 2020). Csx19 has unknown function, but is a signature gene for Type III-D. Furthermore, it is not obvious what subunit(s) is involved in cleavage of target nucleic acids, as the subunits in the Type III-Dv system are unique fusions compared to conventional Type III complexes (Matthias et al., 2014; Scholz et al., 2013; Makarova et al. 2020).

Previous reports have highlighted the evolutionary scenario from multi-gene effectors (III-D1) to the single-subunit Type III-E effectors (Özcan et al., 2021). Recently, a variant III-D system (III-Dv) was described, showing multiple gene fusions, which suggest that it is positioned as an evolutionary intermediate between the multi-subunit and single-subunit effectors (FIG. 6) (Makarova, Wolf, et al., 2020). Interestingly, the III-Dv system has key differences to the other III-D systems in this evolutionary scenario, such as it maintains csx19, cas10, and cas5 similar to III-D1, but includes a large insertion interrupting the terminal cas7 gene, which appears conserved within Type III-E systems (Makarova, Wolf, et al., 2020).

Despite deriving an evolutionary relationship between these different Type III effectors, there is no detailed structural knowledge, nor any proven functions of the Type III-D systems, especially the obscure Type III-Dv systems. Therefore to date, there have been no uses developed for these systems in real world applications.

The present invention aims to address one or more of the above-mentioned limitations in the art.

### SUMMARY OF INVENTION

In another aspect of the present invention there is provided a method of modifying a target nucleic acid, the method comprising contacting the target single-stranded nucleic acid with:
(a) a Type III-D CRISPR-Cas system; and
(b) a guide RNA complementary to the target single-stranded nucleic acid sequence for a time and under conditions sufficient to modify the target single-stranded nucleic acid.

In another aspect of the present invention there is provided a method of modifying a target nucleic acid, the method comprising contacting the target single-stranded nucleic acid with:
(a) a Type III-Dv CRISPR-Cas system comprising:
   (i) a Cas7-Cas5-Cas11 fusion subunit;
   (ii) a Cas7-Cas7 fusion subunit;
   (iii) a Cas7-insertion subunit;
   (iv) a Cas10 subunit;
   (v) Csx19 subunit; and
(b) a guide RNA complementary to the target single-stranded nucleic acid sequence for a time and under conditions sufficient to modify the target single-stranded nucleic acid.
(a)

In yet another aspect of the present invention there is provided a method of detecting a target single-stranded nucleic acid in a sample, the method comprising:
(a) contacting the sample with a complex comprising:
   (i) a Type III-DCRISPR-Cas system comprising
      (1) a Cas7-Cas5-Cas11 fusion subunit;
      (2) a cas7-Cas7 fusion subunit;
      (3) a Cas7-insertion subunit;
      (4) a Cas10 subunit;
      (5) a Csx19 subunit; and
   (ii) and a guide RNA which is complementary to a recognition sequence in the target single-stranded nucleic acid
   to form a reaction mix;
(b) incubating the reaction mix from (a) for a time and under conditions sufficient to allow the complex to bind to the target nucleic acid if present, and produce at least one cyclic oligoadenylate (coA);
(c) contacting the reaction mix from (b) with a nuclease and one or more nucleic acid probes, wherein the nuclease is activated by the at least one coA;
(d) incubating the reaction mix from (c) for a time and under conditions sufficient to cleave the one or more nucleic acid probes to produce one or more cleaved nucleic acid probes; and
(e) determining whether one or more cleaved nucleic acid probes is present in the sample.

In yet another aspect of the present invention there is provided a modified Type III-Dv CRISPR-Cas system comprising:
a Cas 10 subunit, a Csx19 subunit, a Cas7-Cas7 fusion subunit, a Cas7-Cas5-Cas11 fusion subunit, and a Cas7-insertion subunit, wherein at least one of the Cas7 containing subunits is modified to have a reduced ribonuclease activity.

In yet another aspect of the present invention there is provided a modified Type III-D CRISPR-Cas system comprising:
a Cas10 subunit, a Csx19 subunit, a Cas7-Cas7 fusion subunit, a Cas7-Cas5-Cas11 fusion subunit, and a Cas7-insertion subunit, wherein the Cas7-Cas5-Cas11 fusion subunit comprises a mutation at an amino acid residue that is, or is equivalent to, D26 in SEQ ID NO: 4.

In yet another aspect of the present invention there is provided a modified Type III-D CRISPR-Cas system comprising:
a Cas10 subunit, a Csx19 subunit, a Cas7-Cas7 fusion subunit, a Cas7-Cas5-Cas11 fusion subunit, and a Cas7-insertion subunit, wherein the Cas7-Cas7 fusion subunit comprises a mutation at an amino acid residue that is, or is equivalent to, D33 in SEQ ID NO: 6.

In yet another aspect of the present invention there is provided a modified Type III-D CRISPR-Cas system comprising:
a Cas10 subunit, a Csx19 subunit, a Cas7-Cas7 fusion subunit, a Cas7-Cas5-Cas11 fusion subunit, and a Cas7-insertion subunit, wherein the Cas7-Cas7 fusion subunit comprises a mutation at an amino acid residue that is, or is equivalent to, D246 in SEQ ID NO: 6.

In yet another aspect of the present invention there is provided a modified Type III-D CRISPR-Cas system comprising:
a Cas10 subunit, a Csx19 subunit, a Cas7-Cas7 fusion subunit, a Cas7-Cas5-Cas11 fusion subunit, and a Cas7-insertion subunit, wherein the Cas7-Cas7 fusion subunit comprises a mutation at an amino acid residue that is, or is equivalent to, D33 in SEQ ID NO: 6 and a mutation to an amino acid residue that is, or is equivalent to, D246 in SEQ ID NO: 6.

In yet another aspect of the present invention there is provided a modified Type III-D CRISPR-Cas system comprising:
a Cas10 subunit, a Csx19 subunit, a Cas7-Cas7 fusion subunit, a Cas7-Cas5-Cas11 fusion subunit, and a Cas7-insertion subunit, wherein the Cas7-Cas7 fusion subunit comprises a mutation at an amino acid residue that is, or is equivalent to, D33 in SEQ ID NO: 6, a mutation to an amino acid residue that is, or is equivalent to, D246 in SEQ ID NO: 6 and a mutation to an amino acid residue that is, or equivalent to, D26 in SEQ ID NO: 4.

In yet another aspect of the present invention there is provided a modified Type III-D CRISPR-Cas system comprising:
a Cas10 subunit, a Csx19 subunit, a Cas7-Cas7 fusion subunit, a Cas7-Cas5-Cas11 fusion subunit, and a Cas7-insertion subunit, wherein the Cas10 subunit is modified to have a reduced deoxyribonuclease activity and/or is modified to reduce cyclic oligoadenylate production.

In yet another aspect of the present invention there is provided a modified Type III-D CRISPR-Cas system comprising:
a Cas10 subunit, a Csx19 subunit, a Cas7-Cas7 fusion subunit, a Cas7-Cas5-Cas11 fusion subunit, and a Cas7-insertion subunit, wherein the Cas10 subunit comprises mutations at amino acid residues that are, or are equivalent to, H337 and D338 in SEQ ID NO: 2.

In yet another aspect of the present invention there is provided a fusion protein comprising a Cas7-Cas7 fusion subunit, a Cas7-Cas5-Cas11 fusion subunit, and a Cas7-insertion subunit.

In yet another aspect of the present invention there is provided one or more nucleic acids encoding a modified Type III-D CRISPR-Cas system as described herein.

In another aspect of the present invention there is provided a nucleic acid encoding a nuclease which is activated by at least one cyclic oligoadenylate.

In yet another aspect of the present invention there is provided a nucleic acid encoding a guide RNA.

In yet another aspect of the present invention there is provided a vector (e.g. expression vector), phage or virus comprising one or more nucleic acids as described herein.

In yet another aspect of the present invention there is provided a host cell comprising one or more nucleic acids as described herein, or a vector, expression vector, phage or virus as described herein, and optionally a nuclease which is activated by cyclic oligoadenylates, or a nucleic acid encoding such a nuclease, or a guide RNA or a nucleic acid encoding a guide RNA.

### BRIEF DESCRIPTION OF THE FIGURES

FIG. 1 shows stoichiometry and architecture of the Synechocystis CRISPR-Cas Type III-Dv complex. (a) Gene organization of the Type III-Dv operon. (b) TBE-Urea PAGE analysis of crRNA length for the Type III-Dv complex. Product is 37-nt long. (c) SDS-PAGE of the purified Type III-Dv complex after size-exclusion chromatography. (d) Native MS-MS of the Type III-D complex. Peaks correspond to the full WT complex (circle), ΔCsx19 (square), ΔCas10 (green triangle), and ΔCas7-2× (red triangle). (e) Cryo-EM map of Type III-D binary complex. (f) Atomic model of the Type III-D binary complex. (g) Models of each subunit in the Type III-D complex.

FIG. 2 shows crRNA seed region initiates RNA target binding. (a) Structure of the Type III-Dv (ternary) complex bound to a target RNA, with the cryo-EM map on the left and atomic model on the right. (b) Surface representation of the Type III-Dv complex highlights the buried surface of the crRNA with the exception of a seed region that is exposed by the insertion domain of Cas7-insertion. (c) Exposed residues in the crRNA seed region stabilized by residues in the insertion domain. (d) The crRNA seed region sits in a positively charged pocket of the insertion domain. (e) A salt bridge between D616 and R400/K396 blocks RNA target binding at this region, presumably requiring seeding first. (f) Separation of the salt bridge in e to accommodate the RNA target. (g) Modevector map showing the conformational change in the insertion domain of Cas7-insertion, allowing the salt bridge to break apart, as seen in (g).

FIG. 3 shows RNA targeting by Type III-Dv CRISPR-Cas system. (a) Representation of the crRNA-RNA target duplex. The observed bases (black) and 3¢ bases of the target not tracible (gray) are shown. The 5¢ bases or the target not tracible are not shown. The crRNA 5¢ handle is highlighted (purple shade). Cleavage positions (red arrows) and respective catalytic Cas7 domains are indicated. (b) Schematic of labeled RNA targets and cleavage locations. (c) Cleavage of the RNA target with a 5′-FAM and 3′-FAM label. Products are visualized via TBE-Urea PAGE. (d) RNA cleavage time course with a 5′-IRD800 labelled RNA target across 75 minutes. (e) RNA cleavage time course with a 3′FAM labelled RNA target across 75 minutes. (f) Active site aspartates for each Cas7-Cas5-Cas11 and Cas7-2x, each positioned at the kinked scissile phosphate. The inactive D616 of Cas7-insertion is also shown. (g) 5′IRD800-labelled and h, 3′FAM-labelled RNA cleavage analysis after mutagenesis of the 3 active site residues of Cas7-Cas5-Cas11, Cas7-2x.1, and Cas7-2x.2. (i) Active site of Cas7-Cas5-Cas11 for ssRNA cleavage. Dashed arrows show coordination of a coordinated water molecule.

FIG. 4 shows Cas10 activation by non-self RNA binding. (a) Path of the RNA target strand gets directed through Cas10 rather than into the anti-repeat pocket. (b) Upon binding a non-self ssRNA target, an activating helix in Cas10 gets pushed by the target RNA into an active conformation. (c,d) Cyclic oligoadenylate (PDB: 607b) bound to the Type III-A Cas10 subunit (Csm1) fit into our Type III-D target-bound and binary models, respectively.

FIG. 5 shows in silico model prediction of Type III-E and Type III-D Cas proteins. (a) Type III-A Csm complex atomic model, colored by subunits. (b) Type III-Dv atomic model, colored by domains. (c) Alphafold 2 structure prediction of the D. ishimotonii (Cas7-11) Type III-E effector protein, colored by domains. Linkers are colored in gold. (d) Structural alignment of Cas7-2x.1 D33 of Type III-Dv with Cas7.2 D429 of D. ishimotonii Type III-E. (e) Structural alignment of Cas7-2x.2 D246 of Type III-Dv with Cas7.3 D654 of D. ishimotonii Type III-E. (f) Alphafold 2 predicted models of the a single polypeptide Type III-Dv containing proteins (Cas7-Cas5-Cas11)-(Cas7-2x)-(Cas7-insertion). Linkers from the Type III-E model between Cas11 and Cas7-2x, as well as between Cas7-2× and Cas7-insertion are in pink. Linkers in the Cas7-Cas5-Cas11 subunit are in gold.

FIG. 6 shows proposed evolution between Type III-D variants to Type III-E.

FIG. 7 shows purification of the Type III-Dv effector complex. (a) Size-exclusion chromatograph of the WT Type III-Dv complex bound to crRNA. Black peak corresponds to ˜330 kDa. Grey peaks represent standardized molecular weights.

FIG. 8 shows EM validation of Type III-Dv binary (left) and ternary (right) maps. (a) Representative micrographs. Scale bar is shown at 100 nm. (b) FSC plot of the two maps based on the 0.143 gold standard of two half maps. (c) Euler angular distribution showing distribution of orientations that contribute to the final map. (d) Guinier plots to show BFactor sharpening calculation for final EM maps. (e) Final sharpened EM maps from cryoSPARC v2 at 2.52 Å and 2.77 Å, respectively.

FIG. 9 shows representative cryo-EM density of the Type III-Dv complex.

FIG. 10 shows subunit comparisons between Type III-Dv and Type III-A/B homologues. (a) crRNA-target duplex of the Type III-Dv ternary complex is stabilized by a positive patch on the Cas11 domain of Cas7-Cas5-Cas11. (a) Structural alignments between the Type III-Dv Cas7 domain of Cas7-Cas5-Cas11 with Cmr4 (Cas7) of the Type III-B (peach) and Csm3 of the Type III-A complex (white); (b) Type III-Dv Cas5 domain of Cas7-Cas5-Cas11 with Cas5 (Csm4) of the Type III-A complex (magenta) and Cas5 (Cmr3) of the Type III-B complex (beige); (c) Type III-Dv Cas10 subunit with Cas10 (Csm1) of the Type III-A complex (yellow) and Cas10 (Cmr2) of the Type III-B complex (cyan). (d) HD domain comparison between Csm1 (Cas10) of Type III-A with the putative Type III-Dv HD site.

FIG. 11 shows Type III-Dv interactions with the crRNA. (a) crRNA trajectory through the Type III-Dv effector complex. (b) 3′ crRNA capping by Phe307 of Cas7-insertion. (c) 5′ crRNA capping by Phe71 of Csx19. (d) Arg145 of Csx19 interacting with G4 of the crRNA, upstream of the 5′ crRNA handle. (e) crRNA geometry comparison between Type III-Dv and other Type I and Type III systems.

FIG. 12 shows purification of mutant Type III-Dv effector complexes. (a) Size-exclusion chromatograph of the ΔCsx19 type Type III-Dv complex bound to crRNA (red trace). Black peak corresponds to wild-type Type III-Dv complex. Grey peaks represent standardized molecular weights. (b) SDS-PAGE of the ΔCsx19 from the two broad peaks seen in (a) no complex appears to form. (c) Purification of Cas7 active site mutants for RNA cleavage analysis.

FIG. 13 shows Type III-Dv interactions with the crRNA. crRNA-target duplex of the III-Dv ternary complex is stabilized by a positive patch on the Cas11 domain of Cas7-Cas5-Cas11.

FIG. 14 shows Serratia NucC is a predicted nuclease that binds cA₃. (A) Schematic of the Type III-A CRISPR-Cas operon from Serratia sp. ATCC 39006. (B) Multiple Sequence Alignment (MUSCLE) of NucC homologs: Ser, Serratia Type III-A CRISPR-Cas-associated NucC; Vm, Vibrio metoecus sp. RC341 Type III-B CRISPR-Cas-associated NucC; Ec, E. coli MS115-1 CBASS-associated NucC; Pa, P. aeruginosa ATCC27853 CBASS-associated NucC. The percentage of amino acid similarity is indicated in different shades of purple (Score Matrix: Blosum62; Threshold: 1). The percentage of protein sequence identity is indicated for each protein sequence compared to Serratia NucC. Conserved cA₃-binding binding residues (R63, Y91 and T236) and active site motif (ID-₃₀ExK, where ‘x’ represents any amino acid) are highlighted in grey.

FIG. 15 shows Serratia NucC is a cA₃-activated dsDNase able to degrade Serratia and jumbo phage genomes in vitro. In vitro NucC cleavage of (A) Serratia and (B) jumbo phage (PCH45) gDNA, (C) plasmid and (D) a PCR product for 60 min with cA₃. In (A), NucC active site mutants D83N, E114N and K116L were shown to disrupt Serratia gDNA degradation. (E) Distribution of in vitro NucC cleavage sites in plasmid pPF1043 based on deep-sequencing of 5′-ends of DNA degradation products (n=3,807,021 cleavage sites). (F) Cleavage site preference of NucC (WebLogo) from in vitro degradation products from (E), where ‘n’ represents any nucleotide. Light purple indicates the sequenced strand and purple arrows indicate the cleavage position between nucleotide (nt) positions 10 and 11. (G) To verify the cleavage site, 200 bp synthetic dsDNA products with or without the specified core or full sites were designed to generate products of 50 and 150 bp when cleaved by NucC. (H) In vitro NucC cleavage of synthetic oligonucleotides described in (G).

FIG. 16 shows the jumbo phage DNA-containing protein shell excludes NucC but degrades the bacterial genome. (A) Total DNA extracted at various time points throughout a single round of jumbo phage infection and analysed via gel electrophoresis. (B) Percentage of mapped reads to Serratia (chromosome), jumbo phage genome (phage) and pPF1467 (plasmid) at 40 and 60 min post-infection of wild-type +CRISPR cells resulting from deep sequencing of degradation products. The data shown represents three biological triplicates plotted as the mean±standard deviation. (C) Confocal microscopy of wild-type Serratia with a spacer targeting the jumbo phage (+CRISPR) in the absence (left) and presence (right) of jumbo phage infection. Membranes (magenta) and DNA (blue) were stained with FM-4-64 and DAPI, respectively. Nucleus-like structures form as indicated with arrows. NucC (mEGFP, green) is excluded from DNA foci (arrows) in infected cells (+phage). Scale bars, 1 μm. Quantifications show the fluorescence intensity distributions of the NucC (green) and DNA (blue) across the length of single cells. Images are typical representatives from three biologically independent experiments. (D) DAPI fluorescence monitored in-CRISPR and +CRISPR cells upon jumbo phage (PCH45) infection. Quantifications show the fluorescence intensity distributions of DNA (blue) across the length of single cells.

FIG. 17 shows the Alphafold (Jumper, 2021) predicted structure of a fusion protein comprising “core” subunits of a Type III-Dv system comprising Cas7-5-11, Cas7-2× and Cas7-insert tethered together by two linkers as already shown in FIG. 5, but shown here in more detail. The predicted structure is remarkably similar to the structure solved above. The Cas protein subunits and the linkers are indicated. Note, this construct includes the removal of the first 113 residues of the Cas7-insert subunit. This 113 residue region was not observed in the structure (possibly due to flexibility) and it has been confirmed that this portion can be removed and the effector remains active in cleaving RNA target.

FIG. 18 shows Cas7 domain active sites in the Type III-Dv complex. (a) Cas7-Cas5-Cas11 (D26), (b) Cas7-2x.1 (D33), and (c) Cas7-2x.2 (D246) active sites with EM density.

FIG. 19 shows RNA targeting by the Δ104 Cas7-insertion Type III-Dv complex. Site 1, 2, and 3 correspond to cleavage by Cas7-2x.2, Cas7-2x.1, and Cas7-Cas5-Cas11, respectively. The RNA was labelled at the 5′ end with an IRD800 label.

FIG. 20 shows (A) CRISPR-Cas Type III-Dv effector complex in combination with a NucC DNA nuclease specifically recognises a target RNA and triggers cleavage of a gDNA substrate, (B) CRISPR-Cas Type III-Dv effector complex in combination with a NucC DNA nuclease and fluorescent reporter DNA probe 1 detects target RNA shown by increased fluorescence of cleaved DNA probe, and (C) modified CRISPR-Cas Type III-Dv effector complex with inactive Cas7 subunits in combination with NucC DNA nuclease and fluorescent DNA probe 2 specifically detects target RNA shown by increased fluorescence of cleaved DNA probe.

FIG. 21 shows Type III-Dv represses gene transcription in HEK293 cells. (A) Confocal images at 100× magnification of HEK293 cells transfected with a Type III-Dv expression vector codon optimized for human cells (pPF3610). Total cells in the field of view stained by a DNA stain Hoechst are shown in (i). Panel (ii) shows red fluorescence indicative of red fluorescent protein (RFP) which shows HEK293 cells expressing the III-Dv complex. A Western blot of total cell lysate of HEK293 cells transfected with III-Dv is shown in (iii), indicating expression of the III-Dv complex. (B) CDS of the fluorescent reporter Venus (pPF3328) showing spacer location for III-Dv targeting. (C) Flow cytometry data of HEK293 cells that have been co-transfected with a plasmid expressing Venus (pPF3328) and Type III-Dv vectors with either control spacers or spacers targeting the kozak (protein translation initiation site) and CDS (coding sequence) of Venus (yellow fluorescent protein). Data in (i) shows median fluorescent intensity (MFI) of Venus in RFP positive cells. Statistics were calculated using a one-way ANOVA multiple comparison relative to MFI of Venus in cells transfected with a control spacer, stars represent a significant difference p<0.0001. MFI of Venus in RFP+ cells are then plotted as normalized repression in (ii), data is normalized to the mean MFI for the control spacer and plotted as a percentage. (D) Confocal images at 100× magnification of HEK293 cells co-transfected with a plasmid expressing Venus (pPF3328) and either a Type III-D vector with a control spacer (control spacer 2) or a spacer targeting the CDS of Venus (S2). The first panel shows total cell population, with cells stained with a Hoechst DNA stain; the second panel shows RFP positive cells, indicative of cells expressing Type III-Dv complex, indicated by black arrows; the third panel shows Venus positive cells indicated by grey arrows. Circled cells indicate co-transfected cells expressing both RFP and Venus.

FIG. 22 shows MAP2 depletion using Type III-Dv system in DRG sensory neurons. (A) (i) Schematics showing target of CRISPR-Cas Type III-Dv guides for control and MAP2-specific constructs. Type III-Dv-MAP2-Guide-3 was also designed with a double and triple insert repeat sequence (III-Dv-MAP2-3_2 and III-Dv-MAP2-3_3 respectively); (A) (ii) representative images of 5 DIV DRG sensory neurons expressing III-Dv, III-vD-scrambled control (scControl), III-Dv-control; and (A) (iii) MAP2-specific CasIII-Dv-miniRFP constructs III-Dv-MAP2-1, III-Dv-MAP2-2, III-Dv-MAP2-3, III-Dv-MAP2-3_2, III-Dv-MAP2-3_3, and III-Dv-MAP2-4. Neurons were fixed and immunostained for endogenous MAP2. Arrows indicate areas of high MAP2 intensity. Scale bar 10 μm. (B) Quantification of MAP2 integrated density (arbitrary units) at the cell body sensory neurons expressing III-Dv (n=49), III-Dv-scControl (n=44), III-Dv-Control-1 (n=41), III-Dv-MAP2-1 (n=85), III-Dv-MAP2-2 (n=101), III-Dv-MAP2-3 (n=121), III-Dv-MAP2-3_2 (n=126), III-Dv-MAP2-3_3 (n=92), III-Dv-MAP2-4 (n=128). (C) Average fluorescence intensity of MAP2 in axons of sensory neurons expressing III-Dv (n=50), III-Dv-scControl (n=50), III-Dv-Control-1 (n=46), III-Dv-MAP2-1 (n=107), III-Dv-MAP2-2 (n=118), III-Dv-MAP2-3 (n=135), III-Dv-MAP2-3_2 (n=125), III-Dv-MAP2-3_3 (n=106), III-Dv-MAP2-4 (n=137). Mean+SEM. * p<0.05, ** p<0.01. One-way ANOVA in (C); three independent experiments.

FIG. 23 shows single fusion Type III-Dv represses gene transcription in HEK293 cells. (A) Flow cytometry data of HEK293 cells that have been co-transfected with a plasmid expressing Venus (pPF3328) and single fusion Type III-Dv vectors with either control spacer or spacers targeting the kozak (protein translation initiation site) and CDS (coding sequence) of Venus (yellow fluorescent protein). Data shows median fluorescent intensity (MFI) of Venus in RFP positive cells. Statistics were calculated using a one-way ANOVA multiple comparison relative to MFI of Venus in cells transfected with a control spacer, stars represent a significant difference p<0.0001. (B) Normalized MFI levels of targeting spacers compared to the control.

### GENERAL DEFINITIONS

As used herein, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ≠1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

Unless specifically defined otherwise, all technical and scientific terms used herein shall be taken to have the same meaning as commonly understood by one of ordinary skill in the art (for example, in immunology, immunohistochemistry, protein chemistry, molecular genetics, synthetic biology and biochemistry).

Throughout this specification, unless specifically stated otherwise, or the context requires otherwise, reference to a single step, composition of matter, group of steps or group of compositions of matter shall be taken to encompass one and a plurality (i.e., one or more) of those steps, compositions of matter, groups of steps or group of compositions of matter.

### Selected Definitions

The term “cell” as used herein refers to a prokaryotic or eukaryotic cell and is not limited. A cell may be derived from any bacteria, archaea, plant, animal, or yeast. A cell may be derived from a vertebrate or non-vertebrate animal. A cell may be derived from a non-human or human animal. A cell may be mammalian or non-mammalian.

The term “adjacent” as used herein means next to a location, which may be directly next to, indirectly next to, or proximal to a location. When used with reference to a nucleic acid sequence, ‘adjacent’ may mean directly upstream or downstream of a location, with no nucleotide bases between the nucleic acid sequence and the location, or may mean proximal to a location with a few nucleotide bases between the nucleic acid sequence and the location, such as below 10 nucleotide bases for example.

The terms “base pairing affinity” and “complementarity” as used herein may be used interchangeably and refer to the ability of a nucleic acid to form hydrogen bond(s) with another nucleic acid sequence by either traditional Watson-Crick base pairing or other non-traditional types. The terms “complementary” or “complementarity,” as used herein, refer to the natural binding of polynucleotides under permissive salt and temperature conditions by base-pairing. For example, the sequence “A-G-T” binds to the complementary sequence “T-C-A.” Complementarity between two single-stranded molecules may be “partial,” in which only some of the nucleotides bind, or it may be complete when total complementarity exists between the single-stranded molecules. The degree of complementarity between nucleic acid strands has significant effects on the efficiency and strength of hybridization between nucleic acid strands.

The terms “percent sequence identity” or “percent identity” as used herein refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some examples, “percent identity” can refer to the percentage of identical amino acids in an amino acid sequence. As used herein “sequence identity” refers to the extent to which two optimally aligned polynucleotide or peptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). As used herein, the phrase “substantially identical,” or “substantial identity” in the context of two nucleic acid molecules, nucleotide sequences or protein sequences, refers to two or more sequences or sub-sequences that have at least about 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In particular examples, substantial identity can refer to two or more sequences or sub-sequences that have at least about 80%, at least about 85%, at least about 90%, at least about 95, 96, 96, 97, 98, or 99% identity.

Throughout this specification in any context, optimal alignment may be determined using, for example, any of the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). For purposes of this invention “percent identity” may also be determined using BLASTX version 2.0 for translated nucleotide sequences and BLASTN version 2.0 for polynucleotide sequences.

The term “perfectly complementary” as used herein means about 100% nucleotide or amino acid residues are complementary. Suitably that all the contiguous residues of a nucleic acid sequence will hydrogen bond with the same number of contiguous residues in a second nucleic acid sequence.

The term “substantially complementary” as used herein means at least about 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residues are complementary, when compared and aligned for maximum correspondence, as measured using one of the following sequence comparison algorithms or by visual inspection. Suitably at least a percentage proportion of the contiguous residues of a nucleic acid sequence will hydrogen bond with the same number of contiguous residues in a second nucleic acid sequence. This may also correspond to nucleic acids that hybridize under stringent conditions.

The terms “hybridization”, “hybridize”, “hybridizing”, and grammatical variations thereof as used herein, refer to the binding of two complementary nucleotide sequences or substantially complementary sequences in which some mismatched base pairs are present. The conditions for hybridization are well known in the art and vary based on the length of the nucleotide sequences and the degree of complementarity between the nucleotide sequences. In some examples, the conditions of hybridization can be high stringency, or they can be low stringency depending on the amount of complementarity and the length of the sequences to be hybridized.

The term “stringent conditions” for hybridization as used herein refer to conditions under which a nucleic acid having complementarity to a target sequence predominantly hybridizes with the target sequence, and substantially does not hybridize to non-target sequences. Stringent conditions are generally sequence-dependent and vary depending on a number of factors. In general, the longer the sequence, the higher the temperature at which the sequence specifically hybridizes to its target sequence. Non-limiting examples of stringent conditions surrounding the nucleic acids, temperature, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, NY, 2001); and Tijssen, Laboratory Techniques in Biochemistry and Molecular Biology-Hybridization with Nucleic Acid Probes Part I, Chapter 2 (Elsevier, New York, 1993). The Tm is the temperature at which more than 50% of a given strand of a nucleic acid molecule is hybridized to its complementary strand. The following is an exemplary set of hybridization conditions and is not limiting:

Very High Stringency (allows sequences that share at least 90% identity to hybridize) Hybridization: 5×SSC at 65° C. for 16 hours; wash twice: 2×SSC at room temperature (RT) for 15 minutes each; wash twice: 0.5×SSC at 65° C. for 20 minutes each.

High Stringency (allows sequences that share at least 80%> identity to hybridize) Hybridization: 5×-6×SSC at 65° C.-70° C. for 16-20 hours; wash twice: 2×SSC at RT for 5-20 minutes each; wash twice: lx SSC at 55° C.-70° C. for 30 minutes each.

Low Stringency (allows sequences that share at least 50%> identity to hybridize); hybridization: 6×SSC at RT to 55° C. for 16-20 hours; wash at least twice: 2×-3×SSC at RT to 55° C. for 20-30 minutes each.

Methods performed according to the present invention may be in vitro, for example they are performed using a synthetic mix of the reaction components in a suitable buffer system. In some in vitro examples there is used a cell-free transcription/translation system.

Methods performed according to the present invention may be employed occurring ex vivo, for example in a cell or cell culture. In ex vivo treatments, diseased cells may be removed from the body, treated with the products/methods of the invention, and then transplanted back into the patient. Ex vivo modification has an advantage of allowing the target cell population to be well defined and the specific dosage of therapeutic molecules delivered to cells to be specified.

In vivo examples are also provided. In vivo modification can be used advantageously from this disclosure and the knowledge in the art.

A “fragment” or “portion” of a nucleic acid will be understood to mean a nucleotide sequence of reduced length relative (e.g., reduced by 1, 2, 3, 4, 5, 6, 7, 8, 9, 10, 11, 12, 13, 14, 15, 16, 17, 18, 19, 20 or more nucleotides) to a reference nucleic acid or nucleotide sequence and comprising a nucleotide sequence of contiguous nucleotides that are identical or almost identical (e.g., 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Such a nucleic acid fragment or portion according to the invention may be, where appropriate, included in a larger polynucleotide of which it is a constituent. In some examples, a fragment of a polynucleotide can be a fragment that encodes a polypeptide that retains its function which may be termed a ‘functional fragment’.

A “native” or “wild type” or unmodified nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is a mRNA that is naturally occurring in or endogenous to the organism. A “homologous” nucleic acid is a nucleic acid naturally associated with a host cell into which it is introduced.

As used herein, the terms “nucleic acid,” “nucleic acid molecule,” “nucleic acid construct,” “nucleotide sequence” and “polynucleotide” refer to single-stranded or double-stranded nucleic acids, such as RNA or DNA that is linear or branched, single or double-stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. The nucleic acid constructs of the present disclosure can be DNA or RNA, but are preferably DNA. Thus, although the nucleic acid constructs of this invention may be described and used in the form of DNA, depending on the intended use, they may also be described and used in the form of RNA.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single-stranded or double-stranded. The terms “nucleotide sequence” “nucleic acid,” “nucleic acid molecule,” “nucleic acid construct,” “oligonucleotide,” and “polynucleotide” are also used interchangeably herein to refer to a heteropolymer of nucleotides. Except as otherwise indicated, nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end. Thus, for example, an element in the 3′ region of a polynucleotide can be located anywhere from the first nucleotide located at the 3′ end of the polynucleotide to the nucleotide located halfway through the polynucleotide. An element that is described as being “at the 5′end” or “at the 3′end” of a polynucleotide (5′ to 3′) refers to an element located immediately adjacent to (upstream of) the first nucleotide at the 5′ end of the polynucleotide, or immediately adjacent to (downstream of) the last nucleotide located at the 3′ end of the polynucleotide, respectively.

The term “identity” and “identical” and grammatical variations thereof, as used herein, mean that two or more referenced entities are the same (e.g., nucleic acid or amino acid sequences). Thus, where two sequences are identical, they have the same nucleic acid sequence or the same amino acid sequence. The identity can be over a defined area, e.g. over at least 22, 23, 24, 25 or 26 contiguous nucleic acids of the parent nucleic acid sequence, or over at least 22, 23, 24, 25 or 26 contiguous amino acid residues of a parent peptide sequence, or whichever alignment is the best fit with gaps permitted.

Identity can be determined by comparing each position in aligned sequences. A degree of identity between nucleic acid or amino acid sequences is a function of the number of identical or matching nucleic acids or amino acids at positions shared by the sequences, i.e. over a specified region. Optimal alignment of sequences for comparisons of identity may be conducted using a variety of algorithms, as are known in the art, including the Clustal Omega program available at the website location at www.ebi.ac.uk/Tools/mas/clustalo/, the local homology algorithm of Smith and Waterman, 1981, Adv. Appl. Math 2:482, the homology alignment algorithm of Needleman and Wunsch, 1970, J. Mol. Biol. 48:443, the search for similarity method of Pearson and Lipman, 1988, Proc. Natl. Acad. Sci. USA 85:2444, and the computerized implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at the website located at www.ncbi.nlm.nih.gov). Such algorithms that calculate percent sequence identity (homology) generally account for sequence gaps and mismatches over the comparison region or area. For example, a BLAST (e.g., BLAST 2.0) search algorithm (see, e.g., Altschul et al., J. Mol. Biol. 215:403 (1990), publicly available through NCBI) has exemplary search parameters as follows: Mismatch-2; gap open 5; gap extension 2. For polypeptide sequence comparisons, a BLASTP algorithm is typically used in combination with a scoring matrix, such as PAM 100, PAM 250, BLOSUM 62 or BLOSUM 50. FASTA (e.g., FASTA2 and FASTA3) and SSEARCH sequence comparison programs are also used to quantitate the extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981). Programs for quantitating protein structural similarity using Delaunay-based topological mapping have also been developed (Bostick et al., Biochem Biophys Res Commun. 304:320 (2003)).

### DETAILED DESCRIPTION

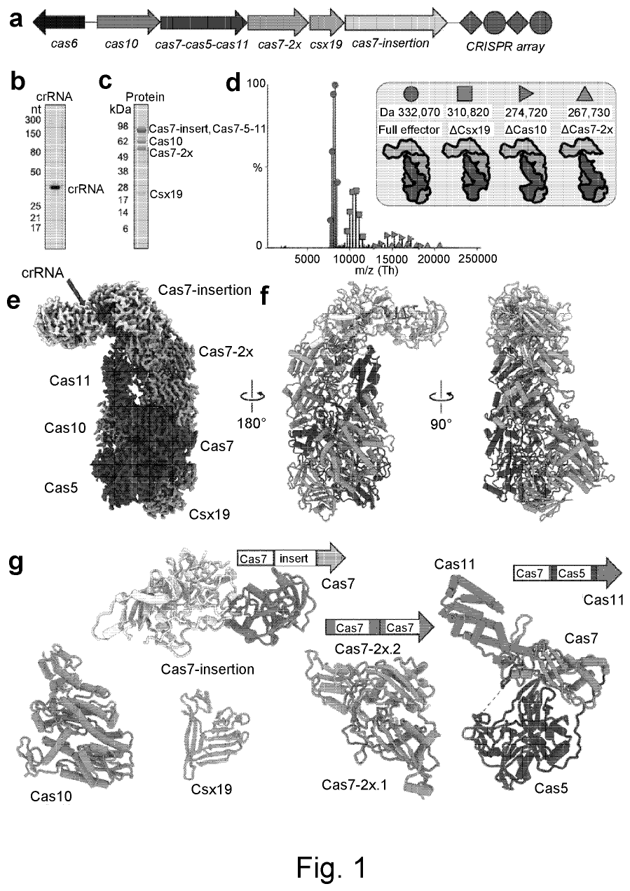

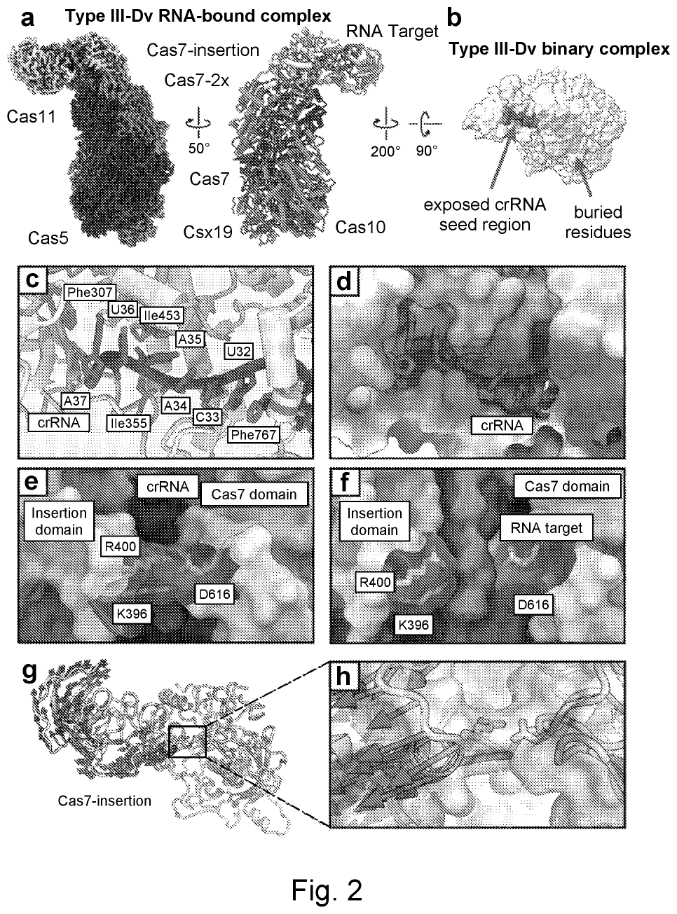

The present inventors have expressed and purified a Type III-Dv effector complex. They have determined the cryoelectron microscopy structures in an apo and RNA-target bound state to produce two cryo-electron microscopy (cryo-EM) structures of the Type III-Dv (binary) surveillance complex, and the RNA target-bound (ternary) effector complex at 2.5- and 2.8-Å resolution, respectively. Refer to Example 1 read in conjunction with FIGS. 1 and 2. These structures shed important insight into the mechanisms of RNA targeting by the Type III-Dv complex. The inventors have discovered that the Type III-Dv complex forms a large structure composed of a single copy of each subunit. Csx19 is at the base of the complex and interacts with Cas10, the Cas5 domain of the Cas5-Cas7-Cas11 subunit, and the crRNA, which suggests Csx19 having a role in crRNA support. The structure reveals a unique architecture where the Cas7-insertion subunit at the top of the complex resembles a “hammer-head” that causes the bound crRNA to bend nearly 90° as it follows the path of this subunit.

The inventors have further shown in experiments that the Type III-Dv complex cleaves target RNA. They have discovered that the complex binds target RNA, and the mechanism of cleavage is different to other CRISPR-Cas systems. From in vitro cleavage assays and the structural information (summarised in FIGS. 1-3), the inventors have identified several ribonucleolytic active sites located in the Cas7 containing subunits. They have found that the Cas7 domains of Cas5-Cas7-Cas11, and Cas7-Cas7 make interactions with target RNA that undertake ribonucleolytic cleavage, but the Cas7-insertion is inactive. Furthermore, they have successfully modified the system at each of these identified ribonucleolytic active sites to demonstrate its use in programmable RNA cleavage at three separate active sites across three unique Cas7 domains. Because there is only one copy of each subunit, it is possible to programme how many cleavage events occur. Based on structural and biochemical data, the inventors have elucidated systems that can cut RNA either once or not at all by modifying the active sites. This provides capability to generate site-specific cleavage at discrete locations on RNA. This is not possible for conventional Type III complexes because they contain multiple Cas7 subunits generated from the same gene; as such these complexes cannot be manipulated to cleave at only one location. Therefore the inventors have realised the use of the Type III-D system in programmable RNA cleavage.

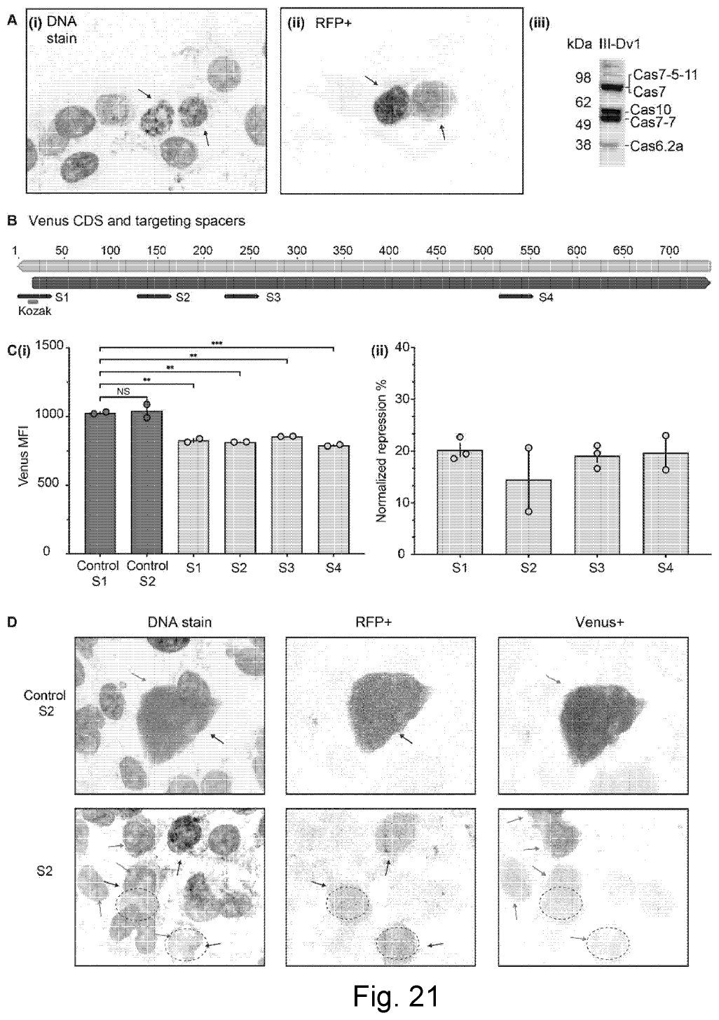

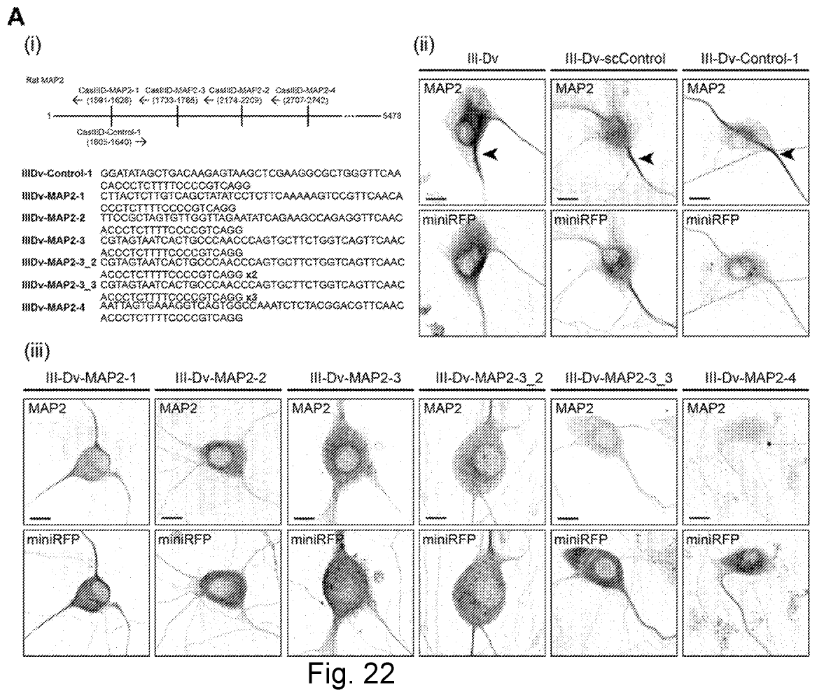

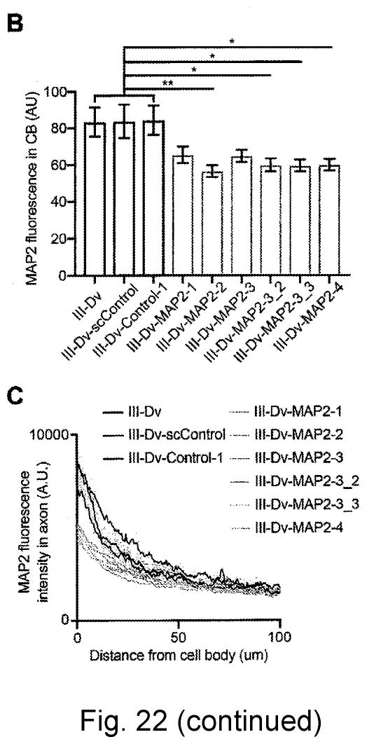

Importantly, the present inventors have shown that Type III-Dv CRISPR-Cas system derived from the cyanobacteria Synechocystis sp. PCC6803 may be successfully transfected into mammalian cells lines, including both a HEK293 cell line and primary sensory neuronal cells, and that successful transfection resulted in targeted gene knock-down via a fluorescence report construct (HEK293) and an endogenous gene (MAP2 in neuronal cells). Refer to Examples 5 and 6, read in conjunction with FIGS. 21 and 22. These data demonstrate a potential (and powerful) utility of Type III-Dv CRISPR-Cas systems as described herein for targeted gene silencing in humans. This has wide-reaching implications for different disease states where gene expression or specific sequences present in an RNA are an underlying cause of disease etiology and pathogenesis.

The inventors have further demonstrated how structural rearrangements between the binary and ternary complex of Type III-Dv system allow for activation of the palm domain of Cas10 to prompt cOA production and ssDNA cleavage when bound to RNA. Refer to Example 4. This function can be used to detect RNA in samples via the cleavage of DNA probes by an accessory nuclease. The inventors have realised that modification of the Cas7 containing subunits as mentioned above, so as not to cleave RNA, will aid in continual production of cOAs for prolonged activation of the accessory nuclease. Enhanced nuclease activity can enhance the sensitivity of the output signal in a diagnostic assay for detecting RNA. The inventors have further modified the HD domain of the Cas10 subunit to inhibit ssDNA cleavage. This modification will prevent the Type III-Dv complex from inadvertently cleaving the DNA probes used in a (e.g. diagnostic) detection assay, and therefore improve the specificity and sensitivity of the detection assay. Therefore, the inventors have realised an application of the Type III-D CRISPR-Cas system in improved RNA detection.

The subunits involved in RNA cleavage are unique to the Type III-Dv complex and the active sites is not obvious without in depth work to obtain structural information that has been carried out by the inventors. Furthermore, the demonstration of RNA cleavage using this system, and the subsequent modification to remove RNA cleaving function, highlights the use of the system in programmable RNA cleavage at particular sites, but also its use as an RNA detection system which has the potential to be more sensitive than other Type III CRISPR-Cas systems.

Further features and examples of the aspects of the invention will now be described under the headed sections below. Any feature or example within these sections may be combined with any aspect in any workable combination.

### Type III-D CRISPR-Cas System

In preferred examples of the present invention the Type III-D CRISPR-Cas system is a variant Type III-D CRISPR-Cas system or Type III-Dv CRISPR-Cas system. It should, however, be appreciated that any reference herein to ‘the system’ in context may refer to either of the Type III-D CRISPR-Cas system or the Type III-Dv CRISPR-Cas system.

Suitably the system is composed of one or more of the following protein domains: Cas7, Cas5, Cas11, Cas10, Csx19 and Cas6. Suitably the system comprises at least the following protein domains: Cas7, Cas5 and Cas11. Suitably in such examples, the system may be used for methods of modification. Suitably, the system comprises at least the following protein domains: Cas7, Cas5, Cas11, Cas10, and Csx19. Suitably in such examples, the system may be used for methods of modification or methods of detection as described herein.

Preferably the system comprises: Cas7, Cas5, Cas11, Cas10, Csx19 and Cas6.

Suitably Cas6 may be associated with the Type III-D CRISPR-Cas system, but is not part of the final active complex. Suitably Cas6 may be present initially during formation of the system, suitably Cas6 is not present once the Type III-D CRISPR-Cas system is formed. Suitably therefore the initial system may be composed of one or more of the following protein domains: Cas7, Cas5, Cas11, Cas10, Csx19 and Cas6. Suitably the final system may be composed of one or more of the following protein domains: Cas7, Cas5, Cas11, Cas10, and Csx19.

Suitably the system comprises plurality of Cas7 proteins. Suitably the Cas7 proteins are present in the system as fusions with other Cas proteins. Suitably therefore the Cas7 proteins are present in subunits. Each subunit may suitably comprise one or more Cas7 proteins fused to one or more other Cas proteins as listed above.

Suitably the system comprises the following subunits: a Cas7-Cas7 fusion protein, a Cas7-Cas5-Cas11 fusion (also referred to as Cas7-5-11) protein, and a Cas7 protein with an insertion. Suitably therefore the system is a Type III-Dv CRISPR-Cas system. Suitably a minimal form of the Type III-Dv CRISPR-Cas system may comprise only the following subunits: a Cas7-Cas7 fusion protein, a Cas7-Cas5-Cas11 fusion (also referred to as Cas7-5-11) protein, and a Cas7 protein with an insertion. Suitably, as noted above, such a minimal form of the system may be used in methods of modification as described herein.

Suitably there is one copy of each subunit present in the system. Suitably one copy of each of the following subunits: a Cas7-Cas7 fusion protein, a Cas7-Cas5-Cas11 fusion (also referred to as Cas7-5-11) protein, and a Cas7 protein with an insertion.

In one embodiment, the Type III-Dv CRISPR-Cas system comprises the following proteins: Cas10, Csx19, Cas7-Cas7 fusion protein, Cas7-Cas5-Cas11 fusion protein, and Cas7 protein with an insertion, which may equally be referred to as ‘subunits’ herein. Suitably, as noted above, such a system may be used in methods of modification or methods of detection as described herein.

In one example, the Type III-Dv CRISPR-Cas system consists of the following proteins/subunits: Cas10, Csx19, Cas7-Cas7 fusion protein, Cas7-Cas5-Cas11 fusion protein, and Cas7 protein with an insertion.

Suitably, as explained above, Cas6 may be associated with the Type III-Dv CRISPR-Cas system, and therefore may be present in the methods of the present invention.

Suitably the Type III-D CRISPR-Cas system comprises at least one Cas7 protein, suitably multiple Cas7 proteins as explained above. Suitably the Cas7 or each Cas7 protein contained within the Cas7-Cas7 fusion protein and the Cas7-Cas5-Cas11 fusion protein carries out cleavage of single-stranded nucleic acids, for example ribonucleic acids. Suitably the Cas7 or each Cas7 protein may be active or inactive. In some examples, it may be useful for the Cas7 or each Cas7 protein to be modified such that it is nuclease deficient, in other words inactive. In some examples, it may be useful for the Cas7 or each Cas7 protein to be wild type, in other words active. Suitably in methods of modifying a target single stranded nucleic acid as described herein, at least one Cas7 protein is active, suitably at least one Cas7 protein of the Cas7-Cas7 fusion subunit and the Cas7-Cas5-Cas11 fusion subunit is active. Suitably in methods of detecting a single stranded nucleic acid as described herein, the Cas7 or each Cas7 is inactive, or at least nuclease deficient. Suitably the Cas7 or each Cas7 protein of the Cas7-Cas7 fusion subunit and the Cas7-Cas5-Cas11 fusion subunit is modified to be inactive, or at least nuclease deficient. Further details on such modified forms of Cas7 are provided below.

Suitably the Type III-D CRISPR-Cas system, including the Type III-Dv CRISPR-Cas system, comprises a Cas7 protein. Suitably the Cas7 protein cleaves single stranded nucleic acids. Suitably the Cas7 protein is comprised within the Cas7-Cas7, Cas7-Cas5-Cas11 or Cas7 with insertion fusion proteins. Suitably therefore the Cas7 protein is comprised within SEQ ID NO: 4, 6 or 10, or a functional fragment thereof, an orthologue or homologue thereof, or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% identity to SEQ ID NO: 4, 6 or 10, provided that it retains its nuclease activity.

Preferably, as described above, the Cas7 or each Cas7 protein may exist as a fusion protein i.e. a subunit.

Suitably the Cas7-Cas7 fusion protein comprises a sequence set forth in SEQ ID NO:6 or a functional fragment thereof, or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% identity thereto.

Suitably the Cas7-Cas5-Cas11 fusion protein comprises a sequence set forth in SEQ ID NO:4 or a functional fragment thereof, or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% identity thereto.

Suitably the Cas7 protein with an insertion comprises a sequence set forth in SEQ ID NO: 10 or a functional fragment thereof, or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% identity thereto.

Suitably the Type III-D CRISPR-Cas system comprises a Cas5 protein. Suitably the Cas5 protein binds and stabilises the guide RNA. Suitably the Cas5 protein is comprised within the Cas7-Cas5-Cas11 fusion protein. Suitably therefore the Cas5 protein is comprised within SEQ ID NO:4 or a functional fragment thereof, an orthologue or homologue thereof, or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% identity thereto.

Suitably the Type III-D CRISPR-Cas system comprises a Cas11 protein. Suitably the Cas11 protein is a stabilising protein. Suitably the Cas11 protein is comprised within the Cas7-Cas5-Cas11 fusion protein. Suitably therefore the Cas11 protein is comprised within SEQ ID NO:4 or a functional fragment thereof, an orthologue or homologue thereof, or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% identity thereto.

Suitably the Type III-D CRISPR-Cas system comprises a Cas10 protein. Suitably the Cas 10 protein carries out single stranded deoxyribonucleic acid cleavage and produces cyclic oligoadenylates (cOAs). Suitably the Cas10 protein comprises a nuclease domain and a palm domain. Suitably the nuclease domain carries out single stranded deoxyribonucleic acid cleavage and the palm domain produces cyclic oligoadenylates. Suitably the Cas10 protein may be active or partially or entirely inactive, in particular with regard to nuclease activity and/or activity of the palm domain. In some examples, it may be useful for the Cas10 protein to be modified such that it is nuclease deficient, in other words nuclease inactive. In some examples, it may be useful for the Cas10 protein to be modified such that the palm domain is partially or completely inactive. In some examples, it may be useful for the Cas10 protein to be wild type, in other words fully active. Suitably in methods of detecting a single stranded nucleic acid as described herein, the Cas10 is inactive, or nuclease deficient. Suitably in other methods as described herein, the Cas10 palm domain is inactive, which reduces the likelihood of cyclic oligoadenylates causing collateral damage to adjacent single stranded deoxyribonucleic acids via accessory DNA nucleases. Further details on such modified forms of Cas10 and their uses are provided below.

Suitably the Cas10 protein comprises a sequence set forth in SEQ ID NO:2 or a functional fragment thereof, an orthologue or homologue thereof, or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% identity thereto.

Suitably the Type III-D CRISPR-Cas system comprises a Csx19 protein. Suitably the Csx19 protein stabilises the crRNA. Suitably the Csx19 protein comprises a sequence set forth in SEQ ID NO:8 or a functional fragment thereof, an orthologue or homologue thereof, or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% identity thereto.

Suitably the Type III-D CRISPR-Cas system is associated with a Cas6 protein. Suitably the Cas6 protein processes the crRNA. Cas6 is not typically part of the final effector complex. Suitably the Cas6 protein comprises a sequence set forth in SEQ ID NO: 12 or a functional fragment thereof, an orthologue or homologue thereof, or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% identity thereto.

Suitable Cas proteins may be derived from any bacterial or archaeal species. Examples of suitable species include: Microcystis aeruginosa, Acetohalobium arabaticum, Ammonifex degensii, Anabaena cylindrica, Anabaena variabilis, Caldicellulosiruptor lactoaceticus, Caldilinea aerophila, Clostridium algicarnis, Crinalium epipsammum, Cyanothece sp., Cylindrospermum stagnale, Haloquadratum walsbyi, Halorubrum lacusprofundi, Methanocaldococcus vulcanius, Methanospirillum hungatei, Natrialba asiatica, Natronomonas pharaonis, Nostoc punctiforme, Phormidesmis priestleyi, Crematoria acuminata, Picrophilus torridus, Spirochaeta thermophila, Stanieria cyanosphaera, Sulfolobus acidocaldarius, Sulfolobus islandicus, Synechocystis sp., Thermacetogenium phaeum, Thermofilum pendens, etc.

Suitably the Cas proteins used in the present invention are derived from a cyanobacterium. Suitably the Cas proteins used in the present invention are derived from Synechocystis sp. Suitably the Cas proteins used in the present invention are derived from strain Synechocystis sp. PCC 6803.

The Type III-D or Type III-Dv CRISPR-Cas system may be used in any of the methods herein.

### Cas Subunit Fusion Proteins

In some examples of the present invention the Type III-D CRISPR-Cas system comprises a synthetic fusion protein, the synthetic fusion protein comprising a fusion of two or more Cas proteins that normally constitute the wild type Type III-D CRISPR-Cas system. In some examples all of the Cas proteins that normally constitute the Type III-D CRISPR-Cas system can be fused together.

In other examples only some of the Cas proteins can be fused together, for example those Cas proteins considered to form the core of the Type III-D CRISPR-Cas system. Cas proteins are suitably fused via linkers, which may be of any suitable length.

In some examples the Type III-D CRISPR-Cas system is a Type III-Dv CRISPR-Cas system in which two or more Cas proteins have been fused, preferably via linkers. It will be appreciated various linker sequences can be used, and longer linkers may be advantageous in some circumstances to provide additional flexibility. Suitably two or more of the Csx19 subunit, the Cas10 subunit, the Cas7-Cas5-Cas11 subunit, the Cas7-Cas7 subunit and the Cas7-insertion subunit can be fused together to form a synthetic fusion protein. It will be appreciated that modified Cas proteins as discussed herein can be used in place of the wild type Cas proteins (or Cas fusion protein subunits).

In some examples, the Type III-Dv CRISPR-Cas system comprises a synthetic fusion protein comprising Cas7-Cas5-Cas11, Cas7-Cas7 and Cas7-insertion. These are suitably tethered together by linkers. Further Cas proteins, e.g. from the Type III-Dv CRISPR-Cas system, e.g. one or more of the Csx19, and Cas10 subunits, can also be integrated into this synthetic fusion protein. Again, these additional subunits are suitably tethered by linkers.

In some examples, the synthetic fusion protein comprises the general structure:
(Cas7-Cas5-Cas11)-linker-(Cas7-Cas7)-linker-(Cas7-insertion).

It will be appreciated that modified Cas proteins as discussed herein can be used in place of the wild type Cas proteins. Accordingly, (Cas7-Cas5-Cas11), (Cas7-Cas7) and (Cas7-insertion) represent the wild type forms of these Cas proteins and also functional variants thereof, e.g. modified forms as discussed herein.

In some examples the synthetic fusion protein comprises the structure:
(Csx19)-linker-(Cas10)-linker-(Cas7-Cas5-Cas11)-linker-(Cas7-Cas7)-linker-(Cas7-insertion); or
(Cas10)-linker-(Csx19)-linker-(Cas7-Cas5-Cas11)-linker-(Cas7-Cas7)-linker-(Cas7-insertion).

Again, it will be appreciated that modified Cas proteins as discussed above can be used in place of the wild type Cas proteins. Accordingly, (Csx19), (Cas10), (Cas7-Cas5-Cas11), (Cas7-Cas7) and (Cas7-insertion) represent the wild type forms of these Cas proteins and also functional variants thereof, e.g. modified forms as discussed and described herein.

Furthermore, the order of the Cas proteins in any other abovementioned structures can be altered and suitable linkers can be used to allow for assembly of the active conformation.

In some examples, the synthetic fusion protein comprises a sequence according to SEQ ID NO: 28, or a functional variant thereof. Suitably the functional variant comprises a sequence which is at least 60%, 70%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 28. SEQ ID NO: 28 represents a fusion having the structure (Cas7-Cas5-Cas11)-linker-(Cas7-Cas7)-linker-(Cas7-insertion).

### Guide RNA

A ‘guide RNA’ in the present context refers to an RNA molecule that is able to bind to (form a complex with) the Type III-D CRISPR-Cas system and direct it to target (typically single stranded) nucleic acid. Typically, it forms a complex with the relevant target recognition Cas proteins of the Type III-D CRISPR-Cas system.

Suitably the methods of the invention may comprise one, or more than one guide RNA. Suitably each guide RNA may target a different nucleic acid sequence.

Guide RNAs are typically crRNAs, and crRNAs for Type III-D CRISPR-Cas systems have been described in the art (Scholz et al 2013, PMID: 23441196 PMCID: PMC3575380 DOI: 10.1371/journal.pone.0056470).

Methods of producing guide RNAs are also well known in the art, including direct expression of mature crRNAs or through expression and processing of an immature or pre-crRNA form that is then processed to form mature gRNA. Any suitable approach can be used to produce a suitable guide RNA for the various aspects and examples described herein.

Suitably the guide RNA comprises a recognition sequence which is complementary to the target nucleic acid. This may also be known as a spacer or protospacer sequence. Suitably the recognition sequence may be from about 20 nucleotides to about 70 nucleotides in length, (e.g.) about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 nucleotides in length). Suitably the recognition sequence is about 20-40 nucleotides in length. Suitably longer complementary sequences provide higher sequence specificity to the guide RNA and a higher stability.

Suitably the complementarity between the recognition sequence and that target nucleic acid is sufficient for the recognition sequence of the guide RNA to hybridise to the target nucleic acid and direct sequence-specific binding of the CRISPR Type III-D complex to the target nucleic acid.

Suitably the recognition sequence (spacer) may be fully complementary to a target nucleic acid (e.g., 100% complementary to a target sequence across its full length). In some examples, the recognition sequence may be substantially complementary (e.g., at least about 80% complementary (e.g., about 80%, 81%, 82%, 83%, 84%, 85%, 86%, 87%, 88%, 89%, 90%, 91%, 92%, 93%, 94%, 95%, 96%, 97%, 98%, 98.5%, 99%, 99.5%, or more complementary)) to a target nucleic acid. Thus, in some examples, a recognition sequence may have one, two, three, four, five or more mismatches that may be contiguous or non-contiguous as compared to a target nucleic acid.

Suitably the complementarity between the recognition sequence and the target nucleic acid is at least 95%, 96%, 97%, 98%, 98.5%, 99%, 99.5% or 100%.

When the Type III-D CRISPR-Cas system is a Type III-Dv CRISPR-Cas system the guide RNA can be a mature crRNA. In some examples the mature crRNA is approximately 37 nucleotides in length. However, other lengths can also be functional, for example from 30-50 nucleotides in length, from 32-45 nucleotides in length, for example from 35-40 nucleotides in length. However, it will be appreciated that any length of crRNA that is capable of complexing with the Type III-Dv CRISPR-Cas system and guiding it to a target nucleic acid can be used.

The guide RNA can also be provided as an immature or pre-crRNA (also referred to as an unprocessed guide RNA) that is further processed to produce a mature crRNA. In wild type Type III-Dv CRISPR-Cas systems, immature crRNA is processed to a 37 nt mature form (e.g. SEQ ID NO: 35), which is made up of 8 nucleotides from the 5′ repeat handle and 29 nucleotides from the spacer. To elaborate, in cells the repeat-spacer-repeat sequence is processed by Cas6 which cleaves 8 nucleotides from the end of every repeat (this 5′ 8 nucleotides is the repeat handle; the total length of this intermediate form varies depending on the spacer length). This intermediate (e.g. see SEQ ID NO: 23) is further processed into the mature crRNA (e.g. SEQ ID NO: 35) by currently unknown nucleases to provide the mature crRNA. For the Type III-Dv system described in the specific examples herein, the total length is typically 37 nucleotides.

When the Type III-D CRISPR-Cas system is a Type III-Dv CRISPR-Cas system the recognition sequence (spacer) may suitably be approximately 29 nucleotides in length. However, other lengths can also be functional, for example from 30-50 nucleotides in length, from 32-45 nucleotides in length, for example from 35-40 nucleotides in length.

Suitably, in addition to the recognition sequence, the guide RNA further comprises one or more Cas binding sequences. Interactions between guide RNA and the components of a Type III-Dv CRISPR-Cas system are discussed herein.

An exemplary guide RNA is set forth in SEQ ID NO: 35, below. It will be appreciated that the target specificity can be modified by changing the recognition sequence (spacer).

Accordingly, a more general exemplary guide RNA can have the following sequence:

ACUGAAACNNNNNNNNNNNNNNNNNNNNNNNNNNNNN (SEQ ID NO: 34),

wherein N represents any nucleotide.

Modifications in the 5′ repeat region of the guide RNA can be tolerated to some extent. Thus, by way of example, the guide RNA may have 1, 2, 3, 4, 5 or 6 or more changes in the 5′ repeat region, provided the guide RNA retains the ability to bind to the Type III-Dv CRISPR-Cas system and guide it to a target nucleic acid.

It is important to note that for Type III-D CRISPR-Cas systems there is generally no requirement for a protospacer adjacent motif (PAM) or protospacer flanking sequence (PFS) for target nucleic acid binding. Advantageously, this provides greater flexibility in target sequence choice than many other CRISPR-Cas systems.

### Methods of Modification

Some methods of the present invention relate to the modification of a target single stranded nucleic acid using the Type III-D, suitably a Type III-Dv, CRISPR Cas system.

Upon contacting the target single stranded nucleic acid with the complex, the complex is cultured or incubated for a time and under conditions suitable for modification of the target nucleic acid to occur.

Suitably if contacting occurs in a cell free system, then the complex and the target single stranded nucleic acid are cultured or incubated together under suitable cell free conditions for modification to occur at the target sequence.

Suitable cell free culture techniques are well known to the skilled person.

Suitably if contacting occurs within a cell then after introduction of the complex and optionally the target single stranded nucleic acid into the cell, the cell is cultured for a time and under conditions suitable for modification to occur at the target sequence. Suitably the target single stranded nucleic acid may already exist in the cell, and may be endogenous to the cell.

Suitably the culture conditions may be determined by the skilled person according to the type of cell and species of cell which harbours the complex. Suitable cell culture techniques are known to the skilled person as noted above.

Suitably therefore, the methods according to the present invention may comprise a step of culturing the complex and the target nucleic acid for a time and under conditions suitable to allow modification to occur.

Suitably the modification is cleavage, suitably cleavage of the target nucleic acid. Suitably the cleavage is single-stranded cleavage of a single-stranded nucleic acid sequence. Preferably therefore, the method is a method of cleavage. Suitably in methods directed towards modification of a single stranded nucleic acid sequence, single strand cleavage takes place. Suitably carried out by one or more of the Cas7 proteins of the Type III-D CRISPR Cas system.

Suitably therefore a functional Type III-D CRISPR Cas system is used in methods according to the present invention which is capable of cleaving a single stranded nucleic acid sequence in at least one position. Suitably a functional Type III-D CRISPR Cas system is used which is capable of cleaving the single stranded nucleic acid sequence in multiple positions, suitably in up to three positions. Suitably therefore the methods according to the present invention are directed to modification of a target single stranded nucleic acid at multiple positions, suitably a method of cleaving a target single stranded nucleic acid at multiple positions, suitably at up to three different positions.

Suitably two Cas7 containing subunits of the Type III-D CRISPR Cas system are capable of cleaving ribonucleic acids; the Cas7-Cas7 fusion subunit and the Cas7-Cas5-Cas11 fusion subunit. Suitably the Cas7-Cas7 fusion subunit is capable of cleaving ribonucleic acids at two sites. Suitably the Cas7-Cas7 fusion subunit cleaves the target ribonucleic acid at positions complementary to positions 26 and 20 from the 5′ end of the guide RNA. Suitably the Cas7-Cas5-Cas11 fusion subunit is capable of cleaving ribonucleic acids at a single site. Suitably the Cas7-Cas5-Cas11 fusion subunit cleaves the target ribonucleic acid at a position complementary to position 14 from the 5′ end of the guide RNA. Suitably therefore, the Type III-D CRISPR Cas system is capable of cleaving ribonucleic acids at up to three positions. Suitably said cleavage positions are complementary to positions 14, 20 and 26 from the 5′ end of the guide RNA.

Suitably therefore, the methods according to the present invention may involve modification of a target ribonucleic acid, suitably a method of cleaving a target ribonucleic acid, at one or more positions complementary to positions 14, 20 and 26 from the 5′ end of the guide RNA. Suitably therefore, the method may be a method of modifying a target ribonucleic acid, suitably a method of cleaving a target ribonucleic acid, at positions complementary to positions 26 and 20 from the 5′ end of the guide RNA, positions 26 and 14 from the 5′ end of the guide RNA, positions 20 and 14 from the 5′ end of the guide RNA, positions 14, 20 and 26 from the 5′ end of the guide RNA, position 26 from the 5′ end of the guide RNA, position 20 from the 5′ end of the guide RNA, or position 14 from the 5′ end of the guide RNA.

In certain examples, the Cas7 proteins of the Type III-D CRISPR Cas system may be modified to reduce nuclease i.e. cleavage activity. In particular, the Cas7 proteins within the Cas7-Cas7 fusion subunit and the Cas7-Cas5-Cas11 fusion subunit may be modified to reduce nuclease activity. Accordingly, different cleavage patterns and positions may be chosen by modifying said subunits to prevent cleavage at one or more of the positions listed above.

Suitably therefore, the method of modifying, for example cleaving, a target single stranded nucleic acid may comprise a Type III-D CRISPR Cas system having at least one modified Cas7-containing subunit. In an example, at least one modified Cas7-containing subunit has reduced nuclease activity. This includes, without limitation, a modified Cas7-Cas7 fusion subunit and/or a modified Cas7-Cas5-Cas11 fusion subunit having reduced nuclease activity.

Suitably cleavage is effected by aspartate residues present in the Cas7 proteins of the Cas7-Cas7 fusion subunit and the Cas7-Cas5-Cas11 fusion subunit. Suitably D26 of the Cas7-Cas5-Cas11 fusion subunit according to SEQ ID NO: 4, or a position corresponding thereto, effects cleavage of a target ribonucleic acid sequence. Suitably D246 and D33 of the Cas7-Cas7 fusion subunit according to SEQ ID NO: 6, or positions corresponding thereto, effect cleavage of a target ribonucleic acid sequence.

Suitably the cleavage sites of the target single stranded nucleic acid sequence can be controlled by modifying the Cas7 proteins of the Cas7-Cas7 fusion subunit and the Cas7-Cas5-Cas11 fusion subunit at one or more of these aspartate residues, or by making other modifications that reduce or eliminate activity at the active site (e.g. by disrupting its structure), in any combination. Suitably any one of these aspartate residues may be modified to reduce nuclease activity of the Type III-D CRISPR Cas system. Suitably a modification may alternatively or additionally be made elsewhere in the subunit which inactivates any one of the active nuclease sites. Suitably any one of these aspartate residues, or any other one or more amino acids in the relevant subunit which inactivates any one of the active nuclease sites, may be modified to prevent cleavage of a target single stranded nucleic acid by the Type III-D CRISPR Cas system. Suitable modifications to the Cas7 containing subunits are explained elsewhere herein.

Suitably therefore the method of modifying, preferably cleaving, a target single stranded nucleic acid may comprise a Type III-D CRISPR Cas system having a modified Cas7-Cas7 fusion subunit, suitably modified to inactivate the nuclease active site at positions D246 and/or D33 of SEQ ID NO: 6, or positions corresponding thereto. Suitably therefore the method of modifying, preferably cleaving, a target single stranded nucleic acid may comprise a Type III-D CRISPR Cas system having a modified Cas7-Cas7 fusion subunit, suitably modified to inactivate the nuclease active site at position D246 of SEQ ID NO: 6, or a position corresponding thereto. Suitably in such an example, the method may be a method of cleaving a target single stranded nucleic acid, at positions complementary to positions 20 and 14 from the 5′ end of the guide RNA. Suitably no cleavage takes place at a position complementary to position 26 from the 5′ end of the guide RNA. Suitably therefore the method of modifying, preferably cleaving, a target single stranded nucleic acid may comprise a Type III-D CRISPR Cas system having a modified Cas7-Cas7 fusion subunit, suitably modified to inactivate the nuclease active site at position D33 of SEQ ID NO: 6, or positions corresponding thereto. Suitably in such an example, the method may be a method of cleaving a target single stranded nucleic acid, at positions complementary to positions 14 and 26 from the 5′ end of the guide RNA. Suitably no cleavage takes place at a position complementary to position 20 from the 5′ end of the guide RNA.

Suitably therefore the method of modifying, preferably cleaving, a target single stranded nucleic acid may comprise a Type III-D CRISPR Cas system having a modified Cas7-Cas5-Cas11 fusion subunit, suitably modified to inactivate the nuclease active site at position D26 of SEQ ID NO: 4, or a position corresponding thereto. Suitably in such an example, the method may be a method of cleaving a target single stranded nucleic acid, at positions complementary to positions 20 and 26 from the 5′ end oof the guide RNA. Suitably no cleavage takes place at a position complementary to position 14 from the 5′ end of the guide RNA.

Advantageously therefore, cleavage of a target single stranded nucleic acid sequence may be effected at one, two or three different positions as desired, by using modified versions of the Type III-D CRISPR Cas system in which the Cas7-Cas7 fusion subunit and the Cas7-Cas5-Cas11 fusion subunit have been modified to affect (e.g. reduce or eliminate) their nuclease activity.

Suitably any method of modification described herein may comprise more than one Type III-D CRISPR Cas system. Suitably in some examples a plurality of Type III-D CRISPR Cas systems may be used in any one method, suitably in any one step of modification. Suitably each Type III-D CRISPR Cas system may be targeted to a different target nucleic acid sequence. Suitably in some examples a pair of Type III-D CRISPR Cas systems may be used.

Suitably the guide RNA hybridises to the target single stranded nucleic acid sequence, and interacts with the complex of Cas proteins to target them to the correct target nucleic acid. Then the cleavage domains of the complex, i.e. Cas7 proteins/subunits, cleave the target single stranded nucleic acid at the or each cleavage site described above.

Suitably after cleavage has occurred, expression of the cleaved single stranded nucleic acid is inhibited. For example, translation of the RNA into a protein is inhibited.

More than one guide RNA can be used in order to target more than one target stranded nucleic acid sequence.

### Modified Cas7

The Type III-D CRISPR-Cas system of the present invention may comprise a modified Cas protein in which a Cas7 domain or subunit has been modified. In particular, any Cas protein that contains a Cas7 domain may be modified to reduce its nuclease activity or to eliminate nuclease activity of the Cas7 domain entirely.

Any Cas7 domain containing Cas protein subunit of a Type III-D CRISPR-Cas system can be modified within the Cas7 domain, e.g. to reduce the nuclease activity of the Cas7 domain.

Where a Cas protein subunit of a Type III-D CRISPR-Cas system contains more than one Cas7 domain, one or more of the Cas7 domains may be modified to reduce nuclease activity or eliminate nuclease activity of the Cas7 domain entirely. In some examples all of the Cas7 domains may be modified to reduce nuclease activity or eliminate nuclease activity of the Cas7 domain entirely.

It will be apparent that any Cas7 domain can be modified at an active nuclease site in order to reduce nuclease activity or eliminate nuclease activity of the Cas7 domain entirely.

In some examples of the invention, the RNA nuclease activity of one or more Cas7 domains is reduced by at least 10%, 20%, 30%, 40%, 50%, 60%, 70%, 80%, 90%, or 100% compared to the wild type or unmodified Cas7 domain. Activity can be assessed by the ability of the modified Cas7 domain to cleave suitable target RNA in equivalent conditions to wild type Cas7. In some preferred examples of the present invention the RNA nuclease activity of Cas7 domain has been eliminated, thus producing a Cas7 domain which is unable to cleave a target RNA. In some examples the nuclease activity of all Cas7 domains in a Cas protein have been reduced as discussed above.

In some examples of the present invention, the total RNA nuclease activity of the Type III-D CRISPR-Cas system domain is reduced by at least 10%, 20%, 30%, 40%, 50%, 60%, 70%, 80%, 90%, or 100% compared to the wild type or unmodified Type III-D CRISPR-Cas system. Activity can be assessed by the ability of the modified Type III-D CRISPR-Cas system to cleave suitable target RNA in equivalent conditions to wild type Type III-D CRISPR-Cas system. In some preferred examples of the present invention the RNA nuclease activity of Type III-D CRISPR-Cas system has been eliminated, thus producing a Type III-D CRISPR-Cas system which is unable to cleave a target RNA.

Considering a Type III-Dv CRISPR-Cas system, it will be apparent that there are multiple Cas7 domains present. In particular, Cas7 domains are present in the Cas7-Cas5-Cas11 subunit (one Cas7 domain), the Cas7-Cas7 subunit (two Cas7 domains) and in the Cas7-insert subunit (one Cas7 domain). However, as discussed below, the Cas7 domain in the Cas7-insert subunit is inactive. Accordingly, there are three active Cas7 domains. In some examples the Type III-Dv CRISPR-Cas system may be modified to reduce or eliminate nuclease activity in one, two or all three of these domains.

In some examples the Cas7-Cas5-Cas11 subunit is modified to reduce or eliminate nuclease activity. The sequence of the Cas7-Cas5-Cas11 is provided in SEQ ID NO: 4. A key residue responsible for cleavage is indicated in bold (D26). For example, a modification can be made at position D26 with reference to SEQ ID NO: 4, or a corresponding position in any other Cas7-Cas5-Cas11 subunit (e.g. an orthologue or homologue from another strain or species). For example, D26 (with reference to SEQ ID NO: 4, or a corresponding amino acid) can be modified to alanine or another suitable amino acid that reduces or eliminates nuclease activity. In some examples a modified Cas7-Cas5-Cas11 subunit suitably comprises a D26A modification (with reference to SEQ ID NO: 4, or a corresponding amino acid). Other modification, such as deletions or insertions, that disrupt nuclease activity could of course be made.

An exemplary modified Cas7-Cas5-Cas11 subunit is set forth in SEQ ID NO: 18, and the DNA sequence encoding this modified Cas7-Cas5-Cas11 subunit is set forth in SEQ ID NO: 17. Accordingly, in some examples the modified Cas7-Cas5-Cas11 subunit comprises a sequence according to SEQ ID NO: 18, or a functional variant thereof, said functional variant retaining the inactivated nuclease activity. Suitably the functional variant comprises a sequence which is 60%, 70%, 80%, 81%, 82%, 83%, 84%, 85% 86%, 87%, 88%, 89%, 90%, 91%, 92%, 93%, 94%, 95%, 96%, 97%, 98% or 99% identical to SEQ ID NO: 18. The invention also provides a nucleic acid encoding such a modified Cas7-Cas5-Cas11 subunit, e.g. SEQ ID NO: 17 or a sequence which is 60%, 70%, 80%, 81%, 82%, 83%, 84%, 85% 86%, 87%, 88%, 89%, 90%, 91%, 92%, 93%, 94%, 95%, 96%, 97%, 98% or 99% identical to SEQ ID NO: 17.

In some examples the Cas7-Cas7 subunit (also referred to as Cas7_2x) is modified to reduce or eliminate nuclease activity. The Cas7-Cas7 subunit contains two active Cas7 domains. The sequence of the Cas7-Cas7 subunit is provided in SEQ ID NO: 6. Two key residues responsible for cleavage are indicated in bold (D33 and D246). For example, a modification can be made at position D33, D246 or both D33 and D246 with reference to SEQ ID NO: 6, or at a corresponding position in any other Cas7-Cas7 subunit (e.g. an orthologue or homologue from another strain or species). For example, D33, D246 or both D33 and D246 (with reference to SEQ ID NO: 6, or corresponding amino acids) can be modified to alanine or another suitable amino acid that reduces or eliminates nuclease activity. In some examples a modified Cas7-Cas7 subunit suitably comprises a D33A modification, or equivalent modifications in any other Cas7-Cas7 subunit (e.g. an orthologue or homologue from another strain or species). In some examples a modified Cas7-Cas7 subunit suitably comprises a D246A modification, or equivalent modifications in any other Cas7-Cas7 subunit (e.g. an orthologue or homologue from another strain or species). In some examples a modified Cas7-Cas7 subunit suitably comprises D33A and D256A modifications, or equivalent modifications in any other Cas7-Cas7 subunit (e.g. an orthologue or homologue from another strain or species). Other modification, such as deletions or insertions, that disrupt nuclease activity could of course be made.

An exemplary modified Cas7-Cas7 subunit in which D33 has been modified is set forth in SEQ ID NO: 20, and the DNA sequence encoding this modified Cas7-Cas7 subunit is set forth in SEQ ID NO: 19. Accordingly, in some examples the modified Cas7-Cas7 subunit comprises a sequence according to SEQ ID NO: 20, or a functional variant thereof, said functional variant retaining the inactivated nuclease activity. Suitably the functional variant comprises a sequence which is 60%, 70%, 80%, 81%, 82%, 83%, 84%, 85% 86%, 87%, 88%, 89%, 90%, 91%, 92%, 93%, 94%, 95%, 96%, 97%, 98% or 99% identical to SEQ ID NO:20. The invention also provides a nucleic acid encoding such a modified Cas7-Cas7 subunit, e.g. SEQ ID NO: 19 or a sequence which is 60%, 70%, 80%, 81%, 82%, 83%, 84%, 85% 86%, 87%, 88%, 89%, 90%, 91%, 92%, 93%, 94%, 95%, 96%, 97%, 98% or 99% identical to SEQ ID NO: 19.

An exemplary modified Cas7-Cas7 subunit in which D246 has been modified is set out in SEQ ID NO: 22, and the DNA sequence encoding this modified Cas7-Cas7 subunit is set out in SEQ ID NO: 21. Accordingly, in some examples the modified Cas7-Cas7 subunit comprises a sequence according to SEQ ID NO: 22, or a functional variant thereof, said functional variant retaining the inactivated nuclease activity. Suitably the functional variant comprises a sequence which is 60%, 70%, 80%, 81%, 82%, 83%, 84%, 85% 86%, 87%, 88%, 89%, 90%, 91%, 92%, 93%, 94%, 95%, 96%, 97%, 98% or 99% identical to SEQ ID NO:22. The invention also provides a nucleic acid encoding such a modified Cas7-Cas7 subunit, e.g. SEQ ID NO: 21 or a sequence which is 60%, 70%, 80%, 81%, 82%, 83%, 84%, 85% 86%, 87%, 88%, 89%, 90%, 91%, 92%, 93%, 94%, 95%, 96%, 97%, 98% or 99% identical to SEQ ID NO: 21.

Considering the Type III-Dv CRISPR-Cas system, it may comprise a modified Cas7-Cas7 subunit or a modified Cas7-Cas5-Cas11 subunit or both a modified Cas7-Cas7 subunit and a modified Cas7-Cas5-Cas11 subunit. Accordingly, the nuclease activity at one, two or three of the active Cas7 domains can be reduced or eliminated. In other words, the modified Type III-Dv CRISPR-Cas system may have modified RNA nuclease activity at:
the D26 position of the Cas7-Cas5-Cas11 subunit;
the D33 position of the Cas7-Cas7 subunit;
the D246 position of the Cas7-Cas7 subunit;
the D26 position of the Cas7-Cas5-Cas11 subunit and the D33 position of the Cas7-Cas7 subunit;
the D26 position of the Cas7-Cas5-Cas11 subunit and the D246 position of the Cas7-Cas7 subunit;
the D33 and the D246 position of the Cas7-Cas7 subunit; or
the D26 position of the Cas7-Cas5-Cas11 subunit and the D33 and the D246 positions of the Cas7-Cas7 subunit, all with reference to SEQ ID NO: 4 and SEQ ID NO: 6 or at corresponding positions in any other Cas7-Cas5-Cas11 Cas7-Cas7 subunits (e.g. an orthologue or homologue from another strain or species).

A modified Type III-D CRISPR-Cas system which has been altered to reduce nuclease activity (e.g. eliminating nuclease activity at one or two positions) may be useful to control cleavage of single stranded nucleic acids. A modified Type III-D CRISPR-Cas system which has been modified to substantially or completely eliminate nuclease activity may be particularly useful for methods of detection so that target RNA is not cleaved and the Type III-D CRISPR-Cas complex stays bound for longer; this may, for example, allow for greater production of cOAs.

### Modified Cas10

In some examples, the Cas10 subunit may be modified to reduce nuclease activity.

Accordingly, in some examples, the present invention contemplates Type III-Dv CRISPR-Cas systems (and methods of their use) in which the Cas10 subunit has been modified, in particular to reduce nuclease activity, suitably to reduce DNA nuclease activity.

In some examples of the invention, the DNA nuclease activity of Cas10 has been reduced by at least 10%, 20%, 30%, 40%, 50%, 60%, 70%, 80%, 90%, or 100% compared to wild type or unmodified Cas10. Activity can be assessed by the ability of the modified Cas10 to cleave suitable target ssDNA in equivalent conditions to wild type Cas10. In some preferred examples of the invention the DNA nuclease activity of Cas10 has been eliminated, thus producing Cas10 which is unable to cleave SSDNA.

Cas10 cleaves ssDNA via an HD domain (see SEQ ID NO: 2). In certain examples, the HD domain can be altered to reduce or eliminate nuclease activity. For example, the HD domain of Cas10 can be modified at one or both amino acid positions of the HD motif (e.g. H337 and D338 in SEQ ID NO: 2), or equivalent modifications in any other Cas10 subunit (e.g. an orthologue or homologue from another strain or species). For example, one or both of the amino acids in the HD motif (H337 and D338 in SEQ ID NO: 2) can be modified to alanine or another suitable amino acid. Accordingly, in some examples a modified Cas10 is suitably modified at H337, D338 or both H337 and D338 with reference to in SEQ ID NO: 2, or corresponding positions in any other Cas10 (e.g. an orthologue or homologue from another strain or species), so as to partially or completely deactivate the HD domain. In some examples a modified Cas10 suitably comprises a H337A modification, a D338A modification or both H337A and D338A modifications, with reference to in SEQ ID NO: 2, or corresponding positions in any other Cas10.

An exemplary modified form of Cas10 in which the HD nuclease domain has been inactivated (dead HD Cas10) is set forth in SEQ ID NO: 14. The DNA sequence encoding this modified Cas10 is set forth in SEQ ID NO: 13. Here the HD dinucleotide motif has been converted to AA. It will be appreciated that other modifications to reduce or eliminate nuclease activity of Cas10 can be made (e.g.) based on substitution, deletion or addition mutations.

Cas10 having reduced DNA nuclease activity may be beneficial in certain contexts. For example, where Cas10 having reduced or eliminated DNA nuclease activity is used in a method of the present invention, it may prevent undesirable cleavage of ssDNA. This is particularly relevant where DNA probes are used, but may be useful in other contexts, e.g., in the context of in vivo mRNA knockdown, DNase activity is typically undesirable to avoid unintended DNA cleavage.

In some examples, the Cas10 subunit may be modified to reduce palm domain activity, in particular to reduce production of cyclic oligoadenylates (cOAs).

In some examples of the invention, the palm domain activity of Cas10 has been reduced by at least 10%, 20%, 30%, 40%, 50%, 60%, 70%, 80%, 90%, or 100% compared to wild type or unmodified Cas10. Activity can be assessed by the ability of the modified Cas10 to produce cOAs in equivalent conditions to wild type Cas10. In some preferred examples of the invention, the palm domain activity of Cas10 has been eliminated, thus producing Cas10 which is unable to produce cOAs.

The palm motif of Cas10 is set forth in SEQ ID NO: 2 below. In certain examples, the palm domain can be altered to reduce or eliminate its activity. For example, the palm domain of Cas10 can be modified at one or more amino acid positions of the palm motif (e.g. G306, G307, D308 and D309 in SEQ ID NO: 2). For example, one or both of the amino acids in the palm motif (D308 and D309 in SEQ ID NO: 2) can be modified to alanine or another suitable amino acid. Accordingly, in some examples a modified Cas10 is suitably modified at one or more of G306, G307, D308 and D309, with reference to in SEQ ID NO: 2, or corresponding positions in any other Cas10 (e.g. an orthologue or homologue from another strain or species), so as to partially or completely deactivate the palm domain. In some examples a modified Cas10 is suitably modified at D308, D309 or both D308 and D309, with reference to in SEQ ID NO: 2, or corresponding positions in any other Cas10, so as to partially or completely deactivate the palm domain. In some examples a modified Cas10 suitably comprises a D308A modification, a D309A modification or both D308A and D309A modifications, with reference to SEQ ID NO: 2, or corresponding positions in any other Cas10.

An exemplary modified form of Cas10 in which the palm domain has been inactivated (dead palm Cas10) is set forth in SEQ ID NO: 16. The DNA sequence encoding this modified Cas10 is set forth in SEQ ID NO: 15. Here the DD amino acids of the palm motif have been converted to AA. It will be appreciated that other modifications to reduce or eliminate nuclease activity of Cas10 can be made. In some examples the modified Cas10 comprises a sequence set forth in SEQ ID NO: 16, or a functional variant thereof, said functional variant retaining the inactivated nuclease activity. Suitably the functional variant comprises a sequence which is 60%, 70%, 80%, 90%, 95% or 99% identical to SEQ ID NO: 16. The invention also provides a nucleic acid encoding such a modified Cas10, e.g. SEQ ID NO: 15 or a sequence which is 60%, 70%, 80%, 90%, 95% or 99% identical to SEQ ID NO: 15.

Modified Cas10 having a palm domain with reduced or eliminated palm activity (i.e. cOA production) may be particularly useful for stopping unwanted nuclease activity. As described elsewhere herein, cOA activity stimulates accessory nuclease activity, which may be undesirable in some circumstances. For example, in a situation when a system as described herein is being used to target and cleave only single stranded nucleic acids within a cell, cleavage by accessory nucleases may be undesirable.

In some examples the Cas10 may be modified to reduce or eliminate both nuclease activity and palm activity.

### A Modified Type III-D CRISPR-Cas System

Suitably the present invention makes use of a novel CRISPR-Cas system, suitably which comprises unique Cas7 containing protein subunits. Suitably the system comprises a Cas10 protein, a Csx19 protein, a Cas7-Cas7 fusion, a Cas7-Cas5-Cas11 fusion, and a Cas7 protein with an insertion as claimed. Preferably the Type III-D CRISPR-Cas system is a Type III-Dv system.

In some examples of the present invention, a modified Type III-D CRISPR-Cas system may be used. By modified it is meant that one or more of the components of the system have been changed such that the system is different to that of a reference wild type system. In some examples, components of the system such as Cas proteins may be removed entirely. In some examples, the polypeptide sequences forming one or more of the proteins used in the system have been mutated such that one or more amino acid residues are different to those of a reference wild type polypeptide sequence.

Suitably, as described above, any of the Cas7 proteins in the system may be modified, suitably they may be modified to reduce nuclease activity. Suitably the Cas7 proteins are modified to reduce their ability to cleave single stranded nucleic acids. Suitable modifications to the Cas7 proteins are described above. Suitably the modified Cas7-Cas5-Cas11 fusion subunit may comprise a sequence according to SEQ ID NO: 18. Suitably the modified Cas7-Cas7 fusion subunit may comprise a sequence according to SEQ ID NO: 20 or 22.

Suitably such modified forms of each Cas7 containing subunit may be used in any of the methods described herein, including in a method of single stranded nucleic acid modification or in a method of single stranded nucleic acid detection as described herein.

Suitably therefore in a further aspect of the invention, there is provided use of a modified Type III-D CRISPR-Cas system as described herein for modifying single stranded nucleic acids. Suitably therefore in a further aspect of the invention, there is provided use of a modified Type III-D CRISPR-Cas system as described herein for detecting single stranded nucleic acids.

Suitably, a system comprising at least one modified Cas7 containing subunit is useful to control modification, suitably cleavage, of single stranded nucleic acids. Suitably more than one of the Cas7 containing subunits may be modified in any combination to control cleavage of ribonucleic acids at up to three positions, as explained hereinabove. Suitably any of the Cas7 containing subunits of the system selected from the Cas7-Cas7 fusion subunit, and/or the Cas7-Cas5-Cas11 fusion subunit may be modified to reduce ribonuclease activity. Suitably any of the Cas7 proteins within the Cas7-Cas7 fusion subunit, and/or the Cas7-Cas5-Cas11 fusion subunit may be modified to reduce ribonuclease activity. Suitably however, at least one Cas7 protein selected from those within the Cas7-Cas7 fusion subunit and/or the Cas7-Cas5-Cas11 fusion subunit remains active, and unmodified, so that single stranded nucleic acid cleavage may still occur in at least one position.

Suitably a system comprising modified Cas7 containing subunits is useful for detection of single stranded nucleic acids so that target nucleic acids are not cleaved and the entire Type III-D CRISPR-Cas complex stays bound to the target for longer. Suitably therefore in methods of detecting single stranded nucleic acids, each Cas7 containing subunit is modified to reduce ribonuclease activity. Suitably each Cas7 containing subunit having an active Cas7 is modified to reduce ribonuclease activity. Suitably therefore in methods of detecting single stranded nucleic acids, each of the Cas7 proteins within the Cas7-Cas7 fusion subunit, and the Cas7-Cas5-Cas11 fusion subunit are modified to reduce ribonuclease activity as explained elsewhere herein.

Suitably, as described above, the Cas10 protein in the system may be modified, suitably it may be modified to reduce ssDNA nuclease activity. Suitably to reduce its ability to cleave single stranded nucleic acids. Suitably therefore the Cas10 protein may comprise a sequence according to SEQ ID NO: 14. Alternatively or additionally it may be modified to reduce its cyclic oligoadenylate production. Suitably therefore the Cas10 protein may comprise a sequence set forth in SEQ ID NO: 16.

Suitably such a modified form of Cas10 protein may be used in any of the methods described herein, including in a method of single stranded nucleic acid modification or in a method of single stranded nucleic acid detection as described herein.

Suitably therefore in a further aspect of the invention, there is provided use of a modified Type III-D CRISPR-Cas system as described herein for modifying single stranded nucleic acids. Suitably therefore in a further aspect of the invention, there is provided use of a modified Type III-D CRISPR-Cas system as described herein for detecting single stranded nucleic acids.

Suitably, a system comprising a modified Cas10 protein which has been modified to reduce deoxyribonuclease activity is useful to aid detection of single stranded nucleic acids. Suitably the reduced nuclease activity prevents the Cas10 protein accidentally cleaving the DNA probes which are used in exemplary methods of detecting single stranded nucleic acids. Suitably a system comprising a modified Cas10 protein which has been modified to reduce deoxyribonuclease activity may also be used in a method of modifying single stranded nucleic acids, because the ability to cleave double stranded nucleic acids is not required in such a method. Suitably, a system comprising a modified Cas10 protein which has been modified to reduce its cyclic oligoadenylate production is not used in a method of detection of single stranded nucleic acids.

In some examples, the system may comprise both a modified Cas7 containing subunit and a modified Cas10 protein. Suitably therefore in a further aspect of the invention there is provided a modified Type III-D CRISPR-Cas system comprising: a Cas10 subunit, a Csx19 subunit, a Cas7-Cas7 fusion subunit, a Cas7-Cas5-Cas11 fusion subunit, and a Cas7-insertion subunit, wherein at least one of the Cas7 containing subunits is modified to have a reduced ribonuclease activity, and wherein the Cas10 subunit is modified to have a reduced deoxyribonuclease activity and/or is modified to reduce cyclic oligoadenylate production.

In one example, there is provided a modified Type III-D CRISPR-Cas system comprising: a Cas10 subunit, a Csx19 subunit, a Cas7-Cas7 fusion subunit, a Cas7-Cas5-Cas11 fusion subunit, and a Cas7-insertion subunit, wherein at least one of the Cas7 containing subunits is modified to have a reduced ribonuclease activity, and wherein the Cas10 subunit is modified to have a reduced deoxyribonuclease activity. Suitably such a system may be used in a method of modifying single stranded nucleic acids, or in a method of detecting single stranded nucleic acids. Suitably therefore in a further aspect of the invention, there is provided use of such a modified Type III-D CRISPR-Cas system for modifying single stranded nucleic acids. Suitably therefore in a further aspect of the invention, there is provided use of such a modified Type III-D CRISPR-Cas system for detecting single stranded nucleic acids.

In one example, there is provided a modified Type III-D CRISPR-Cas system comprising: a Cas10 subunit, a Csx19 subunit, a Cas7-Cas7 fusion subunit, a Cas7-Cas5-Cas11 fusion subunit, and a Cas7-insertion subunit, wherein each of the Cas7 containing subunits is modified to have a reduced ribonuclease activity, and wherein the Cas10 subunit is modified to have a reduced deoxyribonuclease activity. Suitably such a system may be used in a method of detecting single stranded nucleic acids. Suitably therefore in a further aspect of the invention, there is provided use of such a modified Type III-D CRISPR-Cas system for detecting single stranded nucleic acids.

Suitably, other modifications may be present in any of the Cas proteins of the Type III-D CRISPR-Cas system described herein. By modifications it is meant deletions, insertions, substitutions, truncations etc. in the amino acid sequence encoding the protein, which mean that the amino acid sequence of the protein is different to that of the corresponding wild type protein. Suitably any such modifications may be present, in any number, as long as the protein remains functional. Suitably any modifications may be present, as long as each Cas protein comprises at least 70% identity with the reference sequences identified for each Cas protein or an orthologue or homologue thereof, hereinabove.

### Target Single Stranded Nucleic Acid

Essentially any single stranded nucleic acid can be targeted by the Type III-D CRISPR-Cas complex or modified forms thereof. Suitably the target single stranded nucleic acid is RNA and/or ssDNA. RNA is a particularly preferred target single stranded nucleic acid, particularly when the system is a Type III-Dv CRISPR Cas system.

A target RNA may include mRNA, and non-coding RNAs such as tRNA, rRNA, sRNA, siRNA, IRNA, miRNA, lncRNA, genomic RNA (e.g. RNA viral genome), and synthetic RNA. In some preferred examples the target RNA is mRNA. In some examples the target RNA is in vivo, ex vivo or in vitro.

The target single stranded nucleic acid can have essentially any sequence. As will be apparent from previous discussions, targeting specificity of the Type III-D CRISPR-Cas complex is determined by the guide RNA sequence. There is no requirement for a PAM or PFS motif.

The target site in a target single-stranded nucleic acid can be located in an intragenic region, an intergenic region, a coding region, a non-coding region or a regulatory region of a target nucleic acid.

The target site in a target single-stranded nucleic acid may be RNA specific e.g. in a mature RNA, at a splice junctions, in a polyA region, etc.

The target site in a target single-stranded nucleic acid may be located in a target gene.

Where a method is intended to cleave a target single-stranded nucleic acid, the target site may be in within gene, or within the transcript from a gene, of which it is desirable to decrease/inhibit expression. For example, the gene may be one the expression of which causes or contributes to a disease or undesirable physiological condition. The target site may be located in a sequence in vivo, ex vivo or in vitro. A target gene, or transcript thereof, may be located within a target organism or cell. The organism may be a bacterium, a virus, an archaeon, a fungus, plant, or an animal.

In some examples the single stranded target nucleic acid is RNA and/or ssDNA in vitro, for example in a sample (e.g. in a biological sample) in vitro. Such a target single stranded nucleic acid can be detected and/or cleaved using the Type III-D CRISPR-Cas complexes discussed herein, e.g. using one or more methods as discussed herein.

By way of non-limiting example, in some examples a nuclease deactivated Type III-Dv CRISPR-Cas complex as disclosed herein could be directed to an mRNA in order to bind to a target site (e.g. a ribosome binding site such as a Kozak sequence or an internal ribosomal entry site (IRES)) to inhibit translation while leaving the RNA intact. In other examples, Type III-Dv CRISPR-Cas complex having nuclease activity could be directed to an mRNA and bind to a target site in a translated region to precisely truncate the RNA, e.g. to alter the protein produced. In other examples, Type III-Dv CRISPR-Cas complex having nuclease activity could be directed to an mRNA to bind an RNA region where cleavage effects mRNA stability, thus modifying the stability of the targeted mRNA.

### Contacting

The methods of the invention comprise contacting the target nucleic acid with a Type III-D CRISPR Cas complex. Suitably the step of contacting may comprise contacting the target nucleic acid with the complex in vitro, in vivo, or in a cell in vitro/ex vivo.

As used herein, “contact,” contacting,” “contacted,” and grammatical variations thereof, refers to placing the components of a desired reaction together for a time and under conditions suitable for carrying out the desired reaction. The methods and conditions for carrying out such reactions are well known in the art (See, e.g., Gasiunas et al. (2012) Proc. Natl. Acad. Sci. 109:E2579-E2586; M. R. Green and J. Sambrook (2012) Molecular Cloning: A Laboratory Manual. 4th Ed., Cold Spring Harbor Laboratory Press, Cold Spring Harbor, NY).

Suitably the methods may be performed in a cell-free system in vitro.

Alternatively, the methods may be performed in a cell, in vitro, ex vivo, or in vivo.

Suitably when the methods are performed in a cell, the method comprises introducing the Type III-D CRISPR Cas complex into the cell, suitably introducing the Cas proteins and the guide RNA into the cell. Suitably, the Cas proteins may be introduced into the cell as one or more proteins, or as one or more nucleic acids encoding the Cas proteins, suitably which may be DNA. Suitably the guide RNA may be introduced into the cell as one or more nucleic acids encoding the guide RNA, suitably which may be RNA or DNA.

In some examples, the Cas proteins can be introduced as a DNA sequence encoding the Cas proteins upon a vector, or as a protein, whereas the guide RNA can be introduced either as a DNA sequence encoding the guide RNA upon a vector, or in the form of RNA, e.g. an in vitro transcript.

Suitably the Cas proteins or one or more nucleic acids encoding them, or the guide RNA or one or more nucleic acids encoding it may be introduced into the cell simultaneously, separately, or sequentially.

Alternatively, the Cas proteins and guide RNA may be contacted to form a complex in vitro which complex may then be introduced into the cell.

Suitably the one or more nucleic acids may be comprised on one or more vectors as described below.

In some examples, the one or more nucleic acids of the invention may be stably or transiently introduced into a cell.

The terms “Introducing,” “introduce,” “introduced” (and grammatical variations thereof) in the context of a nucleic acid or protein and a cell means presenting the nucleic acid sequence or protein of interest to the cell (e.g., host cell) in such a manner that the nucleic acid sequence or protein gains access to the interior of a cell and includes such terms as “conjugation”, “transformation,” “transfection,” and/or “transduction.” The terms “conjugation”, “transformation,” “transfection,” and “transduction” as used herein refer to the introduction of a heterologous nucleic acid or protein into a cell. Such introduction into a cell may be stable or transient. Thus, in some examples, a host cell or host organism is stably transformed with the nucleic acids. In other examples, a host cell or host organism is transiently transformed with the nucleic acids.

As used herein, the term “stably introduced” means that the nucleic acid sequence is stably incorporated into the genome of the cell, and thus the cell is stably transformed with the polynucleotide. When a nucleic acid is stably transformed and therefore integrated into a cell, the integrated nucleic acid is capable of being inherited by the progeny thereof, more particularly, by the progeny of multiple successive generations. “Transient transformation” in the context of a nucleic acid sequence means that a polynucleotide is introduced into the cell and does not integrate into the genome of the cell.

Suitably introducing the one or more nucleic acids into the cell may be by transformation or transduction. Suitably the one or more nucleic acid sequences can be introduced into a cell in a single transformation event, in separate transformation events.

Suitably methods of transfection or transformation may include calcium-phosphate mediated, electroporation, liposome mediated, exosome mediated, gene gun, microinjection, agrobacterium mediated transfection or transformation, for example. Suitable methods for carrying out such transfection will be known to a person skilled in the art, and are further described below.

For comprehensive reviews about procedures for getting proteins or nucleic acids into cells the context of this invention, see Marschall A L J, Frenzel A, Schirrmann T, et al. “Targeting antibodies to the cytoplasm” mAbs. (2011) 3:3-16; Gu Z, Biswas A, Zhao M, Tang Y “Tailoring nanocarriers for intracellular protein delivery” Chem. Soc. Rev. (2011) 40:3638-3655. Du J, Jin J, Yan M, Lu Y “Synthetic nanocarriers for intracellular protein delivery” Curr. Drug Metab. (2012) 13:82-92.

Various physical methods of disrupting the cell membrane are useful, such as microinjection and electroporation (see Zhang Y, Yu L-C. “Microinjection as a tool of mechanical delivery” Curr. Opin. Biotechnol. (2008) 19:506-510) have been proposed for delivering compounds ranging from small molecules to proteins. Sharei A, Zoldan J, Adamo A, et al. “A vector-free microfluidic platform for intracellular delivery” Proc. Natl. Acad. Sci. (2013) 110:2082-2087 describes a microfluidic device that transiently disrupts the plasma membrane through physical constriction. Silicon “nanowires” that pierce the cell membrane have also been reported Shalek A K, Robinson J T, Karp E S, et al. “Vertical silicon nanowires as a universal platform for delivering biomolecules into living cells” Proc. Natl. Acad. Sci. (2010) 107:1870-1875.

There are also peptide-based strategies using cell penetrating peptides (CPP) which can enhance permeability of the nucleic acids or proteins. For example the TAT peptide can be covalently coupled. Also, an amphiphilic CPP Pep-1 can noncovalently complex and translocate peptide and protein cargos Morris M C, Depollier J, Mery J, et al. “A peptide carrier for the delivery of biologically active proteins into mammalian cells” Nat. Biotechnol. (2001) 19:1173-1176.

Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024.

There is also for example substance P (SP), an 11-residue neuropeptide which can be conjugated to the nucleic acids or proteins (Harford-Wright E, Lewis K M, Vink R, Ghabriel M N. “Evaluating the role of substance P in the growth of brain tumors” Neuroscience (2014) 261:85-94.

There are also various pore- or channel-forming proteins of bacterial origin which may be used to translocate nucleic acids or proteins into cells. Chatterjee S, Chaudhury S, McShan A C, et al. “Structure and biophysics of Type III secretion in bacteria. Biochemistry (Mosc)” (2013) 52:2508-2517 teaches a sophisticated secretion system which transport proteins directly from the bacterial cytoplasm to the eukaryotic host. Doerner J F, Febvay S, Clapham D E. “Controlled delivery of bioactive molecules into live cells using the bacterial mechanosensitive channel MscL” Nat. Commun. (2012) 3:990 describes functional expression of an engineered bacterial channel (MscL) in mammalian cells, the opening and closing of which could be controlled chemically. Alternatively, the cholesterol-dependent cytolysin (CDC) family of pore-forming toxins, which are capable of forming macropores up to 30 nm in diameter may be useful as “reversible permeabilization” reagents for delivering nucleic acids or proteins into cells. (See Dunstone M A, Tweten R K. “Packing a punch: the mechanism of pore formation by cholesterol dependent cytolysins and membrane attack complex/perforin-like proteins” Curr. Opin. Struct. Biol. (2012) 22:342-349; Provoda C J, Stier E M, Lee K-D. “Tumor cell killing enabled by listeriolysin O-liposome-mediated delivery of the protein toxin gelonin.” J. Biol. Chem. (2003) 278:35102-35108; and Pirie C M, Liu D V, Wittrup K D. “Targeted cytolysins synergistically potentiate cytoplasmic delivery of gelonin immunotoxin” Mol. Cancer Ther. (2013) 12:1774-1782.

In addition to pore- or channel-forming proteins, the membrane-translocating domains of bacterial toxins have been proposed as a modular tool that can be fused to, and enhance the intracellular delivery of, other proteins (see Sandvig K, van Deurs B. “Membrane traffic exploited by protein toxins” Annu. Rev. Cell. Dev. Biol. (2002) 18:1-24; Johannes L, Romer W. “Shiga toxins—from cell biology to biomedical applications” Nat. Rev. Microbiol. (2010) 8:105-116.

Additionally, Lawrence M S, Phillips K J, Liu D R. “Supercharging proteins can impart unusual resilience” J. Am. Chem. Soc. (2007) 129:10110-10112 provides “supercharged” GFP, a variant engineered to have high net positive charge (+36), and certain human proteins with naturally high positive charge (see Cronican J J, Thompson D B, Beier K T, et al. “Potent delivery of functional proteins into mammalian cells in vitro and in vivo using a supercharged protein” ACS Chem. Biol. (2010) 5:747-752; or Cronican J J, Beier K T, Davis T N, et al. “A class of human proteins that deliver functional proteins into mammalian cells in vitro and in vivo” Chem. Biol. (2011) 18:833-838 have been reported to translocate across the cell membrane.

There are also virus-based strategies for packaging of the proteins or nucleic acids into virus-like particles (see Kaczmarczyk S J, Sitaraman K, Young H A, et al. Protein delivery using engineered virus-like particles. Proc. Natl. Acad. Sci. (2011) 108:16998-17003) or attaching them to an engineered bacteriophage T4 head (see Tao P, Mahalingam M, Marasa B S, et al. “In vitro and in vivo delivery of genes and proteins using the bacteriophage T4 DNA packaging machine” Proc. Natl. Acad. Sci. (2013) 110:5846-5851) has been reported to enhance cytosolic delivery.

Further, there are lipid and polymer-based strategies. The proteins or nucleic acids of the invention may be encapsulated in liposomes (see Torchilin V. Intracellular delivery of protein and peptide therapeutics. Drug Discov Today Technol. (2008) 5:e95-e103) or complexed with lipids. Regarding the latter strategy, lipid formulations that have been successful in the transfection of DNA may be used. For example, a formulation based on a mixture of cationic and neutral lipids.

Similarly, polymer-based formulations that have been successfully used for nucleic acid transfections have also been examined for their ability to “transfect” proteins. For example, polyethylenimine (PEI) or poly-β-amino esters (PBAEs) which may be in the form of biodegradable nanoparticles.

Also inorganic material-based strategies may be used; for example including silica, carbon nanotubes, quantum dots, or gold nanoparticles.

Another method is available which is induced transduction by osmocytosis and propanebetaine ((iTOP) (see D'Astolfo, D. S. et al. Efficient intracellular delivery of native proteins. Cell 161, 674-690 (2015). This method allows efficient delivery of CRISPR-Cas complexes into a wide variety of primary cell types. The iTOP approach enables virus-free transduction of native proteins and does not rely on additional peptide tags, which may interfere with protein function or editing efficiency and is particularly effective for transduction of cell types that are refractory to other delivery methods. For more information see Wen Y. Wu (2018) Nature Chem Biol. 14:642-651.

In one embodiment, one or more nucleic acids encoding Cas proteins or guide RNA of the Type III-D CRISPR Cas complex may be introduced into the cell by conjugation. In one embodiment, conjugation is carried out by transfer of genetic material from one bacterium to another through direct contact. Suitably therefore a donor bacterium is prepared comprising the one or more nucleic acids encoding Cas proteins and comprising a nucleic acid sequence encoding the conjugative machinery. Suitably the donor bacterium delivers the one or more nucleic acids encoding Cas proteins to other cells, suitably other bacterial cells. Such conjugation techniques are described in Woodall C. A. (2003) DNA Transfer by Bacterial Conjugation. In: Casali N., Preston A. (eds) E. coli Plasmid Vectors. Methods in Molecular Biology, vol 235. Humana Press. https://doi.org/10.1385/1-59259-409-3:61, for example.

### Incubating

Upon contacting the target nucleic acid sequence with the Type III-D CRISPR Cas complex, the system is cultured or incubated for a time and under conditions sufficient for targeting to occur at the target sequence. Suitably therefore the methods may comprise step of culturing or incubating the complex and the target nucleic acid.

Suitably if contacting occurs in a cell free system, then the complex and the target nucleic acid are cultured or incubated under suitable cell free conditions for targeting to occur at the target sequence.

Suitable cell free culture techniques are known to the skilled person. For example, using the conditions defined in commercial cell-free kits available from myTXTL, Arbor Biosciences, or PUREsystem.

Suitably if contacting occurs within a cell, then after introduction of the complex and the target nucleic acid into the cell, the cell is cultured under suitable conditions for targeting to occur at the target sequence.

Suitably the culture conditions are determined by the skilled person according to the type of cell and species of cell which harbours the complex. Suitable cell culture techniques are known to the skilled person. For example, suitable mammalian cell culture conditions may be found in Phelan, K. and May, K. M. 2017. Mammalian cell tissue culture techniques. Current Protocols in Molecular Biology, 117, A.3F.1-A.3F.23. doi: 10.1002/cpmb.31

### Method of Detecting

The present invention further relates to a method of detecting a target single-stranded nucleic acid in a sample.

Suitably the sample may be a biological sample. Suitably the sample may be a biological fluid such as blood, plasma, sputum, saliva, CSF and the like. Suitably therefore the method may be a method of detecting or tracking a nucleic acid sequence in a biological fluid. Suitably the sample may be a cell or may be a cell lysate. Suitably the cell may be in vitro or may be within an organism in vivo. Suitably therefore the method may be a method of detecting or tracking a target nucleic acid sequence in a cell.

Suitably the method comprises a first step of contacting the sample with: a Type III-D CRISPR Cas system, and a guide RNA complementary to a target sequence in the single-stranded nucleic acid. Suitably the Type III-D CRISPR Cas system and the guide RNA form a complex. Suitably contacting is described elsewhere herein.

Suitably the complex may comprise one or more modified Cas proteins, suitably one or more nuclease deficient Cas proteins as described hereinabove. Suitably the complex may comprise one or more ribonuclease deficient Cas7 proteins or Cas7 containing subunits and/or a deoxyribonuclease deficient Cas 10 protein. Suitably the use of nuclease deficient Cas proteins means that complex will still bind at the target single stranded nucleic acid sequence but cleavage does not occur, and furthermore that the DNA probes used in the method will not be inadvertently cleaved. In one example, the complex used in the method comprises a Cas10 protein which has been modified to reduce its deoxyribonuclease activity, and each Cas7 protein in the Cas7-Cas7 fusion subunit and the Cas7-Cas5-Cas11 fusion subunit has been modified to reduce its ribonuclease activity.

Suitably the guide RNA may be complementary to a target sequence in the target single-stranded nucleic acid. Suitably in methods where it is desired to detect a single-stranded nucleic acid, the guide RNA is complementary to a target sequence in the single-stranded nucleic acid.

Suitably the method comprises a second step of incubating the sample with the complex for a time and under conditions suitable to allow the complex to bind to the target nucleic acid if present, and produce cyclic oligoadenylates. Suitably if incubating occurs in a cell free system, then the complex and the sample are incubated under suitable cell free conditions for binding to occur at the target nucleic acid. Suitable cell free incubation techniques are known to the skilled person.

Suitably if incubating occurs within a cell, then after introduction of the complex into the cell, the cell is incubated for time and under conditions sufficient for binding to occur at the target nucleic acid.

Suitably the incubation conditions are determined by the skilled person according to the type of cell and species of cell which harbours the complex. Suitable cell culture techniques are known to the skilled person.

Preferably the method of detection is carried out in a cell free system.

Suitably binding of the complex to a target nucleic acid sequence in a sample causes the complex to produce cyclic oligoadenylates, suitably it causes the Cas10 protein of the complex to produce cyclic oligoadenylates, suitably it causes the palm domain of the Cas10 protein of the complex to produce cyclic oligoadenylates.

Suitably the palm domain of the Cas10 protein produces a plurality of cyclic oligoadenylates (otherwise referred to herein as cOAs). Suitably the palm domain of the Cas10 protein may produce any type of cyclic oligoadenylate, suitably of any length, suitably selected from cA₂ CA₃, CA₄, CA₅, and cA₆. In one example, the palm domain of the Cas10 protein produces cA₃ cyclic oligoadenylates.

Suitably the production of cyclic oligoadenylates in the presence of the target nucleic acid then causes the activation of a nuclease which is capable of cleaving associated nucleic acid probes. The nuclease may be a DNA nuclease, or it may be an RNA nuclease.

Suitably therefore the method further comprises a second step of contacting and a second step of incubating. Suitably the second step of contacting, step (c) comprises contacting the sample with a nuclease (e.g. a DNA nuclease) and one or more nucleic acid probes (e.g. DNA probes). Suitably the second step of incubating, step (d) comprises incubating the sample with the nuclease and one or more probes for a suitable period of time to allow the nuclease to bind to the cyclic oligoadenylates, if present, and cleave the one or more probes to produce one or more cleaved probes. That is, in the absence of COAs, the nuclease is inactive; conversely the production of cOAs activates the nuclease and it will then target the nucleic acid probe. While DNA nucleases and DNA probes are typically preferred, in some cases RNA nucleases and RNA probes may be of interest.

Suitably contacting is described elsewhere herein, and suitably incubating is described hereinabove.

Suitably the DNA nuclease may be any DNA nuclease which is activated by cyclic oligoadenylates. Suitably the DNA nuclease is activated by binding to the cyclic oligoadenylates. Suitably any DNA nuclease which is activated by the cyclic oligoadenylates that are produced by the Type III-D CRISPR Cas complex may be used in the methods according to this aspect of the present invention. In some cases, the DNA nuclease may comprise a CARF domain or be a NucC protein. In one embodiment, the DNA nuclease is activated by, and suitably binds to, cA₃ cyclic oligoadenylates.

Suitably the RNA nuclease may be any RNA nuclease which is activated by cyclic oligoadenylates. Suitably the RNA nuclease is activated by binding to the cyclic oligoadenylates. Suitably any RNA nuclease which is activated by the cyclic oligoadenylates that are produced by the Type III-D CRISPR Cas complex may be used in the method. In some cases, the RNA nuclease may comprise a CARF domain. In one embodiment, the RNA nuclease is activated by, and suitably binds to, cA₃ cyclic oligoadenylates.

Suitably the DNA nuclease is a DNA nuclease from microorganisms of the genus Pseudomonas or Serratia. Preferably the DNA nuclease is from microorganisms of the genus Serratia. In one embodiment, the DNA nuclease is from Serratia sp. ATCC 39006.

Suitably the nuclease may be a NucC nuclease, a Csm6 nuclease, a Card1 nuclease or a Can2 nuclease. Preferably the nuclease is a DNA nuclease. Preferably the DNA nuclease is a NucC nuclease.

Suitably the NucC nuclease binds cA₃ cyclic oligoadenylates, and is suitably activated. Suitably the NucC nuclease is then capable of cleaving double stranded DNA.

In one embodiment, the DNA nuclease is a NucC nuclease from Serratia sp. ATCC 39006. Suitably, therefore, the DNA nuclease comprises the sequence according SEQ ID NO: 30, or an orthologue or homologue thereof, or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% identity thereto and retaining nuclease functionality. Suitably, the DNA nuclease consists of the sequence according SEQ ID NO: 30.

Suitably, in some preferred examples the probe is a double stranded DNA probe. However, in some examples the probe is a single stranded DNA probe.

Suitably, the DNA probe comprises a sequence which is recognised by the DNA nuclease used in the method. Suitably the probe comprises a recognition motif, suitably the DNA nuclease is capable of recognising and binding to the recognition motif, which may be a core motif or a long motif. In some preferred examples, the recognition motif is a longer motif, which may be beneficial for specific cleavage.

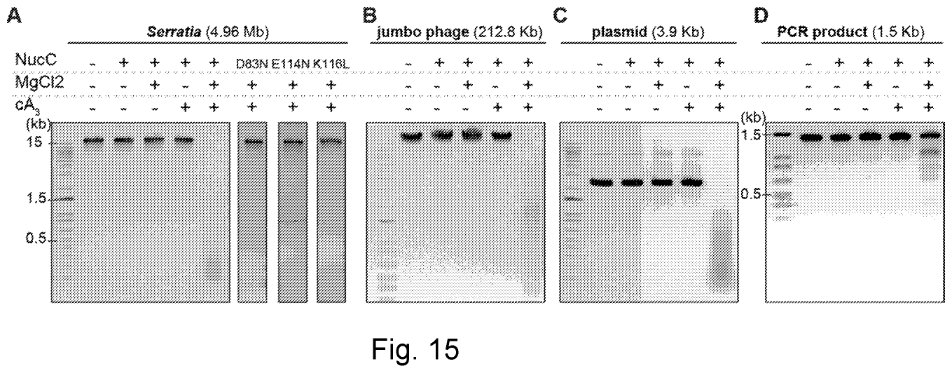

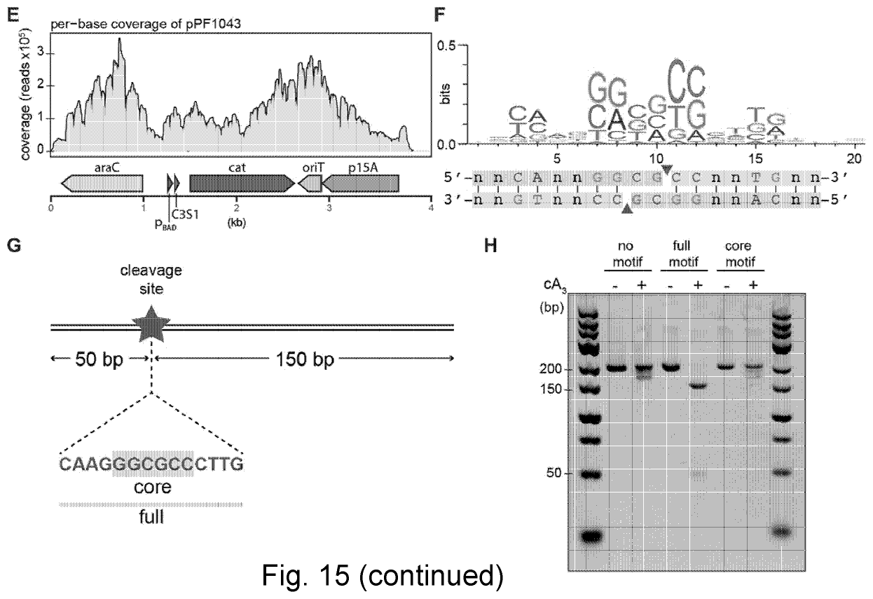

Suitably the recognition motif comprises at least the following sequence: GGCGCC (SEQ ID NO: 37). Suitably this may be termed the ‘core’ recognition motif. Suitably, in some examples, the recognition motif may comprise the following sequence: CAAGGGCGCCCTTG (SEQ ID NO: 38). Suitably this may be termed a ‘long’ recognition motif. Variants of these specific recognition motifs may also be recognised by NucC, and in particular deep sequencing data also proves that there are a range of sites as illustrated in the weblogo of FIG. 15. Accordingly, variations of the recognition motifs of SEQ ID NOS: 37 and 38 may also be present in the DNA probe. For example, sequences which contain changes at 1, 2, 3, 4, 5, or 6 positions when compared to SEQ ID NOs: 37 and 38 may be used as recognition motifs within a probe, provided that they are still recognised by NucC and the probe is cleaved.

Suitably therefore the DNA probe comprises a sequence according to SEQ ID NO:37 or SEQ ID NO: 38.

An example of a DNA probe which may be used in the method of the invention is provided in SEQ ID NO: 31.

Suitably the NucC nuclease from Serratia sp. ATCC 39006 recognises the recognition motif of SEQ ID NO: 37 or 38 and cleaves it. Suitably the NucC nuclease from Serratia sp. ATCC 39006 recognises the recognition motif present in any of the DNA probes used in the method and cleaves them. Suitably to produce one or more cleaved DNA probes in the sample.

Suitably the probe is labelled. Suitably therefore, the probe further comprises one or more of a fluorophore, quencher, donor or accepter linked thereto.

In some examples, the probe comprises a fluorophore and a quencher; or a donor and acceptor linked thereto. Suitably in such an embodiment, the probe comprises a fluorophore and a quencher linked to either end thereof. Alternatively, in such an embodiment, the probe comprises a donor and acceptor linked to either end thereof.

Suitably when the fluorophore and quencher are in proximity to each other, no fluorescence is detected (i.e. there is fluorescence resonance energy transfer between the fluorophore and quencher molecules). Suitably when the fluorophore and quencher are separated, the fluorophore will fluoresce. Suitably therefore when the DNA nuclease binds and cleaves the one or more labelled DNA probes, fluorescence is observed and can be detected. In such an embodiment, the determining step (e) may comprise detecting whether there is fluorescence, suitably detecting if there is an increase in fluorescence in the sample. Suitably if fluorescence is detected, or increased, determining that the target nucleic acid is present in the sample.

Suitably when the donor and the accepter are in proximity to each other, fluorescence is detected. Suitably when the donor and acceptor are separated, fluorescence is not detected. Suitably therefore when the DNA nuclease cleaves the one or more labelled probes, fluorescence is not observed and can no longer be detected. In such an embodiment, the determining step (e) may comprise detecting whether there is fluorescence in the sample, suitably detecting if there is a decrease in fluorescence in the sample. Suitably if there is no fluorescence, or decreased fluorescence, determining that the target nucleic acid is present in the sample.

Other means of detecting the presence or absence of cleaved probes are possible using known techniques in the art. For example, the probes may be biotinylated and when cleaved they may be captured on a lateral flow assay.

Suitably the step of detection may be carried out by a method relevant for detection of the probes that have been used. For example, in cases where the one or more probes comprises a fluorescent protein then detection may be carried out by observing the sample, suitably observing the sample under a microscope or using a fluorescent plate reader such as Varioskan Lux from ThermoFisher Scientific.

Suitably, detecting the one or more cleaved probes comprises observing fluorescence in the sample. Suitably, detecting the one or more cleaved probes comprises observing fluorescence in the sample using a microscope, or using a fluorescent plate reader such as Varioskan Lux from ThermoFisher Scientific. Suitably, not detecting the one or more cleaved probes comprises observing an absence of fluorescence in the sample. Suitably, not detecting the one or more cleaved probes comprises observing an absence of fluorescence in the sample using a microscope or using a fluorescent plate reader such as Varioskan Lux from ThermoFisher Scientific.

### Nucleic Acids

Nucleic acid sequences encoding the Type III-D CRISPR-Cas complex used in the present invention or the modified Type III-D CRISPR-Cas complex or components thereof (e.g. one or more Cas protein and/or one or more guide RNA) are provided herein. These nucleic acid sequences may be provided for introduction into a cell in order to form the complex and in order to carry out the methods of the invention within a cell.

Suitably the Cas protein of the Type III-D CRISPR-Cas system may be introduced into the cell as a protein, or as one or more nucleic acids encoding the or each Cas protein, suitably which may be DNA. Suitably at least one guide RNA may be introduced into the cell as one or more nucleic acids encoding the guide RNA, suitably which may be RNA or DNA. Suitably more than one Cas protein may be encoded on one nucleic acid sequence. Suitably the nucleic acid sequences encoding each Cas protein are linked to each other, suitably in any order. Suitably by a sequence encoding a cleavable linker. Suitably by a sequence encoding a cleavable peptide. Suitably the cleavable linkers are between each nucleic acid sequence encoding each Cas protein. Suitably the guide RNA may also be encoded on the same nucleic acid. Alternatively, each Cas protein may be encoded on a separate nucleic acid. Suitably the guide RNA may be encoded on a separate nucleic acid.

One example of nucleic acids encoding a Type III-D CRISPR-Cas complex are those nucleic acids set forth in SEQ ID Nos 1, 3, 5, 7, 9 and 11 which encode the Cas proteins from a wild type Type III-Dv CRISPR-Cas complex from Synechocystis sp. PCC 6803, a sequence encoding SEQ ID NO: 35 or 23 which are exemplary processed and unprocessed guide RNA sequences, respectively.

Alternatively, the Type III-D CRISPR Cas complex may comprise one or more modified Cas proteins, as described elsewhere herein. Suitably the nucleic acid sequence encoding a modified cas 10 is set forth in SEQ ID NO: 13 or 15. Suitably the nucleic acid sequence encoding a modified Cas7-5-11 is set forth in SEQ ID NO: 17. Suitably the nucleic acid sequence encoding a modified Cas7-Cas7 is set forth in SEQ ID NO: 19 or 21.

Suitably when methods are performed in a eukaryotic cell, the one or more nucleic acids encoding the Cas proteins further comprise nuclear localising sequences (NLS). Suitable nuclear localisation sequences are known in the art. Suitably the one or more nucleic acids may comprise two NLS. Suitably a first NLS at the 5′ end of each nucleic acid sequence and a second NLS at the 3′ end of each nucleic acid sequence.

In some examples each nucleic acid of the invention may be regarded as an “expression cassette” or may be comprised within an expression cassette. As used herein, “expression cassette” means a recombinant nucleic acid construct comprising a nucleic acid sequence of interest (e.g., the polynucleotides encoding Cas polypeptides, and/or guide RNAs of the invention), wherein said nucleic acid sequence of interest is operably linked with at least one regulatory sequence (e.g., a promoter). Thus, some aspects of the invention provide expression cassettes designed to express the nucleic acids of the invention. Suitably comprised on a vector. Suitably any features of the vector described below may also be regarded as features of an expression cassette. Suitable regulatory sequences are defined hereinbelow.

### Vectors

Generally, the term “vector” herein refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art.

Suitably one or more vectors may comprise one or more of the nucleic acids described herein which encode one or more Cas protein of the Type III-D CRISPR-Cas systems disclosed herein. Suitably one or more vectors may comprise one or more nucleic acids described herein that encode the or each guide RNA. Suitably the same vector may comprise one or more of the nucleic acids described herein which encode one or more of the Cas proteins or modified Cas proteins and one or more nucleic acids described herein that encode the or each guide RNA.

Suitably two or more of the nucleic acids encoding the Cas proteins are comprised on a single vector, suitably all of the nucleic acids encoding the Cas proteins are comprised on a single vector.

Suitably when several nucleic acids encoding the Cas proteins are comprised on a single vector, they are linked to each other, suitably in any order. Suitably they may be linked by sequence encoding cleavable linkers. Suitably by cleavable peptides as described above. Suitable cleavable linkers may comprise a 2A self-cleaving peptide, T2A, P2A, E2A, F2A, for example.

Suitably the one or more nucleic acids encoding the Cas proteins and one or more nucleic acids encoding the or each guide RNA may be comprised on the same vector or comprised on separate vectors.

Some vectors are able to direct expression of genes to which they are operatively-linked. Such vectors are “expression vectors” and there will usually be regulatory elements, which may be selected on the basis of the host cells in which the expression takes place. This means the nucleic acid to be expressed is operably linked to the regulatory elements thereby resulting in expression of the nucleotide sequence whether in an in vitro transcription/translation system or in a host cell when the vector is introduced into the host cell.

Suitably the one or more vectors comprising nucleic acids encoding the Cas proteins and one or more nucleic acids encoding the guide RNA further comprise one or more regulatory sequences. Suitably the regulatory sequences are operably linked to the nucleic acids encoding the Cas proteins and to the nucleic acids encoding the or each guide RNA.

Suitably therefore the vector or vectors may comprise an expression cassette as defined hereinabove.

By “operably linked” or “operably associated” as used herein, it is meant that the indicated elements are functionally related to each other, and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, sequences can be present between a promoter and a nucleotide sequence, and the promoter can still be considered “operably linked” to the nucleotide sequence.

Suitable regulatory sequences control expression of the nucleic acid sequence and may include promoters, enhancers, terminators, internal ribosomal entry sites (IRES), and other expression control elements (e.g., transcription termination signals, such as polyadenylation signals and poly-U sequences) UTRs, ITRs, introns etc. For more information the average skilled person would refer to, for example, in Goeddel, (1990), Gene Expression Technology in Methods in Enzymology vol 185, Academic Press. Regulatory elements include those giving direct constitutive expression in many types of host cell and those that direct expression of the nucleotide sequence only in certain cells (i.e., tissue-specific regulatory sequences).

A tissue-specific promoter directs expression primarily in a desired tissue of interest, such as blood, specific organs (e.g., liver, pancreas), or particular cell types. Regulatory elements may also direct expression in a temporal-dependent manner, such as in a cell-cycle dependent or developmental stage-dependent manner, which may or may not also be tissue or cell-type specific. A promoter useful with this invention can include, but is not limited to, constitutive, inducible, developmentally regulated, tissue-specific/preferred-promoters, and the like, as described herein.

A regulatory element as used herein can be endogenous or heterologous. In some examples, an endogenous regulatory element derived from the subject organism can be inserted into a genetic context in which it does not naturally occur (e.g., a different position in the genome than as found in nature), thereby producing a recombinant or non-native nucleic acid. In some examples, promoters useful with the nucleic acid sequences described herein may be any combination of heterologous and/or endogenous promoters.

Examples of suitable promoters include pol I, pol II, pol III (e.g. U6 and H1 promoters). Examples of pol II promoters include, but are not limited to, retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-acting promoter, the phosphoglycerol kinase (PGK) promoter, and the EF1α promoter.

Examples of other suitable promoters may be bacterial or phage promoters, such as those described in https://parts.igem.org/Promoters/Catalog. In one embodiment, the promoter may be a Synechocystis promoter, such as the psbA2 promoter for the D1 subunit from Synechocystis. In another embodiment, the promoter may be an E. coli σ70 constitutive promoter.

In some examples, inducible promoters can be used. Examples of inducible promoters include, but are not limited to, tetracycline repressor system promoters, Lac repressor system promoters, arabinose-inducible, copper-inducible system promoters, salicylate-inducible system promoters (e.g., the PR1a system), glucocorticoid-inducible promoters, and ecdysone-inducible system promoters. In one embodiment, the inducible promoter is araBAD arabinose inducible promoter.

Suitably the one or more nucleic acids encoding the Cas proteins are operably linked to a promoter which is a pol II promoter.

Suitably the one or more nucleic acids encoding the or each guide RNA are operably linked to a promoter which is a pol III e.g. U6 or H1 promoter.

As well as promoters, regulatory elements may include enhancer elements, such as WPRE; CMV enhancers; the R-U5′ segment in LTR of HTLV-I; SV40 enhancer; and the intron sequence between exons 2 and 3 of rabbit β-globin. Suitably some bacterial promoters may comprise binding sites for regulatory elements such as activators. It will be appreciated by those skilled in the art that the design of the expression vector can depend on such factors as the choice of the host cell to be transformed, the level of expression desired, etc.

Suitably the vector may also optionally include a transcriptional and/or translational termination region (i.e., termination region) that is functional in the selected host cell. A variety of transcriptional terminators are available and are responsible for the termination of transcription beyond the heterologous nucleotide sequence of interest and correct mRNA polyadenylation. The termination region may be native to the transcriptional initiation region, may be native to the operably linked nucleic acid sequence, may be native to the host cell, or may be derived from another source (i.e., foreign or heterologous to the promoter, to the nucleic acid sequence, to the host, or any combination thereof).

Suitably the vector may also include a nucleotide sequence for a selectable marker, which can be used to select a transformed host cell. As used herein, “selectable marker” means a nucleotide sequence that when expressed imparts a distinct phenotype to the host cell expressing the marker and thus allows such transformed cells to be distinguished from those that do not have the marker. Such a nucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Of course, many examples of suitable selectable markers are known in the art and can be used in the expression cassettes described herein. In some examples, a selectable marker useful with this invention includes polynucleotide encoding a polypeptide conferring resistance to an antibiotic. Non-limiting examples of antibiotics useful with this invention include ampicillin, kanamycin, streptomycin, spectinomycin, gentamicin, tetracycline, chloramphenicol, and/or erythromycin. Thus, in some examples, a polynucleotide encoding a gene for resistance to an antibiotic may be introduced into the organism, thereby conferring resistance to the antibiotic to that organism.

Non-limiting examples of general classes of vectors include but are not limited to a viral vector, a plasmid vector, a phage vector, a phagemid vector, a cosmid vector, a fosmid vector, a bacteriophage, an artificial chromosome, or an Agrobacterium binary vector in double or single-stranded linear or circular form which may or may not be self-transmissible or mobilizable. A vector as defined herein can transform a prokaryotic or eukaryotic host either by integration into the cellular genome or exist extrachromosomally (e.g. autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells). A plasmid may be vector in accordance with this description, which is a circular double-stranded DNA loop into which additional DNA segments can be inserted, such as by standard molecular cloning techniques.

Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

Suitably the vector used is a plasmid.

Suitably the vector is selected which is suitable for the cell or organism into which vector is to be introduced. Suitably the plasmid is selected which is suitable for the cell or organism into which plasmid is to be introduced.

Suitable plasmids for bacterial expression may include: pQE80L, pACYC-Duet, pSEVA series for example. Suitable plasmids for mammalian expression may include pcDNA3.1+.

Suitably the, or each, vector is for introducing the Cas proteins and guide RNA into a cell such that the methods of the invention can take place within the cell. Suitably therefore the methods may comprise a step of introducing a vector comprising one or more nucleic acids encoding the Cas proteins or modified Cas proteins, and one or more nucleic acids encoding the guide RNAs into a cell, wherein the cell comprises the target nucleic acid sequence.

Suitable means of introducing vectors into cells are the same as the means for introducing nucleic acids into cells as described hereinabove. For example, methods of non-viral delivery of nucleic acids may include lipofection, microinjection, biolistics, virosomes, liposomes, immunoliposomes, polycation or lipid: nucleic acid conjugates, naked DNA, artificial virions, conjugation, and agent-enhanced uptake of DNA.

Suitably after introduction of the, or each, vector into the cell, the Cas proteins and the guide RNA are expressed in the cell. Suitably expression of the Cas proteins and the guide RNA may be induced, suitably induced from the, or each, vector. Suitably therefore the, or each, vector comprises an inducible promoter operably linked to the, or each, nucleic acid sequence encoding the Cas proteins and/or the guide RNA. Suitably the cell may be contacted with an inducer to induce said expression. Suitably the inducer may induce expression of the Cas proteins and/or the guide RNA from the or each vector.

Suitably upon expression of the Cas proteins and the guide RNA, the components assemble into the Type III-D CRISPR-Cas system of the invention.

### Cells

The methods of the present invention may be carried out in a cell, and the Type III-D CRISPR-Cas complex and/or sequences encoding such a complex can be provided in a cell. Therefore, there is provided a cell comprising a Type III-D complex system of the invention, or a modified Type III-D CRISPR-Cas system complex of the invention, comprising a vector of the invention, or comprising a nucleic acid encoding any part of the Type III-D CRISPR-Cas system of the invention. Suitably therefore the cell may be regarded as a host cell.

Suitably the cell may be ex vivo, in vitro, or in vivo.

Suitably the cell may be eukaryotic or prokaryotic. Suitably the cell may be from a bacterium, archaeon, plant, animal, insect or fungi. Suitably the cell is a cyanobacterial cell.

Suitably the cell is an animal cell. Suitably the cell is a mammalian cell. Suitably the cell may be a human or a non-human cell. Suitably the cell may be a non-human mammalian cells. Suitably the cell may be a non-human primate cell.

Suitably the cell may be part of an organism. Suitably the cell may be located within an organism. Suitably the organism may be a prokaryote or a eukaryote. Suitably the organism is a bacterium, a virus, an archaeon, a fungus, plant, or an animal. Suitably the organism may be a host organism.

Thus, the invention includes any animal or cell, produced by the present methods, or a progeny thereof. The progeny may be a clone of the produced plant or animal or may result from sexual reproduction by crossing with other individuals of the same species to introgress further desirable traits into their offspring.

### EXAMPLES

### Example 1: Structural Analysis of Type III-Dv CRISPR-Cas

### Materials and Methods

### Culture Conditions

Refer to Supplementary Table 1 for a list of all strains used in this work. Refer to Tables 2 and 3 for lists of all oligonucleotides and plasmids, respectively.

Unless otherwise noted, Escherichia coli strains were grown at 37° C. in Lysogeny Broth (LB), or on LB-agar (LBA) plates with 1.5% (w/v) agar. Media were supplemented with antibiotics when required as follows: chloramphenicol (Cm; 25 μg/mL), and kanamycin (Km; 50 μg/mL).

### Construction of Plasmids

A plasmid (pPF2434) for expression of Cas10, Cas7-5-11, Cas7-2x, Csx19 and Cas7-insert was constructed by PCR-amplifying their genes (primers PF4851+PF4852) using Synechocystis genomic DNA as template and cloning the product into pRSF-1b via KpnI and PstI restriction sites. The cas10 gene was cloned to incorporate an N-terminal His6 tag followed by TEV protease recognition sequence.

A plasmid (pPF2441) for expression of the first spacer (5′-TGTAGTAGAACCAATCGGGGTCGTCAA TAACTCCCG-3′) and flanking repeatsequences (5′-GTTCAACACCCTCTTTTCCCCGTCAGGGGACTGAAAC-3′) from the Type III-Dv associated CRISPR array was constructed by PCR-amplifying this region from Synechocystis genomic DNA (primers PF4847+PF4848) and cloning the product into pACYCDuet-1 via NdeI and KpnI restriction sites. A plasmid (pPF2442) was constructed for expression of Cas6-2a with the first spacer and flanking repeat sequences by PCR-amplifying cas6-2a (primers PF4849+PF4850) using Synechocystis genomic DNA as template and cloning the product into pPF2441 via NcoI and BamHI restriction sites.

Plasmids pPF3085, pPF3086, pPF3089, pPF3205, and pPF3206 are for expression of mutants Cas7-2x(D29A,D31A,D33A), Cas7-2x(D241A,D246A), ΔCsx19 (nonsense mutation), Cas7-5-11 (D26A) and Cas7-insert (Δ104 N-terminal residues), respectively. Plasmids pPF3085, pPF3086, pPF3089, pPF3205, and pPF3206 were constructed by site-directed mutagenesis through amplifying plasmid pPF2434 with primers PF5991+PF5992, PF5993+PF5994, PF6281+PF6282, PF6423+PF6424, and PF6425+PF6426, respectively. Each were treated with DpnI to remove PCR template, and Gibson assembly to ligate the PCR product into the mutated plasmid.

### Expression and Purification of the Type III-Dv Effector Complex

Type III-Dv complex with N-terminal His6-TEV-Cas10 was expressed in LOBSTR cells containing plasmids pPF2434 and pPF2441. Five hundred mL cultures were induced with 0.5 mM IPTG at OD₆₀₀=0.6 and grown overnight at 18° C. Cells were harvested at 10,000×g for 10 min. The cell pellet was resuspended in 20 ml of lysis buffer (50 mM HEPES-NaOH, pH 7.5, 300 mM KCl, 5% Glycerol, 1 mM DTT, 10 mM imidazole) supplemented with 0.02 mg/mL DNaseI, complete EDTA free protease inhibitor (Roche). Cells were lysed by a French pressure cell press (American Industry Company) at 10,000 psi, and the lysate was clarified by centrifugation at 15,000×g for 15 min. The lysate was applied to a HisTrap affinity column (GE Healthcare) equilibrated in lysate buffer and eluted using a gradient against lysate buffer containing 500 mM imidazole. The fractions containing the Type III-Dv complex were pooled and treated with TEV protease and incubated at 4° C. during overnight dialysis in SEC buffer (10 mM HEPES-NaOH, PH 7.5, 100 mM KCl, 5% Glycerol, 1 mM DTT). The sample was applied to a second HisTrap column; however, due to inefficient TEV cleavage, the complex unexpectedly bound the column and eluted with high imidazole. The complex was further purified by size exclusion chromatography (SEC) on a HiLoad 16/600 Superdex 200 column (GE Healthcare) equilibrated in SEC Buffer). Mutant Type III-Dv complexes were similarly expressed and purified, except TEV protease was omitted. Purified complexes were typically concentrated to 1.5 mg/ml using a centrifugal concentrator (Amicon; 100 kDa MWCO), aliquoted, and stored at −80° C.

### Native Mass Spectrometry

5 μL aliquots of the CRISPR complex solution were buffer exchanged into 100 mM ammonium acetate using Biospin P-6 gel columns (Bio-Rad Laboratories Inc., Hercules, CA) prior to native mass spectrometry. Samples were loaded onto gold/palladium-coated borosilicate static emitters and subjected to electrospray ionization using a source voltage of 1.0-1.3 kV and analyzed in the positive ion mode on a Thermo Scientific Q Exactive Plus UHMR Orbitrap mass spectrometer (Bremen, Germany). Subcomplexes and ejected subunits were produced and measured via quadrupole isolation of the intact complex charge envelope, followed by higher-energy collisional dissociation (HCD) using 290 eV normalized collision energy (NCE). Ion optics and trapping gas pressure were tuned for the transmission and detection of each set of analytes, including the intact complex, subcomplexes, and ejected subunit ions. Native mass spectra were collected by averaging 500 microscans at a resolution of 1,625 at m/z 200. Spectra were deconvoluted using UniDec.

Denaturing liquid chromatography mass spectrometry (LC-MS) was performed on a Dionex UltiMate 3000 nanoLC system coupled to a Thermo Orbitrap Fusion Lumos Tribrid mass spectrometer (San Jose, Ca). The trap column (3 cm) and analytical column (30 cm) were packed in-house with polymer reverse-phase (PLRP) packing material. Approximately 80 ng of the CRISPR complex were injected and subjected to reverse-phase chromatography, utilizing water with 0.1% formic acid as mobile phase A (MPA), and acetonitrile with 0.1% formic acid as mobile phase B (MPB). Forward trapping occurred for 5 minutes at 2% MPB at a flow rate of 5 μL/min at the trap column. Elution onto the analytical column (at 0.3 μL/min) occurred by increasing MPB to 10% over a 3-minute gradient followed by an increase to 35% MPB over 32 minutes. Mass spectra were collected at a resolution of 15,000 at m/z 200, using 5 microscans and an AGC target of 1E6. Spectra were manually averaged over each subunit elution period and deconvoluted with UniDec.

### RNA Cleavage by the Type III-Dv Effector Complex

The RNA substrates contained sequence 5′-CAUGACGGAUCGCGGGAGUUAUUGACGACCCCGAUUGGUUCUACUACAAACGUGAUACUA-3′ (SEQ ID NO: 24), which included sequence complementary to the Type III-Dv crRNA spacer and either had a 5′ (FAM or IRD800 (IRD)) or 3′ (FAM) fluorescent labels.

RNA cleavage assays were conducted in 5 μL of reaction typically containing 200 nM purified Type III-Dv effector complex, 100 nM RNA substrate in final buffer conditions of 6 mM HEPES-NaOH, PH 7.5, 60 mM KCl, 10 mM MgCl₂ or MnCl₂, 3% glycerol, 1 mM DTT. Reactions were incubated at 37° C. for 30 min, or for a different time span as indicated. Reactions were stopped by adding 1 μL 6 M guanidinium thiocyanate and 6 μL 2×RNA loading dye. Samples were heated for 5 min at 95° C. and immediately on ice for 3 min. Samples were analysed on a 1×TBE, 15% acrylamidede, 8M urea denaturing PAGE (Thermo Fisher). Fluorescent probe was imaged using the Odyssey Fc imaging system (LICOR).

### Cryo-EM Grid Preparation and Data Collection

Fully assembled Type III-D binary complex was diluted to a concentration of 0.3 mg/ml in SEC buffer before 2.5 μl of sample was added to a quantifoil 1.2/1.3 grid that was glow discharged for 1 minute. Sample was applied to the grid in an FEI Vitrobot MarkIV kept at 100% humidity and 4° C. before blotting for 5.5 seconds with a force of 0. For the ssRNA target-bound complex, non-self ssRNA was mixed with the binary complex with a 2:1 molar ratio of RNA:Binary complex in SEC buffer to a final protein concentration of 0.3 mg/mL. Grids of the target-bound complex were frozen identical to that of the binary complex. Both grids were loaded to an FEI Titan Krios (Sauer Structural Biology Lab, University of Texas at Austin) operating at 300 kV. Images were taken at a pixel size of 0.81 Å/pixel with a dose rate of 10.6 e⁻/pixel/s for 5 seconds using a Gatan K3 direct electron detector, giving a final dosage of 80.5 e⁻/Å². Data collection was automated using SerialEM using a defocus range of −1.2 to −2.2 μm.

### Cryo-EM Data Processing

Movies from the Gatan K3 were motion corrected using motioncor2, and corrected micrographs were uploaded to cryoSPARC v2. After CTF correction, initial templates for template-based picking were generated using a blob picker and 2D classification. Template-based particle picking resulted in ˜1.89 million particles (binary complex) and ˜1.92 million particles (target-bound complex) being picked.

To continue processing the dataset for the binary complex, I started with one round of 2D classification, sorting out particles to a new subset of ˜926 k particles. I then utilized ab initio reconstruction and subsequent heterogeneous refinement with four classes and selected ˜649 k particles from one of the classes. Particles were then split by exposure groups before performing a final non-uniform (NU) refinement with per-particle defocus optimization, exposure group CTF parameter optimization, and over per-particle scale minimization. The final model yielded from this refinement is composed of ˜649 k particles at 2.5 Å resolution.

For the target-bound complex, ˜1.92 million particles were input into 2D classification and filtering, sorting out particles to a new subset of ˜1.07 million particles. This new subset of data was then input into ab initio reconstruction and heterogeneous refinement with four classes and filtered out ˜453 k particles to a new subset of ˜614 k particles. These particles were split by exposure groups before performing NU refinement with identical settings to the final NU refinement in the binary complex dataset. This refinement yielded a 2.8 Å resolution structure from ˜610 k particles.

### In Silico Subunit Modelling and Refinement:

Protein structures of Type III-D2 Cas7-3x, the Sb-gRAMP Type III-E effector, the D. ishimotonii Type III-E effector, and Type III-D1 Cas7-Cas5 were predicted using Texas Advanced Computing Center Stampede2 computer cluster with AlphaFold2 (Jumper et al., 2021). Structures were predicted using the monomer model preset. The reduced database precision was used for the multiple sequence alignment. The AF2 job run included a relaxation step, resulting in both relaxed and unrelaxed models. Each job was run for a total of 48 hours, yielding 2 to 5 models per protein.

TABLE 1
Strains used for Type III-Dv experiments

| Name | Genotype/Phenotype |
|---|---|
| DH5a | cloning strain. E. coli F⁻, φ80ΔdlacZM15, Δ(lacZYA-argF)U169, endA1, recA1, |
| | hsdR17 (rK⁻ mK⁺), deoR, thi-1, supE44, λ⁻, gyrA96, relA1 |
| LOBSTR | protein expression strain. E. coli B F⁻ ompT, gal, dcm, lon, hsdS_{B}(r_{B}⁻m_{B}⁻), |
| | λ(DE3 [lacI lacUV5-T7p07 ind1 sam7 nin5]) [malB⁺]_{K-12}(λ^{S}) arnA slyD |
| Synechocystis | Glucose tolerant laboratory wild-type strain GT-01 |
| sp. PCC 6803 | |

TABLE 2
Oligonucleotides used for Type III-Dv experiments

| | | SEQ ID | |
|---|---|---|---|
| Name | Sequence (5′-3′) | NO | Description |
| Cloning | | | |
| PF4847 | TATACATATGGCATACAGACTGTT | 72 | F repeats and spacer1 |
| | TTTCAGTGTGATAG | | |
| PF4848 | CGAGGGTACCGGGACTCCAACCC | 73 | R repeats and spacer1 |
| | CCCAAG | | |
| PF4849 | TATACCATGGTGGATCTAAAATCC | 74 | F cas6-2a |
| | TTAGCTG | | |
| PF4850 | ATTCGGATCCTTATTGAACATTGG | 75 | R cas6-2a |
| | CTAAGGC | | |
| PF4851 | TGGGTACCGAAAACCTGTATTTTC | 76 | F cas10 |
| | AGGGCTTTCTAGTTCTAATTGAGA | | |
| | CTTCCGGTAATC | | |
| PF4852 | CGGCCGCAAGCTTGTCGACCTGC | 77 | R cas7-insert |
| | AGTTAACTAGGTTTGATTGGAAAA | | |
| | CTCTGG | | |
| PF5855 | rCrArUrGrArCrGrGrArUrCrGrCrGr | 78 | 60 nt RNA target |
| | GrGrArGrUrUrArUrUrGrArCrGrAr | | |
| | CrCrCrCrGrArUrUrGrGrUrUrCrUr | | |
| | ArCrUrArCrArArArCrGrUrGrArUrA | | |
| | rCrUrA | | |
| PF5991 | GGCGCCGCTGCGACGGCTTTAGC | 79 | F cas7-2x D33A mutant |
| | CCTGGCGGTTAATGGTG | | |
| PF5992 | AAAGCCGTCGCAGCGGCGCCACC | 80 | R cas7-2x D33A mutant |
| | CACACCACCAATG | | |
| PF5993 | GGCTGGACTGGCGATCGCTATTTT | 81 | F cas7-2x D246A mutant |
| | GCCCCTCGTTAGTCAAGTG | | |
| PF5994 | TAGCGATCGCCAGTCCAGCCCCTT | 82 | R cas7-2x D246A mutant |
| | CAGCTTTCACCATGAC | | |
| PF6281 | GCCTAAGTTAGTAACTTTACCACT | 83 | F Δcsx19 mutant |
| | ACCACCAATATGAAATTACCCTC | | |
| PF6282 | GTAAAGTTACTAACTTAGGCGGCC | 84 | R Δcsx19 mutant |
| | TCCTGCTG | | |
| PF6423 | GAACTAGCCAGTGTTGTACAACG | 85 | F cas7-5-11 D26A mutant |
| | GGATGGAG | | |
| PF6424 | TGTACAACACTGGCTAGTTCCCCC | 86 | R cas7-5-11 D26A mutant |
| | CGACCCATG | | |
| PF6425 | AAGTAAAATGACACCCAGAAATGT | 87 | F cas7-insert Δ104 N-term |
| | TAACGCTAGCAAC | | mutant |
| PF6426 | TTCTGGGTGTCATTTTACTTAACC | 88 | R cas7-insert Δ104 N-term |
| | TCCAATTTAATTAAACGTTC | | mutant |
| PF5856 | /5IRD800CWN/rCrArUrGrArCrGr | 89 | 5′-IRD800 60 nt RNA target |
| | GrArUrCrGrCrGrGrGrArGrUrUrAr | | |
| | UrUrGrArCrGrArCrCrCrCrGrArUr | | |
| | UrGrGrUrUrCrUrArCrUrArCrArArA | | |
| | rCrGrUrGrArUrArCrUrA | | |
| PF6575 | /56-FAM/rCrArUrGrArCrGrGrArUr | 90 | 5′-FAM 60 nt RNA target |
| | CrGrCrGrGrGrArGrUrUrArUrUrGr | | |
| | ArCrGrArCrCrCrCrGrArUrUrGrGr | | |
| | UrUrCrUrArCrUrArCrArArArCrGrU | | |
| | rGrArUrArCrUrA | | |
| PF6576 | rCrArUrGrArCrGrGrArUrCrGrCrGr | 91 | 3′-FAM 60 nt RNA target |
| | GrGrArGrUrUrArUrUrGrArCrGrAr | | |
| | CrCrCrCrGrArUrUrGrGrUrUrCrUr | | |
| | ArCrUrArCrArArArCrGrUrGrArUrA | | |
| | rCrUrA/36-FAM/ | | |
| PF6577 | /56-FAM/rCrArUrGrArCrGrGrArUr | 92 | 5′-FAM 43 nt RNA target |
| | CrGrCrGrGrGrArGrUrUrArUrUrGr | | |
| | ArCrGrArCrCrCrCrGrArUrUrGrGr | | |
| | UrUrCrUrA | | |
| PF6578 | /56-FAM/rCrArUrGrArCrGrGrArUr | 93 | 5′-FAM 37 nt RNA target |
| | CrGrCrGrGrGrArGrUrUrArUrUrGr | | |
| | ArCrGrArCrCrCrCrGrArUrUrG | | |
| PF6579 | /56-FAM/rCrArUrGrArCrGrGrArUr | 94 | 5′-FAM 31 nt RNA target |
| | CrGrCrGrGrGrArGrUrUrArUrUrGr | | |
| | ArCrGrArCrCrC | | |
| PF6580 | /56-FAM/rCrArUrGrArCrGrGrArUr | 95 | 5′-FAM 27 nt RNA target |
| | CrGrCrGrGrGrArGrUrUrArUrUrGr | | |
| | ArCrG | | |
| PF6582 | /56-FAM/rCrArUrGrArCrGrGrArUr | 96 | 5′-FAM 60 nt RNA anti- |
| | CrGrCrGrGrGrArGrUrUrArUrUrGr | | repeat |
| | ArCrGrArCrCrCrCrGrArUrUrGrGr | | |
| | UrUrCrUrArCrUrArCrArGrUrUrUr | | |
| | CrArGrUrCrCrCrC | | |
| PF6583 | /5IRD800CWN/rCrArUrGrArCrGr | 97 | 5′-IRD800 37 nt RNA target |
| | GrArUrCrGrCrGrGrGrArGrUrUrAr | | |
| | UrUrGrArCrGrArCrCrCrCrGrArUr | | |
| | UrG | | |

TABLE 3
Plasmids used for the Type III-Dv experiments

| Name | Description |
|---|---|
| pACYCDuet-1 | Two T7/LacO promoters with P15A replicon, Cm^{R} |
| pPF2434 | N-His₆-tagged Cas10, Cas7-5-11, Cas7_2x, Csx19 and Cas7-insert, |
| | pRSF-1b |
| pPF2441 | Spacer1 of Synechocystis Type III-Dv CRISPR array, pACYCDuet-1 |
| pPF2442 | Plasmid pPF2434 with Cas6-2a |
| pPF3085 | Modified pPF2434 with Cas7-2x(D29A, D31A, D33A) |
| pPF3086 | Modified pPF2434 with Cas7-2x(D241A, D246A) |
| pPF3089 | Modified pPF2434 without Csx19 |
| pPF3205 | Modified pPF2434 with Cas7-5-11(D26A) |
| pPF3206 | Modified pPF2434 with Cas7-insert(Δ104 N-terminus) |
| pRSF-1b | T7/LacO promoter with RSF1030-derived replicon, Km^{R} |

### Results

The Type III-Dv Effector Forms a 332 kDa Complex with No Repeated Subunits

The operon of the Type III-Dv complex from Synechocystis contains cas10, a cas7-cas5-cas11 fusion, a double cas7 fusion (cas7-2x), csx19, and an insertion-containing cas7 (cas7-ins). Adjacent to the cas operon is cas6-2a, adaptation genes and a CRISPR array containing multiple spacers (FIG. 1a) (Scholz et al., 2013). To determine the composition of the Type III-Dv effector complex, we cloned the cas operon, cas6-2a, and first repeat-spacer-repeat of the CRISPR array from Synechocystis and expressed the operon in E. coli. The complex was purified using metal affinity and size-exclusion chromatography, where it eluted at approximately 330 kDa. Analysis of the purified complex by SDS-PAGE and mass spectrometry confirmed the presence of all proteins except Cas6-2a (FIG. 1c). The observation of Csx19 indicates this protein is a core component of the effector. Analysis of the crRNA length showed a mature crRNA of 37-nt, which agrees with a previous analysis of Type III-Dv crRNAs in Synechocystis (FIG. 1b) (Scholz et al., 2013).

To confirm the composition and stoichiometry of this multi-subunit fusion protein effector, we performed electrospray ionization (ESI) native mass-spectrometry on the purified complex (FIG. 1d). ESI showed one predominant peak corresponding to a native mass of ˜332 kDa, which is in excellent agreement with a complex composed of one Cas7-2x, one Cas7-ins, one Cas7-Cas5-Cas11, one Csx19, one Cas10, and a mature crRNA of 37-nt. Smaller peaks showed subcomplexes lacking either a Cas10, Cas7-2x, or Csx19 subunit, suggesting that these subunits are on the periphery and/or are more likely to dissociate from the complex. The presence of each subunit was confirmed by subjecting the surveillance complex to denaturing top-down analysis with chromatographic separation. Altogether, biochemistry and native mass-spectrometry analyses show that Cas7-Cas5-Cas11 and Cas7-insertion assemble first, capping the two ends of the crRNA, followed by assembly of the Cas7-2× subunit, Csx19, and Cas10. The stoichiometry of the intact CRISPR complex is a heteromeric pentamer bound to the mature crRNA.

### Structural Analysis of the Type III-Dv Binary Complex

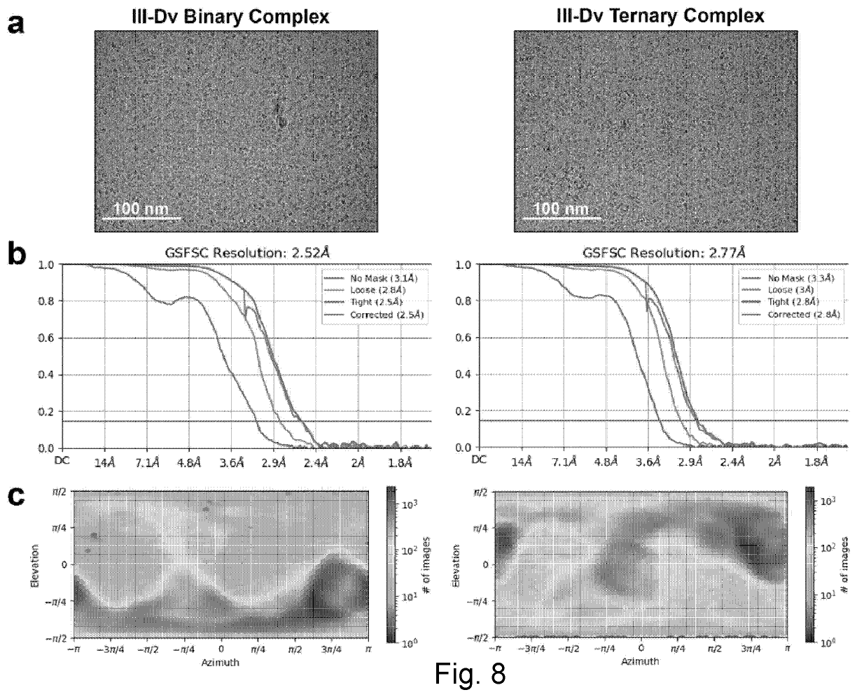

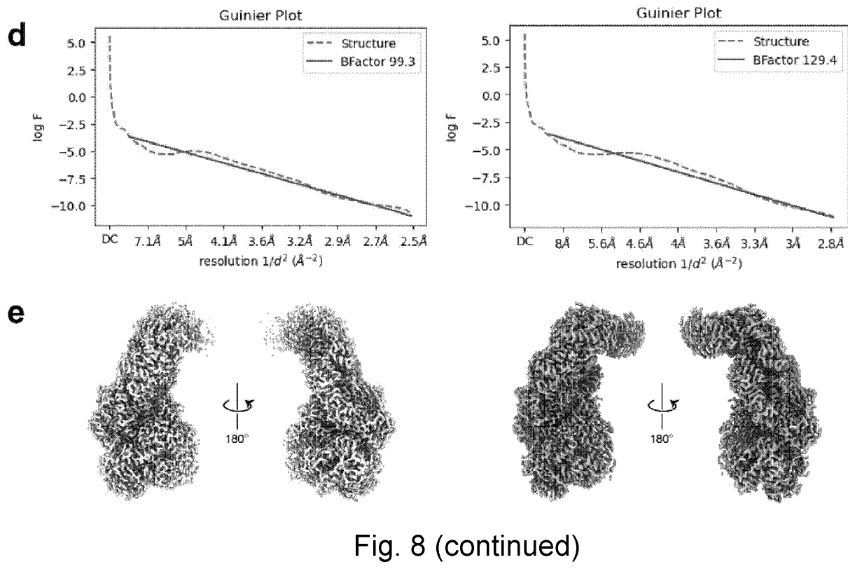

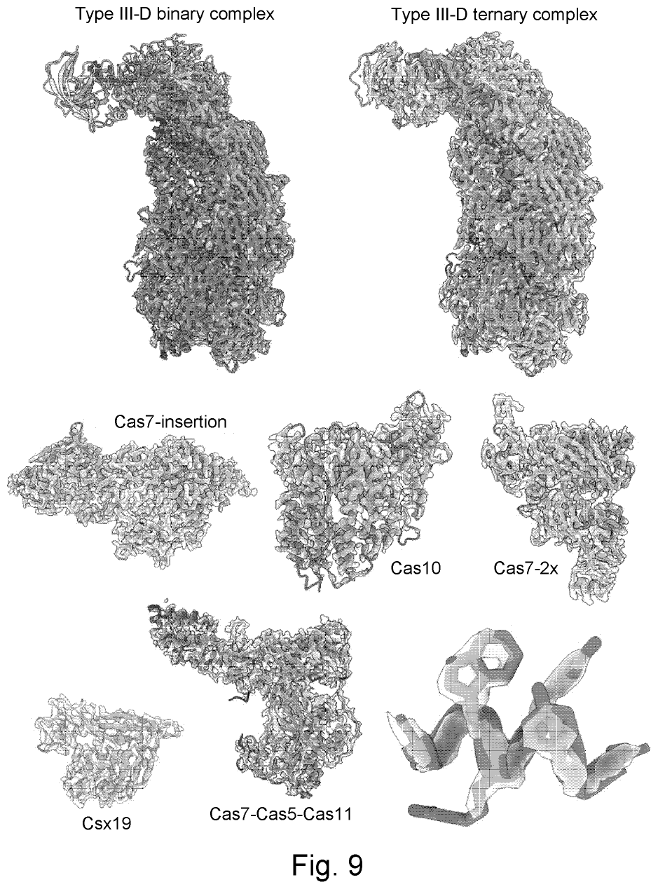

To delineate the architecture of this Type III-Dv effector, we used cryo-EM to determine a 2.5-Å resolution structure of the complex containing the nt crRNA (FIG. 1e,f, FIG. 8). To gain preliminary insights, we generated initial models of each subunit using Alphafold 2. After fitting these subunits into the map, we recognized a strong resemblance to a hammerhead shark, where the head is composed of the insertion containing Cas7, with the insertion domain and another small, uncharacterized domain creating each side of the head at the top of the complex, respectively. Interestingly, one side of the head (amino acids 1-112 of the Cas7-insertion N-terminus) was not observed in the cryo-EM map, likely due to flexibility. The body is composed of intertwined Cas7 and Cas11 domains of one Cas7-Cas5-Cas11 and one Cas7-2× subunit. Despite the Cas7 domains being part of larger fusion proteins or non-canonical subunits, the overall arrangement and assembly of these subunits allows for it to maintain a repeating backbone of Cas7 domains wrapping around the crRNA, forming a major filament, a structural feature conserved across all class I effectors. Sitting at the bottom of the complex, Csx19 nestles next to Cas5, each forming one side of the tail. Cas10 forms the fin, sandwiched between Csx19 and Cas11, forming buried surface area with the bottom Cas7 and Cas5 domains. This structure provides for a detailed understanding of how the domains of each of these fusion proteins are arranged. Interestingly, because the Type III-Dv operon appears to have retained the domain organization of the Type III-D1 operon, there are flexible linkers between the Cas7, Cas5, and Cas11 domains that allow for an unexpected structural organization of these subunits. A loop between the Cas7 and Cas5 domains (residues V221 to P244) and between the Cas5 and Cas11 domains (residues K602-T624) allows this fusion subunit to form an architecture that places Cas7 in the body of the complex, Cas5 below it at the tail, and Cas11 towards the head of the complex, on top of Cas7-different to their arrangement in the operon (FIG. 1a,e). It is tempting to hypothesize that Type III-Dv systems evolved from Type III-D1 systems through generation of gene fusions and long linkers between the domains rather than gene rearrangement followed by gene fusion. These linkers are not conserved, but contain mostly flexible residues.

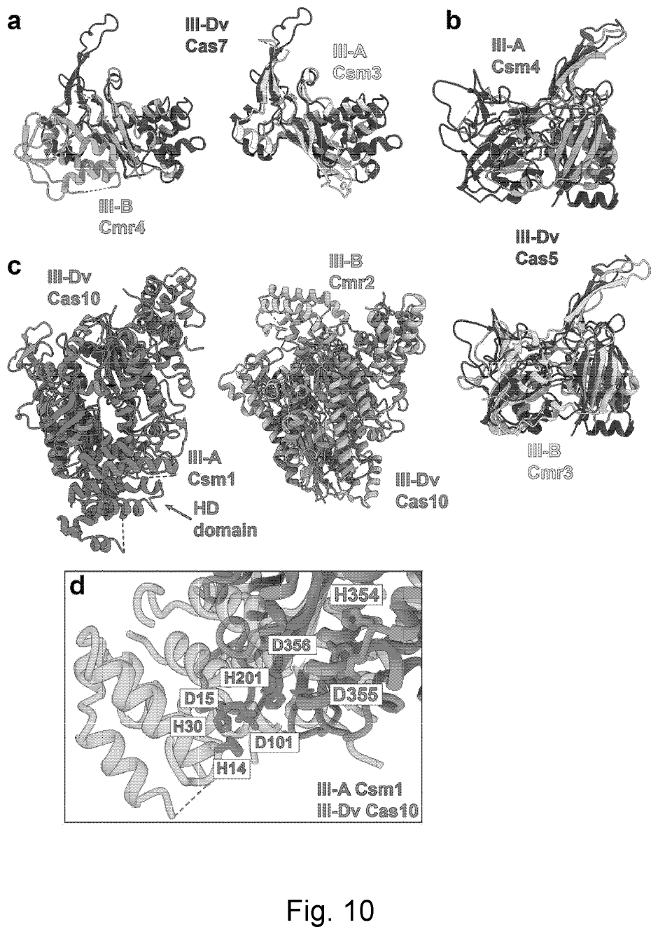

We utilized the Dali web server to search for structural homologues of each of our domains across the entire PDB (Holm, 2020). Cas7 structural alignments revealed that all the Type III-Dv Cas7 domains aligned better with Csm3, the Cas7 subunit from Type III-A, than Cmr4 from Type III-B effectors (FIG. 10). However, structural alignments of Type III-Dv Cas5 domain (Csx10) revealed that it aligned slightly better with the Cas5 homologue of the Type III-B (Cmr3, Z-score 21.9, PDB 3X1L) than Type III-A complex (Csm4, Z-score 15.0, PDB 6xn7) (FIG. 10). Type III-Dv Cas10 also appeared to align better with Type III-B Cas10 (Cmr2, Z-score 21.4, PDB 3w2w) than Type III-A Cas10 (Csm1, Z-score 18.8, PDB 6074) (FIG. 10). When aligned, the HD domain of the Type III-A Csm complex hangs off the periphery of both the Type III-Dv and Type III-B complexes. Despite apparent loss of this HD domain based on Cas10 structural comparisons, previous studies have predicted an HD site for Type III-Dv in Cas10. We were able to locate this putative site in our structure at residues H354, D355, and D356, indicating that Type III-Dv Cas10 does indeed have an HD motif, but loses the canonical domain for this site present in Csm1 (FIG. 10). Our structure also maintains the conserved GGDD motif of the Cas10 active site for cyclic oligoadenylate production from ATP. Running along the Cas7 major filament are the Cas11 domain of Cas7-Cas5-Cas11 and the C-terminus of Cas10. Both domains are highly alpha-helical and resemble conventional small subunit proteins of class 1 complexes. Interestingly, the Cas11 domain extends perpendicular to the trajectory of the Cas10 C-terminal domain, which is opposite to what is observed in Type I and other Type III complexes. Together, this data clearly defines the structural similarities of the Type III-Dv domains with known Type III-A and Type III-B structures, despite many of these proteins being fused and the large insertion in the last Cas7 domain. One notable exception is the Cas7-insert subunit protruding from the effector complex, which has not been previously seen in CRISPR-Cas effectors.

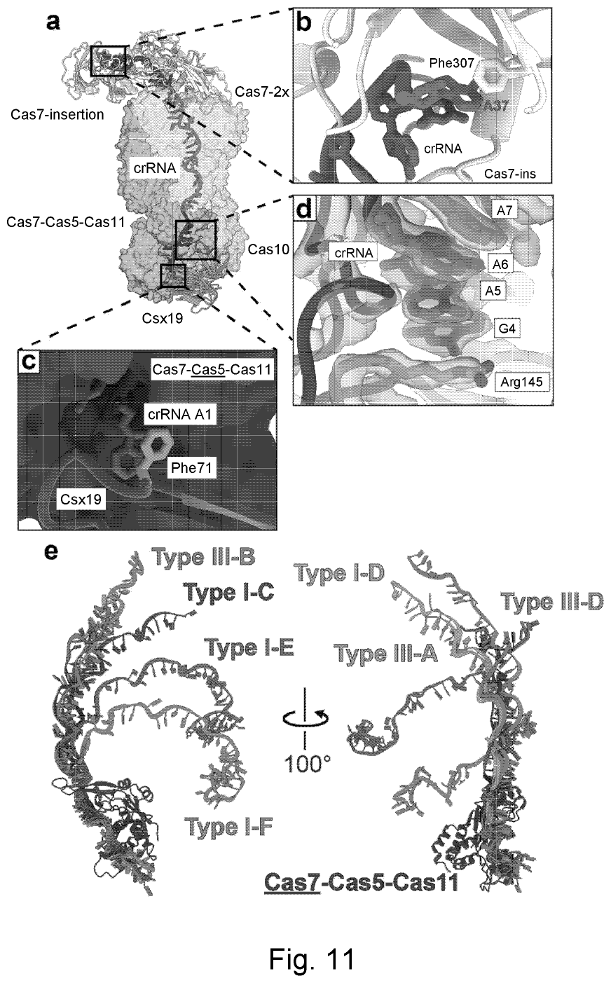

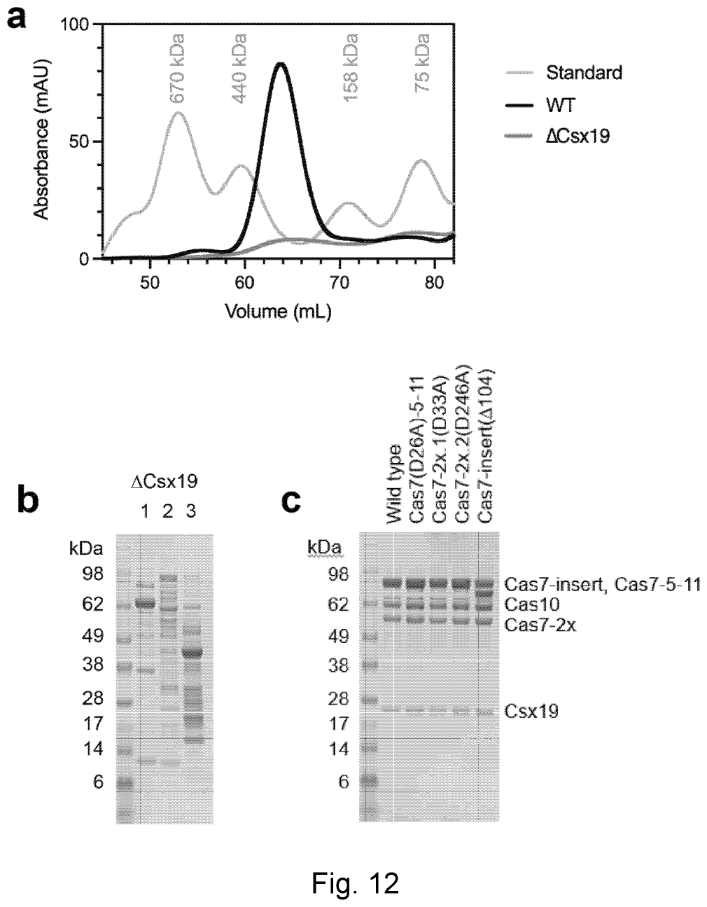

The Csx19 subunit is dominated by B sheets, and residue F71 caps the 8 nt 5′ crRNA handle through base stacking interactions between F71 and A1 of the crRNA (FIG. 11). Cas7-insertion caps the 3′ end of the crRNA through base-stacking between F307 and A37 of the crRNA (FIG. 11). R145 of Csx19 also contacts G4 of the crRNA (−5 position in the 5′ crRNA handle) in a pocket containing the 5′-AAA-3′ tag of the crRNA (positions −2 to −4) (FIG. 11), suggesting a role of this subunit in stabilizing the 5′ end of the crRNA. Despite these contacts, the role of Csx19 remains enigmatic. Interestingly, affinity purification of a ΔCsx19 complex with a N-terminal Cas10 tag did not result in pulldown of complex, indicating that assembly of Csx19 onto the Type III-Dv complex precedes Cas10 binding and is necessary for full complex assembly (FIG. 12). Considering the lack of examples of an insertion domain in Cas7 subunits of other Type III systems that have been structurally characterized, the role of this domain remains unknown.

Structural and Biochemical Basis of ssRNA Targeting and Cleavage by the Type III-Dv Effector

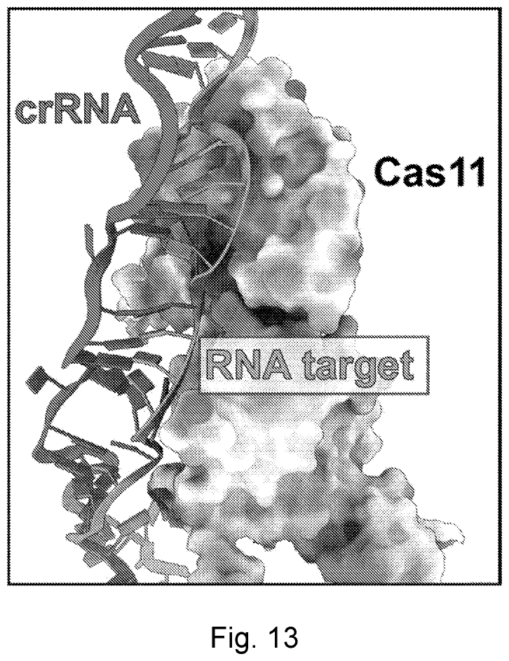

To gain mechanistic insight into RNA targeting by this complex, we again utilized cryo-EM, and solved the structure of the effector bound to target RNA at 2.8-Å resolution (FIG. 2a). The structure aligns near-perfectly to the binary structure, except for conformational changes in Cas10. The RNA target hybridizes along the crRNA backbone using Watson-Crick base pairing and follows the same trajectory as the crRNA. Additionally, the two separate small subunit domains appear to stabilize the phosphodiester backbone of the ssRNA target using a positively charged surface (FIG. 13). As in other class I complexes, every 6th nucleotide of the crRNA and RNA target is flipped out by the β-hairpin thumb domain of each Cas7 domain, except for the Cas7-insertion subunit. Instead, this subunit threads an ordered loop through the crRNA-RNA target duplex. This loop does not create enough steric hindrance to force the crRNA base out, but instead pushes the stacking bases apart, between bases U29 and C30 of the crRNA and G27 and A28 of the RNA target. The lack of a protruding β-hairpin from the Cas7-insertion subunit causes a more rounded kink in the RNA target. This kinked position is only 4-nt upstream of the kinked RNA backbone of the closest Cas7 in Cas7-2x.2.

The Type III-Dv binary structure highlights how the insertion domain of the Cas7-insertion subunit serves as an anchor that pulls the 3′ end of the crRNA spacer into a much different geometry than other Type III and Type I systems (FIG. 2b, FIG. 11). We observe the crRNA to be buried within the protein subunits throughout the entire complex with exception of the small region at the 3′ end of the the crRNA that is anchored by the insertion domain. These six terminal bases of the crRNA (U32-A37) lie in a positive pocket of the insertion domain, positioning the Watson-Crick face of the bases towards the surface, primed for base pairing with an RNA target (FIG. 2c,d). A37 is capped by Phe307 and U32 forms a base-stacking interaction with Phe767, while Ile355 and Ile453 hold the seed region in place. A salt bridge between R400, K396, and D616 within the Cas7-insertion subunit joins the cleft between insertion domain and the Cas7 domain, blocking RNA hybridization with the crRNA (FIG. 2e). Fascinatingly, this salt bridge breaks apart upon RNA target binding, despite appearing to block RNA target binding. We thus hypothesize that the six 3′ terminal bases of the crRNA serve as a seed region for initial binding of an RNA target. Sufficient hybridization between this crRNA seed region and an RNA target thus must initiate conformational changes to open the salt bridge for continued RNA hybridization. Indeed, after analyzing the conformational changes within the Cas7-insertion subunit upon RNA binding, we see a significant shift in the insertion domain, swinging outwards to open the cleft between the insertion domain and Cas7 domain, breaking the salt bridge (FIG. 2g,f). Together, these results highlight a unique RNA target seeding mechanism among Type III effectors, and removal of this insertion domain likely leads to off-target cleavage by forgoing necessary hybridization to the seed region.

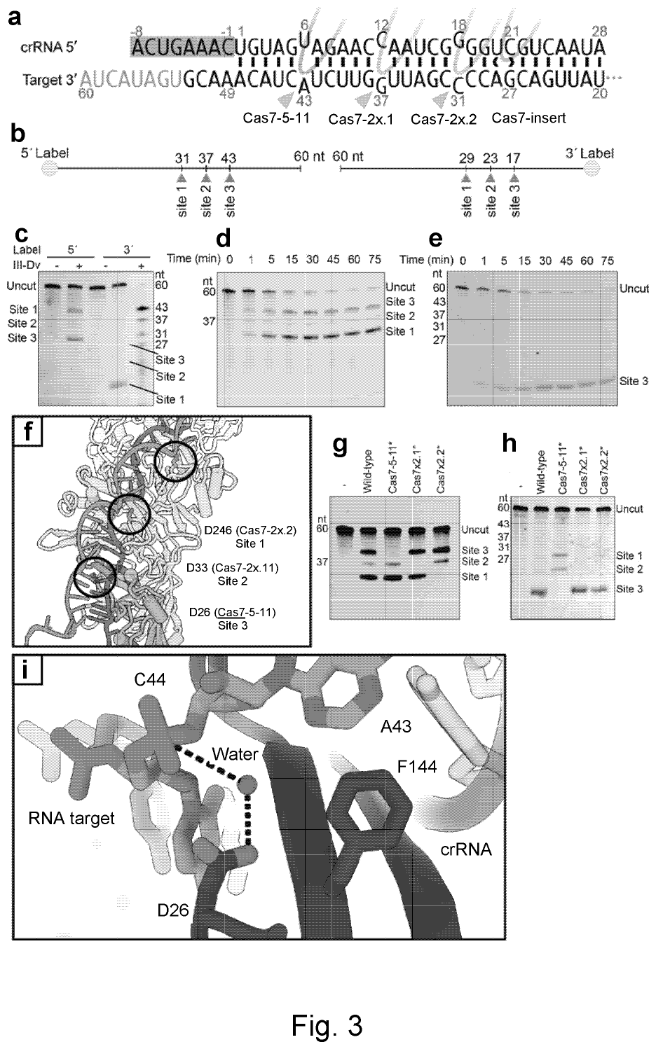

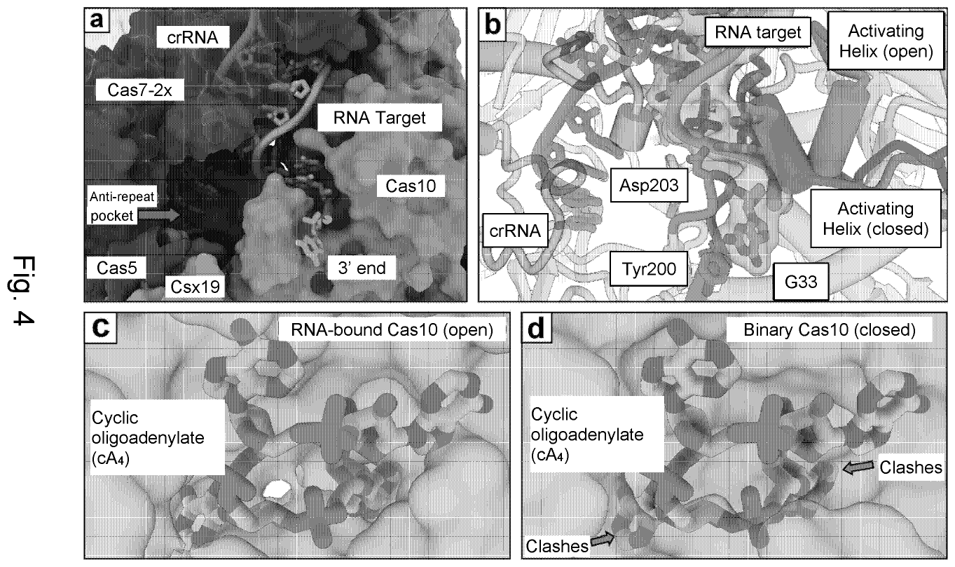

Next, we investigated the activity of the Type III-Dv effector against target RNA. Incubation of the complex with a 5′-fluorescently-labelled 60 nucleotide RNA substrate revealed cleavage of the RNA at positions 31, 37 and 43 nucleotides from the 5′ label (FIG. 3a-d). Interestingly, digestion of the same substrate but with a 3′ fluorescent label revealed only one predominant cleavage event positioned 17 nucleotides from the label (or 43 nucleotides from the 5′ end), suggesting a faster rate of cleavage at this position (FIG. 3c,e). Cleavage was metal-dependent with optimal cleavage occurring with Mg²⁺ and Mn²⁺, and cleavage was observed almost immediately (FIG. 2a). These results revealed three active Cas7 domains that may differ in kinetics.

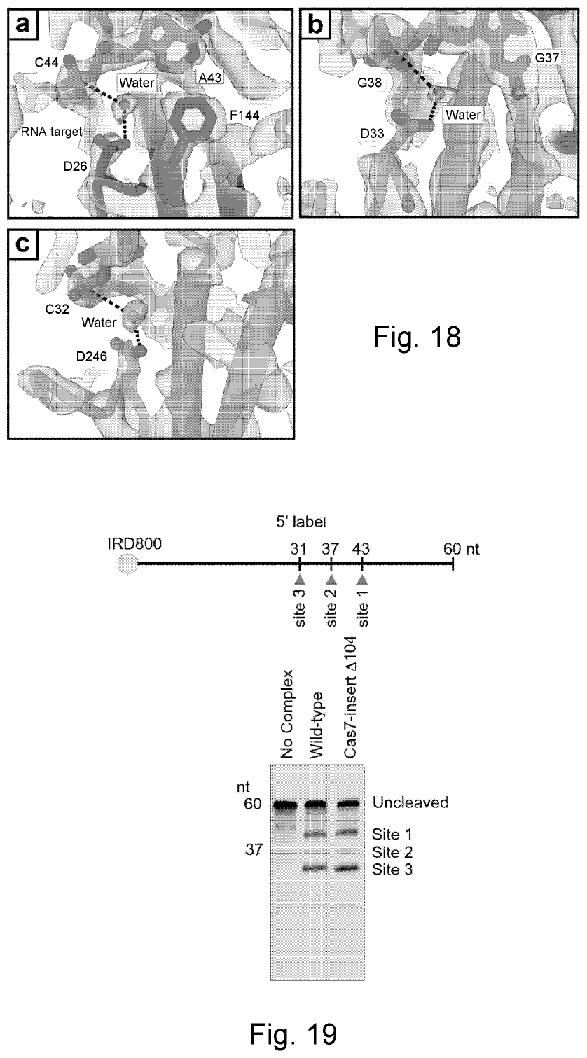

To gain a structural and mechanistic understanding of RNA cleavage by this effector, we first scanned the structure for acidic residues positioned at the kinked phosphodiester backbone of the RNA target. Structural analysis of the Cas7 domains revealed aspartate residues positioned adjacent to the scissile phosphate of the target RNA, corresponding to D26 of Cas7-Cas5-Cas11 (position 43 of the target), D33 of Cas7-2x.1 (position 37 of the target), and D246 of Cas7-2x.2 (position 31 of the target) (FIG. 3f). Interestingly, we found density at each active site that appears positioned between the identified aspartate residue and the scissile phosphate (FIG. 18). Considering these densities were not present in the binary structure and remained unaccounted for after the full Type III-D-target complex was built, as well as the fact that this map was solved without any divalent cations added to the buffer, we have putatively assigned these densities as water molecules.

To confirm the predicted active residues in the three active Cas7 domains, we mutated each aspartate to alanine, expressed and purified each variant, and tested these for cleavage activity against 5′ and 3′ fluorescently labelled RNA substrates (FIG. 3g,h). Mutation of the predicted active aspartate residues in the Cas7 domains successfully disrupted each cleavage event independently of the other. This would allow programming at these discrete and independent cleavage sites and could be exploited to create a Type III-Dv effector as a sequence-specific RNase enzyme.

### Conformational Changes Activate Cas10 for cOA Production

We next sought to understand whether the Type III-Dv complex retains a secondary immune response through activation of Cas10 upon non-self RNA target binding. In our structure, while the target RNA engages in Watson-Crick base pairing along almost the entirety of the crRNA, after position C8 in the crRNA, the target RNA disengages at the anti-tag sequence and is funneled into an exit channel on the surface of Cas10 (FIG. 4a). This is reminiscent of non-self-targeting that occurs within the Cas10 subunits of Type III-A and Type III-B effector complexes (Jia, Mo, et al., 2019; Sofos et al., 2020; You et al., 2019). Comparison of the target-bound complex with the binary complex shows only minor conformational changes throughout the Cas7 backbone. However, there are notable rearrangements in the Cas10 subunit. Closer inspection of the two structures reveals an alpha helix (L238-F245) that must be displaced to accommodate the 3′ end of the target RNA strand through the exit channel within Cas10 (FIG. 4b). This activation helix appears to communicate long-range allosteric changes, which leads to the opening of the cOA active site cleft. Intriguingly, in the target-bound structure, this cleft can perfectly accommodate a cA₄ COA ligand after Csm1 (PDB 607B) is aligned to Cas10 in our structure (FIG. 4c). This same analysis of the binary complex shows that this cleft is closed and the cOA has significant steric clashes with the surrounding protein (FIG. 4d). This suggests that the Cas10 subunit of the Type III-Dv complex is capable of producing cOA messengers to active Csm6 and other nucleases in a secondary immune response.

### In Silico Model Predictions Pave Structural Comparisons to Illustrate Type III Evolution

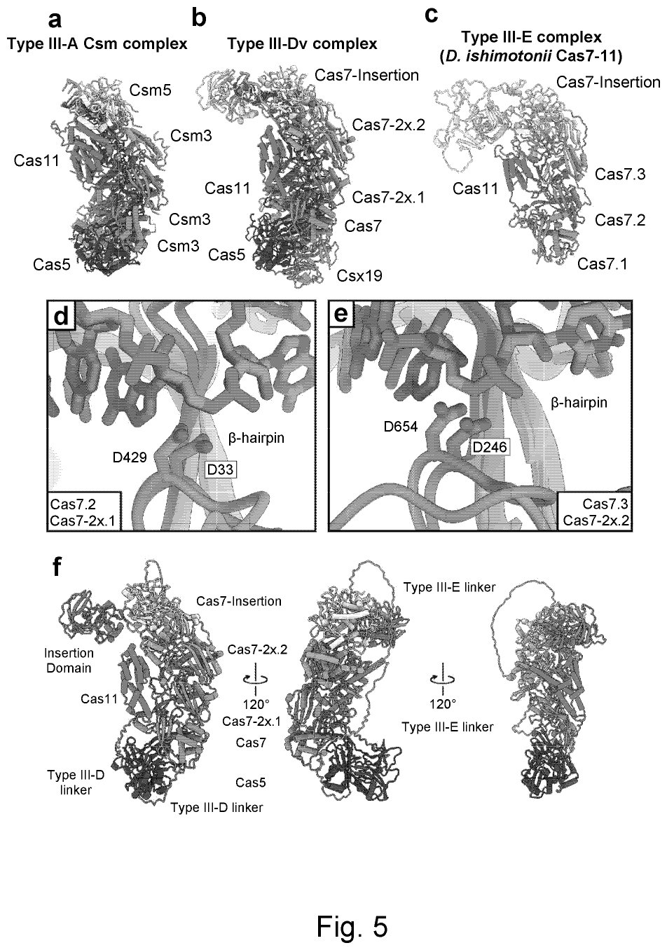

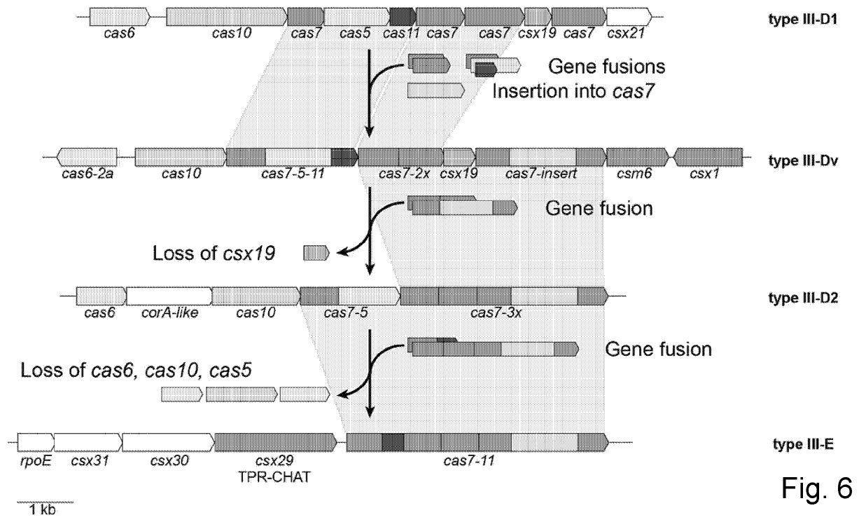

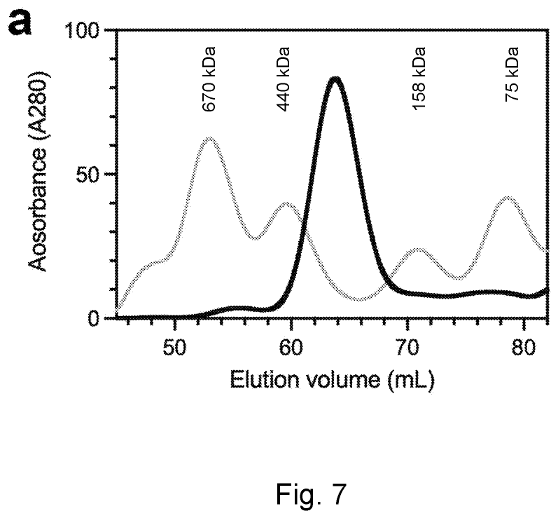

To gain a better understanding of the homology between Type III-D and Type III-E systems, we generated an in silico atomic model of the D. ishimotonii type Type III-E effector using Alphafold2 (Jumper et al., 2021). In our hypothetical evolutionary progression, Type III-D1 appears to have evolved first and contains single cas genes (FIG. 6). The operon consists of four separate cas7 genes, cas10, cas5 (csx10), cas11, and csx19 (Makarova, Wolf, et al., 2020; Rouillon et al., 2013, 2018). Intriguingly, the Type III-D2 system contains fusions of cas7 and cas5 (cas7-cas5) and the three following cas7 genes (cas7-3x) and lacks the csx19 and cas11 genes. Furthermore, Type III-D2 has a large domain inserted in the last cas7 in the operon (cas7-insertion). However, Type III-E does contain cas11, but not cas5 or cas10. These genes are instead fused together into a gene organization of cas7-cas11-cas7-cas 7-(cas7-insertion). Strikingly, the Type III-Dv Cas7-insertion and Cas7.4 of the predicted Type III-E model has the protrusion observed from the insertion domain within Cas7, highlighting this as a conserved structural feature between Type III-Dv, Type III-D2, and Type III-E cas operons containing genetic fusions. The Type III-E Cas7 backbone follows the same architecture and directionality as our Type III-Dv atomic structure, but both differ from the Type III-A Csm complex (FIG. 5a,b,c). Özcan and colleagues highlighted two ssRNA cleavage products from the D. ishimotonii Type III-E, which correspond to the active residues of D429 and D654 (Özcan et al., 2021). Interestingly, when aligned to our Type III-Dv target-bound model, we notice these aspartate residues are positioned right at the scissile phosphodiester bond at positions 31 and 37 of our ssRNA target (FIG. 5d,e). The Cas7 domains that contain these two aspartate residues align with the active site residues D33 and D246 in our Cas7-2x.1 and Cas7-2x.2 domains, respectively. These results paint a clear picture of the conserved structural features between Type III-Dv and Type III-E. However, despite close alignments of the Cas7 backbone subunits, the Type III-E structure is a simplified version of the Type III-Dv complex, with Csx19, Cas5, and Cas10 missing.

Because of the structural analysis of the linkers in the Type III-Dv, we attempted to engineer a single polypeptide Type III-Dv effector using subunits from the Type III-Dv complex with linkers from the Type III-E structural prediction. Because Cas7-Cas5-Cas11 and the two Central Cas7 domains in the Type III-Dv complex were already linked, we linked the C-terminus of the Cas11 domain with the N-terminus of Cas7-2x, as well as the C-terminus of Cas7-2× with the N-terminus of Cas7-insertion with the first 104 N-terminal residues removed, as these residues were not necessary for cleavage of an RNA target (FIG. 19). The residues in these linkers had no sequence conservation and were only characterized by the presence of flexible amino acids, likely aiding assembly of the domains. After linking all the subunits together into one chain, we performed Alphafold 2 on the single polypeptide Type III-Dv protein lacking Cas10 and Csx19. Shockingly, the structural predictions aligned incredibly well with our Type III-Dv structure, suggesting that the domains properly fold and assemble together with these long linkers between them (FIG. 5f). This is the first glimpse into an engineered class 1 CRISPR-Cas effector complex of single polypeptide. This provides an initial blueprint for building user-defined CRISPR-Cas effector complexes for a given activity with the ease of expression and assembly as a single polypeptide.

### Example 2: Nuclease Assays Involving Type III-Dv CRISPR-Cas

### Materials and Methods

### Bacterial Strains and Growth Conditions

Bacterial strains and phages used in this study are summarised in Table 4. Unless otherwise noted, Escherichia coli and Serratia sp. ATCC 39006 strains were grown at 37° C. and 30° C., respectively, either in lysogeny broth (LB) at 180 rpm or on LB-agar (LBA) plates containing 1.5% (w/v) agar. Minimal media contained 40 mM K₂HPO₄, 14.6 mM KH₂PO₄, 0.4 mM MgSO₄, 7.6 mM (NH₄)₂SO₄ and 0.2% (w/v) or 2% (w/v) glucose. When applicable, antibiotics and supplements were added at the following concentrations: ampicillin (Ap), 100 μg/mL; chloramphenicol (Cm), 25 μg/mL; kanamycin (Km), 50 μg/mL; gentamicin (Gm), 15 mg/ml; tetracycline (Tc), 10 μg/mL; δ-aminolevulinic acid (ALA), 50 μg/mL; isopropyl β-D-1-thiogalactopyranoside (IPTG), 50 UM; D-glucose (glu), 0.5% (w/v); L-arabinose (ara), 0.1% (w/v). Bacterial growth was measured as the optical density at 600 nm (OD₆₀₀) using a Jenway 6300 Spectrophotometer.

### DNA Isolation and Manipulation

Oligonucleotides used in this study are listed in Table 5. Plasmid DNA was extracted from overnight cultures using the Zyppy Plasmid Miniprep Kit (Zymo Research) and confirmed by DNA sequencing. Plasmids and their construction details are listed in Table 6. Restriction digests, ligations and E. coli transformations were performed using standard techniques. DNA from PCRs and agarose gels was purified using the Illustra GFX PCR DNA and Gel Band Purification Kit (GE Healthcare). Polymerases, restriction enzymes and T4 ligase were obtained from New England Biolabs or Thermo Fisher Scientific.

### Multiple Sequence Alignment of NucC Homologs

Multiple Sequence Alignment (MUSCLE) were performed with the NucC protein sequences from Serratia Type III-A CRISPR-Cas system, Vibrio metoecus sp. RC341 Type III-B CRISPR-Cas, E. coli MS115-1 CBASS system and P. aeruginosa ATCC27853 CBASS system using Geneious Prime® 2022.1.1, with a Score Matrix of Blosum62 and Threshold of 1.

### NucC Cloning for Protein Expression

The DNA sequence for NucC was amplified by a standard PCR protocol and cloned into pML-1M vector (Addgene 29653) using ligation-independent cloning, obtaining a construct with an N-terminal hexa-histidine tag followed by a TEV cleavage site.

### Protein Expression

For expression of NucC, the plasmid was transformed into Escherichia coli BL21 Star (DE3) and cells were grown in LB+Km to an OD of 0.6. Expression was induced with 0.5 mM IPTG and proteins were expressed for 16 h at 18° C.

### Protein Purification for Nuclease Assays

Cells were harvested and resuspended in lysis buffer (20 mM HEPES pH 7.5, 250 mM KCl, 5% glycerol and 1 mM dithiothreitol (DTT)). Cells were lysed by ultrasonication and the lysate was clarified by centrifugation at 20,000×g for 20 min. The cleared lysate was applied to a 5 mL Ni-NTA cartridge (Qiagen). The column was washed with 3 column volumes of lysis buffer and proteins were eluted stepwise with lysis buffer supplemented with 50 and 250 mM of imidazole. The fractions eluted with 250 mM imidazole were pooled and diluted to a final concentration of 50 mM Imidazole. TEV was added in a 1:50 ratio to allow tag cleavage overnight. The cleavage products were passed through a 5 mL Ni-NTA cartridge (Qiagen) and the column was washed with 5 column volumes of lysis buffer supplemented with 50 mM Imidazole to remove the cleaved tag and the TEV protease. The flow-through and the wash fractions were pooled, concentrated using a 10000 molecular weight cut-off centrifugal filters (Merk Millipore) to a final volume 2 mL and loaded onto a S200 16/600 size-exclusion chromatography column (GE Healthcare) in 20 mM HEPES pH 7.5, 250 mM KCl, 5% glycerol and 1 mM DTT. Purified protein was flash-frozen in liquid nitrogen.

### In Vitro Nuclease Assay

Unless otherwise noted, nucleic acids (˜100 ng) were incubated with 100 nM NucC, 200 nM cA₃ and 10 mM MgCl₂, and supplemented with 10 mM HEPES pH 7.5, 100 mM KCl, 5% glycerol and 1 mM DTT. The total reaction volumes were 8 UL and were incubated at 30° C. for 30 min. The samples were loaded on a 1.2% agarose gel and run for 40 min at 120 V.

### Visualization of Degraded DNA Upon Phage Infection

LacA and ΔnucC (PCF686) harbouring either a non-targeting plasmid (pPF976) or a plasmid with a PCH45 targeting spacer (pPF1467) were grown overnight in 5 mL LB+Km (50 μg/mL) and 100 UM IPTG (for spacer induction) at 30° C. with shaking at 180 RPM. The following day, strains were subcultured to a starting OD₆₀₀=0.05 in 25 mL LB+Km (50 μg/mL) and 100 UM IPTG. Cells were grown approximately 4 h until reaching an OD₆₀₀=0.3. One mL of each culture was removed for gDNA extraction as a pre-infection (time 0) control. Ten mL of each culture was then removed to a universal, and phage PCH45 was added to an MOI=10. Cultures were incubated at 30° C. with 180 RPM shaking and samples were taken at the following time points: 20, 40, 60, 80, and 100 min-post infections. At each time point, 1 mL of culture was removed and pelleted at 17,000×g for 1 min. Supernatant was removed and pellets were washed twice with 1 mL PBS. DNA was then extracted using the DNeasy Blood & Tissue kit (Qiagen) per the manufacturer's instruction. Briefly, each pellet was resuspended in 180 μl Buffer ATL with 20 μl Proteinase K and incubated at 56° C. for 30 min. Following incubation, 4 μl RNase A (10 mg/mL) was added to each tube and incubated at RT for 5 min before proceeding with DNeasy procedure. Purified DNA was eluted with 30 μl TE buffer. Sample concentration was measured using a NanoDrop spectrophotometer and then diluted to a concentration of 20 ng/μl. For each sample, 500 ng was loaded onto a 1% agarose gel made up in TAE buffer and run for 40 min at 100 V.

Isolation of gDNA Degradation Products During Phage Infection

Triplicate overnight cultures of WT harbouring either a non-targeting plasmid (pPF976) or a plasmid with a PCH45 targeting spacer (pPF1467) were grown overnight, subcultured, infected, and grown as above. At each time point, (pre-infection, and 20, 40, 60, 80, and 100 min-post-infection), 1.5 mL of culture was removed and pelleted at 17,000×g for 1 min. Cells were washed and DNA was extracted as described above. DNA was eluted in 50 μl TE buffer. To separate intact genomic fragments from degraded DNA, a right-sided size selection was performed using SPRIselect beads (Beckman Coulter) with a 20 μl elution from the first bead addition (0.6× concentration) to recover genomic fragments, and a 35 μl elution from the second bead addition (1.2× concentration) to recover degraded fragments. To remove any carryover of intact genomic DNA, degraded fragments were further purified with a Pippin Prep (Sage Science) using Range Mode to isolate DNA (100-400 bp) from a 2% agarose gel with EtBr staining. DNA eluted from the Pippin Prep was further cleaned and concentrated using SPRIselect left-sided size selection (2× concentration). DNA was eluted into 18 μl TE buffer and quantified using the Qubit dsDNA HS Assay kit (Thermo Fisher Scientific). DNA isolated from LacA with CRISPR targeting (pPF1467) at 40 min (n=3) and 60 min (n=3) post PCH45 phage infection was used to generate sequencing libraries. These time points were chosen as DNA degradation became visible 40 min-post infection (FIG. 4I).

### Isolation of In Vitro Degraded Plasmid DNA

DNA was degraded as in NucC nuclease assay described above for 30 min. Degraded fragments were isolated using the Pippin Prep and then concentrated using SPRIselect left-sided size selection (2× concentration) as described above. This in vitro degraded DNA was then used to generate DNA sequencing libraries.

### DNA Library Preparation and Sequencing

DNA sequencing libraries were prepared using the Accel-NGS 1S Plus DNA Library Kit (Swift Biosciences) according to the manufacturer's instructions. Because samples were degraded either in vivo (phage infection samples) or in vitro (pPF1043 plasmid degradation), no DNA shearing was performed. The input DNA for each library was between 20-50 ng, and 8 cycles of indexing PCR was performed using the Accel-NGS 1s Unique Dual Indexing Kit. Final libraries were eluted in TE Buffer (Low EDTA-Swift Biosciences), quantified using the Qubit dsDNA HS Assay kit (Thermo Fisher Scientific) and fragment size distribution was determined using a Bioanalyzer High Sensitivity DNA Chip (Agilent). Libraries were diluted to 10 nM and pooled in equal ratios. The pool was then sequenced at Otago Genomics Facility (OGF) using a MiSeq Reagent kit v3 (150 cycle) to generate 2×75 bp paired end reads. Demultiplexing based on index and fastq file generation was performed by OGF as part of the Illumina MiSeq Local Run Manager standard workflow. Approximately 27 million clusters (91.7%) passed filter with an average quality score of 36.8.

### Sequencing Data QC, Read Mapping and Coverage Estimation

Fastq file quality was assessed using FastQC (Andrews, 2010). The first 15 nt of Read 2 were trimmed using cutadapt (Martin, 2011) (−u 15) to remove the low complexity tail added as part of the Accel-NGS 1S Plus DNA Library Kit workflow. Reads were also filtered (−m 61) to discard those <61 nt. FastQC was re-run on trimmed samples to ensure tail removal. Reads were then mapped to the reference genome(s) using bowtie2 (Langmead and Salzberg, 2012) default parameters, specifying paired-mate mapping (−−no-mixed). For in vivo degradation libraries, reads were mapped to a combined reference (LacA, PCH45 and pPF1467) built using bowtie2-build. For the in vitro degradation sample, reads were mapped to a single reference (pPF1043) built using bowtie2-build. Following mapping, SAM files were converted to BAM files using SAMtools (Li et al., 2009). Average and per-base coverage was calculated (for each reference) from indexed BAM files using mosdepth (Pedersen and Quinlan, 2018).

### NucC Cleavage Site Preference Search

To generate a list of sequences to search for NucC cleavage site preference, 20 nt surrounding the first mapped base of Read 1 (9 bases upstream and 10 bases downstream of the 5′ end) were extracted as FASTA files from BAM alignments using BEDtools (Quinlan and Hall, 2010). Only Read 1 was used in the analysis, as the 5′ end of Read 2 contains a variable length low-complexity tag (introduced by the Accel-NGS 1S Plus DNA Library Kit workflow) which required trimming. Therefore, the potential cleavage position in Read 2 is ambiguous. FASTA files were then used to search for a motif for potential NucC recognition or cleavage site preferences using WebLogo (Crooks et al., 2004), where the full set of available sequences was used.

### NucC Localization Microscopy

To visualize NucC localization in LacA cells, an N-terminal NucC-mEGFP fusion was expressed under control of ParaBAD (ara-inducible). Cells harbouring the NucC-mEGFP expression plasmid (pPF2290) harboured a second plasmid, containing either a protospacer matching phage PCH45 (pPF1467) or a control plasmid (pPF976). Overnight cultures grown in LB+Km+Gm were used to seed new 25 ml cultures in 125 mL flasks at starting OD₆₀₀=0.05. Cells were grown in LB+1% ara (w/v) for NucC-mEGFP induction, 100 μM IPTG for protospacer induction, and Km+Gm for plasmid maintenance. Cultures were grown at 30° C. with 180 RPM shaking for ˜3.5 h until reaching exponential phase (OD₆₀₀=0.3). Cultures were then split into 5 mL aliquots in glass universals. For +phage treatments, phage PCH45 (˜1×10¹¹ PFU/mL) was added at an MOI of 50. To -phage treatments, an equivalent volume of phage buffer was added. Infected and non-infected cultures were grown for 50 min at 30° C. with 180 RPM shaking. Following growth, 1.5 mL of each culture was removed to a 1.5 ml microcentrifuge tube and centrifuged at 17,000×g to pellet cells. Each pellet was washed 2× with 500 μl minimal media. Pellets were resuspended in 34 μl minimal media, and 16 μl of stain mix (4,6-diamidino-2-phenylindole (DAPI; final 4 μg/mL) and FM 4-64 (final 12 μg/mL)) was added to each sample. Samples were incubated at RT protected from light for 5 min, then centrifuged for 30 s at 17,000×g. Supernatant was removed, pellets washed with 500 μl minimal media centrifuged for 30 s at 17,000×g. Supernatant was removed, and pellets resuspended in 50 μl minimal media. To prepare samples for imaging, 15 μl of cells was mixed with 15 μl of molten 1.2% agar (in minimal media) on a microscope slide and sealed with a coverslip. Images were acquired as previously described (Malone et al., 2020). Briefly, images were acquired using a CFI Plan APO Lambda 100×1.49 numerical aperture oil objective (Nikon Corporation) on the multimodal imaging platform Dragonfly v.505 (Oxford Instruments). Data were collected in Spinning Disk 40 μm pinhole mode on the iXon888 EMCCD camera with 2× optical magnification using the Fusion Studio v.1.4 software. Z stacks were collected in 0.1 μm increments on the z axis using an Applied Scientific Instrumentation stage with 500 μm piezo z drive. Images were visualized and cropped using Fiji software (Windows 64-bit) and further processed using the Huygens Essential Deconvolution Wizard (Scientific Volume Imaging). Final composite images and fluorescence plot data were generated using Fiji and graphed using Prism v. 9.2.0 (GraphPad).

TABLE 4
Strains used for NucC experiments.

| | |
|---|---|
| Name | Genotype/Phenotype |

Serratia sp. ATCC 39006

| | |
|---|---|
| LacA | lac EMS mutant, pigmented WT |
| PCF686 | lac EMS mutant, pigmented WT ΔnucC |

Escherichia coli

| | |
|---|---|
| DH5a | cloning strain. F⁻, φ80ΔdlacZM15, Δ(lacZYA-argF)U169, |
| | endA1, recA1, hsdR17 (rK⁻ mK⁺), deoR, thi-1, supE44, λ⁻, |
| | gyrA96, relA1 |
| ST18 | auxotrophic donor for biparental conjugation. S17-1 λpir |
| | ΔhemA |
| BL21(DE3) | protein expression strain. Str., B, F⁻, ompT, gal, dcm, lon, |
| | hsdS_{B}(r_{B}⁻ m_{B}⁻), λ(DE3, [lacI, lacUV5-T7p07, ind1, sam7, |
| | nin5]), [malB⁺]_{K-12}(λ^{S}) |

Bacteriophages

| | |
|---|---|
| PCH45 | lytic jumbo phage, family Myoviridae; infects Serratia sp ATCC |
| | 39006 |

TABLE 5
Oligonucleotides used for NucC experiments

| | | | |
|---|---|---|---|
| | | SEQ ID | |
| Name | Sequence (5′-3′) | NO | Description |

Cloning

| | | | |
|---|---|---|---|
| pCGD414- | TACTTCCAATCCAATGCAatgactaa | 39 | fwd primer for cloning Serratia |
| Fwd | tcaggcaaaaaa | | nucC into expression vector, |
| | | | generating pCGD414 |
| pCGD414- | TTATCCACTTCCAATGTTAttattcca | 40 | rev primer for cloning Serratia |
| Rev | gactatctatat | | nucC into expression vector, |
| | | | generating pCGD414 |
| PF4688 | GCGAATTCGAGCTCGGTACCAAA | 41 | fwd primer for amplification of |
| | GAGGAGAAATTAACTATGGTGAG | | gBlock PF3809, overlap with |
| | | | pBAD30 for Gibson assembly |
| | | | (KpnI); pPF2290 cloning |
| PF4689 | CTTTTTTGCCTGATTAGTCATGGA | 42 | rev primer for amplification of |
| | TCCGCCTCCACCG | | gBlock PF3809, overlap with |
| | | | PF4690; pPF2290 cloning |
| PF4690 | AGGGCGGTGGAGGCGGATCCATG | 43 | fwd primer for amplification of |
| | ACTAATCAGGCAAAAAAGTTATC | | Serratia nucC + linker (Glyx5- |
| | | | Ser), overlap with PF4689; |
| | | | pPF2290 cloning |
| PF4691 | CAAAAGGTCATCCACTGCAGTTAT | 44 | rev primer for amplification of |
| | TCCAGACTATCTATATACACCC | | Serratia nucC, overlap with |
| | | | pBAD30 for Gibson assembly |
| | | | (PstI); pPF2290 cloning |
| PF3809 | TCGTCTTCACCTCGAGAAATCAAA | 45 | gBlock template for |
| | GAGGAGAAATTAACTATGGTGAG | | amplification of RBS-mEGFP(no |
| | CAAGGGCGAGGAGCTGTTCACCG | | STOP codon)-linker(Gly5x-Ser); |
| | GGGTGGTGCCCATCCTGGTCGAG | | pPF2290 cloning |
| | CTGGACGGCGACGTAAACGGCCA | | |
| | CAAGTTCAGCGTGTCCGGCGAGG | | |
| | GCGAGGGCGATGCCACCTACGGC | | |
| | AAGCTGACCCTGAAGTTCATCTGC | | |
| | ACCACCGGCAAGCTGCCCGTGCC | | |
| | CTGGCCCACCCTCGTGACCACCCT | | |
| | GACCTACGGCGTGCAGTGCTTCA | | |
| | GCCGCTACCCCGACCACATGAAG | | |
| | CAGCACGACTTCTTCAAGTCCGCC | | |
| | ATGCCCGAAGGCTACGTCCAGGA | | |
| | GCGCACCATCTTCTTCAAGGACGA | | |
| | CGGCAACTACAAGACCCGCGCCG | | |
| | AGGTGAAGTTCGAGGGCGACACC | | |
| | CTGGTGAACCGCATCGAGCTGAA | | |
| | GGGCATCGACTTCAAGGAGGACG | | |
| | GCAACATCCTGGGGCACAAGCTG | | |
| | GAGTACAACTACAACAGCCACAAC | | |
| | GTCTATATCATGGCCGACAAGCA | | |
| | GAAGAACGGCATCAAGGTGAACT | | |
| | TCAAGATCCGCCACAACATCGAG | | |
| | GACGGCAGCGTGCAGCTCGCCGA | | |
| | CCACTACCAGCAGAACACCCCCAT | | |
| | CGGCGACGGCCCCGTGCTGCTGC | | |
| | CCGACAACCACTACCTGAGCACC | | |
| | CAGTCCAAGCTGAGCAAAGACCC | | |
| | CAACGAGAAGCGCGATCACATGG | | |
| | TCCTGCTGGAGTTCGTGACCGCC | | |
| | GCCGGGATCACTCTCGGCATGGA | | |
| | CGAGCTGTACAAGGGCGGTGGAG | | |
| | GCGGATCCCCTGTTGATAGATCCA | | |
| | GTAATGAC | | |
| PF5145 | TATAGAATTCAAAGAGGAGAAATT | 46 | fwd primer for cloning Serratia |
| | AACTATGACTAATCAGGCAAAAAA | | nucC + artificial RBS into |
| | GT | | pPF1618, generating pPF2503 |
| | | | (EcoRI) |
| PF5146 | TATAAAGCTTTTATTCCAGACTATC | 47 | rev primer for cloning Serratia |
| | TATATACACCCGCC | | nucC + artificial RBS into |
| | | | pPF1618, generating pPF2503 |
| | | | (HindIII) |
| PF73 | GACTCTAGACACGTGGAGAAACC | 48 | fwd primer for amplification of a |
| | AAAGCC | | Serratia chromosomic region, |
| | | | generating a 1419 bp product |
| | | | for cleavage assays. Binds XRE |
| | | | family transcriptional regulator |
| | | | CDS |
| PF807 | GATCCCGGGTCAGTTCCTTGCCGT | 49 | rev primer for amplification of a |
| | AGC | | Serratia chromosomic region, |
| | | | generating a 1419 bp product |
| | | | for cleavage assays. Binds |
| | | | DUF165 domain-containing |
| | | | protein CDS |
| PF5539 | CCAGATAAATGCAGTGATTTTTG | 50 | fwd primer for site-directed |
| | | | mutagenesis of Serratia nucC |
| | | | active site (D83N) in pPF2513, |
| | | | generating pPF2669 |
| PF5540 | TGCATTTATCTGGTCGCTG | 51 | rev primer for site-directed |
| | | | mutagenesis of Serratia nucC |
| | | | active site (D83N) in pPF2513, |
| | | | generating pPF2669 |
| PF5541 | GTACTGAATGTTAAACCAACCAT | 52 | fwd primer for site-directed |
| | | | mutagenesis of Serratia nucC |
| | | | active site (E114N) in pPF2513, |
| | | | generating pPF2671 |
| PF5542 | TTAACATTCAGTACCGCGTAC | 53 | rev primer for site-directed |
| | | | mutagenesis of Serratia nucC |
| | | | active site (E114N) in pPF2513, |
| | | | generating pPF2671 |
| PF5543 | GGTTCTTCCAACCATTAATAAAAC | 54 | fwd primer for site-directed |
| | C | | mutagenesis of Serratia nucC |
| | | | active site (K116L) in pPF2513, |
| | | | generating pPF2673 |
| PF5544 | GTTGGAAGAACCTCCAGTA | 55 | rev primer for site-directed |
| | | | mutagenesis of Serratia nucC |
| | | | active site (K116L) in pPF2513, |
| | | | generating pPF2673 |

NucC Cleavage Assays

| | | | |
|---|---|---|---|
| PF6283 | CCCTACGCTCCCTCCAGCGCTGTC | 56 | no motif negative control gBlock |
| | GGGGATATAGTCACTCGGAGTTA | | |
| | GAGAGTTTTAGGATTGATTACTGA | | |
| | ACTCTAGTATGGTAAACTGTGAAA | | |
| | ACTCATAAAGCTGACGAAGTAAAA | | |
| | GAATCAAACTAATAACTCAATCCA | | |
| | GTCTAAAGAGTAGAAAGTTGGTG | | |
| | AAAGATTGTGAGTCAGTCACTTAA | | |
| | TGGTCTTAGA | | |
| PF6284 | CCCTACGCTCCCTCCAGCGCTGTC | 57 | full motif gBlock |
| | GGGGATATAGTCACTCGGCAAGG | | |
| | GCGCCCTTGAGGATTGATTACTGA | | |
| | ACTCTAGTATGGTAAACTGTGAAA | | |
| | ACTCATAAAGCTGACGAAGTAAAA | | |
| | GAATCAAACTAATAACTCAATCCA | | |
| | GTCTAAAGAGTAGAAAGTTGGTG | | |
| | AAAGATTGTGAGTCAGTCACTTAA | | |
| | TGGTCTTAGA | | |
| PF6285 | CCCTACGCTCCCTCCAGCGCTGTC | 58 | core motif gBlock |
| | GGGGATATAGTCACTCGGAGTTG | | |
| | GCGCCTTTTAGGATTGATTACTGA | | |
| | ACTCTAGTATGGTAAACTGTGAAA | | |
| | ACTCATAAAGCTGACGAAGTAAAA | | |
| | GAATCAAACTAATAACTCAATCCA | | |
| | GTCTAAAGAGTAGAAAGTTGGTG | | |
| | AAAGATTGTGAGTCAGTCACTTAA | | |
| | TGGTCTTAGA | | |

Screening

| | | | |
|---|---|---|---|
| PF2202 | TATTGCATGCGGCTGACGATCTG | 59 | chromosomal Serratia nucC, |
| | GCGTC | | fwd primer |
| PF2199 | TCTTGGATCCGCTAGCGGCCTGC | 60 | chromosomal Serratia nucC, rev |
| | CGGAGAAC | | primer |
| PF138 | CACACTTTGCTATGCCATAG | 61 | pPF781-derived plasmids, fwd |
| | | | primer |
| PF1702 | CGAAGACGAAAGGGCCTCGTGAT | 62 | pPF781-derived plasmids, rev |
| | ACGCAAGCTTTATGGCTTGTAAAC | | primer |
| | CGTTTTGTG | | |
| PF4181 | AAAGAAATCATAAAAAATTTATTT | 63 | pPF976-derived plasmids, fwd |
| | GCTTTGTGAGCGGAT | | primer |
| PF3737 | TTTATGCATCTTCAGTCAGGGAGC | 64 | pPF976-derived plasmids, rev |
| | GTC | | primer |
| PF2231 | TTTTACTAGTAGACGTTCAACAAC | 65 | PCH45 capsid gene, fwd primer |
| | GTCATG | | |
| PF2232 | TTTTGGTACCGAAGTTATATTCGC | 66 | PCH45 capsid gene, rev primer |
| | GCGGTG | | |
| PF138 | CACACTTTGCTATGCCATAG | 67 | pPF1618-derived plasmids, fwd |
| | | | primer |
| PF210 | GTCATTACTGGATCTATCAACAGG | 68 | pPF1618-derived plasmids, rev |
| | | | primer |

TABLE 6
Plasmids used for NucC experiments

| Name | Description | Features | Construction |
|---|---|---|---|
| Protein Expression and Purification | | | |
| pPF2007 | template for | pBR322/ori, RP4/oriT, | pQE80I-oriT stuffer derivative |
| | nucC cloning into | ApR, lacI/T5 | |
| | expression | | |
| | vector | | |
| pCGD414 | 6xHis-TEV-NucC | ColE1/ori, f1/ori, KmR, | pCGD414-Fwd + pCGD414-Rev paired |
| | expression | lacI/T7 | in a PCR to amplify nucC from pPF2007 |
| | vector | | and clone it into an expression vector |
| | | | through Ligation Independent Cloning |
| pPF2669 | 6xHis-TEV-NucC | ColE1/ori, f1/ori, KmR, | PF5539 + PF5540 paired in a PCR to |
| | D83N mutant | lacI/T7 | introduce NucC D83N mutation in |
| | expression | | pPF2513 expression vector (7225 bp) |
| | vector | | |
| pPF2671 | 6xHis-TEV-NucC | ColE1/ori, f1/ori, KmR, | PF5541 + PF5542 paired in a PCR to |
| | E114N mutant | lacI/T7 | introduce NucC E114N mutation in |
| | expression | | pPF2513 expression vector (7225 bp) |
| | vector | | |
| pPF2673 | 6xHis-TEV-NucC | ColE1/ori, f1/ori, KmR, | PF5543 + PF5544 paired in a PCR to |
| | K116L mutant | lacI/T7 | introduce NucC K116L mutation in |
| | expression | | pPF2513 expression vector (7225 bp) |
| | vector | | |
| Plasmid Targeting | | | |
| pPF781 | untargeted | p15A/ori, RP4/oriT, | pBAD30 derivative |
| | control for the | CmR, pBAD/araC | |
| | Type III-A | | |
| | system | | |
| pPF1043 | targeted Type | p15A/ori, RP4/oriT, | pPF781 derivative |
| | III-A with | CmR, pBAD/araC | |
| | protospacer | | |
| | complementary | | |
| | to Serratia | | |
| | CRISPR3 spacer | | |
| | 1 | | |
| Phage Targeting | | | |
| pPF976 | Type III-A | pBR322/ori, RP4/oriT, | pMAT16 derivative |
| | repeat-BsaI- | KmR, lacI/T5 | |
| | repeat construct | | |
| | for artificial | | |
| | crRNA | | |
| pPF1467 | anti-PCH45 III-A | pBR322/ori, RP4/oriT, | pPF976 derivative |
| | spacer | KmR, lacI/T5 | |
| | overexpression. | | |
| | III- | | |
| | A_PCH45_PS4 | | |
| | (capsid protein) | | |
| pPF1477 | anti-JS26 III-A | pBR322/ori, RP4/oriT, | pPF976 derivative |
| | spacer | KmR, lacI/T5 | |
| | overexpression. | | |
| | III-A_JS26_PS8 | | |
| | (capsid protein) | | |
| NucC Localization | | | |
| Microscopy | | | |
| pPF2290 | mEGFP-NucC | p15A/ori, RP4/oriT, | Gibson assembly PF4688/PF4689 |
| | expression | GmR, pBAD/araC | (gblockPF3809) + PF4690/PF4691 |
| | vector | | (LacA) |

### Results

Serratia NucC Forms a Hexamer that Binds cA₃

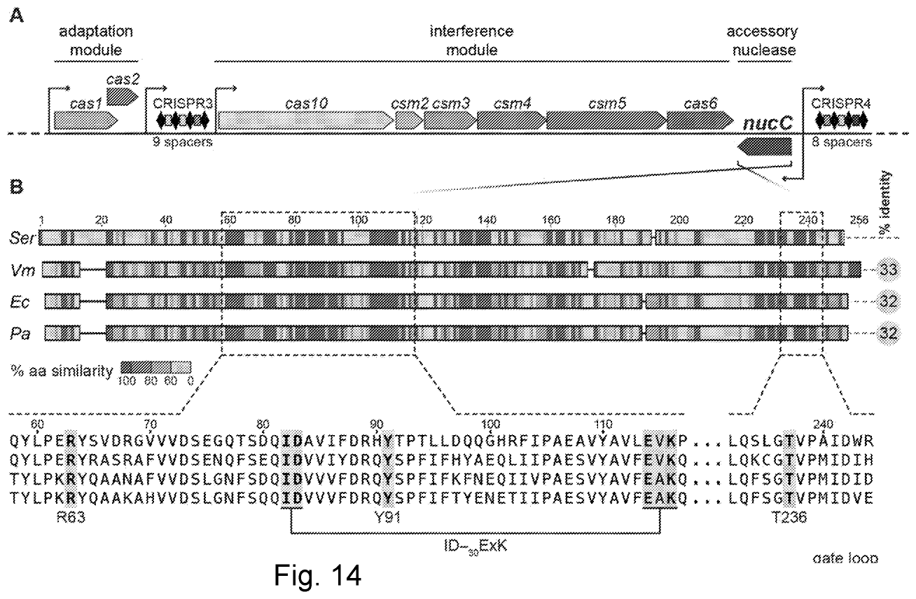

Since resistance against jumbo phage PCH45 required a Serratia Type III-A accessory gene with homology to the NucC nuclease (Malone et al., 2020), we explored its mechanism as part of CRISPR-Cas immunity. Serratia NucC contains 250 amino acids (28.14 kDa) and shares <35% sequence identity to recently characterized NucC proteins from CBASS and a Type III-B CRISPR-Cas system (Lau et al., 2020; Ye et al., 2020) (FIG. 14A-B). Despite the low identity, the active site motif of ID-₃₀ExK in these restriction endonuclease-like fold proteins is conserved in NucC (FIG. 14B), suggesting it may also function as an endonuclease. Many Type III accessory nucleases encode proteins containing an N-terminal CARF (CRISPR-Cas Associated Rossmann Fold) domain that binds cOA messengers and activates a variety of C-terminal effector domains (Makarova et al., 2014; Makarova et al., 2020). In contrast, NucC proteins do not have a CARF domain, are active as hexamers and bind cOAs (Lau et al., 2020).

### Serratia NucC is Activated by cA₃ and Degrades Double-Stranded DNA In Vitro

NucC homologues were previously shown to cleave plasmid and synthetic DNA in vitro when activated by cA₃ (Gruschow et al., 2021; Lau et al., 2020). Given the predicted nuclease activity of Serratia NucC and its role in jumbo phage immunity (Malone et al., 2020), we tested its ability to degrade different nucleic acids in vitro in the presence of cA₃. NucC degraded dsDNA when incubated with cA₃ (FIG. 15). NucC activity was dependent on Mg²⁺ (FIG. 15A-D), which is coordinated by the conserved acidic residues E46, D83 and E114. Notably, NucC degraded both Serratia and jumbo phage (PCH45) genomic DNA (gDNA) upon activation by cA₃, resulting in a smear of smaller DNA products on the gels (FIG. 15A-B). DNA degradation by NucC was dependent on NucC and cA₃ concentration and was imitated within one minute. Mutation of predicted key nuclease active site residues (D83N, E114N and K116L) abolished the DNase activity (FIGS. 14B and 15A). Moreover, NucC was active against both supercoiled plasmid DNA and a linear PCR product (FIG. 15C-D).

The PCR product degradation pattern (FIG. 15D) suggested that NucC might have preferred cleavage site(s). To examine sequence-specificity, we incubated a plasmid with NucC and deep sequenced the resulting short fragments. The κ′-end mapping of the sequencing reads showed a heterogenous distribution of DNA degradation products (FIG. 15E). Alignment of reads in their 5′-end revealed a variable palindromic cleavage site (consensus: CAnnGGCGCCnnTG (SEQ ID NO: 69)), suggesting a model of double-strand cleavage where two NucC-active sites cooperate to cleave both DNA strands (FIG. 15F). To verify the NucC cleavage site directly, in vitro cleavage assays were performed with 200 bp dsDNA fragments that contained the preferred cleavage motif with either the core (nucleotide positions 7-12) or full motif (nucleotide positions 3-16) (FIG. 15G). Cleavage at the predicted site would generate 50 and 150 bp fragments. NucC specifically cleaved dsDNA containing the full predicted motif sequence and only when activated by cA₃ (FIG. 15H). The presence of diversity within the predicted NucC motif indicates it cleaves additional sequences (FIG. 15F). The outermost positions of the full motif (positions 3 and 16) have a preference for pyrimidine: purine (C/T: G/A) pairing, with the same top four pairs (of 16 possible pairings) accounting for 51% of the sequence diversity at those positions. Positions 4 and 15 also had similar conservation of preferred nucleotides, but without a preference for pyrimidines or purines. Supporting the importance of these outer nucleotides for NucC DNA binding and/or cleavage, alteration of these outer residues (positions 3-6 and 13-16), while leaving the core residues (GGCGCC, positions 7-12 (SEQ ID NO:37)) intact, abrogated specific cleavage (FIG. 15H). Together, the NucC hexamer is activated by cA₃ and cleaves double-strand DNA with some sequence-specificity.

### The Jumbo Phage DNA-Containing Nucleus Excludes NucC

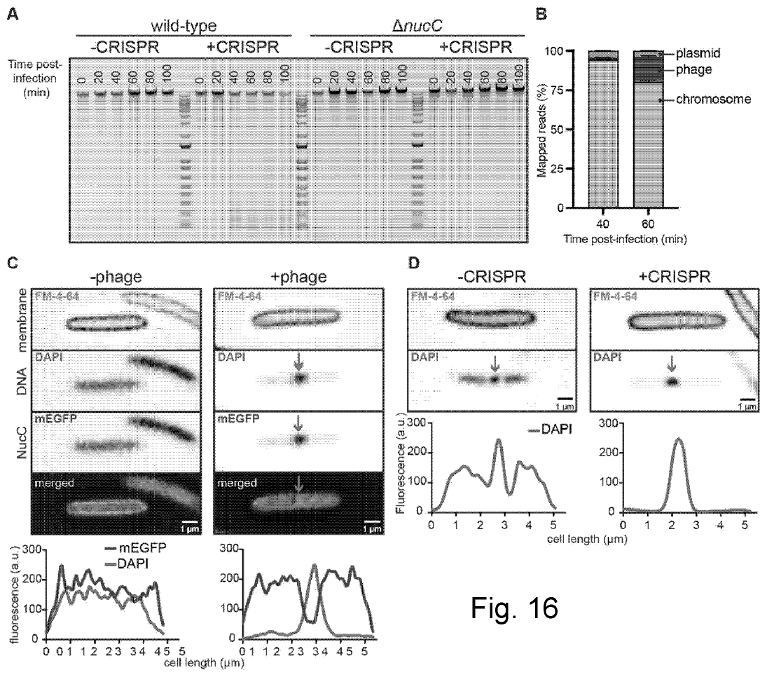

We hypothesised that Type III immunity against jumbo phage infection was provided by NucC-mediated degradation of the bacterial genome and that NucC was unable to access the phage DNA protected in the nucleus-like structure. To test this, we performed phage infection assays and total DNA was extracted at various times throughout a single round of phage infection. Firstly, we analysed the DNA via gel electrophoresis, which revealed no clear reduction in total DNA in phage-sensitive cells upon jumbo phage infection, indicating that the jumbo phage does not visibly degrade host DNA (FIG. 16A). However, in the presence of phage targeting by the Type III system, high molecular weight DNA decreased, and smaller DNA degradation products became visible 40 minutes after phage addition (FIG. 16A). In contrast, no degradation products were observed in the absence of NucC (FIG. 16A), demonstrating that both Type III targeting and NucC were required for DNA degradation during jumbo phage infection. To determine the precise source (chromosome and/or the jumbo phage genome) of the degradation products we isolated the small DNA fragments for deep sequencing. At 40 min post-infection, reads mapped mainly to the Serratia chromosome and plasmid, but not to the jumbo phage genome (FIG. 16B).

We hypothesized that NucC also could not access the nucleus and degrade the jumbo phage genome. To investigate this, we first generated an mEGFP-tagged NucC expression plasmid and demonstrated that it retained interference activity against the jumbo phage. Next, we studied NucC localisation by confocal microscopy during Type III immunity (FIG. 16C). Upon phage infection, we observed circular DNA foci (blue), consistent with phage DNA accumulation within nucleus-like structures, whereas bacterial DNA was evenly distributed in uninfected controls (FIG. 16C). Importantly, during jumbo phage infection, NucC was localized in the cytoplasm (green), external to the phage DNA-containing nucleus (blue) (FIG. 16C). By contrast, NucC was evenly distributed in the uninfected control (FIG. 16C). We also obtained direct evidence within single cells that Type III and NucC activation leads to bacterial genome degradation since bacterial DNA was undetectable upon phage targeting (+CRISPR) (FIG. 16D). In contrast, bacterial DNA was readily detected in the cytoplasm of phage-infected cells lacking Type III targeting (−CRISPR) (FIG. 16D). In addition, jumbo phage DNA enclosed in the nucleus retained a strong fluorescent signal upon Type III activation, indicating its protection from NucC activity (FIG. 16D). In summary, the viral nucleus block NucC from accessing the jumbo phage DNA, but NucC has access to degrade the bacterial genome, triggering abortive infection and arresting phage replication.

### Example 3: Single Fusion Protein System

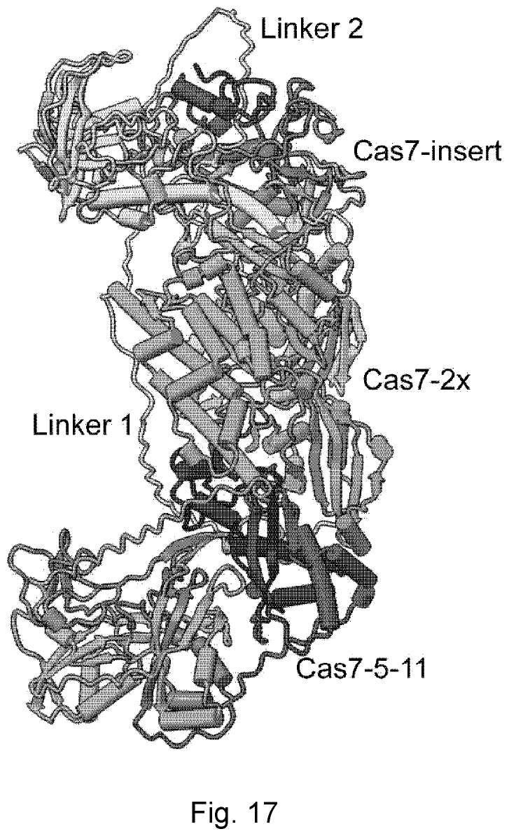

We designed a fusion of several of the Cas protein subunits of a Type III-Dv system, specifically comprising Cas7-5-11, Cas7-2× and Cas7-insert tethered together by two linkers. The amino acid and nucleic acid sequence encoding this fusion protein are set out below (SEQ ID NOs: 28 and 27, respectively). We predicted that this fusion should retain activity. The Alphafold (see, Jumper, J., Evans, R., Pritzel, A. et al. (2021)) predicted structure of this fusion protein is set out in FIGS. 5 and 17. The predicted structure is remarkably similar to the structure solved above. The Cas protein subunits and the linkers are indicated in the figure. This construct includes the removal of the first 113 residues of the Cas7-insert subunit. This 113 residue region was not observed in the structure (possibly due to flexibility) and it has been confirmed separately that this portion can be removed and the effector remains active in cleaving RNA target. It is highly likely that this fusion protein will retain activity. Further Cas proteins can be integrated into this fusion, e.g. the Csx19 or Cas10 subunits, using suitable linkers. The order of subunits in the fusion protein can also be varied.

To exemplify the activity of the single fusion protein, the inventors investigated the ability of the fusion protein to silence gene expression of a fluorescent reporter in HEK293 mammalian cells.

### Materials and Methods

### Construction of Type III-Dv Complex Plasmids for Expression in Mammalian Cells

Vectors used for expression of the single fusion Type III-Dv complex in mammalian cells were synthetically constructed. The cas genes were codon optimized for expression in mammalian cells and ordered as gene-blocks from IDT (Table 11). Gene-blocks were amplified by PCR using the oligonucleotides listed in Table 10. The plasmid was assembled with six gene fragments using a Gibson assembly reaction (NEB). The resulting vector (pPF3612) was confirmed with Oxford nanopore sequencing. Spacers (annealed oligonucleotides in Table 10) were cloned into the entry vector via a BsaI restriction site. Clones were confirmed by Sanger sequencing.

### Cell Culture

Human embryonic kidney cells (HEK293) were cultured in Dulbecco's modified essential medium (DMEM) supplemented with 10% foetal calf serum (FCS; Pan Biotech Aidenbach, Germany) and Pen-Strep (100 U/mL penicillin and 100 μg/mL streptomycin; Gibco) at 37° C. with 5% CO₂. One day prior to transfection, HEK293 cells were seeded into either 12- or 6-well plates at ˜3×10⁵ cells/mL in 10% DMEM without Pen-Strep. HEK293 cells were then transfected with either 1000 or 2500 ng total DNA using Lipofectamine 3000 (Thermo Fisher Scientific, Waltham, MA, USA) as per the manufacturer's protocol. The media was replaced 6-12 hours post-transfection, with 10% FCS/DMEM supplemented with Pen-Strep. Cells were then processed for imaging or flow cytometry 48-hours post-transfection.

### Flow Cytometry

48-hours after transfection, media was removed from cells, they were resuspended in 1 mL wash buffer (PBS pH 7.4, 0.1% w/v BSA, 2 mM EDTA) and then centrifuged at 453×g for 5 min. Cells were washed in this manner in triplicate and then resuspended in 300 μL wash buffer and measured on a LSRFortessa flow cytometer (BD Biosciences) for experiments involving type III-Dv complex and on a Aurora Cytek (Cytek Biosciences) for experiments involving the single fusion type III-Dv complex. Single cell population was selected using FSC and SSC thresholds and then fluorescent intensity of co-transfected cells was determined for Venus (from pPF3328) and the microRFP (from vectors pPF3610 including cloned spacers). For Venus, an excitation wavelength of 488 nm and filter with a bandpass at 530/30 nm was used. For microRFP, a red laser for excitation at 640 nm and a filter with a bandpass at 670/14 nm was used. A total of 50,000 events were recorded for each sample using BD FACSDiva software (v.8, BD Biosciences). Analysis of recorded data was performed using FlowJo software v.10 (BD Biosciences). Cells were gated on SSC-A vs. FSC-A, FSC-H vs. FSC-A and SSC-H vs. SSC-A were used to identify the singlet population of HEK293 cells. Co-transfected singlet cells that were both microRFP and Venus positive had the median fluorescence intensity (MFI) of Venus fluorescence determined. Determined MFIs were plotted and analysed using Prism v. 9.2.0 (Graphpad). Statistical analysis was performed using a one-way ANOVA multiple comparison, comparing treatment with targeting spacers to the non-targeting spacer controls.

### Results

To investigate the activity of a single fusion type III-Dv complex, we tested the ability of the complex to knockdown reporter expression in mammalian cells. The single fusion complex involved subunits Cas7-Cas5-Cas11, Cas7-Cas7 and Cas7-insertion tethered by linkers. The applicants predicted this complex should still bind mRNA and silence expression. Furthermore, the applicants predict the smaller genetic sequences required to express the complex (because cas10 and cas19 are removed) maybe advantageous for packaging in delivery systems for expression in mammalian cells. An entry vector (pPF3612) was constructed through Gibson assembly with gene fragments and confirmed using Oxford Nanopore sequencing. As required, different spacers were added to this entry vector via the BsaI restriction.

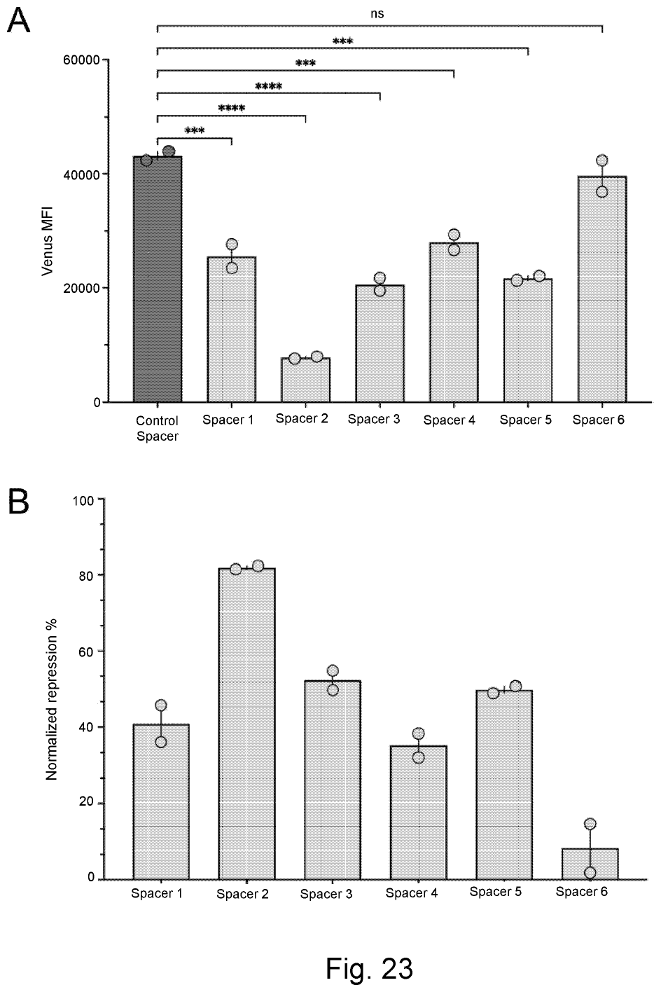

To quantify the knockdown efficiency of single fusion Type III-Dv in mammalian cells, HEK293 cells were co-transfected with a Venus expression plasmid (pPF3328) and single fusion Type III-Dv expression vectors with spacers targeting the kozak and CDS of Venus. FIG. 23 (A) shows five of the six targeting spacers significantly reduced expression of the Venus compared to the non-targeting guide. FIG. 23 (B) presents the data normalized to the control spacer and shows the five spacers repressed the Venus reporter by 40-80%. These data show that the single fusion Type III-Dv complex can effectively target and knockdown gene expression of a fluorescent reporter in mammalian cells. The applicants speculate that the fusion protein is advantageous to full type III complexes (i.e. full type III-Dv complex in Example 5 and the type III-A complex by Colognori et al. 2023) because of a smaller genetic payload and improved assembly of the complex in mammalian cells.

### Example 4: Proposed Uses of the Type III-Dv CRISPR Cas System

The Type III-Dv system can be used for in vitro detection of RNA, or for in vitro or in vivo RNA cleavage.

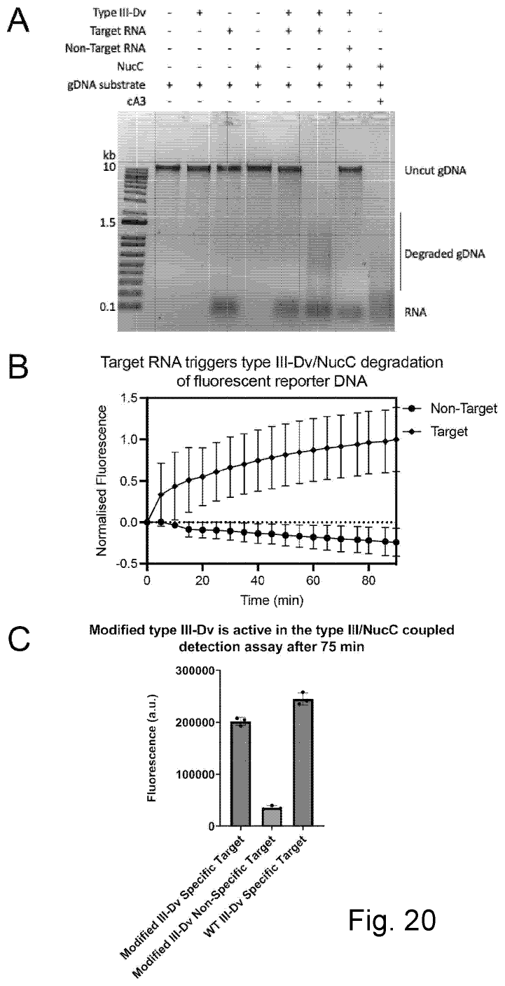

The inventors have shown herein that the Type III-Dv complex can be coupled with a NucC DNase and demonstrated cleavage of substrate DNA reporters (FIG. 20).

To first demonstrate that a coupled type III-Dv/NucC system can detect a specific RNA target and trigger cleavage of a DNA substrate, we detected DNA fragmentation of genomic DNA. Sophisticated screening methods exist for DNA fragmentation analysis including realtime PCR (qPCR), digital PCR (dPCR) and next gen sequencing (NGS), as well as less quantitative measures such as imaging analysis based on COMET testing, or agarose or acrylamide gel electrophoresis and subsequent DNA staining visualisation. All of these tests determine the DNA Fragmentation Index (DFI). Applicants tested whether synthetic induced type III-Dv/NucC DNA cleavage such that a difference in the DFI could be visually detected by standard gel electrophoresis.

FIG. 20 (A) shows an ethidium-bromide stained agarose gel. Only when a specific target RNA was incubated with purified type III-Dv effector complex and purified NucC, was the DNA substrate degraded. Non-target RNA did not trigger DNA fragmentation. The cA₃ molecule could activate NucC to fragment DNA, consistent with the requirement of the type III-Dv complex producing the required signalling molecule. Given the increased DNA degradation is easily visualized by agarose gel electrophoresis with ethidium-bromide staining, it follows that sensitive measures for DNA quantification could be used to produce sensitive outputs.

TABLE 7
Reaction Mix IV

| Component | Concentration in 20 μL | |
|---|---|---|
| Type III-Dv | 450 | nM |
| Buffer¹ | 1X | |
| RNA sample | 10 | μL |
| NucC | 100 | nM |
| Plasmid | 200 | ng |

¹The buffer composition comprises 12.5 mM mM Tris-HCl, pH 8.5, 20 mM NaCl, 20 mM KCl, 10 mM MgCl₂, 5% (v/v) glycerol, 1 mM dithiothreitol, and 500 μM ATP.

In this next example, the applicants used the type III-Dv/NucC system with a short double-stranded DNA probe double labelled with FAM and BlackHole Quencher (IDT) as the reporter. Cleavage of the short dsDNA reporter by NucC leads to liberation of the 6-FAM fluorophore that is otherwise quenched by the proximity of the Iowa Black fluorescent quencher. Fluorescence is then detected and visualised using standard techniques.

### the Reaction Mixture Used is Described in Table 8 Below:

TABLE 8
Reaction Mix I

| Component | Concentration | |
|---|---|---|
| Type III-Dv | 450 | nM |
| Buffer¹ | 1X | |
| RNA sample | 400 | pM |
| NucC | 100 | nM |
| Probe 1 | 150 | nM |

¹The buffer composition comprises 12.5 mM mM Tris-HCI, pH 8.5, 20 mM NaCl, 20 mM KCl, 10 mM MgCl₂, 5% (v/v) glycerol, 1 mM dithiothreitol, and 500 μM ATP.

The reaction was incubated at 30° C. and fluorescence was measured every 5 mins for 90 mins (kinetic readout). The assay was performed in triplicate on a Victor Nivo plate reader (Perkin Elmer) using fluorescence detection (λex/em 485/530 nm) in black 384-well plates.

FIG. 20 (B) shows time-dependent generation of fluorescence output. These data show that the specific target RNA facilitates cleavage of the fluorescent reporter DNA probe (annealed oligonucleotides SEQ ID Nos.: 150 and 151 over time. Moreover, the inventors have demonstrated that RNA detection occurs in a sequence specific manner and can be applied to a high-throughput fluorescent-based reporter setup. Similar methods may be described in Athukoralage et al. 2020; Santiago-Frangos, A., et al. 2021; and Steens, J. A. et al. 2021.

In this next example, the applicants tested modified type III-Dv complex with ablated RNA cleavage activity. The inventors envision that modified Cas7 proteins that do not cleave target RNA would improve the diagnostic sensitivity for detection of RNA. These modified forms of Cas7 are described hereinabove and may be made using known genetic modification techniques in the art.

### The Reaction Mixture Used is Described in Table 9 Below:

TABLE 9
Reaction Mix I

| Component | Concentration | |
|---|---|---|
| Type III-Dv | 240 | nM |
| Buffer¹ | 1X | |
| RNA sample | 33 | nM |
| NucC | 100 | nM |
| Probe 2 | 125 | nM |

¹The buffer composition comprises 12.5 mM mM Tris-HCl, pH 8.5, 20 mM NaCl, 20 mM KCl, 10 mM MgCl₂, 10% (v/v) glycerol, 1 mM dithiothreitol, and 250 μM ATP.

The reaction was incubated at 30° C. and fluorescence was measured after 75 min (endpoint readout). The assay was performed in triplicate on a Victor Nivo plate reader (Perkin Elmer) using fluorescence detection (λex/em 485/530 nm) in black 94-well plates.

FIG. 20 (C) shows modified type III-Dv with inactive Cas7 subunits triggers cleavage to fluorescent reporter DNA probe (annealed oligonucleotides SEQ ID NO 150 and 151). Greater fluorescence, and therefore DNA reporter cleavage, was observed with the specific RNA target compared to the non-specific reporter. The inventors anticipate the modified type III-Dv version would have an improved level of detection at low amounts of RNA sample.

Plasmids comprising nucleic acids encoding for expression of the proteins of the Type III-Dv CRISPR Cas system and the crRNA targeting a gene(s) of interest, together with appropriate expression constructs and components, can be introduced into cells of interest (bacterial, fungal, plant or animal) using transformation techniques known in the art such as electroporation, microinjection, sonication and the like.

Expression of the proteins of the system and the crRNA(s) from said plasmids will lead to the Type III-Dv CRISPR-Cas complex forming in the cell, and binding to and cleaving the target mRNAs in the cell via annealing of the complementary crRNA to the target mRNA sequence. This cleavage could result in specific knockdown of targeted RNAs. Cells or cell populations can then be screened for phenotypes of interest or for the desired knockdown using known techniques in the art. Similar methods may be described in Özcan et al. 2021; or Kato et al 2022.

By using variants or modified forms of the Type III-Dv CRISPR-Cas system that cleave only a single time, which may be produced as explained hereinabove, precise cleavage of an RNA of interest could be achieved.

Using variants that bind RNA but that do not cleave could be used to bind and repress the translation of target RNAs in the manner known as CRISPR interference. In addition, Type III-Dv could be used to block the binding of RNA binding proteins to target RNAs and therefore assess the role of those RNA binding proteins using known techniques in the art.

### Example 5: mRNA Targeting and Repression of a Reporter Gene in HEK293 Cells

### Materials and Methods

### Construction of Type III-Dv Complex Plasmids for Expression in Mammalian Cells

Vectors used for expression of the Type III-Dv complex in mammalian cells were synthetically constructed. The cas genes were codon optimized for expression in mammalian cells and ordered as gene-blocks from IDT (Table 11). Gene-blocks were amplified by PCR using the oligonucleotides listed in Table 10. The plasmid was assembled with eight gene fragments using a Gibson assembly reaction (NEB). The resulting vector (pPF3610) was confirmed with Oxford nanopore sequencing. Spacers (annealed oligonucleotides in Table 10) were cloned into the entry vector via a BsaI restriction site. Clones were confirmed by Sanger sequencing.

### Cell Culture

Human embryonic kidney cells (HEK293) were cultured in Dulbecco's modified essential medium (DMEM) supplemented with 10% foetal calf serum (FCS; Pan Biotech Aidenbach, Germany) and Pen-Strep (100 U/mL penicillin and 100 μg/mL streptomycin; Gibco) at 37° C. with 5% CO₂. One day prior to transfection, HEK293 cells were seeded into either 12- or 6-well plates at ˜3×10⁵ cells/mL in 10% DMEM without Pen-Strep. HEK293 cells were then transfected with either 1000 or 2500 ng total DNA using Lipofectamine 3000 (Thermo Fisher Scientific, Waltham, MA, USA) as per the manufacturer's protocol. The media was replaced 6-12 hours post-transfection, with 10% FCS/DMEM supplemented with Pen-Strep. Cells were then processed for imaging or flow cytometry 48-hours post-transfection.

### Confocal Microscopy

To image transfected HEK293 cells, cells were seeded onto glass coverslips in 12-well plates. After 48-hour of transfection, cells were fixed in 4% paraformaldehyde, then washed twice with PBS pH 7.4 before being stained with Hoechst 33342 (Thermo Fisher Scientific, Waltham, MA, USA) and washed again in PBS pH 7.4 followed by a final wash in distilled water. Coverslips were then mounted onto microscope slides using Fluorsave (Merck Millipore). Images were acquired using a CFI Plan APO Lambda ×100 1.49 numerical aperture oil objective (Nikon Corporation) on the multimodal imaging platform Dragonfly v.505 (Oxford Instruments) equipped with 405, 488, 561 and 637 nm lasers built on a Nikon Ti2-E microscope body with Perfect Focus System (Nikon Corporation). Data was collected in Spinning Disk 40 μm pinhole mode on the iXon888 EMCCD camera with ×2 optical magnification using the Fusion Studio Software v.1.4 (Andor Oxford Instruments). Z stacks were collected with 0.1 μm increments on the z-axis using an Applied Scientific Instrumentation stage with 500 μm piezo z drive. Images were visualized and cropped using Fiji Software (Windows 64-bit). Final composite images and fluorescence plot data were generated using Fiji Software (Windows 64-bit).

### Western Blot Analysis

Cells were transfected with 2500 ng of pPF3610 including spacer 2 targeting Venus (S2). After 48-hours, media was removed, cells were washed once in PBS supplemented with BSA and EDTA prior to being pelleted by centrifugation at 453×g for 5 minutes. Cells were then lysed using RIPA lysis buffer (0.02% azide, 150 mM NaCl, 0.25% CHAPS, 0.5% Triton-X100, 100 mM Tris, pH 8.0 along with freshly added complete protease inhibitor (Roche)). The total protein in the cell lysate was determined by Qubit (Thermo Fisher). A total of 26 μL of protein lysate was separated by Bolt 4-12% Bis-Tris Plus gels (Invitrogen) and transferred onto a Nitrocellulose membrane (Protran, Amersham, Auckland, NZ). Membranes were blocked with 2% skim milk powder/PBS (Sigma) overnight before being stained with mouse monoclonal anti-FLAG (1:1000 dilution) primary antibody for 2 hours. The membrane was then washed and stained with rabbit anti-mouse IgG (1:10,000 dilution) secondary antibody. The membrane was scanned using an Odyssey Fc Imaging System (LI-COR Biosciences, Germany) and was analyzed using Image Studio Lite software.

### Flow Cytometry

48-hours after transfection, media was removed from cells, they were resuspended in 1 mL wash buffer (PBS pH 7.4, 0.1% w/v BSA, 2 mM EDTA) and then centrifuged at 453×g for 5 min. Cells were washed in this manner in triplicate and then resuspended in 300 μL wash buffer and measured on a LSRFortessa flow cytometer (BD Biosciences). Single cell population was selected using FSC and SSC thresholds and then fluorescent intensity of co-transfected cells was determined for Venus (from pPF3328) and the microRFP (from vectors pPF3610 including cloned spacers). For Venus, an excitation wavelength of 488 nm and filter with a bandpass at 530/30 nm was used. For microRFP, a red laser for excitation at 640 nm and a filter with a bandpass at 670/14 nm was used. A total of 50,000 events were recorded for each sample using BD FACSDiva software (v.8, BD Biosciences). Analysis of recorded data was performed using FlowJo software v.10 (BD Biosciences). Cells were gated on SSC-A vs. FSC-A, FSC-H vs. FSC-A and SSC-H vs. SSC-A were used to identify the singlet population of HEK293 cells. Co-transfected singlet cells that were both microRFP and Venus positive had the median fluorescence intensity (MFI) of Venus fluorescence determined. Determined MFIs were plotted and analysed using Prism v. 9.2.0 (Graphpad). Statistical analysis was performed using a one-way ANOVA multiple comparison, comparing treatment with targeting spacers to the non-targeting spacer controls.

TABLE 10
Oligonucleotides used in this study.

| | | | SEQ ID |
|---|---|---|---|
| Name | Sequence (5′-3′) | Notes | NO: |
| PF7106 | TAGTCTAGAGGATCATAATCAGCCATAC | F ori and III-Dv | 148 |
| | | repeat | |
| PF7107 | TAATACGGTTATCCACAGAATCAGG | R ori and III-Dv | 149 |
| | | repeat | |
| PF7155 | GCCAACGCCAATCACAAGAACCAGGGCGA | F amplify gene | 98 |
| | GGAAGGCAGAGGAAGCCTACTTAC | downstream | |
| | | hCas10(III-Dv) | |
| PF7156 | CTCGCCCTGGTTCTTGTGATTG | R hCas10(III-Dv) | 99 |
| PF7157 | GAGAGCAACCAGCAGTCTCAAGGAGCCGC | F amplify gene | 100 |
| | TGAAGGCAGAGGAAGCCTACTG | downstream hCas7- | |
| | | 5-11(III-Dv) | |
| PF7158 | AGCGGCTCCTTGAGACTGC | R hCas7-5-11(III- | 101 |
| | | Dv) | |
| PF7159 | CCTATCGACCTGTGCCAACAGGAAGCTGC | F amplify gene | 102 |
| | TGAAGGCAGAGGAAGCCTACTG | downstream hCas7- | |
| | | 7(III-Dv) | |
| PF7160 | AGCAGCTTCCTGTTGGCAC | R hCas7-7(III-Dv) | 103 |
| PF7161 | GACGAGCGGCTGATCAAGCTGGAAGTGAA | F amplify gene | 104 |
| | GGAAGGCAGAGGAAGCCTACTG | downstream | |
| | | hCsx19(III-Dv) | |
| PF7162 | CTTCACTTCCAGCTTGATCAGCC | R hCsx19(III-Dv) | 105 |
| PF7163 | TGGTATGGCTGATTATGATCCTCTAGACTA | R hCas7-insertion, | 106 |
| | ACTAGGCTTGATAGGGAAAGACTGG | with polyA overhang | |
| | | for cloning upstream | |
| | | pU6 | |
| PF7164 | AGATCCGCTAGGGATCCGCCGCCACCATG | F hCas 10d, adjacent | 107 |
| | GCTCACCATCACCATCATCACAG | CMV, minus 2A site | |
| | | with 30nt overhang | |
| PF7165 | CTGGAGAATTCACCGGTGCCGCCACCATG | F hMicroRFP, | 108 |
| | GCCAATCTGGATAAGATGCTGAACAC | adjacent CMV, 30 nt | |
| | | overhang | |
| PF7166 | TATCCCCTGATTCTGTGGATAACCGTATTA | R polyA, with Pu6 | 109 |
| | CGGCAGTGAAAAAAATGCTTTATTTG | overhang, 30 nt. | |
| PF7167 | TGGTATGGCTGATTATGATCCTCTAGACTA | R Csx19 onto Pu6 | 110 |
| | CTTCACTTCCAGCTTGATCAGCC | | |
| PF7168 | CGGGCAAAGAGAGCCCTGGCTAACGTGCA | F amplify gene | 111 |
| | GGAAGGCAGAGGAAGCCTAC | downstream of | |
| | | Cas6-2a (III-Dv) | |
| PF7169 | AGATCCGCTAGGGATCCGCCGCCACCATG | F FLAG-NLS, | 112 |
| | GCTGATTACAAGGATGACGATGACAAGAT | adjacent CMV, for | |
| | GG | single-effector with | |
| | | 30 nt overhang | |
| PF7170 | AGCGGCTCCCTGGCTCTGCTGGTTGCTCT | R Cas7-5-11 for | 154 |
| | CGTTCTC | single effector | |
| PF7171 | GAGAGCAACCAGCAGAGCCAGGGAGCCG | F Cas7-7 for single | 155 |
| | | effector | |
| PF7172 | CGACTCCCAGCGTTCCCACGGTC | R Cas7-7 for single | 156 |
| | | effector | |
| PF7173 | AGAAGATGACCGTGGGAACGCTG | F Cas7-insertion for | 157 |
| | | single effector | |
| PF7204 | aaacCTTCTCCTTTAGACACCATGGTGGCG | F Mammalian III-Dv | 113 |
| | ACCGGTAGCGGTTCAACACCCTCTTTTC | Spacer (target | |
| | CCCGTCAGGGGACTG | venus kozak 1) + | |
| | | Repeat, clone into | |
| | | BsaI | |
| PF7205 | gtttCAGTCCCCTGACGGGGAAAAGAGG | R Mammalian III-Dv | 114 |
| | GTGTTGAACCGCTACCGGTCGCCACCATG | Spacer (target | |
| | GTGTCTAAAGGAGAAG | venus kozak 1) + | |
| | | Repeat, clone into | |
| | | BsaI | |
| PF7206 | aaacGCTTCATATGGTCTGGATATCTGGCA | F Mammalian III-Dv | 115 |
| | AAACACTGGAGTTCAACACCCTCTTTTCC | Spacer (target | |
| | CCGTCAGGGGACTG | venus cds 2) + | |
| | | Repeat, clone into | |
| | | BsaI | |
| PF7207 | gtttCAGTCCCCTGACGGGGAAAAGAGG | R Mammalian III-Dv | 116 |
| | GTGTTGAACTCCAGTGTTTTGCCAGATAT | Spacer (target | |
| | CCAGACCATATGAAGC | venus cds 2) + | |
| | | Repeat, clone into | |
| | | BsaI | |
| PF7208 | aaacGTTGTACCCCACCATCTTCAATGTTAT | F Mammalian III-Dv | 117 |
| | GGCGTATTTGTTCAACACCCTCTTTTCCC | Spacer (target | |
| | CGTCAGGGGACTG | venus cds 3) + | |
| | | Repeat, clone into | |
| | | BsaI | |
| PF7209 | gtttCAGTCCCCTGACGGGGAAAAGAGG | R Mammalian III-Dv | 118 |
| | GTGTTGAACAAATACGCCATAACATTGAA | Spacer (target | |
| | GATGGTGGGGTACAAC | venus cds 3) + | |
| | | Repeat, clone into | |
| | | BsaI | |
| PF7210 | aaacGCATATCAGCTTCAACGTGAGTTTGC | F Mammalian III-Dv | 119 |
| | CATAGGTGGCGTTCAACACCCTCTTTTCC | Spacer (target | |
| | CCGTCAGGGGACTG | venus cds 4) + | |
| | | Repeat, clone into | |
| | | BsaI | |
| PF7211 | gtttCAGTCCCCTGACGGGGAAAAGAGG | R Mammalian III-Dv | 120 |
| | GTGTTGAACGCCACCTATGGCAAACTCAC | Spacer (target | |
| | GTTGAAGCTGATATGC | venus cds 4) + | |
| | | Repeat, clone into | |
| | | BsaI | |
| PF7301 | aaacGAGGATTACACGGCATAGGTCAGCCT | F Mammalian III-Dv | 121 |
| | AAGTCATCGAGTTCAACACCCTCTTTTCC | Spacer (target | |
| | CCGTCAGGGGACTG | random control 1) + | |
| | | Repeat, clone into | |
| | | BsaI | |
| PF7302 | gtttCAGTCCCCTGACGGGGAAAAGAGG | R Mammalian III-Dv | 122 |
| | GTGTTGAACTCGATGACTTAGGCTGACCT | Spacer (target | |
| | ATGCCGTGTAATCCTC | random control 1) + | |
| | | Repeat, clone into | |
| | | BsaI | |
| PF7303 | aaacGCTAGGATATAATGCTGAGGACCTGA | F Mammalian III-Dv | 123 |
| | ACTCGTACTGGTTCAACACCCTCTTTTCC | Spacer (target | |
| | CCGTCAGGGGACTG | random control 2) + | |
| | | Repeat, clone into | |
| | | BsaI | |
| PF7304 | gtttCAGTCCCCTGACGGGGAAAAGAGG | R Mammalian III-Dv | 124 |
| | GTGTTGAACCAGTACGAGTTCAGGTCCTC | Spacer (target | |
| | AGCATTATATCCTAGC | random control 2) + | |
| | | Repeat, clone into | |
| | | BsaI | |

TABLE 11
Gene blocks used to construct vectors

| | | | SEQ |
|---|---|---|---|
| | | | ID |
| Name | Sequence (5′-3′) | Notes | NO: |
| PF7091 | CCATGGTGGCGGCACCGGTGAATTCTCCAGGCGATCTGACGGTTCACTA | bidirectional | 125 |
| | AACGAGCTCTGCTTATATAGGCCTCCCACCGTACACGCCACCTCGACATA | CMV fragment | |
| | CTCGAGTAGTTATTAATAGTAATCAATTACGGGGTCATTAGTTCATAGCCC | A | |
| | ATATATGGAGTTCCGCGTTACATAACTTACGGTAAATGGCCCGCCTGGCT | | |
| | GACCGCCCAACGACCCCCGCCCATTGACGTCAATAATGACGTATGTTCCC | | |
| | ATAGTAA | | |
| PF7092 | GACGTATGTTCCCATAGTAACGCCAATAGGGACTTTCCATTGACGTCAAT | bidirectional | 126 |
| | GGGTGGAGTATTTACGGTAAACTGCCCACTTGGCAGTACATCAAGTGTAT | CMV fragment | |
| | CATATGCCAAGTACGCCCCCTATTG | B | |
| PF7093 | GCCAAGTACGCCCCCTATTGACGTCAATGACGGTAAATGGCCCGCCTGG | bidirectional | 127 |
| | CATTATGCCCAGTACATGACCTTATGGGACTTTCCTACTTGGCAGTACATC | CMV fragment | |
| | TACGTATTAGTCATCGCTATTACCATGGTGATGCGGTTTTGGCAGTACATC | C | |
| | AATGGGCGTGGATAGCGGTTTGACTCACGGGGATTTCCAAGTCTCCACCC | | |
| | CATTGACGTCAATGGGAGTTTGTTTTGGCACCAAAATCAACGGGACTTTC | | |
| | CAAAATGTCGTAACAACTCCGCCCCATTGACGCAAATGGGCGGTAGGCGT | | |
| | GTACGGTGGGAGGTCTATATAAGCAGAGCTGGTTTAGTGAACCGTCAGAT | | |
| | CCGCTAGGGATCCGCCGCCACCATGG | | |
| PF7145 | GAAGGCAGAGGAAGCCTACTTACATGCGGTGATGTTGAGGAAAATCCGG | 6803_III-Dv | 128 |
| | GTCCAGATTACAAGGATGACGATGACAAGATGGCACCGAAGAAAAAACG | cas7-5-11 | |
| | TAAGGTGCGTGGTATGCGGGGCATCGAGATCACAATCACCATGCAGTCT | (2A-FLAG- | |
| | GATTGGCACGTGGGCACAGGCATGGGCAGAGGAGAGCTGGACAGCGTG | NLS- | |
| | GTTCAGCGGGATGGCGACAATCTGCCATACATCCCTGGAAAGACCCTGAC | humanised | |
| | TGGCATCCTGCGGGACAGCTGTGAACAGGTGGCCCTGGGCCTGGACAAC | Cas7) | |
| | GGCCAAACAAGAGGACTGTGGCACGGCTGGATCAACTTCATCTTCGGCG | | |
| | ACCAGCCTGCCCTGGCTCAGGGTGCCATCGAACCAGAGCCACGGCCTGC | | |
| | TCTGATTGCAATCGGATCCGCTCACCTGGATCCTAAGCTGAAGGCCGCCT | | |
| | TCCAGGGCAAGAAGCAGCTGCAGGAGGCCATCGCTTTTATGAAGCCCGG | | |
| | CGTGGCCATCGATGCCATTACAGGCACCGCCAAGAAGGATTTCCTGAGAT | | |
| | TCGAGGAAGTCGTCAGACTGGGTGCTAAGCTGACCGCCGAGGTGGAACT | | |
| | TAACCTGCCAGACAATCTGTCTGAAACCAACAAAAAAGTGATAGCTGGCA | | |
| | TCCTGGCTAGCGGTGCCAAGCTGACCGAGCGGCTGGGCGGAAAGCGGA | | |
| | GAAGAGGCAACGGCAGATGCGAGCTGAAGTTCAGCGGCTACAGCGATCA | | |
| | ACAAATCCAGTGGCTGAAAGACAACTACCAAAGCGTGGACCAGCCCCCTA | | |
| | AGTACCAGCAGAACAAGCTGCAGAGTGCGGGCGACAATCCTGAGCAGCA | | |
| | GCCACCTTGGCATATCATCCCTCTGACCATCAAAACCCTGAGCCCTGTGG | | |
| | TGCTGCCTGCTAGAACAGTGGGCAACGTGGTGGAATGCCTGGACTACAT | | |
| | CCCTGGCAGATACCTGCTGGGCTACATCCACAAAACACTGGGAGAATACT | | |
| | TCGACGTGTCACAGGCCATTGCGGCCGGAGATCTGATCATTACCAATGCC | | |
| | ACAATCAAGATCGACGGCAAAGCCGGAAGGGCCACCCCTTTCTGCCTGTT | | |
| | TGGCGAGAAGCTCGATGGCGGCCTTGGCAAAGGCAAGGGCGTGTACAAC | | |
| | CGGTTTCAGGAAAGCGAGCCTGACGGCATCCAGCTGAAGGGCGAACGG | | |
| | GGAGGCTATGTGGGCCAGTTCGAGCAAGAGCAGAGAAATCTGCCCAACA | | |
| | CCGGCAAGATCAACAGCGAACTGTTCACCCACAACACAATCCAAGATGAT | | |
| | GTGCAGAGGCCTACCTCCGACGTGGGAGGCGTCTACAGCTACGAGGCCA | | |
| | TCATTGCCGGACAGACATTCGTGGCTGAGCTGAGACTGCCCGATTCTCTG | | |
| | GTGAAGCAGATCACCAGCAAGAACAAGAACTGGCAGGCCCAGCTGAAGG | | |
| | CAACCATCAGAATCGGCCAGTCCAAGAAGGACCAGTACGGCAAAATCGA | | |
| | AGTGACCTCTGGCAACAGCGCTGATCTGCCTAAGCCTACCGGCAACAACA | | |
| | AGACCCTGAGCATCTGGTTCCTGTCCGACATTCTGCTGAGAGGCGACAGA | | |
| | CTCAACTTCAATGCCACACCAGACGACCTGAAGAAATACCTGGAGAACGC | | |
| | CCTGGATATCAAGCTGAAGGAACGGTCCGACAACGACCTGATCTGCATCG | | |
| | CTCTGCGGAGCCAGCGGACAGAGAGCTGGCAGGTGAGATGGGGCCTGC | | |
| | CTAGACCCAGCCTGGTGGGATGGCAAGCCGGCTCTTGTCTGATCTACGA | | |
| | CATCGAGTCCGGCACCGTGAACGCCGAGAAACTCCAGGAGCTGATGATC | | |
| | ACCGGCATCGGGGATAGATGCACCGAGGGCTATGGCCAGATCGGCTTCA | | |
| | ACGACCCCCTCCTGAGCGCCAGCCTGGGCAAGCTGACCGCCAAGCCTAA | | |
| | GGCCAGCAACAACCAGTCCCAGAATTCTCAGTCTAACCCCCTGCCCACGA | | |
| | ACCACCCTACACAGGACTACGCCAGACTGATCGAGAAGGCCGCCTGGCG | | |
| | GGAAGCTATTCAGAACAAGGCTCTGGCCCTGGCCTCTAGCCGCGCCAAA | | |
| | AGAGAGGAAATCCTGGGCATCAAGATCATGGGCAAGGACAGCCAACCTA | | |
| | CCATGACCCAGCTGGGCGGATTTAGATCTGTGCTGAAAAGGCTGCACAG | | |
| | CAGAAACAACAGAGATATCGTGACAGGCTATCTGACAGCACTTGAGCAG | | |
| | GTCAGCAATAGAAAGGAAAAGTGGTCCAATACCAGCCAGGGCCTGACCA | | |
| | AGATCCGCAACCTGGTGACCCAGGAGAACCTTATCTGGAACCACCTGGAC | | |
| | ATCGACTTCTCTCCTCTGACAATCACGCAGAACGGCGTTAACCAGCTGAA | | |
| | GAGCGAGCTGTGGGCCGAAGCCGTGCGGACCCTGGTCGACGCCATCATC | | |
| | CGGGGCCACAAGCGGGACCTGGAAAAGGCCCAGGAGAACGAGAGCAAC | | |
| | CAGCAGTCTCAAGGAGCCGCT | | |
| PF7146 | GAAGGCAGAGGAAGCCTACTGACCTGTGGCGACGTCGAGGAAAATCCTG | 6803_III-Dv | 129 |
| | GTCCAGACTATAAGGACGACGACGACAAGATGGCTCCGAAAAAGAAGCG | cas7-7 (2A- | |
| | TAAGGTCCGTGGCATGGCCAGAAAGGTGACAACCAGATGGAAGATCACC | FLAG-NLS- | |
| | GGAACACTGATCGCCGAGACACCTCTGCACATCGGAGGAGTTGGTGGTG | humanised | |
| | ATGCCGATACCGACCTGGCACTGGCTGTTAACGGTGCTGGTGAGTACTAC | Cas) | |
| | GTTCCTGGTACCAGCCTGGCCGGAGCTCTGAGGGGGTGGATGACCCAGC | | |
| | TGCTGAACAATGACGAGAGCCAGATCAAGGACCTGTGGGGCGACCACCT | | |
| | GGACGCTAAAAGAGGCGCCAGCTTTGTGATCGTGGACGACGCCGTGATC | | |
| | CACATCCCAAACAACGCGGACGTGGAAATCCGGGAAGGAGTGGGCATCG | | |
| | ATAGACATTTCGGCACCGCCGCCAACGGCTTCAAGTACAGTAGAGCCGTG | | |
| | ATCCCTAAGGGCAGCAAGTTCAAGCTGCCTCTGACCTTCGACTCCCAAGA | | |
| | TGACGGACTGCCTAATGCTCTGATTCAGCTGCTCTGTGCTCTGGAAGCCG | | |
| | GAGACATTCGCCTGGGAGCTGCAAAGACACGGGGTCTTGGAAGAATCAA | | |
| | GCTGGATGACCTGAAGCTGAAGAGCTTTGCCCTGGATAAGCCCGAGGGC | | |
| | ATTTTCTCCGCCCTGCTGGATCAAGGTAAGAAACTGGATTGGAACCAGCT | | |
| | TAAGGCCAATGTGACTTACCAGAGCCCTCCTTACCTGGGCATCAGCATCA | | |
| | CATGGAATCCTAAGGATCCTGTGATGGTGAAGGCCGAGGGCGATGGCCT | | |
| | GGCCATCGACATCCTGCCCCTGGTGTCTCAGGTTGGCTCTGATGTGCGGT | | |
| | TCGTCATCCCCGGCAGCAGCATCAAGGGAATTCTGCGGACCCAGGCCGA | | |
| | GCGGATTATCAGAACCATCTGCCAGAGCAACGGCAGCGAGAAGAACTTC | | |
| | CTGGAACAGCTAAGAATCAACCTGGTTAACGAGCTGTTCGGCTCCGCCTC | | |
| | TCTGAGCCAAAAGCAGAACGGCAAGGACATCGACCTGGGAAAAATCGGC | | |
| | GCCCTGGCCGTGAACGACTGCTTCAGCAGCCTGTCTATGACACCCGACCA | | |
| | GTGGAAAGCCGTGGAAAACGCCACAGAGATGACCGGAAATCTGCAACCA | | |
| | GCCCTGAAGCAGGCCACCGGATATCCTAATAACATCAGCCAAGCTTATAA | | |
| | GGTGCTGCAGCCTGCCATGCACGTGGCCGTCGACAGATGGACCGGTGGA | | |
| | GCCGCTGAGGGCATGCTGTACAGCGTGCTGGAACCCATCGGCGTGACAT | | |
| | GGGAGCCCATCCAGGTGCACCTGGACATCGCTAGACTGAAAAACTACTAC | | |
| | CACGGCAAAGAGGAAAAGCTGAAACCTGCTATCGCCCTGCTGCTGCTGG | | |
| | TGCTCAGAGATCTGGCTAACAAGAAGATCCCCGTGGGCTACGGCACCAA | | |
| | CCGGGGCATGGGCACCATCACCGTGTCCCAGATCACCCTGAACGGCAAG | | |
| | GCTCTGCCTACAGAGCTGGAACCACTGAACAAAACCATGACCTGTCCTAA | | |
| | CCTGACAGACCTGGATGAGGCCTTTAGACAGGACCTGTCTACAGCCTGG | | |
| | AAGGAATGGATCGCCGATCCTATCGACCTGTGCCAACAGGAAGCTGCT | | |
| PF7147 | AGCAGAGCCAGGGAGCCGCTCTGAAGATCACAAGGCGCATCCTGGGCGA | 6803_III-Dv | 130 |
| | CGCAGAGTTCCACGGCAAGCCCGACAGACTGGAAAAGAGCCGCAGCGTG | Cas7-7 with | |
| | TCTATCGGCTCTGTGCTGATGGCCAGAAAGGTGACAACCAGATGGAAGAT | linkers for | |
| | CACCGGAACACTGATCGCCGAGACACCTCTGCACATCGGAGGAGTTGGT | singer effector | |
| | GGTGATGCCGATACCGACCTGGCACTGGCTGTTAACGGTGCTGGTGAGT | (humanised) | |
| | ACTACGTTCCTGGTACCAGCCTGGCCGGAGCTCTGAGGGGGTGGATGAC | | |
| | CCAGCTGCTGAACAATGACGAGAGCCAGATCAAGGACCTGTGGGGCGAC | | |
| | CACCTGGACGCTAAAAGAGGCGCCAGCTTTGTGATCGTGGACGACGCCG | | |
| | TGATCCACATCCCAAACAACGCGGACGTGGAAATCCGGGAAGGAGTGGG | | |
| | CATCGATAGACATTTCGGCACCGCCGCCAACGGCTTCAAGTACAGTAGAG | | |
| | CCGTGATCCCTAAGGGCAGCAAGTTCAAGCTGCCTCTGACCTTCGACTCC | | |
| | CAAGATGACGGACTGCCTAATGCTCTGATTCAGCTGCTCTGTGCTCTGGA | | |
| | AGCCGGAGACATTCGCCTGGGAGCTGCAAAGACACGGGGTCTTGGAAGA | | |
| | ATCAAGCTGGATGACCTGAAGCTGAAGAGCTTTGCCCTGGATAAGCCCGA | | |
| | GGGCATTTTCTCCGCCCTGCTGGATCAAGGTAAGAAACTGGATTGGAACC | | |
| | AGCTTAAGGCCAATGTGACTTACCAGAGCCCTCCTTACCTGGGCATCAGC | | |
| | ATCACATGGAATCCTAAGGATCCTGTGATGGTGAAGGCCGAGGGCGATG | | |
| | GCCTGGCCATCGACATCCTGCCCCTGGTGTCTCAGGTTGGCTCTGATGTG | | |
| | CGGTTCGTCATCCCCGGCAGCAGCATCAAGGGAATTCTGCGGACCCAGG | | |
| | CCGAGCGGATTATCAGAACCATCTGCCAGAGCAACGGCAGCGAGAAGAA | | |
| | CTTCCTGGAACAGCTAAGAATCAACCTGGTTAACGAGCTGTTCGGCTCCG | | |
| | CCTCTCTGAGCCAAAAGCAGAACGGCAAGGACATCGACCTGGGAAAAAT | | |
| | CGGCGCCCTGGCCGTGAACGACTGCTTCAGCAGCCTGTCTATGACACCC | | |
| | GACCAGTGGAAAGCCGTGGAAAACGCCACAGAGATGACCGGAAATCTGC | | |
| | AACCAGCCCTGAAGCAGGCCACCGGATATCCTAATAACATCAGCCAAGCT | | |
| | TATAAGGTGCTGCAGCCTGCCATGCACGTGGCCGTCGACAGATGGACCG | | |
| | GTGGAGCCGCTGAGGGCATGCTGTACAGCGTGCTGGAACCCATCGGCGT | | |
| | GACATGGGAGCCCATCCAGGTGCACCTGGACATCGCTAGACTGAAAAAC | | |
| | TACTACCACGGCAAAGAGGAAAAGCTGAAACCTGCTATCGCCCTGCTGCT | | |
| | GCTGGTGCTCAGAGATCTGGCTAACAAGAAGATCCCCGTGGGCTACGGC | | |
| | ACCAACCGGGGCATGGGCACCATCACCGTGTCCCAGATCACCCTGAACG | | |
| | GCAAGGCTCTGCCTACAGAGCTGGAACCACTGAACAAAACCATGACCTGT | | |
| | CCTAACCTGACAGACCTGGATGAGGCCTTTAGACAGGACCTGTCTACAGC | | |
| | CTGGAAGGAATGGATCGCCGATCCTATCGACCTGTGCCAGCAGGAGGCT | | |
| | GCTCTCGGCAACCCCAAAGGCCAAGAGCTTAAACTGGATCCTCCATCCGC | | |
| | TGACGCCACCCAGGCTGGCGTGCCCGCGCAACAGAATGCCGCCAAGACA | | |
| | CAGGCTCAGGGAGCCCAGGAGAAGATGACCGTGGGAACGCTGGG | | |
| PF7148 | GAAGGCAGAGGAAGCCTACTGACATGCGGAGATGTGGAAGAGAACCCCG | 6803_III-Dv | 131 |
| | GACCTGACTACAAGGACGACGACGACAAGATGGCCCCTAAGAAGAAACG | cas7-insertion | |
| | GAAGGTGCGGGGCATGACCGTGGGAACGCTGGGAGTCGTGGGCAGCGC | (2A-FLAG- | |
| | CAAGAACCTGAAACTGCAGCTGAGCTTCATTAACACCAGACAGCAGTACG | NLS- | |
| | TGCAGATCACTCTGTTCGAGAGAAACAGCTTTAAGGTGGCCGAAGAAGAA | humanised | |
| | TTCAGCACAGAGCTGGTGGAAATAATCAAAACCGCCCTGCCTACACTTAA | Cas) | |
| | GAACAAGAAAGTGGAATTCGAGGAGGACGGCGACCAGATCAAGCAGATC | | |
| | AGAGAGAAGGGCCAGGCCTGGGTGGGCGCCGCTGAGCAGATCGCCCCT | | |
| | TATGTGCTGCCCAGCGGAAATATCACAGAAACCCCTAGGAATGTGAACGC | | |
| | CAGCAACTTCCACAATCCTTACAACTTCGTGCCCGCTCTGCCCAGAGATG | | |
| | GCATCACCGGCGATCTGGGCGATTGCGCCCCTGCTGGCCACAGCTACTA | | |
| | TCACGGCGACAAGTACAGCGGCAGGATTGCCGTGAAACTGACAACCGTG | | |
| | ACACCTCTGCTGATCCCCGACGCTAGCAAGGAAGAGATCAACAATAATCA | | |
| | CAAGACCTACCCCGTGCGGATCGGCAAAGATGGCAAGCCCTACCTGCCA | | |
| | CCAACATCTATTAAGGGCATGCTGAGAAGCGCCTACGAGGCCGTTACCAA | | |
| | CAGCCGGCTGGCCGTGTTCGAGGACCACGACAGCCGCCTGGCTTATAGA | | |
| | ATGCCTGCCACCATGGGACTGCAGATGGTGCCTGCCAGAATCGAGGGCG | | |
| | ATAATATCGTGCTGTACCCCGGCACCTCTCGGATCGGCAACAACGGCCG | | |
| | GCCTGCTAATAACGACCCTATGTACGCCGCCTGGCTGCCTTACTACCAGA | | |
| | ACAGAATCGCCTACGACGGCTCTAGAGATTACCAGATGGCCGAGCACGG | | |
| | CGACCATGTGCGGTTCTGGGCCGAAAGATACACCCGAGGCAACTTTTGTT | | |
| | ACTGGAGAGTGCGCCAGATCGCAAGACATAACCAGAACCTGGGTAACAG | | |
| | ACCTGAGAGAGGCCGGAACTACGGCCAACACCACAGCACCGGCGTGATC | | |
| | GAGCAGTTCGAAGGCTTCGTGTACAAGACAAACAAAAACATCGGCAACAA | | |
| | GCACGACGAGAGAGTTTTCATCATCGACCGGGAGTCCATCGAAATCCCTC | | |
| | TCAGCCGGGATCTCCGGCGGAAGTGGCGGGAACTGATCACCAGCTACCA | | |
| | GGAGATCCACAAGAAGGAAGTGGATAGAGGAGATACAGGCCCTTCCGCC | | |
| | GTGAACGGCGCCGTGTGGAGCCGACAGATCATCGCTGATGAGAGCGAGC | | |
| | GGAACCTGAGCGACGGCACCCTGTGCTACGCCCACGTGAAGAAAGAGGA | | |
| | CGGCCAGTACAAGATCCTGAACCTGTACCCCGTGATGATCACCAGAGGCC | | |
| | TGTACGAGATCGCCCCTGTGGACCTGCTGGACGAGACACTGAAGCCTGC | | |
| | AACCGACAAGAAGCAACTGAGCCCTGCCGACAGAGTGTTTGGATGGGTT | | |
| | AACCAGAGAGGAAACGGATGTTATAAAGGCCAGCTGAGAATCCACTCTGT | | |
| | GACCTGCCAGCACGATGATGCCATTGATGACTTCGGCAATCAGAATTTCA | | |
| | GCGTGCCACTGGCCATCCTGGGCCAGCCCAAGCCAGAACAGGCCAGATT | | |
| | CTACTGCGCCGACGACCGGAAGGGAATCCCCCTGGAAGACGGCTACGAC | | |
| | AGAGACGACGGCTACTCTGATAGCGAGCAGGGCCTGCGAGGCAGGAAG | | |
| | GTCTACCCCCACCACAAAGGACTGCCAAACGGCTACTGGTCCAACCCCAC | | |
| | AGAAGATAGATCTCAGCAGGCGATCCAGGGCCACTACCAAGAGTACAGA | | |
| | AGACCCAAGAAGGACGGCCTGGAACAAAGAGACGACCAGAACCGGAGC | | |
| | GTGAAGGGCTGGGTCAAACCTCTCACAGAGTTCACCTTCGAGATCGACGT | | |
| | GACAAACCTGTCCGAGGTGGAACTGGGCGCTCTGCTCTGGCTGCTGACC | | |
| | CTGCCAGATCTGCACTTCCACCGGCTGGGCGGCGGAAAGCCTCTGGGTT | | |
| | TCGGCAGCGTGCGGCTGGACATTGACCCCGATAAGACCGACCTGAGAAA | | |
| | TGGCGCCGGCTGGCGAGATTACTACGGCTCGCTGCTCGAGACAAGCCAG | | |
| | CCTGACTTTACCACCCTGATCAGCCAGTGGATCAACGCCTTCCAGACCGC | | |
| | CGTGAAGGAAGAGTACGGATCCAGCAGCTTCGACCAAGTGACCTTTATCA | | |
| | AGGCCAGCGGCCAAAGCCTGCAGGGCTTCCACGACAATGCTTCTATCCAT | | |
| | TATCCTAGATCCACCCCTGAGCCTAAGCCTGACGGCGAGGCTTTTAAGTG | | |
| | GTTTGTGGCCAACGAGAAGGGGAGAAGACTGGCCCTGCCGGCCCTGGAA | | |
| | AAGAGCCAGTCTTTCCCTATCAAGCCTAGTTAGTCTAGAGGATCATAATCA | | |
| | GCCATACCACATTTGTAGAGGTTTTACTTGCTTTAAAAAACCTCCCACACC | | |
| | TCCCCCTGAACCTGAAACATAAAATGAATGCAATTGTTGTTGTTAACTTGT | | |
| | TTATTGCAGCTTATAATGGTTACAAATAAAGCAATAGCATCACAAATTTCA | | |
| | CAAATAAAGCATTTTTTTCACTGCCGTAATACGGTTATCCACAGAATCAGG | | |
| | GGATAACGCAGGAAAGAAACTAGT | | |
| PF7149 | GAAGGCAGAGGAAGCCTACTTACATGCGGCGATGTGGAAGAAAACCCCG | 6803_III-Dv | 132 |
| | GCCCTCACCATCACCATCATCACAGCGGAGACTACAAGGATGACGATGAT | cas10 (2A-His- | |
| | AAGATGTTCCTGGTGCTGATCGAAACCAGCGGCAACCAGCACTTCATCTT | FLAG- | |
| | CAGCACCAACAAACTGCGGGAAAACATCGGCGCCAGCGAGCTGACCTAC | humanised | |
| | CTGGCAACCACCGAGATCCTCTTCCAGGGCGTGGACCGGGTCTTTCAGA | Cas) (minus | |
| | CCAATTACTACGACCAGTGGAGCGACACCAACAGCCTGAACTTCCTGGCA | NLS) | |
| | GATAGCAAGCTGAACCCCGCCATCGACGACCCCAAGAACAACGCCGATA | | |
| | TCGAGATCCTGCTGGCCACAAGCGGAAAGGCCATCGCCCTGGTGAAGGA | | |
| | GGAAGGCAAGGCCAAGCAGCTGATCAAAGAGGTGACTAAGCAGGCTCTG | | |
| | ATTAACGCTCCTGGACTGGAAATTGGCGGCATCTACGTGAACTGCAACTG | | |
| | GCAGGACAAGCTGGGCGTCGCGAAGGCCGTGAAAGAAGCTCACAAGCAA | | |
| | TTTGAGGTGAACCGGGCCAAAAGAGCCGGCGCTAATGGCAGGTTCCTGC | | |
| | GGCTGCCAATCGCTGCTGGCTGCTCTGTGTCCGAGCTGCCTGCTTCTGAT | | |
| | TTTGACTACAACGCTGACGGCGACAAGATCCCTGTCTCCACCGTGTCTAA | | |
| | AGTGAAGAGAGAGACAGCCAAAAGCGCTAAAAAGCGGCTGAGAAGCGTG | | |
| | GATGGCAGACTTGTTAATGACCTGGCTCAGCTGGAAAAATCATTCGACGA | | |
| | ACTGGATTGGCTGGCCGTGGTGCACGCCGACGGCAACGGCCTGGGCCA | | |
| | GATCCTCCTGAGCCTGGAAAAATACATCGGAGAGCAGACCAACCGGAAC | | |
| | TACATCGATAAGTACCGGAGACTGTCTCTTGCTCTGGACAACTGCACCAT | | |
| | CAACGCCTTTAAGATGGCCATCGCTGTGTTCAAGGAAGATAGCAAGAAGA | | |
| | TCGACCTGCCTATCGTGCCTCTGATCCTGGGAGGAGATGACCTGACAGTG | | |
| | ATCTGTAGGGGCGATTACGCCCTGGAGTTCACCAGAGAGTTCCTGGAGG | | |
| | CCTTCGAGGGCCAGACAGAGACACACGACGACATCAAGGTGATCGCCCA | | |
| | GAAAGCCTTCGGTGTGGACAGACTGTCCGCCTGCGCCGGCATCAGCATC | | |
| | ATCAAGCCTCACTTCCCCTTCAGCGTGGCCTATACACTGGCCGAAAGACT | | |
| | GATCAAGAGCGCCAAGGAGGTGAAGCAGAAGGTGACCGTTACCAATTCT | | |
| | AGCCCTATCACCCCTTTTCCATGTAGCGCCATTGATTTCCACATCCTGTAC | | |
| | GACAGCAGCGGCATCGACTTTGATAGAATCAGAGAGAAGCTGCGGCCTG | | |
| | AGGATAACACAGAACTGTACAACAGACCCTACGTGGTCACCGCCGCCGA | | |
| | AAACCTGAGCCAGGCCCAAGGCTACGAGTGGTCCCAAGCCCACTCCCTG | | |
| | CAGACCCTGGCGGACAGAGTGTCCTACCTGCGCAGCGAGGACGGCGAA | | |
| | GGCAAGTCTGCCCTGCCCAGCAGCCAGAGCCACGCCCTGAGAACAGCCC | | |
| | TGTATCTGGAAAAGAATGAAGCCGACGCCCAGTACAGCCTGATCTCTCAA | | |
| | AGATACAAGATCTTGAAGAACTTCGCCGAGGACGGCGAGAACAAGTCTCT | | |
| | GTTCCATCTGGAAAATGGAAAGTACGTGACCCGGTTCCTCGATGCCCTCG | | |
| | ACGCCAAGGACTTCTTCGCCAACGCCAATCACAAGAACCAGGGCGAG | | |
| PF7150 | GAAGGCAGAGGAAGCCTACTTACATGCGGCGATGTGGAAGAAAACCCCG | 6803_III-Dv | 133 |
| | GCCCTCACCATCACCATCATCACAGCGGAGACTACAAGGATGACGATGAT | cas10 | |
| | AAGATGTTCCTGGTGCTGATCGAAACCAGCGGCAACCAGCACTTCATCTT | (mutated HD | |
| | CAGCACCAACAAACTGCGGGAAAACATCGGCGCCAGCGAGCTGACCTAC | and palm) | |
| | CTGGCAACCACCGAGATCCTCTTCCAGGGCGTGGACCGGGTCTTTCAGA | (2A-His-FLAG- | |
| | CCAATTACTACGACCAGTGGAGCGACACCAACAGCCTGAACTTCCTGGCA | humanised | |
| | GATAGCAAGCTGAACCCCGCCATCGACGACCCCAAGAACAACGCCGATA | Cas) (minus | |
| | TCGAGATCCTGCTGGCCACAAGCGGAAAGGCCATCGCCCTGGTGAAGGA | NLS) | |
| | GGAAGGCAAGGCCAAGCAGCTGATCAAAGAGGTGACTAAGCAGGCTCTG | | |
| | ATTAACGCTCCTGGACTGGAAATTGGCGGCATCTACGTGAACTGCAACTG | | |
| | GCAGGACAAGCTGGGCGTCGCGAAGGCCGTGAAAGAAGCTCACAAGCAA | | |
| | TTTGAGGTGAACCGGGCCAAAAGAGCCGGCGCTAATGGCAGGTTCCTGC | | |
| | GGCTGCCAATCGCTGCTGGCTGCTCTGTGTCCGAGCTGCCTGCTTCTGAT | | |
| | TTTGACTACAACGCTGACGGCGACAAGATCCCTGTCTCCACCGTGTCTAA | | |
| | AGTGAAGAGAGAGACAGCCAAAAGCGCTAAAAAGCGGCTGAGAAGCGTG | | |
| | GATGGCAGACTTGTTAATGACCTGGCTCAGCTGGAAAAATCATTCGACGA | | |
| | ACTGGATTGGCTGGCCGTGGTGCACGCCGACGGCAACGGCCTGGGCCA | | |
| | GATCCTCCTGAGCCTGGAAAAATACATCGGAGAGCAGACCAACCGGAAC | | |
| | TACATCGATAAGTACCGGAGACTGTCTCTTGCTCTGGACAACTGCACCAT | | |
| | CAACGCCTTTAAGATGGCCATCGCTGTGTTCAAGGAAGATAGCAAGAAGA | | |
| | TCGACCTGCCTATCGTGCCTCTGATCCTGGGTGGAGCTGCCCTGACAGTG | | |
| | ATCTGTAGGGGCGATTACGCCCTGGAGTTCACCAGAGAGTTCCTGGAGG | | |
| | CCTTCGAGGGCCAGACAGAGACAGCCGCTGACATCAAGGTGATCGCCCA | | |
| | GAAAGCCTTCGGTGTGGACAGACTGTCCGCCTGCGCCGGCATCAGCATC | | |
| | ATCAAGCCTCACTTCCCCTTCAGCGTGGCCTATACACTGGCCGAAAGACT | | |
| | GATCAAGAGCGCCAAGGAGGTGAAGCAGAAGGTGACCGTTACCAATTCT | | |
| | AGCCCTATCACCCCTTTTCCATGTAGCGCCATTGATTTCCACATCCTGTAC | | |
| | GACAGCAGCGGCATCGACTTTGATAGAATCAGAGAGAAGCTGCGGCCTG | | |
| | AGGATAACACAGAACTGTACAACAGACCCTACGTGGTCACCGCCGCCGA | | |
| | AAACCTGAGCCAGGCCCAAGGCTACGAGTGGTCCCAAGCCCACTCCCTG | | |
| | CAGACCCTGGCGGACAGAGTGTCCTACCTGCGCAGCGAGGACGGCGAA | | |
| | GGCAAGTCTGCCCTGCCCAGCAGCCAGAGCCACGCCCTGAGAACAGCCC | | |
| | TGTATCTGGAAAAGAATGAAGCCGACGCCCAGTACAGCCTGATCTCTCAA | | |
| | AGATACAAGATCTTGAAGAACTTCGCCGAGGACGGCGAGAACAAGTCTCT | | |
| | GTTCCATCTGGAAAATGGAAAGTACGTGACCCGGTTCCTCGATGCCCTCG | | |
| | ACGCCAAGGACTTCTTCGCCAACGCCAATCACAAGAACCAGGGCGAG | | |
| PF7152 | TAATACGGTTATCCACAGAATCAGGGGATAACGCAGGAAAGAAACTAGTG | Pu6, III-Dv | 134 |
| | AGGGCCTATTTCCCATGATTCCTTCATATTTGCATATACGATACAAGGCTG | repeat, Ori, | |
| | TTAGAGAGATAATTAGAATTAATTTGACTGTAAACACAAAGATATTAGTAC | ApR | |
| | AAAATACGTGACGTAGAAAGTAATAATTTCTTGGGTAGTTTGCAGTTTTAA | | |
| | AATTATGTTTTAAAATGGACTATCATATGCTTACCGTAACTTGAAAGTATTT | | |
| | CGATTTCTTGGCTTTATATATCTTGTGGAAAGGACGAAACACCGTTTCACA | | |
| | CACTCGAGATCTGTTCAACACCCTCTTTTCCCCGTCAGGGGACTGAAACT | | |
| | GAGACCTTTCACACAGGAAACAGTTTTTTTACATGTGAGCAAAAGGCCAG | | |
| | CAAAAGGCCAGGAACCGTAAAAAGGCCGCGTTGCTGGCGTTTTTCCATAG | | |
| | GCTCCGCCCCCCTGACGAGCATCACAAAAATCGACGCTCAAGTCAGAGGT | | |
| | GGCGAAACCCGACAGGACTATAAAGATACCAGGCGTTTCCCCCTGGAAG | | |
| | CTCCCTCGTGCGCTCTCCTGTTCCGACCCTGCCGCTTACCGGATACCTGT | | |
| | CCGCCTTTCTCCCTTCGGGAAGCGTGGCGCTTTCTCATAGCTCACGCTGT | | |
| | AGGTATCTCAGTTCGGTGTAGGTCGTTCGCTCCAAGCTGGGCTGTGTGCA | | |
| | CGAACCCCCCGTTCAGCCCGACCGCTGCGCCTTATCCGGTAACTATCGTC | | |
| | TTGAGTCCAACCCGGTAAGACACGACTTATCGCCACTGGCAGCAGCCACT | | |
| | GGTAACAGGATTAGCAGAGCGAGGTATGTAGGCGGTGCTACAGAGTTCT | | |
| | TGAAGTGGTGGCCTAACTACGGCTACACTAGAAGAACAGTATTTGGTATC | | |
| | TGCGCTCTGCTGAAGCCAGTTACCTTCGGAAAAAGAGTTGGTAGCTCTTG | | |
| | ATCCGGCAAACAAACCACCGCTGGTAGCGGTGGTTTTTTTGTTTGCAAGC | | |
| | AGCAGATTACGCGCAGAAAAAAAGGATCTCAAGAAGATCCTTTGATCTTT | | |
| | TCTACGGGGTCTGACGCTCAGTGGAACGAAAACTCACGTTAAGGGATTTT | | |
| | GGTCATGAGATTATCAAAAAGGATCTTCACCTAGATCCTTTTAAATTAAAA | | |
| | ATGAAGTTTTAAATCAATCTAAAGTATATATGAGTAAACTTGGTCTGACAG | | |
| | TTACCAATGCTTAATCAGTGAGGCACCTATCTCAGCGATCTGTCTATTTCG | | |
| | TTCATCCATAGTTGCCTGACTCCCCGTCGTGTAGATAACTACGATACGGG | | |
| | AGGGCTTACCATCTGGCCCCAGTGCTGCAATGATACCGCGTGACCCACG | | |
| | CTCACCGGCTCCAGATTTATCAGCAATAAACCAGCCAGCCGGAAGGGCC | | |
| | GAGCGCAGAAGTGGTCCTGCAACTTTATCCGCCTCCATCCAGTCTATTAA | | |
| | TTGTTGCCGGGAAGCTAGAGTAAGTAGTTCGCCAGTTAATAGTTTGCGCA | | |
| | ACGTTGTTGCCATTGCTACAGGCATCGTGGTGTCACGCTCGTCGTTTGGT | | |
| | ATGGCTTCATTCAGCTCCGGTTCCCAACGATCAAGGCGAGTTACATGATC | | |
| | CCCCATGTTGTGCAAAAAAGCGGTTAGCTCCTTCGGTCCTCCGATCGTTG | | |
| | TCAGAAGTAAGTTGGCCGCAGTGTTATCACTCATGGTTATGGCAGCACTG | | |
| | CATAATTCTCTTACTGTCATGCCATCCGTAAGATGCTTTTCTGTGACTGGT | | |
| | GAGTACTCAACCAAGTCATTCTGAGAATAGTGTATGCGGCGACCGAGTTG | | |
| | CTCTTGCCCGGCGTCAACACGGGATAATACCGCGCCACATAGCAGAACTT | | |
| | TAAAAGTGCTCATCATTGGAAAACGTTCTTCGGGGCGAAAACTCTCAAGG | | |
| | ATCTTACCGCTGTTGAGATCCAGTTCGATGTAACCCACTCGTGCACCCAA | | |
| | CTGATCTTCAGCATCTTTTACTTTCACCAGCGTTTCTGGGTGAGCAAAAAC | | |
| | AGGAAGGCAAAATGCCGCAAAAAAGGGAATAAGGGCGACACGGAAATGT | | |
| | TGAATACTCATACTCTTCCTTTTTCAATATTATTGAAGCATTTATCAGGGTT | | |
| | ATTGTCTCATGAGCGGATACATATTTGAATGTATTTAGAAAAATAAACAAA | | |
| | TAGGGGTTCCGCGCACATTTCCCCGAAAAGTGCCACCTGACGTCGGCAG | | |
| | TGAAAAAAATGCTTTATTTGTGAAATTTGTGATGCTATTGCTTTATTTGTAA | | |
| | CCATTATAAGCTGCAATAAACAAGTTAACAACAACAATTGCATTCATTTTAT | | |
| | GTTTCAGGTTCAGGGGGAGGTGTGGGAGGTTTTTTAAAGCAAGTAAAACC | | |
| | TCTACAAATGTGGTATGGCTGATTATGATCCTCTAGACTA | | |
| PF7153 | ATGGCCAATCTGGATAAGATGCTGAACACCACCGTGACCGAGGTGCGGC | RFP_hCas6- | 131 |
| | AGTTCCTGCAAGTGGACAGAGTGTGTGTGTTCCAGTTCGAGGAAGATTAC | 2a_stop (2A- | |
| | TCTGGCGTGGTGGTCGTCGAGGCCGTTGACGACCGGTGGATCAGCATCC | FLAG-NLS- | |
| | TGAAGACCCAGGTGCGCGACAGATACTTCATGGAAACAAGAGGCGAAGA | humanised | |
| | GTACTCCCACGGAAGATATCAGGCCATCGCCGACATCTACACCGCCAACC | NucC) | |
| | TGACCGAGTGCTACAGAGATCTGCTGACACAGTTTCAGGTGCGGGCCAT | | |
| | CCTGGCCGTGCCCATCCTGCAGGGCAAGAAGCTGTGGGGCCTGCTCGTG | | |
| | GCCCACCAGCTGGCTGCTCCTAGACAGTGGCAGACATGGGAGATCGACT | | |
| | TCCTGAAACAGCAAGCTGTGGTGGTGGGCATCGCCATTCAGCAGAGCGA | | |
| | AGGCAGAGGAAGCCTACTGACGTGTGGAGACGTGGAAGAGAACCCAGG | | |
| | CCCTGACTACAAGGATGATGATGATAAGATGGCCCCTAAGAAGAAGAGAA | | |
| | AGGTGCGCGGCATGGTCGACCTGAAGAGCCTGGCTGGCGCCGAAATGGT | | |
| | GGGCCTCAGATGGCAGCTGAGATTCGACCGGCCTTGCCGCCTGGAGAGC | | |
| | CACTACGTGAAAGGTCTGCATGCCTGGTTCCTGCATCAGGTGCAGGCCAT | | |
| | TGACCCCGACGTGTCTGCCTGGCTGCACGACGGCCAAGGCGAGAAGCCT | | |
| | TTCACCATCAGCAGATTGATCGGCCCTACACTGTGGCAGGAGGGCCACT | | |
| | GGCACTGGCAAATCAACAAAACCTACCACTGGCAGCTGAACCTGCTGAGC | | |
| | GGCGCCCTGATCGAGGCCCTGCAGCCTTGGCTGGCTAGACTGCCAAACA | | |
| | AGATCGTTCTGGCCAGACAGACACTGTGGGTGGAAGCTGTGGACTGCTA | | |
| | CCTGGCCCCTCACAACTACCAGCAGCTGTGGCCTCAAGGAGCCCTGCCTA | | |
| | GACGGCAAGAATTTACCTTTACAAGCCCCACCAGCTTCAGAAGACAGGGC | | |
| | AACCACTATCCTCTGCCGGAACCTAGGAACGTGCTCCAGTCCTACCTGCG | | |
| | GAGATGGAATGACTTCAGCGGCCTGGCCTTCGAGCCAGAGCCTTTCCTG | | |
| | GACTACTGGGTGCCCCAGAATGTGGTCATCGACCGGCACTGGCTGGAAA | | |
| | GCGTGAAGACCACCGCCGGAAAGCAGGGGAGCGTGGTGGGCTTCGTGG | | |
| | GCGCCGTGTCTCTTGTGCTGACACCCCAGGCCAGAAACGACGGCGATGA | | |
| | CTACGGAAGACTGTTTCACGCGCTGTGTAGATACGGCCCCTATTGCGGCA | | |
| | CCGGCCACAAGACAACCTTCGGCCTGGGCCAGACCATGGCCGGCTGGGC | | |
| | CACACCTGATCTGAAAACCTTCGCCTGTCTGCAAGAAGATCTGCAGACCC | | |
| | AGGTGCTGACACAGAGAATCGATCAGTGCGCCTCTCTACTGCTGGCTCAG | | |
| | AGACAGCGCACCGGAGGACAGCGGGCTCAGGAGATCTGCCACACCCTG | | |
| | GCCACCATCTTCGTGAGACGGGAACAGGGCGAGTCCCTGCAGGAGATCG | | |
| | CCCTGGATCTGCAGCTGCCCTACGAGACAGCCCGGACCTACAGCAAGCG | | |
| | GGCAAAGAGAGCCCTGGCTAACGTGCAGTAGTCTAGAGGATCATAATCA | | |
| | GCCATACCACATTTGTAGAGGTTTTACTTGCTTTAAAAAACCTCCCACACC | | |
| | TCCCCCTGAACCTGAAACATAAAATGAATGCAATTGTTGTTGTTAACTTGT | | |
| | TTATTGCAGCTTATAATGGTTACAAATAAAGCAATAGCATCACAAATTTCA | | |
| | CAAATAAAGCATTTTTTTCACTGCCGTAATACGGTTATCCACAGAATCAGG | | |
| | GGATAACGCAGGAAAGAAACTAGT | | |
| PF7154 | GAAGGCAGAGGAAGCCTACTGACCTGCGGCGATGTGGAAGAGAACCCCG | 6803_III-Dv | 136 |
| | GCCCTGACTACAAGGACGATGATGACAAGATGGCCCCTAAGAAGAAACG | csx19 (2A- | |
| | GAAAGTGCGGGGAATGCCTGCTGGCGGAAGACTGATGAAGAACCTTTAT | FLAG-NLS- | |
| | CACTACCATCAGTACGAGATCACACTGGAATCCGCCGTGGATAGCTGTAA | humanised | |
| | AAACCACCTGCAGGCCGCTATCGGCCTGCTGTACAGCCCTCAGAAGTGC | Cas) | |
| | GAGCTGGTGAAACTGGACAACAGCGGCAAGCTGGTCGACAGCTACAACC | | |
| | GGCTGAAGTTCAACAACCTGGGCGTGTTTGAGGCCAGATTCTTCAACCTC | | |
| | AACTGCGAACTGAGATGGGTTAACGAGTCTAATGGCAACGGAACAGCCG | | |
| | TGCTGCTGAGCGAATCTGATATCACCCTGACCGGCTTCGAGAAGGGCCT | | |
| | GCAAGAGTTCATCACCGCCATTGATCAGCAGTACCTGCTGTGGGGCGAG | | |
| | CCTGCCAAGCACCCCCCCAACGCCGACGGCTGGCAGCGGCTGGCCGAAG | | |
| | CTAGAATCGGAAAGCTGGACATCCCTCTGGATAATCCTCTGAAACCAAAG | | |
| | GACAGAGTGTTTCTGACCAGCGAGGAATACATCGCCGAGGTGGACGACT | | |
| | TCGGCAATTGCGCCGTGATCGACGAGCGGCTGATCAAGCTGGAAGTGAA | | |
| | G | | |
| PF7152 | TAATACGGTTATCCACAGAATCAGGGGATAACGCAGGAAAGAAACTAGTG | Pu6, III-Dv | 137 |
| | AGGGCCTATTTCCCATGATTCCTTCATATTTGCATATACGATACAAGGCTG | repeat, Ori, | |
| | TTAGAGAGATAATTAGAATTAATTTGACTGTAAACACAAAGATATTAGTAC | ApR | |
| | AAAATACGTGACGTAGAAAGTAATAATTTCTTGGGTAGTTTGCAGTTTTAA | | |
| | AATTATGTTTTAAAATGGACTATCATATGCTTACCGTAACTTGAAAGTATTT | | |
| | CGATTTCTTGGCTTTATATATCTTGTGGAAAGGACGAAACACCGTTTCACA | | |
| | CACTCGAGATCTGTTCAACACCCTCTTTTCCCCGTCAGGGGACTGAAACT | | |
| | GAGACCTTTCACACAGGAAACAGTTTTTTTACATGTGAGCAAAAGGCCAG | | |
| | CAAAAGGCCAGGAACCGTAAAAAGGCCGCGTTGCTGGCGTTTTTCCATAG | | |
| | GCTCCGCCCCCCTGACGAGCATCACAAAAATCGACGCTCAAGTCAGAGGT | | |
| | GGCGAAACCCGACAGGACTATAAAGATACCAGGCGTTTCCCCCTGGAAG | | |
| | CTCCCTCGTGCGCTCTCCTGTTCCGACCCTGCCGCTTACCGGATACCTGT | | |
| | CCGCCTTTCTCCCTTCGGGAAGCGTGGCGCTTTCTCATAGCTCACGCTGT | | |
| | AGGTATCTCAGTTCGGTGTAGGTCGTTCGCTCCAAGCTGGGCTGTGTGCA | | |
| | CGAACCCCCCGTTCAGCCCGACCGCTGCGCCTTATCCGGTAACTATCGTC | | |
| | TTGAGTCCAACCCGGTAAGACACGACTTATCGCCACTGGCAGCAGCCACT | | |
| | GGTAACAGGATTAGCAGAGCGAGGTATGTAGGCGGTGCTACAGAGTTCT | | |
| | TGAAGTGGTGGCCTAACTACGGCTACACTAGAAGAACAGTATTTGGTATC | | |
| | TGCGCTCTGCTGAAGCCAGTTACCTTCGGAAAAAGAGTTGGTAGCTCTTG | | |
| | ATCCGGCAAACAAACCACCGCTGGTAGCGGTGGTTTTTTTGTTTGCAAGC | | |
| | AGCAGATTACGCGCAGAAAAAAAGGATCTCAAGAAGATCCTTTGATCTTT | | |
| | TCTACGGGGTCTGACGCTCAGTGGAACGAAAACTCACGTTAAGGGATTTT | | |
| | GGTCATGAGATTATCAAAAAGGATCTTCACCTAGATCCTITTAAATTAAAA | | |
| | ATGAAGTTTTAAATCAATCTAAAGTATATATGAGTAAACTTGGTCTGACAG | | |
| | TTACCAATGCTTAATCAGTGAGGCACCTATCTCAGCGATCTGTCTATTTCG | | |
| | TTCATCCATAGTTGCCTGACTCCCCGTCGTGTAGATAACTACGATACGGG | | |
| | AGGGCTTACCATCTGGCCCCAGTGCTGCAATGATACCGCGTGACCCACG | | |
| | CTCACCGGCTCCAGATTTATCAGCAATAAACCAGCCAGCCGGAAGGGCC | | |
| | GAGCGCAGAAGTGGTCCTGCAACTTTATCCGCCTCCATCCAGTCTATTAA | | |
| | TTGTTGCCGGGAAGCTAGAGTAAGTAGTTCGCCAGTTAATAGTTTGCGCA | | |
| | ACGTTGTTGCCATTGCTACAGGCATCGTGGTGTCACGCTCGTCGTTTGGT | | |
| | ATGGCTTCATTCAGCTCCGGTTCCCAACGATCAAGGCGAGTTACATGATC | | |
| | CCCCATGTTGTGCAAAAAAGCGGTTAGCTCCTTCGGTCCTCCGATCGTTG | | |
| | TCAGAAGTAAGTTGGCCGCAGTGTTATCACTCATGGTTATGGCAGCACTG | | |
| | CATAATTCTCTTACTGTCATGCCATCCGTAAGATGCTTTTCTGTGACTGGT | | |
| | GAGTACTCAACCAAGTCATTCTGAGAATAGTGTATGCGGCGACCGAGTTG | | |
| | CTCTTGCCCGGCGTCAACACGGGATAATACCGCGCCACATAGCAGAACTT | | |
| | TAAAAGTGCTCATCATTGGAAAACGTTCTTCGGGGCGAAAACTCTCAAGG | | |
| | ATCTTACCGCTGTTGAGATCCAGTTCGATGTAACCCACTCGTGCACCCAA | | |
| | CTGATCTTCAGCATCTTTTACTTTCACCAGCGTTTCTGGGTGAGCAAAAAC | | |
| | AGGAAGGCAAAATGCCGCAAAAAAGGGAATAAGGGCGACACGGAAATGT | | |
| | TGAATACTCATACTCTTCCTTTTTCAATATTATTGAAGCATTTATCAGGGTT | | |
| | ATTGTCTCATGAGCGGATACATATTTGAATGTATTTAGAAAAATAAACAAA | | |
| | TAGGGGTTCCGCGCACATTTCCCCGAAAAGTGCCACCTGACGTCGGCAG | | |
| | TGAAAAAAATGCTTTATTTGTGAAATTTGTGATGCTATTGCTTTATTTGTAA | | |
| | CCATTATAAGCTGCAATAAACAAGTTAACAACAACAATTGCATTCATTTTAT | | |
| | GTTTCAGGTTCAGGGGGAGGTGTGGGAGGTTTTTTAAAGCAAGTAAAACC | | |
| | TCTACAAATGTGGTATGGCTGATTATGATCCTCTAGACTA | | |

### Results

To establish the gene knockdown efficacy of Type III-Dv CRISPR-Cas system in mammalian cells, cas sequences were codon optimized for expression in human cells. Each gene fragment was then ordered as gene-blocks and PCR amplified with suitable primers and fragments were combined to form the plasmid by Gibson assembly. The entry vector (pPF3610) was confirmed using Oxford Nanopore sequencing. As required, different spacers were added to this entry vector as follows: appropriate oligonucleotides were annealed, cloned into a BsaI restriction site of pPF3610 and confirmed with Sanger sequencing.

To confirm that the Type III-Dv mammalian expression vectors could be transfected into mammalian cells, HEK293 cells were transfected using lipofectamine and 1 μg of vector DNA. 48-hours after transfection, cells were fixed and then visualized using confocal microscopy. FIG. 21A (i) shows confocal images of HEK293 cells stained with the DNA nuclei stain Hoechst, while FIG. 21A (ii) shows HEK293 cells with red fluorescence. Red fluorescence is indicative of cells transfected with the Type III-Dv expression vector, which has microRFP co-expressed. For further confirmation of Type III-Dv complex expression, cells transfected with pPF3610 for 48-hours were lysed for analysis via Western blot that detected the FLAG tags on each Cas protein. Chemiluminescent imaging of the membrane is shown in FIG. 21A (iii), which shows each of the individual Cas proteins in the III-Dv complex at their expected sizes, except Csx19 was not apparent. Confocal and Western blot data show that transfection of HEK293 cells with pPF3610 results in the expression of Type III-Dv complex.

To quantify the knockdown efficiency of Type III-Dv in mammalian cells, HEK293 cells were co-transfected with a Venus expression plasmid (pPF3328) and Type III-Dv expression vectors with spacers targeting the kozak and CDS of Venus (FIG. 21B). 48-hours after transfection, cells were washed and then run on a flow cytometer measuring forward and side scatter along with yellow (Venus) and red fluorescence (Type III-Dv expression vector). Singlet cells that showed red fluorescence (RFP+) then had the median fluorescent intensity (MFI) of Venus analyzed to determine the effect of Type III-Dv on gene expression. Results from this analysis are shown in FIG. 21C. Relative to the non-targeting control spacers, spacers targeting RNA of Venus showed an average reduction of ˜20% YFP MFI. These data show that the Type III-Dv can effectively target and knockdown gene expression of a fluorescent reporter in mammalian cells. This is comparable to reporter knockdown by a different Type III system in HEK293 cells (Colognori et al. 2023).

Visual confirmation of flow cytometry data was achieved by co-transfecting HEK293 cells under similar conditions as stated above, then fixing cells and imaging them with confocal microscopy. Representative images from this analysis are shown in FIG. 21D. The first panel shows Hoechst-stained cells for cells co-transfected with Type III-Dv vectors and Venus, which indicates the total cell population in the field of view. In the second panel, cells transfected with the Type III-Dv expression vector with control spacer 2 or targeting spacer (control S2 or S2) show red fluorescence, which is due to the presence of microRFP from the Type III-Dv expression vector. HEK293 cells transfected with Venus (pPF3328) showed green fluorescence, as indicated by arrows. In the field of view for cells co-transfected with Type III-Dv containing a control spacer and Venus, one cell is observed to show both green and red fluorescence indicating co-transfection of the vectors (circled in respective panels). The level of green fluorescence of this cell is high, indicating that no significant knockdown of Venus gene expression has occurred. In contrast, spacer 2 (S2) which targets the CDS of the fluorescent reporter Venus shows attenuation of fluorescence in cells co-transfected with the III-Dv expression vector and Venus. This result indicates the presence of Type III-Dv complex with spacers targeting the CDS of Venus reduced the levels of Venus expression, consistent with the flow cytometry data.

The data presented in this example shows that Type III-Dv CRISPR-Cas complex can be expressed in mammalian cells and used as an effective gene silencing tool (i.e. ‘CRISPRi’) for specific targeting of mRNAs to repress gene/protein expression. Applicants observed approximately 20% knockdown efficiency when targeting either the kozak sequence or CDS of a fluorescent reporter expressed from a strong promoter.

### Example 6: Endogenous mRNA Targeting and Gene Repression of MAP2 in DRG Sensory Neurons

### Materials and Methods

### Construction of Type III-Dv Vectors for Expression in DRG Sensory Neurons

Guides (annealed oligonucleotides, see Table 12) were cloned into the vector pPF3610 using a BsaI restriction site. Clones were confirmed by Sanger sequencing.

### DRG Neuron Culture and Electroporation

DRG sensory neuron cultures were grown as previously described (Gumy et al 2017). Briefly, whole DRG were isolated from adult female Sprague Dawley rats (10 weeks or older). DRG neurons were dissociated with 2 mg/ml collagenase type IV (Worthington Biochemical Corp), 1 mg/ml trypsin (Sigma-Aldrich) and mechanical trituration. Dissociated neurons were plated on glass coverslips pre-treated with 20 mg/ml poly-D-lysine (Sigma-Aldrich) and 10 mg/ml laminin (Sigma-Aldrich), and neurons were grown in neuron culture media containing DMEM (Thermo Fisher Scientific), 1% FBS (Thermo Fisher Scientific) and 1% Pen-Strep-fungizone (Sigma-Aldrich) at 37° C. in 5% CO₂.

Transfection of neurons was performed using the Neon electroporator system (Thermo Fisher Scientific). Neurons were electroporated in suspension, in a 10 ml volume containing ˜1×10⁵ cells, with 1 mg DNA. For the first 24 hours, transfected neurons were grown in antibiotic-free neuron culture media. Neurons were fixed at 5 DIV using 4% paraformaldehyde (PFA; Sigma-Aldrich).

### Fixed-Cell Imaging

Images were acquired as z-stack acquisitions using an Andor Dragonfly spinning disk confocal on a Nikon Ti2-E inverted microscope with 60×1.49 N.A. or 100×1.45 N.A. oil-immersion objectives and an Andor iXon Ultra EMCCD camera, using Fusion 2.3.0.36 Software (Andor Technology Limited). Images were scaled, analysed and prepared in ImageJ (NIH) and Adobe Illustrator CS6 (Adobe Inc).

### Quantification of Immunofluorescence

Images were acquired using the same exposure settings and fluorescence intensity was maintained below saturation threshold. For fluorescence intensity measurements along the axon, line profiles were generated by tracing a segmented line starting at the border point where the cell body ends and the axon begins, and plotting to a distance of 100 mm into the axon. Fluorescence values were represented as arbitrary units (A.U). For fluorescence intensity in cell bodies, integrated densities were calculated (intensity/mm², AU). All fluorescence measures were obtained using Fiji (NIH) and averaged over several cells and a minimum of three experimental replicates.

TABLE 12
Oligonucleotides used for guides

| | | | SEQ ID |
|---|---|---|---|
| Name | Sequence (5′-3′) | Notes | NO: |
| PF7350 | aaacCTTACTCTTGTCAGCTATATCCTCTTC | F MAP2 Spacer 1 + | 138 |
| | AAAAAGTCCGTTCAACACCCTCTTTTCCC | Repeat, clone into | |
| | CGTCAGGGGACTG | BsaI | |
| PF7351 | gtttCAGTCCCCTGACGGGGAAAAGAGG | R MAP2 Spacer 1 + | 139 |
| | GTGTTGAACGGACTTTTTGAAGAGGATAT | Repeat, clone into | |
| | AGCTGACAAGAGTAAG | BsaI | |
| PF7352 | aaacTTCCGCTAGTGTTGGTTAGAATATCAG | F MAP2 Spacer 2 + | 140 |
| | AAGCCAGAGGTTCAACACCCTCTTTTCC | Repeat, clone into | |
| | CCGTCAGGGGACTG | BsaI | |
| PF7353 | gtttCAGTCCCCTGACGGGGAAAAGAGG | R MAP2 Spacer 2 + | 141 |
| | GTGTTGAACCTCTGGCTTCTGATATTCTA | Repeat, clone into | |
| | ACCAACACTAGCGGAA | BsaI | |
| PF7354 | aaacCGTAGTAATCACTGCCCAACCCAGTG | F MAP2 Spacer 3 + | 142 |
| | CTTCTGGTCAGTTCAACACCCTCTTTTCC | Repeat, clone into | |
| | CCGTCAGGGGACTG | BsaI | |
| PF7355 | gtttCAGTCCCCTGACGGGGAAAAGAGG | R MAP2 Spacer 3 + | 143 |
| | GTGTTGAACTGACCAGAAGCACTGGGTT | Repeat, clone into | |
| | GGGCAGTGATTACTACG | BsaI | |
| PF7356 | aaacAATTAGTGAAAGGTCAGTGGCCAAAT | F MAP2 Spacer 4 + | 144 |
| | CTCTACGGACGTTCAACACCCTCTTTTCC | Repeat, clone into | |
| | CCGTCAGGGGACTG | BsaI | |
| PF7357 | gtttCAGTCCCCTGACGGGGAAAAGAGG | R MAP2 Spacer | 145 |
| | GTGTTGAACGTCCGTAGAGATTTGGCCAC | scControl + Repeat, | |
| | TGACCTTTCACTAATT | clone into BsaI | |
| PF7358 | aaacGGATATAGCTGACAAGAGTAAGCTCG | F MAP2 Spacer 1 + | 146 |
| | AAGGCGCTGGGTTCAACACCCTCTTTTC | Repeat, clone into | |
| | CCCGTCAGGGGACTG | BsaI | |
| PF7359 | gtttCAGTCCCCTGACGGGGAAAAGAGG | R MAP2 Spacer | 147 |
| | GTGTTGAACCCAGCGCCTTCGAGCTTACT | scControl + Repeat, | |
| | CTTGTCAGCTATATCC | clone into BsaI | |
| PF7301 | aaacGAGGATTACACGGCATAGGTCAGCCT | F MAP2 Spacer | 121 |
| | AAGTCATCGAGTTCAACACCCTCTTTTCC | control-1 + Repeat, | |
| | CCGTCAGGGGACTG | clone into BsaI | |
| PF7302 | gtttCAGTCCCCTGACGGGGAAAAGAGG | R MAP2 Spacer | 122 |
| | GTGTTGAACTCGATGACTTAGGCTGACCT | control-1 + Repeat, | |
| | ATGCCGTGTAATCCTC | clone into BsaI | |

### Results

The role of MAP2 in axon trafficking of sensory neurons has previously been investigated by depleting DRG neurons of MAP2 using short hairpin RNAs (shRNAs) (Gumy et al 2017). To test the ability of the Type III-Dv system to target endogenous genes in primary cells, we chose to target MAP2 in DRG neurons. We designed and cloned MAP2-targeting Type III-Dv constructs with guides targeting various regions of rat MAP2 (FIG. 22A (i)). A control targeting the reverse sequence of MAP2-1 guide was included (III-Dv-Control-1) as well as a scrambled sequence control (III-Dv-scControl). To test whether the addition of double or triple copies of spacers enhanced repression of protein expression compared to that of constructs containing single insert repeats, one of the targeting guides (MAP2 guide 3) was cloned as single, double and triple spacer-repeat units (III-Dv-MAP2-3, I-IIDv-MAP2-3_2 and III-Dv-MAP2-3_3 respectively).

To establish whether constructs reduced MAP2 expression in DRG sensory neurons, we transfected these plasmids into rat sensory neurons in vitro, fixed cells after 5 days and immunostained them for endogenous MAP2. FIG. 22A(ii,iii) depicts endogenous levels of MAP2 in the cell body and axons of neurons transfected with miniRFP-tagged Type III-Dv constructs. MAP2 expression is prominent at the proximal axon of DRG sensory neurons (Gumy et al 2017). In the presence of Type III-Dv-MAP2 guides, particularly guides 2 and 3 (with double and triple spacer inserts) and guide 4, MAP2 fluorescence in the cell body was significantly reduced compared to controls (FIG. 22B), indicating these constructs can deplete MAP2 in sensory neurons. Additionally, analysis of MAP2 fluorescence intensity along the axon demonstrated a significant reduction of MAP2 in the proximal axon of sensory neurons transfected with Type III-Dv MAP2-guides compared to controls (FIG. 22C). Using MAP2 and DRGs as a model, these data confirm that the Type III-Dv CRISPR-Cas system can specifically target endogenous mRNAs in primary cells and decrease protein expression. Use of different guides should allow the Type III-Dv complex to target specific mRNAs in any cell.

### REFERENCES

1. Athukoralage et al. 2020. The dynamic interplay of host and viral enzymes in Type III CRISPRmediated cyclic nucleotide signaling. DOI: https://doi.org/10.7554/eLife.55852
2. Barrangou, R., Fremaux, C., Deveau, H., Richards, M., Boyaval, P., Moineau, S., Romero, D. A., & Horvath, P. (2007). CRISPR provides acquired resistance against viruses in prokaryotes. Science, 315(5819), 1709-1712. https://doi.org/10.1126/SCIENCE. 1138140/SUPPL_FILE/BARRANGOU.SOM.PDF
3. Brouns, S. J. J., Jore, M. M., Lundgren, M., Westra, E. R., Slijkhuis, R. J. H., Snijders, A. P. L., Dickman, M. J., Makarova, K. S., Koonin, E. v., & van der Oost, J. (2008). Small CRISPR RNAS guide antiviral defense in prokaryotes. Science, 321(5891), 960-964. https://doi.org/10.1126/SCIENCE. 1159689/SUPPL_FILE/BROUNS.SOM.PDF
4. Colognori, D., Trinidad, M., Doudna, J. A. (2023). Precise transcript targeting by CRISPR-Csm complexes. Nat. Biotechnol. https://doi.org/10.1038/s41587-022-01649-9
5. Crooks, G. E., Hon, G., Chandonia, J. M., and Brenner, S. E. (2004). WebLogo: a sequence logo generator. Genome Res 14, 1188-1190
6. Gruschow, S., Athukoralage, J. S., Graham, S., Hoogeboom, T., and White, M. F. (2019). Cyclic oligoadenylate signalling mediates Mycobacterium tuberculosis CRISPR defence. Nucleic Acids Res 47, 9259-9270.
7. Gumy, L. F., Katrukha, E. A., Grigoriev, I., Jaarsma, D., Kapitein, L. C., Akhmanova, A., Hoogenraad, C. C. (2017). MAP2 Defines a Pre-axonal Filtering Zone to Regulate KIF1-versus KIF5-Dependent Cargo Transport in Sensory Neurons. Neuron 94:347-362.e7
8. Holm, L. (2020). Using Dali for Protein Structure Comparison. Methods in Molecular Biology (Clifton, N.J.), 2112, 29-42. https://doi.org/10.1007/978-1-0716-0270-6_3
9. Jia, N., Jones, R., Sukenick, G., & Patel, D. J. (2019). Second Messenger cA4 Formation within the Composite Csm1 Palm Pocket of Type III-A CRISPR-Cas Csm Complex and Its Release Path. Molecular Cell, 75(5), 933-943.e6. https://doi.org/10.1016/J.MOLCEL.2019.06.013
10. Jia, N., Mo, C. Y., Wang, C., Eng, E. T., Marraffini, L. A., & Patel, D. J. (2019). Type III-A CRISPR-Cas Csm Complexes: Assembly, Periodic RNA Cleavage, DNase Activity Regulation, and Autoimmunity. Molecular Cell, 73(2), 264-277.e5. https://doi.org/10.1016/J.MOLCEL.2018.11.007
11. Jumper, J., Evans, R., Pritzel, A., Green, T., Figurnov, M., Ronneberger, O., Tunyasuvunakool, K., Bates, R., Žídek, A., Potapenko, A., Bridgland, A., Meyer, C., Kohl, S. A. A., Ballard, A. J., Cowie, A., Romera-Paredes, B., Nikolov, S., Jain, R., Adler, J., . . . . Hassabis, D. (2021). Highly accurate protein structure prediction with AlphaFold. Nature 2021 596:7873, 596(7873), 583-589. https://doi.org/10.1038/s41586-021-03819-2
12. Kato, K., Zhou, W., Okazaki, S., Isayama, Y., Nishizawa, T., Gootenberg, J. S., Abudayyeh, O. O., & Nishimasu, H. (2022). Structure and engineering of the Type III-E CRISPR-Cas7-11 effector complex. Cell. https://doi.org/10.1016/J.CELL.2022.05.003
13. Kazlauskiene, M., Kostiuk, G., Venclovas, Č., Tamulaitis, G., & Siksnys, V. (2017). A cyclic oligonucleotide signaling pathway in Type III CRISPR-Cas systems. Science (New York, N.Y.), 357(6351), 605-609. https://doi.org/10.1126/SCIENCE.AA00100
14. Langmead, B., and Salzberg, S. L. (2012). Fast gapped-read alignment with Bowtie 2. Nat Methods 9, 357-359.
15. Lau, R. K., Ye, Q., Birkholz, E. A., Berg, K. R., Patel, L., Mathews, I. T., Watrous, J. D., Ego, K., Whiteley, A. T., Lowey, B., et al. (2020). Structure and Mechanism of a Cyclic Trinucleotide-Activated Bacterial Endonuclease Mediating Bacteriophage Immunity. Mol Cell.
16. Li, H., Handsaker, B., Wysoker, A., Fennell, T., Ruan, J., Homer, N., Marth, G., Abecasis, G., Durbin, R., and Genome Project Data Processing, S. (2009). The Sequence Alignment/Map format and SAMtools. Bioinformatics 25, 2078-2079.
17. Makarova, K. S., Anantharaman, V., Grishin, N. V., Koonin, E. V., and Aravind, L. (2014). CARF and WYL domains: ligand-binding regulators of prokaryotic defense systems. Front Genet 5, 102.
18. Makarova, K. S., Timinskas, A., Wolf, Y. I., Gussow, A. B., Siksnys, V., Venclovas, Č., & Koonin, E. v. (2020). Evolutionary and functional classification of the CARF domain superfamily, key sensors in prokaryotic antivirus defense. Nucleic Acids Research, 48(16), 8828-8847. https://doi.org/10.1093/NAR/GKAA635
19. Makarova, K. S., Wolf, Y. I., Iranzo, J., Shmakov, S. A., Alkhnbashi, O. S., Brouns, S. J. J., Charpentier, E., Cheng, D., Haft, D. H., Horvath, P., Moineau, S., Mojica, F. J. M., Scott, D., Shah, S. A., Siksnys, V., Terns, M. P., Venclovas, C., White, M. F., Yakunin, A. F., . . . . Koonin, E. v. (2020). Evolutionary classification of CRISPR-Cas systems: a burst of class 2 and derived variants. Nature Reviews Microbiology, 18(2), 67-83. https://doi.org/10.1038/s41579-019-0299-x
20. Malone, L. M., Warring, S. L., Jackson, S. A., Warnecke, C., Gardner, P. P., Gumy, L. F., and Fineran, P. C. (2020). A jumbo phage that forms a nucleus-like structure evades CRISPR-Cas DNA targeting but is vulnerable to Type III RNA-based immunity. Nat Microbiol 5, 48-55.
21. Martin, M. (2011). Cutadapt removes adapter sequences from high-throughput sequencing reads. 2011 17, 3.
22. McBride, T. M., Schwartz, E. A., Kumar, A., Taylor, D. W., Fineran, P. C., & Fagerlund, R. D. (2020). Diverse CRISPR-Cas Complexes Require Independent Translation of Small and Large Subunits from a Single Gene. Molecular Cell, 80(6), 971-979.e7. https://doi.org/10.1016/j.molcel.2020.11.003
23. Niewoehner, O., Garcia-Doval, C., Rostøl, J. T., Berk, C., Schwede, F., Bigler, L., Hall, J., Marraffini, L. A., & Jinek, M. (2017). Type III CRISPR-Cas systems produce cyclic oligoadenylate second messengers. Nature 2017 548:7669, 548(7669), 543-548. https://doi.org/10.1038/nature23467
24. Osawa, T., Inanaga, H., Sato, C., & Numata, T. (2015). Crystal structure of the crispr-cas RNA silencing cmr complex bound to a target analog. Molecular Cell, 58(3), 418-430. https://doi.org/10.1016/J.MOLCEL.2015.03.018/ATTACHMENT/C1CE883A-DDFA-4127-97C9-2E47A42DAA26/MMC1.PDF
25. Özcan, A., Krajeski, R., Ioannidi, E., Lee, B., Gardner, A., Makarova, K. S., Koonin, E. v., Abudayyeh, O. O., & Gootenberg, J. S. (2021). Programmable RNA targeting with the single-protein CRISPR effector Cas7-11. Nature 2021 597:7878, 597(7878), 720-725. https://doi.org/10.1038/s41586-021-03886-5
26. Pedersen, B. S., and Quinlan, A. R. (2018). Mosdepth: quick coverage calculation for genomes and exomes. Bioinformatics 34, 867-868.
27. Quinlan, A. R., and Hall, I. M. (2010). BEDTools: a flexible suite of utilities for comparing genomic features. Bioinformatics 26, 841-842.
28. Rouillon, C., Athukoralage, J. S., Graham, S., Gruschow, S., & White, M. F. (2018). Control of cyclic oligoadenylate synthesis in a Type III CRISPR system. ELife, 7. https://doi.org/10.7554/ELIFE.36734
29. Rouillon, C., Zhou, M., Zhang, J., Politis, A., Beilsten-Edmands, V., Cannone, G., Graham, S., Robinson, C. v., Spagnolo, L., & White, M. F. (2013). Structure of the CRISPR interference complex CSM reveals key similarities with cascade. Molecular Cell, 52(1), 124-134. https://doi.org/10.1016/J.MOLCEL.2013.08.020
30. Santiago-Frangos, A., et al. (2021) Intrinsic signal amplification by Type III CRISPR-Cas systems provides a sequence-specific SARS-COV-2 diagnostic. Cell Rep Med, 2, 100319.
31. Scholz, I., Lange, S. J., Hein, S., Hess, W. R., & Backofen, R. (2013). CRISPR-Cas systems in the cyanobacterium Synechocystis sp. PCC6803 exhibit distinct processing pathways involving at least two and Cas6 a Cmr2 protein. PloS One, 8(2). https://doi.org/10.1371/JOURNAL.PONE.0056470
32. Schwartz, E. A., Mcbride, T. M., Bravo, J. P. K., Wrapp, D., Fineran, P. C., Fagerlund, R. D., Taylor, D. W., & Bravo, J. P. K. D. (2022). Structural rearrangements allow nucleic acid discrimination by type I-D Cascade. Nature Communications 2022 13:1, 13(1), 1-11. https://doi.org/10.1038/s41467-022-30402-8
33. Sofos, N., Feng, M., Stella, S., Pape, T., Fuglsang, A., Lin, J., Huang, Q., Li, Y., She, Q., & Montoya, G. (2020). Structures of the Cmr-β Complex Reveal the Regulation of the Immunity Mechanism of Type III-B CRISPR-Cas. Molecular Cell, 79(5), 741-757.e7. https://doi.org/10.1016/J.MOLCEL.2020.07.008/ATTACHMENT/F41BE4AB-8F85-4789-8A88-80AA23A8D4D2/MMC2.PDF
34. Staals, R. H. J., Agari, Y., Maki-Yonekura, S., Zhu, Y., Taylor, D. W., vanDuijn, E., Barendregt, A., Vlot, M., Koehorst, J. J., Sakamoto, K., Masuda, A., Dohmae, N., Schaap, P. J., Doudna, J. A., Heck, A. J. R., Yonekura, K., van der Oost, J., & Shinkai, A. (2013). Structure and activity of the RNA-targeting Type III-B CRISPR-Cas complex of Thermus thermophilus. Molecular Cell, 52(1), 135. https://doi.org/10.1016/J.MOLCEL.2013.09.013
35. Steens, J. A. et al. (2021) SCOPE enables Type III CRISPR-Cas diagnostics using flexible targeting and stringent CARF ribonuclease activation. Nat. Commun., 12, 5033.
36. Tamulaitis, G., Kazlauskiene, M., Manakova, E., Venclovas, Č., Nwokeoji, A. O., Dickman, M. J., Horvath, P., & Siksnys, V. (2014). Programmable RNA Shredding by the Type III-A CRISPR-Cas System of Streptococcus thermophilus. Molecular Cell, 56(4), 506-517. https://doi.org/10.1016/J.MOLCEL.2014.09.027
37. van Beljouw, S. P. B., Haagsma, A. C., Rodríguez-Molina, A., van den Berg, D. F., Vink, J. N. A., & Brouns, S. J. J. (2021). The gRAMP CRISPR-Cas effector is an RNA endonuclease complexed with a caspase-like peptidase. Science (New York, N.Y.), 373(6561), 1349-1353. https://doi.org/10.1126/SCIENCE.ABK2718
38. Ye, Q., Lau, R. K., Mathews, I. T., Birkholz, E. A., Watrous, J. D., Azimi, C. S., Pogliano, J., Jain, M., and Corbett, K. D. (2020). HORMA Domain Proteins and a Trip13-like ATPase Regulate Bacterial cGAS-like Enzymes to Mediate Bacteriophage Immunity. Mol Cell 77, 709-722 e707.
39. You, L., Ma, J., Wang, J., Artamonova, D., Wang, M., Liu, L., Xiang, H., Severinov, K., Zhang, X., & Wang, Y. (2019). Structure Studies of the CRISPR-Csm Complex Reveal Mechanism of Co-transcriptional Interference. Cell, 176(1-2), 239-253.e16. https://doi.org/10.1016/J.CELL.2018.10.052
40. Jumper, J., Evans, R., Pritzel, A. et al. “Highly accurate protein structure prediction with AlphaFold”. Nature 596, 583-589 (2021). https://doi.org/10.1038/s41586-021-03819-2)

SEQUENCES

SEQ ID NO: 1. cas10 DNA sequence (GenBank: BAD01969.1)
ATGTTTCTAGTTCTAATTGAGACTTCCGGTAATCAGCATTTTATTTTCTCGACTAATAAACTAAGGGAAAAT
ATTGGTGCATCAGAGTTGACCTATCTTGCTACAACGGAAATATTGTTCCAGGGGGTGGATAGGGTTTTCCAGACT
AACTACTATGACCAATGGTCTGACACAAACTCCCTAAATTTTTTGGCAGATAGTAAGCTTAATCCCGCCATTGATG
ATCCTAAAAATAACGCTGACATTGAAATTTTATTGGCTACCTCTGGAAAGGCGATCGCCCTGGTGAAAGAAGAGG
GCAAGGCTAAACAATTAATTAAAGAAGTTACCAAGCAGGCCCTAATCAATGCCCCGGGTTTAGAAATTGGTGGTA
TTTATGTGAATTGTAATTGGCAAGATAAATTAGGGGTTGCCAAAGCAGTTAAAGAAGCCCATAAACAGTTCGAAG
TAAATAGGGCTAAACGGGCTGGGGCTAATGGTCGCTTTTTGCGGTTACCGATCGCCGCTGGGTGCAGTGTAAGT
GAATTGCCTGCCTCTGATTTTGACTATAATGCCGATGGTGACAAGATTCCTGTTTCTACAGTCAGTAAAGTTAAAC
GGGAGACTGCGAAATCTGCCAAAAAACGTTTGCGGAGCGTTGATGGTCGGCTAGTTAACGACCTAGCACAATTA
GAAAAGTCCTTTGACGAATTAGATTGGTTAGCAGTGGTCCATGCCGATGGTAATGGTTTGGGGCAAATTTTACTA
AGTCTTGAGAAATATATTGGTGAGCAAACAAACCGCAATTATATTGATAAATATCGTAGACTTTCTTTAGCCCTGG
ATAACTGCACCATCAACGCTITTAAAATGGCGATCGCTGTCTTCAAAGAAGATTCCAAAAAAATTGATTTACCCAT
TGTCCCATTGATTTTAGGTGGAGATGACCTAACGGTAATTTGTCGGGGGGACTACGCCCTAGAATTCACCAGGG
AATTTCTTGAAGCATTTGAAGGGCAGACAGAAACACATGATGATATCAAAGTAATAGCCCAAAAAGCCTTTGGCG
TTGATCGCCTTTCTGCCTGCGCTGGGATCAGTATTATTAAGCCCCATTTTCCCTTCTCTGTTGCCTATACTTTGGC
GGAAAGATTAATTAAATCAGCTAAGGAGGTCAAACAAAAAGTTACTGTGACAAATAGTTCGCCAATAACTCCTTT
TCCCTGCTCTGCCATTGATTTTCATATTCTCTATGACAGTAGCGGCATTGATTTTGACCGTATTCGTGAAAAATTA
CGGCCGGAAGATAATACCGAGCTTTACAACCGTCCCTATGTGGTGACAGCAGCGGAGAACCTCAGCCAAGCCCA
GGGTTATGAATGGTCCCAGGCCCACAGTTTGCAAACACTAGCGGATCGGGTTAGTTATTTACGTTCCGAAGATG
GGGAAGGAAAATCTGCATTACCCAGCAGTCAAAGCCATGCCCTACGAACGGCATTGTACCTAGAGAAAAATGAA
GCAGACGCTCAATATAGCTTAATTAGCCAACGCTACAAAATTCTCAAAAACTTTGCGGAGGACGGAGAGAATAAA
TCACTATTTCATCTCGAAAATGGCAAGTACGTCACCAGATTTTTAGATGCACTGGATGCCAAAGATTTTTTTGCTA
ACGCTAACCATAAAAACCAAGGAGAATAA
SEQ ID NO: 2. Cas10 protein sequence (GenBank: BAD01969.1) HD and palm domains are
in bold
MFLVLIETSGNQHFIFSTNKLRENIGASELTYLATTEILFQGVDRVFQTNYYDQWSDTNSLNFLADSKLNPAID
DPKNNADIEILLATSGKAIALVKEEGKAKQLIKEVTKQALINAPGLEIGGIYVNCNWQDKLGVAKAVKEAHKQFEVNR
AKRAGANGRFLRLPIAAGCSVSELPASDFDYNADGDKIPVSTVSKVKRETAKSAKKRLRSVDGRLVNDLAQLEKSFD
ELDWLAVVHADGNGLGQILLSLEKYIGEQTNRNYIDKYRRLSLALDNCTINAFKMAIAVFKEDSKKIDLPIVPLILGGD
DLTVICRGDYALEFTREFLEAFEGQTETHDDIKVIAQKAFGVDRLSACAGISIIKPHFPFSVAYTLAERLIKSAKEVKQ
KVTVTNSSPITPFPCSAIDFHILYDSSGIDFDRIREKLRPEDNTELYNRPYVVTAAENLSQAQGYEWSQAHSLQTLAD
RVSYLRSEDGEGKSALPSSQSHALRTALYLEKNEADAQYSLISQRYKILKNFAEDGENKSLFHLENGKYVTRFLDALD
AKDFFANANHKNQGE
SEQ ID NO: 3. Cas7-5-11 DNA sequence (GenBank: BAD01968.1)
ATGCGAGGAATTGAGATAACCATAACCATGCAGAGTGATTGGCACGTTGGCACTGGCATGGGTCGGGGG
GAACTGGACAGTGTTGTACAACGGGATGGAGATAATCTGCCCTATATTCCCGGCAAAACCTTAACAGGTATTCTG
CGGGATAGCTGTGAACAGGTTGCCCTAGGTTTAGATAATGGTCAAACCCGAGGGCTTTGGCATGGGTGGATTAA
TITTATTTTTGGCGATCAACCTGCCCTAGCTCAAGGAGCTATTGAGCCAGAACCTAGACCTGCCCTAATCGCCAT
TGGTTCTGCACACCTTGACCCTAAGTTAAAAGCGGCTTTTCAGGGCAAAAAACAATTGCAAGAGGCGATCGCCTT
TATGAAGCCAGGGGTGGCTATCGATGCAATCACGGGCACAGCTAAGAAAGATTTTTTACGCTTTGAAGAAGTAG
TTCGTTTGGGAGCGAAATTAACTGCGGAAGTTGAGTTAAATTTACCCGATAATTTGAGCGAAACCAATAAAAAAG
TTATTGCTGGTATTTTAGCCAGTGGAGCAAAGTTAACCGAGAGATTAGGCGGTAAACGTCGCCGGGGCAATGGG
CGCTGTGAATTAAAATTTAGTGGTTATTCTGATCAACAAATTCAATGGTTGAAAGACAATTATCAATCTGTTGATC
AACCACCTAAGTATCAACAAAATAAATTACAATCTGCCGGAGATAATCCAGAACAGCAACCCCCTTGGCATATTA
TTCCCTTAACCATTAAAACCCTTTCTCCTGTTGTTTTACCAGCTCGTACAGTCGGTAACGTTGTCGAATGTTTAGA
CTATATTCCCGGGCGTTATCTACTGGGCTATATTCACAAAACCCTAGGGGAATATTTCGACGTTAGTCAGGCAAT
CGCCGCTGGGGATTTAATTATTACCAATGCCACGATAAAAATTGATGGTAAAGCAGGACGAGCTACCCCATTTTG
TTTGTTTGGGGAAAAACTAGATGGAGGATTAGGTAAAGGTAAAGGAGTTTATAACCGTTTCCAAGAATCGGAAC
CTGATGGCATTCAATTAAAGGGAGAACGGGGCGGCTATGTTGGCCAATTTGAACAGGAGCAAAGGAATCTGCCA
AATACGGGGAAAATTAATTCAGAGTTATTTACCCATAACACCATTCAAGATGATGTCCAGCGGCCCACCAGTGAT
GTGGGGGGAGTTTATAGCTATGAAGCTATTATAGCCGGACAAACATTCGTCGCTGAGTTACGTTTACCAGATAG
CTTAGTCAAGCAAATTACAAGCAAAAATAAAAATTGGCAAGCTCAACTAAAAGCTACAATTCGCATTGGTCAGTC
TAAAAAAGATCAGTATGGCAAAATCGAAGTTACGTCGGGAAACTCTGCTGATTTGCCTAAGCCTACGGGCAACA
ATAAAACTCTTTCTATTTGGTTCTTATCCGATATCCTTCTCCGAGGCGATCGCCTAAATTTTAATGCTACTCCGGA
TGATCTCAAAAAATACTTAGAAAATGCTCTGGATATCAAGCTCAAAGAACGATCAGACAATGATTTAATTTGCATT
GCTCTCCGTTCCCAGCGGACAGAATCCTGGCAAGTACGGTGGGGTTTACCCCGGCCATCTCTAGTGGGTTGGCA
AGCTGGTAGTTGTCTGATTTATGACATTGAATCTGGCACTGTTAATGCCGAAAAATTGCAAGAATTAATGATCAC
CGGCATTGGCGATCGGTGTACAGAGGGTTACGGTCAAATCGGTTTTAACGATCCATTACTTTCGGCTTCCCTAGG
AAAGTTGACAGCTAAGCCTAAAGCTTCTAACAATCAGTCCCAAAACAGCCAATCCAACCCATTACCCACTAATCAT
CCTACCCAAGATTATGCTCGATTAATTGAAAAAGCGGCTTGGCGGGAAGCAATTCAAAATAAAGCCTTAGCCTTG
GCATCTAGCCGAGCGAAACGGGAAGAAATTTTAGGCATTAAAATTATGGGAAAAGATAGTCAACCCACCATGAC
TCAATTAGGAGGATTTCGCTCCGTATTAAAACGGCTACACTCAAGAAATAATCGAGATATTGTCACAGGTTATTTA
ACAGCTCTAGAGCAGGTTTCTAATCGAAAAGAAAAATGGAGTAATACCAGCCAAGGATTAACTAAAATTCGTAAT
TTAGTCACCCAGGAAAATCTCATTTGGAATCATCTTGATATTGATTTTTCGCCGTTAACTATTACCCAAAATGGTG
TTAATCAGCTAAAGTCTGAACTTTGGGCGGAAGCAGTGCGAACCCTTGTTGACGCTATCATTCGGGGTCATAAAC
GGGACTTAGAAAAAGCTCAAGAAAACGAATCTAATCAACAGTCACAGGGAGCAGCTTAA
SEQ ID NO: 4. Cas7-5-11 protein sequence (GenBank: BAD01968.1) Cleavage residue is
in bold
MRGIEITITMQSDWHVGTGMGRGELDSVVQRDGDNLPYIPGKTLTGILRDSCEQVALGLDNGQTRGLWHG
WINFIFGDQPALAQGAIEPEPRPALIAIGSAHLDPKLKAAFQGKKQLQEAIAFMKPGVAIDAITGTAKKDFLRFEEVVR
LGAKLTAEVELNLPDNLSETNKKVIAGILASGAKLTERLGGKRRRGNGRCELKFSGYSDQQIQWLKDNYQSVDQPP
KYQQNKLQSAGDNPEQQPPWHIIPLTIKTLSPVVLPARTVGNVVECLDYIPGRYLLGYIHKTLGEYFDVSQAIAAGDLI
ITNATIKIDGKAGRATPFCLFGEKLDGGLGKGKGVYNRFQESEPDGIQLKGERGGYVGQFEQEQRNLPNTGKINSEL
FTHNTIQDDVQRPTSDVGGVYSYEAIIAGQTFVAELRLPDSLVKQITSKNKNWQAQLKATIRIGQSKKDQYGKIEVT
SGNSADLPKPTGNNKTLSIWFLSDILLRGDRLNFNATPDDLKKYLENALDIKLKERSDNDLICIALRSQRTESWQVR
WGLPRPSLVGWQAGSCLIYDIESGTVNAEKLQELMITGIGDRCTEGYGQIGFNDPLLSASLGKLTAKPKASNNQSQ
NSQSNPLPTNHPTQDYARLIEKAAWREAIQNKALALASSRAKREEILGIKIMGKDSQPTMTQLGGFRSVLKRLHSRN
NRDIVTGYLTALEQVSNRKEKWSNTSQGLTKIRNLVTQENLIWNHLDIDFSPLTITQNGVNQLKSELWAEAVRTLVD
AIIRGHKRDLEKAQENESNQQSQGAA
SEQ ID NO: 5. Cas7_2x DNA sequence (GenBank: BAD01967.1)
ATGGCTAGAAAAGTTACTACACGCTGGAAAATTACAGGCACATTAATTGCAGAAACCCCTTTACACATTGG
TGGTGTGGGTGGCGACGCTGATACGGATTTAGCCCTGGCGGTTAATGGTGCGGGTGAATATTATGTGCCAGGG
ACAAGTTTAGCCGGTGCTCTGCGGGGTTGGATGACCCAGTTATTGAATAATGATGAGTCCCAAATTAAAGATCTT
TGGGGTGATCATTTAGATGCAAAACGGGGAGCTAGCTTTGTTATTGTTGACGATGCGGTTATCCATATACCCAAT
AATGCTGATGTTGAAATTAGGGAGGGTGTTGGCATCGATCGCCATTTTGGAACCGCCGCCAATGGGTTTAAATA
TAGCCGAGCAGTTATTCCCAAGGGTTCTAAATTTAAATTGCCATTAACTTTTGACAGTCAAGATGATGGGCTACC
GAATGCGTTGATTCAATTGTTGTGTGCCTTAGAAGCAGGGGATATTCGCCTTGGGGCCGCAAAAACCCGGGGTT
TAGGTCGCATTAAACTAGATGATTTAAAGTTAAAATCCTTTGCTTTAGATAAACCAGAAGGTATTTTTTCTGCTTTA
TTAGACCAAGGTAAAAAATTAGATTGGAATCAATTAAAAGCAAACGTTACCTACCAGTCTCCTCCCTATCTAGGTA
TTAGTATTACCTGGAATCCCAAAGATCCCGTCATGGTGAAAGCTGAAGGGGATGGACTGGCGATCGATATTTTG
CCCCTCGTTAGTCAAGTGGGAAGTGATGTTCGATTTGTCATTCCCGGCAGTTCCATTAAGGGGATTTTACGAACC
CAGGCTGAACGTATTATTCGTACTATTTGCCAGTCTAATGGTTCTGAGAAAAACTTCCTAGAACAATTACGAATCA
ATCTGGTTAATGAATTATTTGGGTCTGCTTCTTTGAGCCAAAAACAAAATGGCAAGGATATAGATCTGGGTAAAA
TCGGAGCCTTGGCAGTGAATGATTGTTTTTCTAGTTTATCCATGACCCCAGATCAATGGAAAGCGGTAGAGAATG
CCACGGAGATGACGGGGAATTTACAGCCTGCTCTTAAACAAGCTACGGGTTATCCCAATAATATTAGCCAAGCTT
ACAAAGTACTTCAACCGGCCATGCACGTCGCTGTAGATCGGTGGACAGGGGGAGCTGCCGAAGGAATGCTTTA
CAGCGTGCTCGAACCCATTGGGGTCACCTGGGAACCGATCCAAGTTCACTTGGACATTGCCCGTCTCAAAAATT
ATTACCACGGTAAGGAAGAAAAACTTAAACCGGCGATCGCCCTATTGCTTCTTGTATTGCGGGATTTAGCTAACA
AAAAAATTCCCGTAGGCTATGGCACTAACCGCGGTATGGGAACGATTACTGTCAGTCAAATCACCCTCAATGGCA
AAGCCCTCCCCACTGAACTTGAACCTTTAAACAAAACAATGACTTGTCCTAATCTCACCGATCTAGATGAGGCATT
TCGTCAGGACTTAAGCACTGCTTGGAAAGAGTGGATTGCCGATCCCATTGATCTATGCCAGCAGGAGGCCGCCT
AA
SEQ ID NO: 6. Cas7_2x protein sequence (GenBank: BAD01967.1) Cleavage residues are
in bold
MARKVTTRWKITGTLIAETPLHIGGVGGDADTDLALAVNGAGEYYVPGTSLAGALRGWMTQLLNNDESQIK
DLWGDHLDAKRGASFVIVDDAVIHIPNNADVEIREGVGIDRHFGTAANGFKYSRAVIPKGSKFKLPLTFDSQDDGLP
NALIQLLCALEAGDIRLGAAKTRGLGRIKLDDLKLKSFALDKPEGIFSALLDQGKKLDWNQLKANVTYQSPPYLGISIT
WNPKDPVMVKAEGDGLAIDILPLVSQVGSDVRFVIPGSSIKGILRTQAERIIRTICQSNGSEKNFLEQLRINLVNELFG
SASLSQKQNGKDIDLGKIGALAVNDCFSSLSMTPDQWKAVENATEMTGNLQPALKQATGYPNNISQAYKVLQPAM
HVAVDRWTGGAAEGMLYSVLEPIGVTWEPIQVHLDIARLKNYYHGKEEKLKPAIALLLLVLRDLANKKIPVGYGTNRG
MGTITVSQITLNGKALPTELEPLNKTMTCPNLTDLDEAFRQDLSTAWKEWIADPIDLCQQEAA
SEQ ID NO: 7. csx19 DNA sequence (GenBank: BAD01966.1)
ATGCCAGCAGGAGGCCGCCTAATGAAGAACCTTTACCACTACCACCAATATGAAATTACCCTCGAATCCG
CCGTCGATTCTTGCAAAAACCATCTCCAAGCGGCGATCGGGCTGTTGTATTCTCCCCAAAAGTGTGAACTAGTCA
AACTGGATAACTCAGGCAAGTTAGTTGATTCTTACAATCGTCTTAAGTTCAATAACCTAGGCGTATTTGAAGCCC
GCTTCTTTAATCTCAATTGTGAACTGCGATGGGTCAATGAATCTAATGGTAATGGCACTGCCGTCTTGCTTTCAG
AATCGGATATTACCTTAACTGGTTTTGAGAAAGGTTTACAGGAATTTATTACGGCGATCGACCAACAGTATTTACT
CTGGGGTGAACCCGCTAAACATCCCCCTAATGCTGATGGCTGGCAACGACTAGCGGAAGCAAGGATCGGGAAA
CTCGATATTCCCCTCGATAACCCGTTAAAACCCAAAGATCGAGTTTTTCTCACCAGCGAAGAGTACATTGCTGAA
GTAGATGATTTTGGTAATTGTGCCGTTATTGACGAACGTTTAATTAAATTGGAGGTTAAGTAA
SEQ ID NO: 8. Csx19 protein sequence (GenBank: BAD01966.1)
MPAGGRLMKNLYHYHQYEITLESAVDSCKNHLQAAIGLLYSPQKCELVKLDNSGKLVDSYNRLKFNNLGVFE
ARFFNLNCELRWVNESNGNGTAVLLSESDITLTGFEKGLQEFITAIDQQYLLWGEPAKHPPNADGWQRLAEARIGKL
DIPLDNPLKPKDRVFLTSEEYIAEVDDFGNCAVIDERLIKLEVK
SEQ ID NO: 9. Cas7-insert DNA sequence (GenBank: BAD01965.1)
ATGACAGTCGGAACATTGGGCGTTGTTGGCAGTGCTAAAAACCTCAAATTACAACTTAGTITTATCAACAC
AAGGCAACAGTATGTTCAAATAACACTTTTTGAGCGAAATTCTTTTAAGGTTGCTGAGGAAGAATTTTCTACTGAA
CTTGTGGAAATCATTAAAACAGCACTACCAACTCTCAAAAATAAAAAAGTTGAATTTGAGGAAGATGGCGATCAA
ATTAAACAAATCCGAGAAAAAGGTCAAGCTTGGGTTGGTGCCGCAGAACAGATTGCACCTTATGTTCTTCCTTCT
GGAAATATTACTGAAACACCCAGAAATGTTAACGCTAGCAACTTTCATAACCCCTACAACTTTGTCCCAGCCCTAC
CCCGCGATGGCATAACCGGAGATTTAGGCGACTGTGCTCCTGCTGGTCATAGCTATTACCATGGCGATAAATAC
AGCGGCAGAATTGCCGTCAAACTAACAACCGTTACCCCTCTATTGATTCCTGACGCTTCAAAAGAAGAGATAAAT
AACAACCATAAAACCTATCCGGTTCGTATCGGCAAAGATGGCAAGCCCTATCTACCTCCCACTTCCATTAAGGGA
ATGTTGCGCTCTGCCTATGAAGCGGTCACTAATTCCCGCTTAGCCGTGTTTGAAGATCATGACTCTCGCTTGGCC
TATCGAATGCCTGCCACCATGGGATTGCAAATGGTTCCTGCCCGCATTGAAGGTGATAATATTGTTCTTTACCCA
GGAACCTCAAGGATAGGCAATAATGGCCGACCAGCTAACAATGATCCTATGTATGCGGCATGGCTTCCTTACTAT
CAAAATCGTATTGCTTATGATGGTAGTCGTGATTATCAGATGGCTGAGCATGGTGATCATGTCAGATTTTGGGCT
GAGCGATATACCAGAGGAAACTTCTGCTATTGGCGTGTCAGACAAATTGCACGACACAATCAAAATTTAGGTAAT
CGGCCTGAACGAGGACGTAATTACGGTCAACATCATTCAACAGGAGTCATTGAACAATTTGAAGGATTTGTTTAC
AAAACCAATAAAAATATTGGGAATAAACATGACGAACGAGTATTTATTATTGATCGAGAAAGTATCGAAATACCTC
TATCTCGAGATTTACGGCGAAAATGGCGAGAATTAATTACAAGCTATCAGGAAATACACAAAAAGGAAGTTGATA
GAGGTGATACTGGCCCTTCCGCTGTAAATGGGGCTGTTTGGTCACGGCAAATTATTGCAGATGAATCAGAGCGG
AATTTATCGGATGGGACTCTTTGTTATGCTCATGTTAAGAAAGAAGATGGACAGTACAAAATTCTCAATCTTTATC
CTGTAATGATCACACGGGGATTATATGAAATTGCGCCGGTTGACTTATTAGATGAAACCCTAAAGCCTGCGACGG
ATAAAAAGCAACTATCCCCAGCAGACCGCGTATTTGGCTGGGTCAATCAACGGGGCAATGGTTGCTACAAAGGA
CAATTACGAATTCATAGCGTAACTTGCCAACATGATGATGCCATTGATGATTTTGGTAATCAAAATTTCTCTGTTC
CCCTTGCTATTTTGGGACAACCTAAACCAGAACAGGCTCGTTTTTATTGTGCCGATGATCGAAAAGGAATTCCTTT
AGAAGATGGCTATGATCGTGACGACGGCTATAGTGATTCAGAACAAGGCTTGCGAGGACGCAAAGTCTATCCTC
ACCACAAGGGGTTACCAAATGGCTACTGGAGTAATCCAACGGAAGACCGAAGTCAACAAGCTATCCAAGGTCAT
TACCAAGAATATCGTCGTCCTAAAAAGGATGGTCTTGAACAAAGAGATGATCAAAATCGTTCTGTAAAAGGTTGG
GTAAAACCACTGACCGAGTTTACTTTTGAAATTGACGTTACTAATCTTTCGGAAGTTGAGTTAGGTGCTCTATTGT
GGTTGTTAACCTTACCTGATTTGCATTTCCACCGTCTAGGAGGAGGTAAACCGTTAGGTTTTGGTAGTGTTCGTT
TAGATATTGACCCTGACAAGACAGACCTAAGAAATGGGGCAGGATGGCGTGATTATTACGGCTCTTTACTAGAA
ACAAGTCAACCAGATTITACAACTCTAATTAGTCAGTGGATTAATGCTTTTCAAACGGCTGTTAAAGAGGAGTATG
GTAGCAGTAGTTTTGATCAGGTTACTTTCATCAAAGCTTCTGGTCAGAGTCTCCAAGGATTTCATGATAATGCATC
TATCCATTATCCTCGTTCTACTCCTGAGCCCAAGCCAGATGGAGAAGCTTTTAAGTGGTTTGTTGCCAATGAAAA
AGGTCGACGATTAGCCTTGCCAGCGCTGGAAAAATCCCAGAGTTTTCCAATCAAACCTAGTTAA
SEQ ID NO: 10. Cas7-insert protein sequence (GenBank: BAD01965.1)
MTVGTLGVVGSAKNLKLQLSFINTRQQYVQITLFERNSFKVAEEEFSTELVEIIKTALPTLKNKKVEFEEDGDQ
IKQIREKGQAWVGAAEQIAPYVLPSGNITETPRNVNASNFHNPYNFVPALPRDGITGDLGDCAPAGHSYYHGDKYSG
RIAVKLTTVTPLLIPDASKEEINNNHKTYPVRIGKDGKPYLPPTSIKGMLRSAYEAVTNSRLAVFEDHDSRLAYRMPAT
MGLQMVPARIEGDNIVLYPGTSRIGNNGRPANNDPMYAAWLPYYQNRIAYDGSRDYQMAEHGDHVRFWAERYTRG
NFCYWRVRQIARHNQNLGNRPERGRNYGQHHSTGVIEQFEGFVYKTNKNIGNKHDERVFIIDRESIEIPLSRDLRRK
WRELITSYQEIHKKEVDRGDTGPSAVNGAVWSRQIIADESERNLSDGTLCYAHVKKEDGQYKILNLYPVMITRGLYE
IAPVDLLDETLKPATDKKQLSPADRVFGWVNQRGNGCYKGQLRIHSVTCQHDDAIDDFGNQNFSVPLAILGQPKPE
QARFYCADDRKGIPLEDGYDRDDGYSDSEQGLRGRKVYPHHKGLPNGYWSNPTEDRSQQAIQGHYQEYRRPKKD
GLEQRDDQNRSVKGWVKPLTEFTFEIDVTNLSEVELGALLWLLTLPDLHFHRLGGGKPLGFGSVRLDIDPDKTDLRN
GAGWRDYYGSLLETSQPDFTTLISQWINAFQTAVKEEYGSSSFDQVTFIKASGQSLQGFHDNASIHYPRSTPEPKPD
GEAFKWFVANEKGRRLALPALEKSQSFPIKPS
SEQ ID NO: 11. Cas6-2a DNA sequence (GenBank: BAD01970.1)
GTGGTGGATCTAAAATCCTTAGCTGGGGCCGAAATGGTGGGATTACGCTGGCAACTGCGCTTCGACCGC
CCCTGTCGCCTGGAAAGTCATTACGTTAAAGGACTCCATGCTTGGTTTTTGCATCAAGTGCAGGCCATTGATCCC
GATGTTTCTGCCTGGCTCCATGATGGTCAAGGGGAAAAGCCCTTCACCATTTCCCGCCTGATAGGGCCTACCCT
CTGGCAAGAAGGTCATTGGCACTGGCAAATAAATAAGACCTACCATTGGCAATTAAATTTACTATCAGGGGCTTT
AATCGAAGCTTTACAACCTTGGCTAGCCCGTTTGCCAAACAAAATTGTCCTAGCTCGCCAAACATTATGGGTAGA
AGCCGTTGATTGTTACCTAGCCCCCCATAACTATCAACAGTTATGGCCCCAGGGTGCTTTACCCCGACGGCAAGA
GTTTACTTTCACTAGCCCTACCAGTTTCCGTCGCCAAGGCAATCACTATCCGTTACCAGAGCCCCGCAATGTTCT
GCAAAGTTATCTACGGCGTTGGAATGATTTTTCTGGTTTGGCGTTCGAGCCGGAGCCATTTTTGGACTATTGGGT
GCCCCAAAATGTGGTGATCGATCGCCATTGGTTGGAGTCGGTGAAGACCACAGCGGGAAAACAAGGCTCAGTG
GTGGGATTTGTGGGAGCAGTGTCCCTAGTCCTTACGCCCCAGGCCCGTAATGATGGGGATGATTATGGCCGCTT
GTTCCATGCCCTCTGTCGATATGGACCCTACTGTGGCACTGGGCATAAAACCACCTTTGGTTTGGGGCAAACAAT
GGCGGGCTGGGCTACCCCGGACCTAAAAACTTTTGCGTGCCTCCAAGAAGATTTACAGACTCAGGTGTTAACGC
AACGGATAGATCAATGCGCCTCTCTCCTCCTAGCCCAGCGTCAACGGACAGGAGGGCAGAGAGCCCAGGAAAT
TTGCCATACGCTAGCCACTATTTTTGTCCGCCGAGAACAGGGGGAATCATTGCAAGAAATCGCCCTGGATTTACA
GTTACCTTATGAGACAGCCCGCACCTACAGCAAACGAGCTAAGCGGGCCTTAGCCAATGTTCAATAA
SEQ ID NO: 12. Cas6-2a protein sequence (GenBank: BAD01970.1)
VVDLKSLAGAEMVGLRWQLRFDRPCRLESHYVKGLHAWFLHQVQAIDPDVSAWLHDGQGEKPFTISRLIGP
TLWQEGHWHWQINKTYHWQLNLLSGALIEALQPWLARLPNKIVLARQTLWVEAVDCYLAPHNYQQLWPQGALPRR
QEFTFTSPTSFRRQGNHYPLPEPRNVLQSYLRRWNDFSGLAFEPEPFLDYWVPQNVVIDRHWLESVKTTAGKQGSV
VGFVGAVSLVLTPQARNDGDDYGRLFHALCRYGPYCGTGHKTTFGLGQTMAGWATPDLKTFACLQEDLQTQVLTQ
RIDQCASLLLAQRQRTGGQRAQEICHTLATIFVRREQGESLQEIALDLQLPYETARTYSKRAKRALANVQ
Modified Sequences (modified sequence in bold):
SEQ ID NO: 13. Dead HD cas10 DNA sequence (BAD01969.1: c.1009C > G; 1010A > C; 1011T > A;
1013A > C) modified positions are in bold and underlined
ATGTTTCTAGTTCTAATTGAGACTTCCGGTAATCAGCATTTTATTTTCTCGACTAATAAACTAAGGGAAAAT
ATTGGTGCATCAGAGTTGACCTATCTTGCTACAACGGAAATATTGTTCCAGGGGGTGGATAGGGTTTTCCAGACT
AACTACTATGACCAATGGTCTGACACAAACTCCCTAAATTTTTTGGCAGATAGTAAGCTTAATCCCGCCATTGATG
ATCCTAAAAATAACGCTGACATTGAAATTTTATTGGCTACCTCTGGAAAGGCGATCGCCCTGGTGAAAGAAGAGG
GCAAGGCTAAACAATTAATTAAAGAAGTTACCAAGCAGGCCCTAATCAATGCCCCGGGTTTAGAAATTGGTGGTA
TTTATGTGAATTGTAATTGGCAAGATAAATTAGGGGTTGCCAAAGCAGTTAAAGAAGCCCATAAACAGTTCGAAG
TAAATAGGGCTAAACGGGCTGGGGCTAATGGTCGCTTTTTGCGGTTACCGATCGCCGCTGGGTGCAGTGTAAGT
GAATTGCCTGCCTCTGATTTTGACTATAATGCCGATGGTGACAAGATTCCTGTTTCTACAGTCAGTAAAGTTAAAC
GGGAGACTGCGAAATCTGCCAAAAAACGTTTGCGGAGCGTTGATGGTCGGCTAGTTAACGACCTAGCACAATTA
GAAAAGTCCTTTGACGAATTAGATTGGTTAGCAGTGGTCCATGCCGATGGTAATGGTTTGGGGCAAATTTTACTA
AGTCTTGAGAAATATATTGGTGAGCAAACAAACCGCAATTATATTGATAAATATCGTAGACTTTCTTTAGCCCTGG
ATAACTGCACCATCAACGCTTTTAAAATGGCGATCGCTGTCTTCAAAGAAGATTCCAAAAAAATTGATTTACCCAT
TGTCCCATTGATTTTAGGTGGAGATGACCTAACGGTAATTTGTCGGGGGGACTACGCCCTAGAATTCACCAGGG
AATTTCTTGAAGCATTTGAAGGGCAGACAGAAACAGCAGCTGATATCAAAGTAATAGCCCAAAAAGCCTTTGGC
GTTGATCGCCTTTCTGCCTGCGCTGGGATCAGTATTATTAAGCCCCATTTTCCCTTCTCTGTTGCCTATACTTTGG
CGGAAAGATTAATTAAATCAGCTAAGGAGGTCAAACAAAAAGTTACTGTGACAAATAGTTCGCCAATAACTCCTT
TTCCCTGCTCTGCCATTGATTTTCATATTCTCTATGACAGTAGCGGCATTGATTTTGACCGTATTCGTGAAAAATT
ACGGCCGGAAGATAATACCGAGCTTTACAACCGTCCCTATGTGGTGACAGCAGCGGAGAACCTCAGCCAAGCCC
AGGGTTATGAATGGTCCCAGGCCCACAGTTTGCAAACACTAGCGGATCGGGTTAGTTATTTACGTTCCGAAGAT
GGGGAAGGAAAATCTGCATTACCCAGCAGTCAAAGCCATGCCCTACGAACGGCATTGTACCTAGAGAAAAATGA
AGCAGACGCTCAATATAGCTTAATTAGCCAACGCTACAAAATTCTCAAAAACTTTGCGGAGGACGGAGAGAATAA
ATCACTATTTCATCTCGAAAATGGCAAGTACGTCACCAGATTTTTAGATGCACTGGATGCCAAAGATTTTTTTGCT
AACGCTAACCATAAAAACCAAGGAGAATAA
SEQ ID NO: 14. Dead HD Cas10d protein sequence (BAD01969.1: p.H337A; D338A) modified
residues are in bold and underlined
MFLVLIETSGNQHFIFSTNKLRENIGASELTYLATTEILFQGVDRVFQTNYYDQWSDTNSLNFLADSKLNPAID
DPKNNADIEILLATSGKAIALVKEEGKAKQLIKEVTKQALINAPGLEIGGIYVNCNWQDKLGVAKAVKEAHKQFEVNR
AKRAGANGRFLRLPIAAGCSVSELPASDFDYNADGDKIPVSTVSKVKRETAKSAKKRLRSVDGRLVNDLAQLEKSFD
ELDWLAVVHADGNGLGQILLSLEKYIGEQTNRNYIDKYRRLSLALDNCTINAFKMAIAVFKEDSKKIDLPIVPLILGGD
DLTVICRGDYALEFTREFLEAFEGQTETAADIKVIAQKAFGVDRLSACAGISIIKPHFPFSVAYTLAERLIKSAKEVKQK
VTVTNSSPITPFPCSAIDFHILYDSSGIDFDRIREKLRPEDNTELYNRPYVVTAAENLSQAQGYEWSQAHSLQTLADR
VSYLRSEDGEGKSALPSSQSHALRTALYLEKNEADAQYSLISQRYKILKNFAEDGENKSLFHLENGKYVTRFLDALDA
KDFFANANHKNQGE
SEQ ID NO: 15. Dead palm cas10 DNA sequence (BAD01969.1: c.923C > A;926C > A) modified
positions are in bold and underlined.
ATGTTTCTAGTTCTAATTGAGACTTCCGGTAATCAGCATTTTATTTTCTCGACTAATAAACTAAGGGAAAAT
ATTGGTGCATCAGAGTTGACCTATCTTGCTACAACGGAAATATTGTTCCAGGGGGTGGATAGGGTTTTCCAGACT
AACTACTATGACCAATGGTCTGACACAAACTCCCTAAATTTTTTGGCAGATAGTAAGCTTAATCCCGCCATTGATG
ATCCTAAAAATAACGCTGACATTGAAATTTTATTGGCTACCTCTGGAAAGGCGATCGCCCTGGTGAAAGAAGAGG
GCAAGGCTAAACAATTAATTAAAGAAGTTACCAAGCAGGCCCTAATCAATGCCCCGGGTTTAGAAATTGGTGGTA
TTTATGTGAATTGTAATTGGCAAGATAAATTAGGGGTTGCCAAAGCAGTTAAAGAAGCCCATAAACAGTTCGAAG
TAAATAGGGCTAAACGGGCTGGGGCTAATGGTCGCTTTTTGCGGTTACCGATCGCCGCTGGGTGCAGTGTAAGT
GAATTGCCTGCCTCTGATTTTGACTATAATGCCGATGGTGACAAGATTCCTGTTTCTACAGTCAGTAAAGTTAAAC
GGGAGACTGCGAAATCTGCCAAAAAACGTTTGCGGAGCGTTGATGGTCGGCTAGTTAACGACCTAGCACAATTA
GAAAAGTCCTTTGACGAATTAGATTGGTTAGCAGTGGTCCATGCCGATGGTAATGGTTTGGGGCAAATTTTACTA
AGTCTTGAGAAATATATTGGTGAGCAAACAAACCGCAATTATATTGATAAATATCGTAGACTTTCTTTAGCCCTGG
ATAACTGCACCATCAACGCTTTTAAAATGGCGATCGCTGTCTTCAAAGAAGATTCCAAAAAAATTGATTTACCCAT
TGTCCCATTGATTTTAGGTGGAGCTGCCCTAACGGTAATTTGTCGGGGGGACTACGCCCTAGAATTCACCAGGG
AATTTCTTGAAGCATTTGAAGGGCAGACAGAAACACATGATGATATCAAAGTAATAGCCCAAAAAGCCTTTGGCG
TTGATCGCCTTTCTGCCTGCGCTGGGATCAGTATTATTAAGCCCCATTTTCCCTTCTCTGTTGCCTATACTTTGGC
GGAAAGATTAATTAAATCAGCTAAGGAGGTCAAACAAAAAGTTACTGTGACAAATAGTTCGCCAATAACTCCTTT
TCCCTGCTCTGCCATTGATTTTCATATTCTCTATGACAGTAGCGGCATTGATTTTGACCGTATTCGTGAAAAATTA
CGGCCGGAAGATAATACCGAGCTTTACAACCGTCCCTATGTGGTGACAGCAGCGGAGAACCTCAGCCAAGCCCA
GGGTTATGAATGGTCCCAGGCCCACAGTTTGCAAACACTAGCGGATCGGGTTAGTTATTTACGTTCCGAAGATG
GGGAAGGAAAATCTGCATTACCCAGCAGTCAAAGCCATGCCCTACGAACGGCATTGTACCTAGAGAAAAATGAA
GCAGACGCTCAATATAGCTTAATTAGCCAACGCTACAAAATTCTCAAAAACTTTGCGGAGGACGGAGAGAATAAA
TCACTATTTCATCTCGAAAATGGCAAGTACGTCACCAGATTTTTAGATGCACTGGATGCCAAAGATTTTTTTGCTA
ACGCTAACCATAAAAACCAAGGAGAATAA
SEQ ID NO: 16. Dead palm Cas10d protein sequence (BAD01969.1: p.H308A; D309A) modified
residues are in bold and underlined.
MFLVLIETSGNQHFIFSTNKLRENIGASELTYLATTEILFQGVDRVFQTNYYDQWSDTNSLNFLADSKLNPAID
DPKNNADIEILLATSGKAIALVKEEGKAKQLIKEVTKQALINAPGLEIGGIYVNCNWQDKLGVAKAVKEAHKQFEVNR
AKRAGANGRFLRLPIAAGCSVSELPASDFDYNADGDKIPVSTVSKVKRETAKSAKKRLRSVDGRLVNDLAQLEKSFD
ELDWLAVVHADGNGLGQILLSLEKYIGEQTNRNYIDKYRRLSLALDNCTINAFKMAIAVFKEDSKKIDLPIVPLILGGA
ALTVICRGDYALEFTREFLEAFEGQTETHDDIKVIAQKAFGVDRLSACAGISIIKPHFPFSVAYTLAERLIKSAKEVKQK
VTVTNSSPITPFPCSAIDFHILYDSSGIDFDRIREKLRPEDNTELYNRPYVVTAAENLSQAQGYEWSQAHSLQTLADR
VSYLRSEDGEGKSALPSSQSHALRTALYLEKNEADAQYSLISQRYKILKNFAEDGENKSLFHLENGKYVTRFLDALDA
KDFFANANHKNQGE
SEQ ID NO: 17. Dead cas7-5-11 DNA sequence (BAD01968.1: c.77A > C) modified positions
are in bold and underlined.
ATGCGAGGAATTGAGATAACCATAACCATGCAGAGTGATTGGCACGTTGGCACTGGCATGGGTCGGGGG
GAACTGCCAGTGTTGTACAACGGGATGGAGATAATCTGCCCTATATTCCCGGCAAAACCTTAACAGGTATTCTGC
GGGATAGCTGTGAACAGGTTGCCCTAGGTTTAGATAATGGTCAAACCCGAGGGCTTTGGCATGGGTGGATTAAT
TTTATTTTTGGCGATCAACCTGCCCTAGCTCAAGGAGCTATTGAGCCAGAACCTAGACCTGCCCTAATCGCCATT
GGTTCTGCACACCTTGACCCTAAGTTAAAAGCGGCTTTTCAGGGCAAAAAACAATTGCAAGAGGCGATCGCCTTT
ATGAAGCCAGGGGTGGCTATCGATGCAATCACGGGCACAGCTAAGAAAGATTTTTTACGCTTTGAAGAAGTAGT
TCGTTTGGGAGCGAAATTAACTGCGGAAGTTGAGTTAAATTTACCCGATAATTTGAGCGAAACCAATAAAAAAGT
TATTGCTGGTATTTTAGCCAGTGGAGCAAAGTTAACCGAGAGATTAGGCGGTAAACGTCGCCGGGGCAATGGGC
GCTGTGAATTAAAATTTAGTGGTTATTCTGATCAACAAATTCAATGGTTGAAAGACAATTATCAATCTGTTGATCA
ACCACCTAAGTATCAACAAAATAAATTACAATCTGCCGGAGATAATCCAGAACAGCAACCCCCTTGGCATATTATT
CCCTTAACCATTAAAACCCTTTCTCCTGTTGTTTTACCAGCTCGTACAGTCGGTAACGTTGTCGAATGTTTAGACT
ATATTCCCGGGCGTTATCTACTGGGCTATATTCACAAAACCCTAGGGGAATATTTCGACGTTAGTCAGGCAATCG
CCGCTGGGGATTTAATTATTACCAATGCCACGATAAAAATTGATGGTAAAGCAGGACGAGCTACCCCATTTTGTT
TGTTTGGGGAAAAACTAGATGGAGGATTAGGTAAAGGTAAAGGAGTTTATAACCGTTTCCAAGAATCGGAACCT
GATGGCATTCAATTAAAGGGAGAACGGGGCGGCTATGTTGGCCAATTTGAACAGGAGCAAAGGAATCTGCCAAA
TACGGGGAAAATTAATTCAGAGTTATTTACCCATAACACCATTCAAGATGATGTCCAGCGGCCCACCAGTGATGT
GGGGGGAGTTTATAGCTATGAAGCTATTATAGCCGGACAAACATTCGTCGCTGAGTTACGTTTACCAGATAGCTT
AGTCAAGCAAATTACAAGCAAAAATAAAAATTGGCAAGCTCAACTAAAAGCTACAATTCGCATTGGTCAGTCTAA
AAAAGATCAGTATGGCAAAATCGAAGTTACGTCGGGAAACTCTGCTGATTTGCCTAAGCCTACGGGCAACAATA
AAACTCTTTCTATTTGGTTCTTATCCGATATCCTTCTCCGAGGCGATCGCCTAAATTTTAATGCTACTCCGGATGA
TCTCAAAAAATACTTAGAAAATGCTCTGGATATCAAGCTCAAAGAACGATCAGACAATGATTTAATTTGCATTGCT
CTCCGTTCCCAGCGGACAGAATCCTGGCAAGTACGGTGGGGTTTACCCCGGCCATCTCTAGTGGGTTGGCAAGC
TGGTAGTTGTCTGATTTATGACATTGAATCTGGCACTGTTAATGCCGAAAAATTGCAAGAATTAATGATCACCGG
CATTGGCGATCGGTGTACAGAGGGTTACGGTCAAATCGGTTTTAACGATCCATTACTTTCGGCTTCCCTAGGAAA
GTTGACAGCTAAGCCTAAAGCTTCTAACAATCAGTCCCAAAACAGCCAATCCAACCCATTACCCACTAATCATCCT
ACCCAAGATTATGCTCGATTAATTGAAAAAGCGGCTTGGCGGGAAGCAATTCAAAATAAAGCCTTAGCCTTGGCA
TCTAGCCGAGCGAAACGGGAAGAAATTTTAGGCATTAAAATTATGGGAAAAGATAGTCAACCCACCATGACTCAA
TTAGGAGGATTTCGCTCCGTATTAAAACGGCTACACTCAAGAAATAATCGAGATATTGTCACAGGTTATTTAACA
GCTCTAGAGCAGGTTTCTAATCGAAAAGAAAAATGGAGTAATACCAGCCAAGGATTAACTAAAATTCGTAATTTA
GTCACCCAGGAAAATCTCATTTGGAATCATCTTGATATTGATTTTTCGCCGTTAACTATTACCCAAAATGGTGTTA
ATCAGCTAAAGTCTGAACTTTGGGCGGAAGCAGTGCGAACCCTTGTTGACGCTATCATTCGGGGTCATAAACGG
GACTTAGAAAAAGCTCAAGAAAACGAATCTAATCAACAGTCACAGGGAGCAGCTTAA
SEQ ID NO: 18. Dead Cas7-5-11 protein sequence (BAD01968.1: p.D26A) modified residues
are in bold and underlined.
MRGIEITITMQSDWHVGTGMGRGELASVVQRDGDNLPYIPGKTLTGILRDSCEQVALGLDNGQTRGLWHG
WINFIFGDQPALAQGAIEPEPRPALIAIGSAHLDPKLKAAFQGKKQLQEAIAFMKPGVAIDAITGTAKKDFLRFEEVVR
LGAKLTAEVELNLPDNLSETNKKVIAGILASGAKLTERLGGKRRRGNGRCELKFSGYSDQQIQWLKDNYQSVDQPP
KYQQNKLQSAGDNPEQQPPWHIIPLTIKTLSPVVLPARTVGNVVECLDYIPGRYLLGYIHKTLGEYFDVSQAIAAGDLI
ITNATIKIDGKAGRATPFCLFGEKLDGGLGKGKGVYNRFQESEPDGIQLKGERGGYVGQFEQEQRNLPNTGKINSEL
FTHNTIQDDVQRPTSDVGGVYSYEAIIAGQTFVAELRLPDSLVKQITSKNKNWQAQLKATIRIGQSKKDQYGKIEVT
SGNSADLPKPTGNNKTLSIWFLSDILLRGDRLNFNATPDDLKKYLENALDIKLKERSDNDLICIALRSQRTESWQVR
WGLPRPSLVGWQAGSCLIYDIESGTVNAEKLQELMITGIGDRCTEGYGQIGFNDPLLSASLGKLTAKPKASNNQSQ
NSQSNPLPTNHPTQDYARLIEKAAWREAIQNKALALASSRAKREEILGIKIMGKDSQPTMTQLGGFRSVLKRLHSRN
NRDIVTGYLTALEQVSNRKEKWSNTSQGLTKIRNLVTQENLIWNHLDIDFSPLTITQNGVNQLKSELWAEAVRTLVD
AIIRGHKRDLEKAQENESNQQSQGAA
SEQ ID NO: 19. Dead cas7_2x.1 DNA sequence (BAD01967.1: c.98A > C) modified positions
are in bold and underlined.
ATGGCTAGAAAAGTTACTACACGCTGGAAAATTACAGGCACATTAATTGCAGAAACCCCTTTACACATTGG
TGGTGTGGGTGGCGACGCTGATACGGCTTTAGCCCTGGCGGTTAATGGTGCGGGTGAATATTATGTGCCAGGG
ACAAGTTTAGCCGGTGCTCTGCGGGGTTGGATGACCCAGTTATTGAATAATGATGAGTCCCAAATTAAAGATCTT
TGGGGTGATCATTTAGATGCAAAACGGGGAGCTAGCTTTGTTATTGTTGACGATGCGGTTATCCATATACCCAAT
AATGCTGATGTTGAAATTAGGGAGGGTGTTGGCATCGATCGCCATTTTGGAACCGCCGCCAATGGGTTTAAATA
TAGCCGAGCAGTTATTCCCAAGGGTTCTAAATTTAAATTGCCATTAACTTTTGACAGTCAAGATGATGGGCTACC
GAATGCGTTGATTCAATTGTTGTGTGCCTTAGAAGCAGGGGATATTCGCCTTGGGGCCGCAAAAACCCGGGGTT
TAGGTCGCATTAAACTAGATGATTTAAAGTTAAAATCCTTTGCTTTAGATAAACCAGAAGGTATTTTTTCTGCTTTA
TTAGACCAAGGTAAAAAATTAGATTGGAATCAATTAAAAGCAAACGTTACCTACCAGTCTCCTCCCTATCTAGGTA
TTAGTATTACCTGGAATCCCAAAGATCCCGTCATGGTGAAAGCTGAAGGGGATGGACTGGCGATCGATATTTTG
CCCCTCGTTAGTCAAGTGGGAAGTGATGTTCGATTTGTCATTCCCGGCAGTTCCATTAAGGGGATTTTACGAACC
CAGGCTGAACGTATTATTCGTACTATTTGCCAGTCTAATGGTTCTGAGAAAAACTTCCTAGAACAATTACGAATCA
ATCTGGTTAATGAATTATTTGGGTCTGCTTCTTTGAGCCAAAAACAAAATGGCAAGGATATAGATCTGGGTAAAA
TCGGAGCCTTGGCAGTGAATGATTGTTTTTCTAGTTTATCCATGACCCCAGATCAATGGAAAGCGGTAGAGAATG
CCACGGAGATGACGGGGAATTTACAGCCTGCTCTTAAACAAGCTACGGGTTATCCCAATAATATTAGCCAAGCTT
ACAAAGTACTTCAACCGGCCATGCACGTCGCTGTAGATCGGTGGACAGGGGGAGCTGCCGAAGGAATGCTTTA
CAGCGTGCTCGAACCCATTGGGGTCACCTGGGAACCGATCCAAGTTCACTTGGACATTGCCCGTCTCAAAAATT
ATTACCACGGTAAGGAAGAAAAACTTAAACCGGCGATCGCCCTATTGCTTCTTGTATTGCGGGATTTAGCTAACA
AAAAAATTCCCGTAGGCTATGGCACTAACCGCGGTATGGGAACGATTACTGTCAGTCAAATCACCCTCAATGGCA
AAGCCCTCCCCACTGAACTTGAACCTTTAAACAAAACAATGACTTGTCCTAATCTCACCGATCTAGATGAGGCATT
TCGTCAGGACTTAAGCACTGCTTGGAAAGAGTGGATTGCCGATCCCATTGATCTATGCCAGCAGGAGGCCGCCT
AA
SEQ ID NO: 20. Dead Cas7_2x.1 protein sequence (BAD01967.1: p.D33A) modified residue
is in bold and underlined.
MARKVTTRWKITGTLIAETPLHIGGVGGDADTALALAVNGAGEYYVPGTSLAGALRGWMTQLLNNDESQIK
DLWGDHLDAKRGASFVIVDDAVIHIPNNADVEIREGVGIDRHFGTAANGFKYSRAVIPKGSKFKLPLTFDSQDDGLP
NALIQLLCALEAGDIRLGAAKTRGLGRIKLDDLKLKSFALDKPEGIFSALLDQGKKLDWNQLKANVTYQSPPYLGISIT
WNPKDPVMVKAEGDGLAIDILPLVSQVGSDVRFVIPGSSIKGILRTQAERIIRTICQSNGSEKNFLEQLRINLVNELFG
SASLSQKQNGKDIDLGKIGALAVNDCFSSLSMTPDQWKAVENATEMTGNLQPALKQATGYPNNISQAYKVLQPAM
HVAVDRWTGGAAEGMLYSVLEPIGVTWEPIQVHLDIARLKNYYHGKEEKLKPAIALLLLVLRDLANKKIPVGYGTNRG
MGTITVSQITLNGKALPTELEPLNKTMTCPNLTDLDEAFRQDLSTAWKEWIADPIDLCQQEAA
SEQ ID NO: 21. Dead cas7_2x.2 DNA sequence (BAD01967.1: c.737A > C) modified position
is in bold and underlined.
ATGGCTAGAAAAGTTACTACACGCTGGAAAATTACAGGCACATTAATTGCAGAAACCCCTTTACACATTGG
TGGTGTGGGTGGCGACGCTGATACGGATTTAGCCCTGGCGGTTAATGGTGCGGGTGAATATTATGTGCCAGGG
ACAAGTTTAGCCGGTGCTCTGCGGGGTTGGATGACCCAGTTATTGAATAATGATGAGTCCCAAATTAAAGATCTT
TGGGGTGATCATTTAGATGCAAAACGGGGAGCTAGCTTTGTTATTGTTGACGATGCGGTTATCCATATACCCAAT
AATGCTGATGTTGAAATTAGGGAGGGTGTTGGCATCGATCGCCATTTTGGAACCGCCGCCAATGGGTTTAAATA
TAGCCGAGCAGTTATTCCCAAGGGTTCTAAATTTAAATTGCCATTAACTTTTGACAGTCAAGATGATGGGCTACC
GAATGCGTTGATTCAATTGTTGTGTGCCTTAGAAGCAGGGGATATTCGCCTTGGGGCCGCAAAAACCCGGGGTT
TAGGTCGCATTAAACTAGATGATTTAAAGTTAAAATCCTTTGCTTTAGATAAACCAGAAGGTATTTTTTCTGCTTTA
TTAGACCAAGGTAAAAAATTAGATTGGAATCAATTAAAAGCAAACGTTACCTACCAGTCTCCTCCCTATCTAGGTA
TTAGTATTACCTGGAATCCCAAAGATCCCGTCATGGTGAAAGCTGAAGGGGATGGACTGGCGATCGCTATTTTG
CCCCTCGTTAGTCAAGTGGGAAGTGATGTTCGATTTGTCATTCCCGGCAGTTCCATTAAGGGGATTTTACGAACC
CAGGCTGAACGTATTATTCGTACTATTTGCCAGTCTAATGGTTCTGAGAAAAACTTCCTAGAACAATTACGAATCA
ATCTGGTTAATGAATTATTTGGGTCTGCTTCTTTGAGCCAAAAACAAAATGGCAAGGATATAGATCTGGGTAAAA
TCGGAGCCTTGGCAGTGAATGATTGTTTTTCTAGTTTATCCATGACCCCAGATCAATGGAAAGCGGTAGAGAATG
CCACGGAGATGACGGGGAATTTACAGCCTGCTCTTAAACAAGCTACGGGTTATCCCAATAATATTAGCCAAGCTT
ACAAAGTACTTCAACCGGCCATGCACGTCGCTGTAGATCGGTGGACAGGGGGAGCTGCCGAAGGAATGCTTTA
CAGCGTGCTCGAACCCATTGGGGTCACCTGGGAACCGATCCAAGTTCACTTGGACATTGCCCGTCTCAAAAATT
ATTACCACGGTAAGGAAGAAAAACTTAAACCGGCGATCGCCCTATTGCTTCTTGTATTGCGGGATTTAGCTAACA
AAAAAATTCCCGTAGGCTATGGCACTAACCGCGGTATGGGAACGATTACTGTCAGTCAAATCACCCTCAATGGCA
AAGCCCTCCCCACTGAACTTGAACCTTTAAACAAAACAATGACTTGTCCTAATCTCACCGATCTAGATGAGGCATT
TCGTCAGGACTTAAGCACTGCTTGGAAAGAGTGGATTGCCGATCCCATTGATCTATGCCAGCAGGAGGCCGCCT
AA
SEQ ID NO: 22. Dead Cas7_2x.2 protein sequence (BAD01967.1: p.D246A) modified residue
is in bold and underlined
MARKVTTRWKITGTLIAETPLHIGGVGGDADTDLALAVNGAGEYYVPGTSLAGALRGWMTQLLNNDESQIK
DLWGDHLDAKRGASFVIVDDAVIHIPNNADVEIREGVGIDRHFGTAANGFKYSRAVIPKGSKFKLPLTFDSQDDGLP
NALIQLLCALEAGDIRLGAAKTRGLGRIKLDDLKLKSFALDKPEGIFSALLDQGKKLDWNQLKANVTYQSPPYLGISIT
WNPKDPVMVKAEGDGLAIAILPLVSQVGSDVRFVIPGSSIKGILRTQAERIIRTICQSNGSEKNFLEQLRINLVNELFG
SASLSQKQNGKDIDLGKIGALAVNDCFSSLSMTPDQWKAVENATEMTGNLQPALKQATGYPNNISQAYKVLQPAM
HVAVDRWTGGAAEGMLYSVLEPIGVTWEPIQVHLDIARLKNYYHGKEEKLKPAIALLLLVLRDLANKKIPVGYGTNRG
MGTITVSQITLNGKALPTELEPLNKTMTCPNLTDLDEAFRQDLSTAWKEWIADPIDLCQQEAA
SEQ ID NO: 152. Dead cas7_2x.1 and cas7_2x.2 DNA sequence (BAD01967.1: c.98A > C;
c.737A > C) modified positions are in bold and underlined.
ATGGCTAGAAAAGTTACTACACGCTGGAAAATTACAGGCACATTAATTGCAGAAACCCCTTTACACATTGG
TGGTGTGGGTGGCGACGCTGATACGGCTTTAGCCCTGGCGGTTAATGGTGCGGGTGAATATTATGTGCCAGGG
ACAAGTTTAGCCGGTGCTCTGCGGGGTTGGATGACCCAGTTATTGAATAATGATGAGTCCCAAATTAAAGATCTT
TGGGGTGATCATTTAGATGCAAAACGGGGAGCTAGCTTTGTTATTGTTGACGATGCGGTTATCCATATACCCAAT
AATGCTGATGTTGAAATTAGGGAGGGTGTTGGCATCGATCGCCATTTTGGAACCGCCGCCAATGGGTTTAAATA
TAGCCGAGCAGTTATTCCCAAGGGTTCTAAATTTAAATTGCCATTAACTTTTGACAGTCAAGATGATGGGCTACC
GAATGCGTTGATTCAATTGTTGTGTGCCTTAGAAGCAGGGGATATTCGCCTTGGGGCCGCAAAAACCCGGGGTT
TAGGTCGCATTAAACTAGATGATTTAAAGTTAAAATCCTTTGCTTTAGATAAACCAGAAGGTATTTTTTCTGCTTTA
TTAGACCAAGGTAAAAAATTAGATTGGAATCAATTAAAAGCAAACGTTACCTACCAGTCTCCTCCCTATCTAGGTA
TTAGTATTACCTGGAATCCCAAAGATCCCGTCATGGTGAAAGCTGAAGGGGATGGACTGGCGATCGCTATTTTG
CCCCTCGTTAGTCAAGTGGGAAGTGATGTTCGATTTGTCATTCCCGGCAGTTCCATTAAGGGGATTTTACGAACC
CAGGCTGAACGTATTATTCGTACTATTTGCCAGTCTAATGGTTCTGAGAAAAACTTCCTAGAACAATTACGAATCA
ATCTGGTTAATGAATTATTTGGGTCTGCTTCTTTGAGCCAAAAACAAAATGGCAAGGATATAGATCTGGGTAAAA
TCGGAGCCTTGGCAGTGAATGATTGTTTTTCTAGTTTATCCATGACCCCAGATCAATGGAAAGCGGTAGAGAATG
CCACGGAGATGACGGGGAATTTACAGCCTGCTCTTAAACAAGCTACGGGTTATCCCAATAATATTAGCCAAGCTT
ACAAAGTACTTCAACCGGCCATGCACGTCGCTGTAGATCGGTGGACAGGGGGAGCTGCCGAAGGAATGCTTTA
CAGCGTGCTCGAACCCATTGGGGTCACCTGGGAACCGATCCAAGTTCACTTGGACATTGCCCGTCTCAAAAATT
ATTACCACGGTAAGGAAGAAAAACTTAAACCGGCGATCGCCCTATTGCTTCTTGTATTGCGGGATTTAGCTAACA
AAAAAATTCCCGTAGGCTATGGCACTAACCGCGGTATGGGAACGATTACTGTCAGTCAAATCACCCTCAATGGCA
AAGCCCTCCCCACTGAACTTGAACCTTTAAACAAAACAATGACTTGTCCTAATCTCACCGATCTAGATGAGGCATT
TCGTCAGGACTTAAGCACTGCTTGGAAAGAGTGGATTGCCGATCCCATTGATCTATGCCAGCAGGAGGCCGCCT
AA
SEQ ID NO: 153. Dead Cas7_2x.1 and Cas7_2x.2 protein sequence (BAD01967.1: p.D33A;
p.D246A) modified residue is in bold and underlined.
MARKVTTRWKITGTLIAETPLHIGGVGGDADTALALAVNGAGEYYVPGTSLAGALRGWMTQLLNNDESQIK
DLWGDHLDAKRGASFVIVDDAVIHIPNNADVEIREGVGIDRHFGTAANGFKYSRAVIPKGSKFKLPLTFDSQDDGLP
NALIQLLCALEAGDIRLGAAKTRGLGRIKLDDLKLKSFALDKPEGIFSALLDQGKKLDWNQLKANVTYQSPPYLGISIT
WNPKDPVMVKAEGDGLAIAILPLVSQVGSDVRFVIPGSSIKGILRTQAERIIRTICQSNGSEKNFLEQLRINLVNELFG
SASLSQKQNGKDIDLGKIGALAVNDCFSSLSMTPDQWKAVENATEMTGNLQPALKQATGYPNNISQAYKVLQPAM
HVAVDRWTGGAAEGMLYSVLEPIGVTWEPIQVHLDIARLKNYYHGKEEKLKPAIALLLLVLRDLANKKIPVGYGTNRG
MGTITVSQITLNGKALPTELEPLNKTMTCPNLTDLDEAFRQDLSTAWKEWIADPIDLCQQEAA
SEQ ID NO: 23-Example unprocessed guide RNA (spacer bold)
ACUGAAACUGUAGUAGAACCAAUCGGGGUCGUCAAUAACUCCCGGTTCAACACCCTCTTTTCCCCG
TCAGGGG
SEQ ID NO: 35-Example mature guide RNA (spacer bold)
ACUGAAACUGUAGUAGAACCAAUCGGGGUCGUCAAUA
SEQ ID NO: 24 RNA sequence tested (protospacer bold)
CAUGACGGAUCGCGGGAGUUAUUGACGACCCCGAUUGGUUCUACUACAAACGUGAUACUA
SEQ ID NO: 25 CRISPR array spacer
TGTAGTAGAACCAATCGGGGTCGTCAATAACTCCCG
SEQ ID NO: 26 CRISPR array flanking repeat
GTTCAACACCCTCTTTTCCCCGTCAGGGGACTGAAAC
SEQ ID NO: 27. DNA sequence encoding an example single Type III-Dv effector DNA sequence
(GenBank: BAD01968.1; BAD01967.1; BAD01965.1). Linker sequences between subunits in
bold and underlined.
ATGCGAGGAATTGAGATAACCATAACCATGCAGAGTGATTGGCACGTTGGCACTGGCATGGGTCGGGGG
GAACTGGACAGTGTTGTACAACGGGATGGAGATAATCTGCCCTATATTCCCGGCAAAACCTTAACAGGTATTCTG
CGGGATAGCTGTGAACAGGTTGCCCTAGGTTTAGATAATGGTCAAACCCGAGGGCTTTGGCATGGGTGGATTAA
TITTATTTTTGGCGATCAACCTGCCCTAGCTCAAGGAGCTATTGAGCCAGAACCTAGACCTGCCCTAATCGCCAT
TGGTTCTGCACACCTTGACCCTAAGTTAAAAGCGGCTTTTCAGGGCAAAAAACAATTGCAAGAGGCGATCGCCTT
TATGAAGCCAGGGGTGGCTATCGATGCAATCACGGGCACAGCTAAGAAAGATTTTTTACGCTTTGAAGAAGTAG
TTCGTTTGGGAGCGAAATTAACTGCGGAAGTTGAGTTAAATTTACCCGATAATTTGAGCGAAACCAATAAAAAAG
TTATTGCTGGTATTTTAGCCAGTGGAGCAAAGTTAACCGAGAGATTAGGCGGTAAACGTCGCCGGGGCAATGGG
CGCTGTGAATTAAAATTTAGTGGTTATTCTGATCAACAAATTCAATGGTTGAAAGACAATTATCAATCTGTTGATC
AACCACCTAAGTATCAACAAAATAAATTACAATCTGCCGGAGATAATCCAGAACAGCAACCCCCTTGGCATATTA
TTCCCTTAACCATTAAAACCCTTTCTCCTGTTGTTTTACCAGCTCGTACAGTCGGTAACGTTGTCGAATGTTTAGA
CTATATTCCCGGGCGTTATCTACTGGGCTATATTCACAAAACCCTAGGGGAATATTTCGACGTTAGTCAGGCAAT
CGCCGCTGGGGATTTAATTATTACCAATGCCACGATAAAAATTGATGGTAAAGCAGGACGAGCTACCCCATTTTG
TTTGTTTGGGGAAAAACTAGATGGAGGATTAGGTAAAGGTAAAGGAGTTTATAACCGTTTCCAAGAATCGGAAC
CTGATGGCATTCAATTAAAGGGAGAACGGGGCGGCTATGTTGGCCAATTTGAACAGGAGCAAAGGAATCTGCCA
AATACGGGGAAAATTAATTCAGAGTTATTTACCCATAACACCATTCAAGATGATGTCCAGCGGCCCACCAGTGAT
GTGGGGGGAGTTTATAGCTATGAAGCTATTATAGCCGGACAAACATTCGTCGCTGAGTTACGTTTACCAGATAG
CTTAGTCAAGCAAATTACAAGCAAAAATAAAAATTGGCAAGCTCAACTAAAAGCTACAATTCGCATTGGTCAGTC
TAAAAAAGATCAGTATGGCAAAATCGAAGTTACGTCGGGAAACTCTGCTGATTTGCCTAAGCCTACGGGCAACA
ATAAAACTCTTTCTATTTGGTTCTTATCCGATATCCTTCTCCGAGGCGATCGCCTAAATTTTAATGCTACTCCGGA
TGATCTCAAAAAATACTTAGAAAATGCTCTGGATATCAAGCTCAAAGAACGATCAGACAATGATTTAATTTGCATT
GCTCTCCGTTCCCAGCGGACAGAATCCTGGCAAGTACGGTGGGGTTTACCCCGGCCATCTCTAGTGGGTTGGCA
AGCTGGTAGTTGTCTGATTTATGACATTGAATCTGGCACTGTTAATGCCGAAAAATTGCAAGAATTAATGATCAC
CGGCATTGGCGATCGGTGTACAGAGGGTTACGGTCAAATCGGTTTTAACGATCCATTACTTTCGGCTTCCCTAGG
AAAGTTGACAGCTAAGCCTAAAGCTTCTAACAATCAGTCCCAAAACAGCCAATCCAACCCATTACCCACTAATCAT
CCTACCCAAGATTATGCTCGATTAATTGAAAAAGCGGCTTGGCGGGAAGCAATTCAAAATAAAGCCTTAGCCTTG
GCATCTAGCCGAGCGAAACGGGAAGAAATTTTAGGCATTAAAATTATGGGAAAAGATAGTCAACCCACCATGAC
TCAATTAGGAGGATTTCGCTCCGTATTAAAACGGCTACACTCAAGAAATAATCGAGATATTGTCACAGGTTATTTA
ACAGCTCTAGAGCAGGTTTCTAATCGAAAAGAAAAATGGAGTAATACCAGCCAAGGATTAACTAAAATTCGTAAT
TTAGTCACCCAGGAAAATCTCATTTGGAATCATCTTGATATTGATTTTTCGCCGTTAACTATTACCCAAAATGGTG
TTAATCAGCTAAAGTCTGAACTTTGGGCGGAAGCAGTGCGAACCCTTGTTGACGCTATCATTCGGGGTCATAAAC
GGGACTTAGAAAAAGCTCAAGAAAACGAATCTAATCAACAGTCACAGGGAGCAGCTCTGAAAATTACCCGCCG
CATTCTGGGCGATGCGGAATTTCATGGCAAACCGGATCGCCTGGAAAAAAGCCGCAGCGTGAGCATTG
GCAGCGTGCTGATGGCTAGAAAAGTTACTACACGCTGGAAAATTACAGGCACATTAATTGCAGAAACCCCTTTA
CACATTGGTGGTGTGGGTGGCGACGCTGATACGGATTTAGCCCTGGCGGTTAATGGTGCGGGTGAATATTATGT
GCCAGGGACAAGTTTAGCCGGTGCTCTGCGGGGTTGGATGACCCAGTTATTGAATAATGATGAGTCCCAAATTA
AAGATCTTTGGGGTGATCATTTAGATGCAAAACGGGGAGCTAGCTTTGTTATTGTTGACGATGCGGTTATCCATA
TACCCAATAATGCTGATGTTGAAATTAGGGAGGGTGTTGGCATCGATCGCCATTTTGGAACCGCCGCCAATGGG
TTTAAATATAGCCGAGCAGTTATTCCCAAGGGTTCTAAATTTAAATTGCCATTAACTTTTGACAGTCAAGATGATG
GGCTACCGAATGCGTTGATTCAATTGTTGTGTGCCTTAGAAGCAGGGGATATTCGCCTTGGGGCCGCAAAAACC
CGGGGTTTAGGTCGCATTAAACTAGATGATTTAAAGTTAAAATCCTTTGCTTTAGATAAACCAGAAGGTATTTTTT
CTGCTTTATTAGACCAAGGTAAAAAATTAGATTGGAATCAATTAAAAGCAAACGTTACCTACCAGTCTCCTCCCTA
TCTAGGTATTAGTATTACCTGGAATCCCAAAGATCCCGTCATGGTGAAAGCTGAAGGGGATGGACTGGCGATCG
ATATTTTGCCCCTCGTTAGTCAAGTGGGAAGTGATGTTCGATTTGTCATTCCCGGCAGTTCCATTAAGGGGATTT
TACGAACCCAGGCTGAACGTATTATTCGTACTATTTGCCAGTCTAATGGTTCTGAGAAAAACTTCCTAGAACAATT
ACGAATCAATCTGGTTAATGAATTATTTGGGTCTGCTTCTTTGAGCCAAAAACAAAATGGCAAGGATATAGATCT
GGGTAAAATCGGAGCCTTGGCAGTGAATGATTGTTTTTCTAGTTTATCCATGACCCCAGATCAATGGAAAGCGGT
AGAGAATGCCACGGAGATGACGGGGAATTTACAGCCTGCTCTTAAACAAGCTACGGGTTATCCCAATAATATTA
GCCAAGCTTACAAAGTACTTCAACCGGCCATGCACGTCGCTGTAGATCGGTGGACAGGGGGAGCTGCCGAAGG
AATGCTTTACAGCGTGCTCGAACCCATTGGGGTCACCTGGGAACCGATCCAAGTTCACTTGGACATTGCCCGTCT
CAAAAATTATTACCACGGTAAGGAAGAAAAACTTAAACCGGCGATCGCCCTATTGCTTCTTGTATTGCGGGATTT
AGCTAACAAAAAAATTCCCGTAGGCTATGGCACTAACCGCGGTATGGGAACGATTACTGTCAGTCAAATCACCCT
CAATGGCAAAGCCCTCCCCACTGAACTTGAACCTTTAAACAAAACAATGACTTGTCCTAATCTCACCGATCTAGAT
GAGGCATTTCGTCAGGACTTAAGCACTGCTTGGAAAGAGTGGATTGCCGATCCCATTGATCTATGCCAGCAGGA
GGCCGCCCTGGGCAACCCGAAAGGCCAGGAACTGAAACTGGATCCGCCGAGCGCGGATGCGACCCAGG
CGGGCGTGCCGGCGCAGCAGAACGCGGCGAAAACCCAGGCGCAGGGCGCGCAGGAAAAATTTCATAAC
CCCTACAACTTTGTCCCAGCCCTACCCCGCGATGGCATAACCGGAGATTTAGGCGACTGTGCTCCTGCTGGTCA
TAGCTATTACCATGGCGATAAATACAGCGGCAGAATTGCCGTCAAACTAACAACCGTTACCCCTCTATTGATTCC
TGACGCTTCAAAAGAAGAGATAAATAACAACCATAAAACCTATCCGGTTCGTATCGGCAAAGATGGCAAGCCCTA
TCTACCTCCCACTTCCATTAAGGGAATGTTGCGCTCTGCCTATGAAGCGGTCACTAATTCCCGCTTAGCCGTGTT
TGAAGATCATGACTCTCGCTTGGCCTATCGAATGCCTGCCACCATGGGATTGCAAATGGTTCCTGCCCGCATTGA
AGGTGATAATATTGTTCTTTACCCAGGAACCTCAAGGATAGGCAATAATGGCCGACCAGCTAACAATGATCCTAT
GTATGCGGCATGGCTTCCTTACTATCAAAATCGTATTGCTTATGATGGTAGTCGTGATTATCAGATGGCTGAGCA
TGGTGATCATGTCAGATTTTGGGCTGAGCGATATACCAGAGGAAACTTCTGCTATTGGCGTGTCAGACAAATTGC
ACGACACAATCAAAATTTAGGTAATCGGCCTGAACGAGGACGTAATTACGGTCAACATCATTCAACAGGAGTCAT
TGAACAATTTGAAGGATTTGTTTACAAAACCAATAAAAATATTGGGAATAAACATGACGAACGAGTATTTATTATT
GATCGAGAAAGTATCGAAATACCTCTATCTCGAGATTTACGGCGAAAATGGCGAGAATTAATTACAAGCTATCAG
GAAATACACAAAAAGGAAGTTGATAGAGGTGATACTGGCCCTTCCGCTGTAAATGGGGCTGTTTGGTCACGGCA
AATTATTGCAGATGAATCAGAGCGGAATTTATCGGATGGGACTCTTTGTTATGCTCATGTTAAGAAAGAAGATGG
ACAGTACAAAATTCTCAATCTTTATCCTGTAATGATCACACGGGGATTATATGAAATTGCGCCGGTTGACTTATTA
GATGAAACCCTAAAGCCTGCGACGGATAAAAAGCAACTATCCCCAGCAGACCGCGTATTTGGCTGGGTCAATCA
ACGGGGCAATGGTTGCTACAAAGGACAATTACGAATTCATAGCGTAACTTGCCAACATGATGATGCCATTGATGA
TTTTGGTAATCAAAATTTCTCTGTTCCCCTTGCTATTTTGGGACAACCTAAACCAGAACAGGCTCGTTTTTATTGT
GCCGATGATCGAAAAGGAATTCCTTTAGAAGATGGCTATGATCGTGACGACGGCTATAGTGATTCAGAACAAGG
CTTGCGAGGACGCAAAGTCTATCCTCACCACAAGGGGTTACCAAATGGCTACTGGAGTAATCCAACGGAAGACC
GAAGTCAACAAGCTATCCAAGGTCATTACCAAGAATATCGTCGTCCTAAAAAGGATGGTCTTGAACAAAGAGATG
ATCAAAATCGTTCTGTAAAAGGTTGGGTAAAACCACTGACCGAGTTTACTTTTGAAATTGACGTTACTAATCTTTC
GGAAGTTGAGTTAGGTGCTCTATTGTGGTTGTTAACCTTACCTGATTTGCATTTCCACCGTCTAGGAGGAGGTAA
ACCGTTAGGTTTTGGTAGTGTTCGTTTAGATATTGACCCTGACAAGACAGACCTAAGAAATGGGGCAGGATGGC
GTGATTATTACGGCTCTTTACTAGAAACAAGTCAACCAGATTTTACAACTCTAATTAGTCAGTGGATTAATGCTTT
TCAAACGGCTGTTAAAGAGGAGTATGGTAGCAGTAGTTTTGATCAGGTTACTTTCATCAAAGCTTCTGGTCAGAG
TCTCCAAGGATTTCATGATAATGCATCTATCCATTATCCTCGTTCTACTCCTGAGCCCAAGCCAGATGGAGAAGC
TITTAAGTGGTTTGTTGCCAATGAAAAAGGTCGACGATTAGCCTTGCCAGCGCTGGAAAAATCCCAGAGTTTTCC
AATCAAACCTAGTTAA
SEQ ID NO: 28. Example single Type III-Dv effector protein sequence (GenBank: BAD01968.1;
BAD01967.1; BAD01965.1). Linker sequences between subunits in bold and underlined.
MRGIEITITMQSDWHVGTGMGRGELDSVVQRDGDNLPYIPGKTLTGILRDSCEQVALGLDNGQTRGLWHG
WINFIFGDQPALAQGAIEPEPRPALIAIGSAHLDPKLKAAFQGKKQLQEAIAFMKPGVAIDAITGTAKKDFLRFEEVVR
LGAKLTAEVELNLPDNLSETNKKVIAGILASGAKLTERLGGKRRRGNGRCELKFSGYSDQQIQWLKDNYQSVDQPP
KYQQNKLQSAGDNPEQQPPWHIIPLTIKTLSPVVLPARTVGNVVECLDYIPGRYLLGYIHKTLGEYFDVSQAIAAGDLI
ITNATIKIDGKAGRATPFCLFGEKLDGGLGKGKGVYNRFQESEPDGIQLKGERGGYVGQFEQEQRNLPNTGKINSEL
FTHNTIQDDVQRPTSDVGGVYSYEAIIAGQTFVAELRLPDSLVKQITSKNKNWQAQLKATIRIGQSKKDQYGKIEVT
SGNSADLPKPTGNNKTLSIWFLSDILLRGDRLNFNATPDDLKKYLENALDIKLKERSDNDLICIALRSQRTESWQVR
WGLPRPSLVGWQAGSCLIYDIESGTVNAEKLQELMITGIGDRCTEGYGQIGFNDPLLSASLGKLTAKPKASNNQSQ
NSQSNPLPTNHPTQDYARLIEKAAWREAIQNKALALASSRAKREEILGIKIMGKDSQPTMTQLGGFRSVLKRLHSRN
NRDIVTGYLTALEQVSNRKEKWSNTSQGLTKIRNLVTQENLIWNHLDIDFSPLTITQNGVNQLKSELWAEAVRTLVD
AIIRGHKRDLEKAQENESNQQSQGAALKITRRILGDAEFHGKPDRLEKSRSVSIGSVLMARKVTTRWKITGTLI
AETPLHIGGVGGDADTDLALAVNGAGEYYVPGTSLAGALRGWMTQLLNNDESQIKDLWGDHLDAKRGASFVIVDD
AVIHIPNNADVEIREGVGIDRHFGTAANGFKYSRAVIPKGSKFKLPLTFDSQDDGLPNALIQLLCALEAGDIRLGAAKT
RGLGRIKLDDLKLKSFALDKPEGIFSALLDQGKKLDWNQLKANVTYQSPPYLGISITWNPKDPVMVKAEGDGLAIDIL
PLVSQVGSDVRFVIPGSSIKGILRTQAERIIRTICQSNGSEKNFLEQLRINLVNELFGSASLSQKQNGKDIDLGKIGAL
AVNDCFSSLSMTPDQWKAVENATEMTGNLQPALKQATGYPNNISQAYKVLQPAMHVAVDRWTGGAAEGMLYSVL
EPIGVTWEPIQVHLDIARLKNYYHGKEEKLKPAIALLLLVLRDLANKKIPVGYGTNRGMGTITVSQITLNGKALPTELEP
LNKTMTCPNLTDLDEAFRQDLSTAWKEWIADPIDLCQQEAALGNPKGQELKLDPPSADATQAGVPAQQNAAK
TQAQGAQEKFHNPYNFVPALPRDGITGDLGDCAPAGHSYYHGDKYSGRIAVKLTTVTPLLIPDASKEEINNNHKTYP
VRIGKDGKPYLPPTSIKGMLRSAYEAVTNSRLAVFEDHDSRLAYRMPATMGLQMVPARIEGDNIVLYPGTSRIGNNG
RPANNDPMYAAWLPYYQNRIAYDGSRDYQMAEHGDHVRFWAERYTRGNFCYWRVRQIARHNQNLGNRPERGRNY
GQHHSTGVIEQFEGFVYKTNKNIGNKHDERVFIIDRESIEIPLSRDLRRKWRELITSYQEIHKKEVDRGDTGPSAVNG
AVWSRQIIADESERNLSDGTLCYAHVKKEDGQYKILNLYPVMITRGLYEIAPVDLLDETLKPATDKKQLSPADRVFG
WVNQRGNGCYKGQLRIHSVTCQHDDAIDDFGNQNFSVPLAILGQPKPEQARFYCADDRKGIPLEDGYDRDDGYSD
SEQGLRGRKVYPHHKGLPNGYWSNPTEDRSQQAIQGHYQEYRRPKKDGLEQRDDQNRSVKGWVKPLTEFTFEIDV
TNLSEVELGALLWLLTLPDLHFHRLGGGKPLGFGSVRLDIDPDKTDLRNGAGWRDYYGSLLETSQPDFTTLISQWIN
AFQTAVKEEYGSSSFDQVTFIKASGQSLQGFHDNASIHYPRSTPEPKPDGEAFKWFVANEKGRRLALPALEKSQSFP
IKPS
SEQ ID NO: 29. nucC DNA sequence (GenBank: CP025084.1)
ATGACTAATCAGGCAAAAAAGTTATCTAGAATTAATGGTAGGGAGTTTTTAAAACAGTCCTTTAATTTACA
ACAACAACTATTGGCCTCTCAATTAAATTTATCCCGAACGATTACGCATGATGGAACGATGGGGGAGGTTAATGA
AAGTTATTTTTTGAGTATTATCCGCCAGTATTTGCCTGAACGTTACTCGGTTGACCGGGGAGTTGTGGTGGATTC
AGAAGGCCAGACCAGCGACCAGATAGATGCAGTGATTTTTGACCGGCATTACACACCGACATTATTAGACCAAC
AAGGGCACAGGTTTATTCCGGCAGAGGCGGTGTACGCGGTACTGGAGGTTAAACCAACCATTAATAAAACCTAC
CTTGAATATGCAGCCGATAAAGCTGCATCTGTCCGAAAATTATATCGAACCAGTACGGTAATAAAAAATATTTAC
GGTACGGCCAAACCGGTCGAACATTTCCCGATCGTAGCAGGTATTGTGGCGATTGATGTTGAGTGGCAAGACGG
ACTCGGAAAGGCATTTACTGAAAATTTGCAGGCTGTTTCCAGCGATGAAAACCGAAAACTGGATTGCGGTCTGG
CGGTGTCGGGCGCATGTTTTGATAGTTATGATGAGGAAATAAAAATCAGAAGCGGTGAAAATGCATTAATCTTTT
TTCTGTTCCGTTTGCTCGGTAAATTGCAATCATTAGGTACGGTGCCCGCAATTGACTGGCGGGTGTATATAGATA
GTCTGGAATAA
SEQ ID NO: 30. NucC protein sequence (GenBank: CP025084.1)
MTNQAKKLSRINGREFLKQSFNLQQQLLASQLNLSRTITHDGTMGEVNESYFLSIIRQYLPERYSVDRGVVVD
SEGQTSDQIDAVIFDRHYTPTLLDQQGHRFIPAEAVYAVLEVKPTINKTYLEYAADKAASVRKLYRTSTVIKNIYGTAK
PVEHFPIVAGIVAIDVEWQDGLGKAFTENLQAVSSDENRKLDCGLAVSGACFDSYDEEIKIRSGENALIFFLFRLLGK
LQSLGTVPAIDWRVYIDSLE
SEQ ID NO: 31. NucC substrate. Recognition sequence in bold. (Substrate PF6284, FIG 11H).
CCCTACGCTCCCTCCAGCGCTGTCGGGGATATAGTCACTCGGCAAGGGCGCCCTTGAGGATTGATTACT
GAACTCTAGTATGGTAAACTGTGAAAACTCATAAAGCTGACGAAGTAAAAGAATCAAACTAATAACTCAATCCAG
TCTAAAGAGTAGAAAGTTGGTGAAAGATTGTGAGTCAGTCACTTAATGGTCTTAGA
SEQ ID NO: 32. NucC substrate without recognition sequence. (Substrate PF6283, FIG 11H).
CCCTACGCTCCCTCCAGCGCTGTCGGGGATATAGTCACTCGGAGTTAGAGAGTTTTAGGATTGATTACTG
AACTCTAGTATGGTAAACTGTGAAAACTCATAAAGCTGACGAAGTAAAAGAATCAAACTAATAACTCAATCCAGT
CTAAAGAGTAGAAAGTTGGTGAAAGATTGTGAGTCAGTCACTTAATGGTCTTAGA
SEQ ID NO: 33. NucC substrate with core recognition sequence (bold). (Substrate PF6285,
FIG 11H).
CCCTACGCTCCCTCCAGCGCTGTCGGGGATATAGTCACTCGGAGTTGGCGCCTTTTAGGATTGATTACTG
AACTCTAGTATGGTAAACTGTGAAAACTCATAAAGCTGACGAAGTAAAAGAATCAAACTAATAACTCAATCCAGT
CTAAAGAGTAGAAAGTTGGTGAAAGATTGTGAGTCAGTCACTTAATGGTCTTAGA
SEQ ID NO: 37: NucC core recognition motif
GGCGCC
SEQ ID NO: 38: NucC long recognition motif
CAAGGGCGCCCTTG
SEQ ID NO: 69 Nuclease consensus recognition motif
CAnnGGCGCCnnTG
SEQ ID NO: 70: Top oligonucleotide for fluorescent reporter sequence, probe 1
/56-FAM/CTCGGCAAGGGCGCCCTTGAGGAT/3IABkFQ/
SEQ ID NO: 71: Bottom oligonucleotide for fluorescent reporter sequence, probe 1
ATCCTCAAGGGCGCCCTTGCCGAG
SEQ ID NO: 150: Top oligonucleotide for fluorescent reporter sequence, probe 2
/56-FAM/CTCGGCAAGGGCGCCCTTGAGGAT
SEQ ID NO: 151: Bottom oligonucleotide for fluorescent reporter sequence, probe 2
/3IABKFQ/ATCCTCAAGGGCGCCCTTGCCGAG

1. A method of detecting a target single stranded nucleic acid in a sample, the method comprising:
(b) contacting the sample with a complex comprising:
(i) a Type III-D CRISPR-Cas system comprising:
(1) a Cas7-Cas5-Cas11 fusion subunit;
(2) a Cas7-Cas7 fusion subunit;
(3) a Cas7-insertion subunit;
(4) a Cas10 subunit;
(5) a Csx19 subunit; and
(ii) a guide RNA which is complementary to a recognition sequence in the target single stranded nucleic acid;
to form a reaction mix,
(c) incubating the reaction mix from (a) for a time and under conditions sufficient for the complex to bind to the target nucleic acid if present in the sample and produce at least one cyclic oligoadenylate (coA);
(d) contacting the reaction mix from (b) with a nuclease and one or more nucleic acid probes, wherein the nuclease is activated by the at least one coA;
(e) incubating the reaction mix from (c) for a time and under conditions sufficient to cleave the one or more nucleic acid probes to produce one or more cleaved nucleic acid probes; and
(f) determining whether one or more cleaved nucleic acid probes is present in the sample.

2. The method according to claim 1, wherein the Type III-D CRISPR-Cas system further comprises a Cas6 subunit.

3. The method according to claim 1 or claim 2, wherein at least one Cas7 containing subunits selected from the Cas-Cas7 fusion subunit and/or the Cas7-Cas5-Cas11 fusion subunit is modified to have reduced ribonuclease activity relative to an unmodified Cas7 containing subunit.

4. The method according to any one of claims 1 to 3, wherein the Cas10 subunit is modified to have reduced deoxyribonuclease activity.

5. The method according to any one of claims 1 to 4, wherein the Cas7-Cas7 fusion subunit is modified at positions D246 and/or D33 of SEQ ID NO: 6, or positions corresponding thereto.

6. The method according to any one of claims 1 to 5, wherein the Cas7-Cas5-Cas11 fusion subunit is modified at position D26 of SEQ ID NO: 4, or a position corresponding thereto.

7. The method according to any of claims 1 to 6, wherein the Cas10 subunit is modified at positions H337 and/or D338 of SEQ ID NO: 2, or corresponding positions thereto.

8. The method according to any of claims 1 to 7 wherein the target single stranded nucleic acid is a ribose nucleic acid (RNA).

9. The method according to any of claims 1 to 8, wherein the nuclease introduced at step (c) is a DNA nuclease, preferably a NucC nuclease, more preferably from Serratia sp. ATCC 39006.

10. The method according to claim 9, wherein the nuclease comprises the sequence according SEQ ID NO: 30.

11. The method according to any of claims 1 to 10 wherein the Type III-D CRISPR-Cas complex produces cyclic oligoadenylates selected from cA2 cA3, cA4, cA5, and cA6, preferably wherein the Type III-D CRISPR-Cas complex produces cA3 cyclic oligoadenylates.

12. The method according to claim 11 wherein the nuclease specifically binds to cA3 cyclic oligoadenylates.

13. The method according to any of claims 1 to 12 wherein the one or more nucleic acid probes is a deoxyribose nucleic acid probe.

14. The method according to any of claims 1 to 13 wherein the one or more nucleic acid probes comprise a recognition motif recognised and cleaved by the nuclease, preferably the recognition motif is GGCGCC (SEQ ID NO: 37).

15. The method according to any one of claims 1 to 14, wherein:
(a) the Cas7-Cas5-Cas11 fusion subunit comprises an amino acid sequence set forth in SEQ ID NO: 4, or variant sequence which comprises at least 80%, 85%, 90%, 95%, 96%, 97%, 98% or 99% sequence identity to SEQ ID NO: 4; and
(b) the Cas7-Cas7 fusion subunit comprises an amino acid sequence set forth in SEQ ID NO: 6, or variant sequence which comprises at least 80%, 85%, 90%, 95%, 96%, 97%, 98% or 99% sequence identity to SEQ ID NO: 6.

16. The method according to any one of claims 1 to 15, wherein the sample is a biological sample, preferably a biological fluid selected from blood, plasma, sputum, saliva and a central spinal fluid.

17. A modified Type III-D CRISPR-Cas system comprising: a Cas10 subunit, a Csx19 subunit, a Cas7-Cas7 fusion subunit, a Cas7-Cas5-Cas11 fusion subunit, and a Cas7-insertion subunit, wherein:
(a) at least one of the Cas7 containing subunits is modified to have a reduced ribonuclease activity relative to an unmodified Type III-D CRISPR-Cas system; and/or
(b) the Cas10 subunit is modified to have a reduced deoxyribonuclease activity and/or is modified to reduce cyclic oligoadenylate production relative to an unmodified Type III-D CRISPR-Cas system.

18. One or more nucleic acids encoding the modified Type III-D CRISPR-Cas system according to claim 17.

19. A vector, phage or virus comprising the one or more nucleic acids according to claim 18.

20. A host cell comprising the one or more nucleic acids according to claim 18, or the expression vector, phage or virus according to claim 19.


SEQUENCE LISTING (157 sequences)

SEQ ID NO: 1 - DNA, 1677 bp, Synechocystis sp.
  source 1..1677: mol_type = other DNA; strain = PCC 6803
atgtttctagttctaattgagacttccggtaatcagcattttattttctcgactaataaactaagggaaaatattggtgcatcagagttgacctatcttgctacaacggaaatattgttccagggggtggatagggttttccagactaactactatgaccaatggtctgacacaaactccctaaattttttggcagatagtaagcttaatcccgccattgatgatcctaaaaataacgctgacattgaaattttattggctacctctggaaaggcgatcgccctggtgaaagaagagggcaaggctaaacaattaattaaagaagttaccaagcaggccctaatcaatgccccgggtttagaaattggtggtatttatgtgaattgtaattggcaagataaattaggggttgccaaagcagttaaagaagcccataaacagttcgaagtaaatagggctaaacgggctggggctaatggtcgctttttgcggttaccgatcgccgctgggtgcagtgtaagtgaattgcctgcctctgattttgactataatgccgatggtgacaagattcctgtttctacagtcagtaaagttaaacgggagactgcgaaatctgccaaaaaacgtttgcggagcgttgatggtcggctagttaacgacctagcacaattagaaaagtcctttgacgaattagattggttagcagtggtccatgccgatggtaatggtttggggcaaattttactaagtcttgagaaatatattggtgagcaaacaaaccgcaattatattgataaatatcgtagactttctttagccctggataactgcaccatcaacgcttttaaaatggcgatcgctgtcttcaaagaagattccaaaaaaattgatttacccattgtcccattgattttaggtggagatgacctaacggtaatttgtcggggggactacgccctagaattcaccagggaatttcttgaagcatttgaagggcagacagaaacacatgatgatatcaaagtaatagcccaaaaagcctttggcgttgatcgcctttctgcctgcgctgggatcagtattattaagccccattttcccttctctgttgcctatactttggcggaaagattaattaaatcagctaaggaggtcaaacaaaaagttactgtgacaaatagttcgccaataactccttttccctgctctgccattgattttcatattctctatgacagtagcggcattgattttgaccgtattcgtgaaaaattacggccggaagataataccgagctttacaaccgtccctatgtggtgacagcagcggagaacctcagccaagcccagggttatgaatggtcccaggcccacagtttgcaaacactagcggatcgggttagttatttacgttccgaagatggggaaggaaaatctgcattacccagcagtcaaagccatgccctacgaacggcattgtacctagagaaaaatgaagcagacgctcaatatagcttaattagccaacgctacaaaattctcaaaaactttgcggaggacggagagaataaatcactatttcatctcgaaaatggcaagtacgtcaccagatttttagatgcactggatgccaaagatttttttgctaacgctaaccataaaaaccaaggagaataa

SEQ ID NO: 2 - protein, 558 aa, Synechocystis sp.
  REGION 1..558: note = PCC 6803
MFLVLIETSGNQHFIFSTNKLRENIGASELTYLATTEILFQGVDRVFQTNYYDQWSDTNSLNFLADSKLNPAIDDPKNNADIEILLATSGKAIALVKEEGKAKQLIKEVTKQALINAPGLEIGGIYVNCNWQDKLGVAKAVKEAHKQFEVNRAKRAGANGRFLRLPIAAGCSVSELPASDFDYNADGDKIPVSTVSKVKRETAKSAKKRLRSVDGRLVNDLAQLEKSFDELDWLAVVHADGNGLGQILLSLEKYIGEQTNRNYIDKYRRLSLALDNCTINAFKMAIAVFKEDSKKIDLPIVPLILGGDDLTVICRGDYALEFTREFLEAFEGQTETHDDIKVIAQKAFGVDRLSACAGISIIKPHFPFSVAYTLAERLIKSAKEVKQKVTVTNSSPITPFPCSAIDFHILYDSSGIDFDRIREKLRPEDNTELYNRPYVVTAAENLSQAQGYEWSQAHSLQTLADRVSYLRSEDGEGKSALPSSQSHALRTALYLEKNEADAQYSLISQRYKILKNFAEDGENKSLFHLENGKYVTRFLDALDAKDFFANANHKNQGE

SEQ ID NO: 3 - DNA, 2376 bp, Synechocystis sp.
  source 1..2376: mol_type = other DNA; strain = PCC 6803
atgcgaggaattgagataaccataaccatgcagagtgattggcacgttggcactggcatgggtcggggggaactggacagtgttgtacaacgggatggagataatctgccctatattcccggcaaaaccttaacaggtattctgcgggatagctgtgaacaggttgccctaggtttagataatggtcaaacccgagggctttggcatgggtggattaattttatttttggcgatcaacctgccctagctcaaggagctattgagccagaacctagacctgccctaatcgccattggttctgcacaccttgaccctaagttaaaagcggcttttcagggcaaaaaacaattgcaagaggcgatcgcctttatgaagccaggggtggctatcgatgcaatcacgggcacagctaagaaagattttttacgctttgaagaagtagttcgtttgggagcgaaattaactgcggaagttgagttaaatttacccgataatttgagcgaaaccaataaaaaagttattgctggtattttagccagtggagcaaagttaaccgagagattaggcggtaaacgtcgccggggcaatgggcgctgtgaattaaaatttagtggttattctgatcaacaaattcaatggttgaaagacaattatcaatctgttgatcaaccacctaagtatcaacaaaataaattacaatctgccggagataatccagaacagcaacccccttggcatattattcccttaaccattaaaaccctttctcctgttgttttaccagctcgtacagtcggtaacgttgtcgaatgtttagactatattcccgggcgttatctactgggctatattcacaaaaccctaggggaatatttcgacgttagtcaggcaatcgccgctggggatttaattattaccaatgccacgataaaaattgatggtaaagcaggacgagctaccccattttgtttgtttggggaaaaactagatggaggattaggtaaaggtaaaggagtttataaccgtttccaagaatcggaacctgatggcattcaattaaagggagaacggggcggctatgttggccaatttgaacaggagcaaaggaatctgccaaatacggggaaaattaattcagagttatttacccataacaccattcaagatgatgtccagcggcccaccagtgatgtggggggagtttatagctatgaagctattatagccggacaaacattcgtcgctgagttacgtttaccagatagcttagtcaagcaaattacaagcaaaaataaaaattggcaagctcaactaaaagctacaattcgcattggtcagtctaaaaaagatcagtatggcaaaatcgaagttacgtcgggaaactctgctgatttgcctaagcctacgggcaacaataaaactctttctatttggttcttatccgatatccttctccgaggcgatcgcctaaattttaatgctactccggatgatctcaaaaaatacttagaaaatgctctggatatcaagctcaaagaacgatcagacaatgatttaatttgcattgctctccgttcccagcggacagaatcctggcaagtacggtggggtttaccccggccatctctagtgggttggcaagctggtagttgtctgatttatgacattgaatctggcactgttaatgccgaaaaattgcaagaattaatgatcaccggcattggcgatcggtgtacagagggttacggtcaaatcggttttaacgatccattactttcggcttccctaggaaagttgacagctaagcctaaagcttctaacaatcagtcccaaaacagccaatccaacccattacccactaatcatcctacccaagattatgctcgattaattgaaaaagcggcttggcgggaagcaattcaaaataaagccttagccttggcatctagccgagcgaaacgggaagaaattttaggcattaaaattatgggaaaagatagtcaacccaccatgactcaattaggaggatttcgctccgtattaaaacggctacactcaagaaataatcgagatattgtcacaggttatttaacagctctagagcaggtttctaatcgaaaagaaaaatggagtaataccagccaaggattaactaaaattcgtaatttagtcacccaggaaaatctcatttggaatcatcttgatattgatttttcgccgttaactattacccaaaatggtgttaatcagctaaagtctgaactttgggcggaagcagtgcgaacccttgttgacgctatcattcggggtcataaacgggacttagaaaaagctcaagaaaacgaatctaatcaacagtcacagggagcagcttaa

SEQ ID NO: 4 - protein, 791 aa, Synechocystis sp.
  REGION 1..791: note = PCC 6803
MRGIEITITMQSDWHVGTGMGRGELDSVVQRDGDNLPYIPGKTLTGILRDSCEQVALGLDNGQTRGLWHGWINFIFGDQPALAQGAIEPEPRPALIAIGSAHLDPKLKAAFQGKKQLQEAIAFMKPGVAIDAITGTAKKDFLRFEEVVRLGAKLTAEVELNLPDNLSETNKKVIAGILASGAKLTERLGGKRRRGNGRCELKFSGYSDQQIQWLKDNYQSVDQPPKYQQNKLQSAGDNPEQQPPWHIIPLTIKTLSPVVLPARTVGNVVECLDYIPGRYLLGYIHKTLGEYFDVSQAIAAGDLIITNATIKIDGKAGRATPFCLFGEKLDGGLGKGKGVYNRFQESEPDGIQLKGERGGYVGQFEQEQRNLPNTGKINSELFTHNTIQDDVQRPTSDVGGVYSYEAIIAGQTFVAELRLPDSLVKQITSKNKNWQAQLKATIRIGQSKKDQYGKIEVTSGNSADLPKPTGNNKTLSIWFLSDILLRGDRLNFNATPDDLKKYLENALDIKLKERSDNDLICIALRSQRTESWQVRWGLPRPSLVGWQAGSCLIYDIESGTVNAEKLQELMITGIGDRCTEGYGQIGFNDPLLSASLGKLTAKPKASNNQSQNSQSNPLPTNHPTQDYARLIEKAAWREAIQNKALALASSRAKREEILGIKIMGKDSQPTMTQLGGFRSVLKRLHSRNNRDIVTGYLTALEQVSNRKEKWSNTSQGLTKIRNLVTQENLIWNHLDIDFSPLTITQNGVNQLKSELWAEAVRTLVDAIIRGHKRDLEKAQENESNQQSQGAA

SEQ ID NO: 5 - DNA, 1569 bp, Synechocystis sp.
  source 1..1569: mol_type = other DNA; strain = PCC 6803
atggctagaaaagttactacacgctggaaaattacaggcacattaattgcagaaacccctttacacattggtggtgtgggtggcgacgctgatacggatttagccctggcggttaatggtgcgggtgaatattatgtgccagggacaagtttagccggtgctctgcggggttggatgacccagttattgaataatgatgagtcccaaattaaagatctttggggtgatcatttagatgcaaaacggggagctagctttgttattgttgacgatgcggttatccatatacccaataatgctgatgttgaaattagggagggtgttggcatcgatcgccattttggaaccgccgccaatgggtttaaatatagccgagcagttattcccaagggttctaaatttaaattgccattaacttttgacagtcaagatgatgggctaccgaatgcgttgattcaattgttgtgtgccttagaagcaggggatattcgccttggggccgcaaaaacccggggtttaggtcgcattaaactagatgatttaaagttaaaatcctttgctttagataaaccagaaggtattttttctgctttattagaccaaggtaaaaaattagattggaatcaattaaaagcaaacgttacctaccagtctcctccctatctaggtattagtattacctggaatcccaaagatcccgtcatggtgaaagctgaaggggatggactggcgatcgatattttgcccctcgttagtcaagtgggaagtgatgttcgatttgtcattcccggcagttccattaaggggattttacgaacccaggctgaacgtattattcgtactatttgccagtctaatggttctgagaaaaacttcctagaacaattacgaatcaatctggttaatgaattatttgggtctgcttctttgagccaaaaacaaaatggcaaggatatagatctgggtaaaatcggagccttggcagtgaatgattgtttttctagtttatccatgaccccagatcaatggaaagcggtagagaatgccacggagatgacggggaatttacagcctgctcttaaacaagctacgggttatcccaataatattagccaagcttacaaagtacttcaaccggccatgcacgtcgctgtagatcggtggacagggggagctgccgaaggaatgctttacagcgtgctcgaacccattggggtcacctgggaaccgatccaagttcacttggacattgcccgtctcaaaaattattaccacggtaaggaagaaaaacttaaaccggcgatcgccctattgcttcttgtattgcgggatttagctaacaaaaaaattcccgtaggctatggcactaaccgcggtatgggaacgattactgtcagtcaaatcaccctcaatggcaaagccctccccactgaacttgaacctttaaacaaaacaatgacttgtcctaatctcaccgatctagatgaggcatttcgtcaggacttaagcactgcttggaaagagtggattgccgatcccattgatctatgccagcaggaggccgcctaa

SEQ ID NO: 6 - protein, 522 aa, Synechocystis sp.
  REGION 1..522: note = PCC 6803
MARKVTTRWKITGTLIAETPLHIGGVGGDADTDLALAVNGAGEYYVPGTSLAGALRGWMTQLLNNDESQIKDLWGDHLDAKRGASFVIVDDAVIHIPNNADVEIREGVGIDRHFGTAANGFKYSRAVIPKGSKFKLPLTFDSQDDGLPNALIQLLCALEAGDIRLGAAKTRGLGRIKLDDLKLKSFALDKPEGIFSALLDQGKKLDWNQLKANVTYQSPPYLGISITWNPKDPVMVKAEGDGLAIDILPLVSQVGSDVRFVIPGSSIKGILRTQAERIIRTICQSNGSEKNFLEQLRINLVNELFGSASLSQKQNGKDIDLGKIGALAVNDCFSSLSMTPDQWKAVENATEMTGNLQPALKQATGYPNNISQAYKVLQPAMHVAVDRWTGGAAEGMLYSVLEPIGVTWEPIQVHLDIARLKNYYHGKEEKLKPAIALLLLVLRDLANKKIPVGYGTNRGMGTITVSQITLNGKALPTELEPLNKTMTCPNLTDLDEAFRQDLSTAWKEWIADPIDLCQQEAA

SEQ ID NO: 7 - DNA, 582 bp, Synechocystis sp.
  source 1..582: mol_type = other DNA; strain = PCC 6803
atgccagcaggaggccgcctaatgaagaacctttaccactaccaccaatatgaaattaccctcgaatccgccgtcgattcttgcaaaaaccatctccaagcggcgatcgggctgttgtattctccccaaaagtgtgaactagtcaaactggataactcaggcaagttagttgattcttacaatcgtcttaagttcaataacctaggcgtatttgaagcccgcttctttaatctcaattgtgaactgcgatgggtcaatgaatctaatggtaatggcactgccgtcttgctttcagaatcggatattaccttaactggttttgagaaaggtttacaggaatttattacggcgatcgaccaacagtatttactctggggtgaacccgctaaacatccccctaatgctgatggctggcaacgactagcggaagcaaggatcgggaaactcgatattcccctcgataacccgttaaaacccaaagatcgagtttttctcaccagcgaagagtacattgctgaagtagatgattttggtaattgtgccgttattgacgaacgtttaattaaattggaggttaagtaa

SEQ ID NO: 8 - protein, 193 aa, Synechocystis sp.
  REGION 1..193: note = PCC 6803
MPAGGRLMKNLYHYHQYEITLESAVDSCKNHLQAAIGLLYSPQKCELVKLDNSGKLVDSYNRLKFNNLGVFEARFFNLNCELRWVNESNGNGTAVLLSESDITLTGFEKGLQEFITAIDQQYLLWGEPAKHPPNADGWQRLAEARIGKLDIPLDNPLKPKDRVFLTSEEYIAEVDDFGNCAVIDERLIKLEVK

SEQ ID NO: 9 - DNA, 2388 bp, Synechocystis sp.
  source 1..2388: mol_type = genomic DNA; strain = PCC 6803
atgacagtcggaacattgggcgttgttggcagtgctaaaaacctcaaattacaacttagttttatcaacacaaggcaacagtatgttcaaataacactttttgagcgaaattcttttaaggttgctgaggaagaattttctactgaacttgtggaaatcattaaaacagcactaccaactctcaaaaataaaaaagttgaatttgaggaagatggcgatcaaattaaacaaatccgagaaaaaggtcaagcttgggttggtgccgcagaacagattgcaccttatgttcttccttctggaaatattactgaaacacccagaaatgttaacgctagcaactttcataacccctacaactttgtcccagccctaccccgcgatggcataaccggagatttaggcgactgtgctcctgctggtcatagctattaccatggcgataaatacagcggcagaattgccgtcaaactaacaaccgttacccctctattgattcctgacgcttcaaaagaagagataaataacaaccataaaacctatccggttcgtatcggcaaagatggcaagccctatctacctcccacttccattaagggaatgttgcgctctgcctatgaagcggtcactaattcccgcttagccgtgtttgaagatcatgactctcgcttggcctatcgaatgcctgccaccatgggattgcaaatggttcctgcccgcattgaaggtgataatattgttctttacccaggaacctcaaggataggcaataatggccgaccagctaacaatgatcctatgtatgcggcatggcttccttactatcaaaatcgtattgcttatgatggtagtcgtgattatcagatggctgagcatggtgatcatgtcagattttgggctgagcgatataccagaggaaacttctgctattggcgtgtcagacaaattgcacgacacaatcaaaatttaggtaatcggcctgaacgaggacgtaattacggtcaacatcattcaacaggagtcattgaacaatttgaaggatttgtttacaaaaccaataaaaatattgggaataaacatgacgaacgagtatttattattgatcgagaaagtatcgaaatacctctatctcgagatttacggcgaaaatggcgagaattaattacaagctatcaggaaatacacaaaaaggaagttgatagaggtgatactggcccttccgctgtaaatggggctgtttggtcacggcaaattattgcagatgaatcagagcggaatttatcggatgggactctttgttatgctcatgttaagaaagaagatggacagtacaaaattctcaatctttatcctgtaatgatcacacggggattatatgaaattgcgccggttgacttattagatgaaaccctaaagcctgcgacggataaaaagcaactatccccagcagaccgcgtatttggctgggtcaatcaacggggcaatggttgctacaaaggacaattacgaattcatagcgtaacttgccaacatgatgatgccattgatgattttggtaatcaaaatttctctgttccccttgctattttgggacaacctaaaccagaacaggctcgtttttattgtgccgatgatcgaaaaggaattcctttagaagatggctatgatcgtgacgacggctatagtgattcagaacaaggcttgcgaggacgcaaagtctatcctcaccacaaggggttaccaaatggctactggagtaatccaacggaagaccgaagtcaacaagctatccaaggtcattaccaagaatatcgtcgtcctaaaaaggatggtcttgaacaaagagatgatcaaaatcgttctgtaaaaggttgggtaaaaccactgaccgagtttacttttgaaattgacgttactaatctttcggaagttgagttaggtgctctattgtggttgttaaccttacctgatttgcatttccaccgtctaggaggaggtaaaccgttaggttttggtagtgttcgtttagatattgaccctgacaagacagacctaagaaatggggcaggatggcgtgattattacggctctttactagaaacaagtcaaccagattttacaactctaattagtcagtggattaatgcttttcaaacggctgttaaagaggagtatggtagcagtagttttgatcaggttactttcatcaaagcttctggtcagagtctccaaggatttcatgataatgcatctatccattatcctcgttctactcctgagcccaagccagatggagaagcttttaagtggtttgttgccaatgaaaaaggtcgacgattagccttgccagcgctggaaaaatcccagagttttccaatcaaacctagttaa

SEQ ID NO: 10 - protein, 795 aa, Synechocystis sp.
  REGION 1..795: note = PCC 6803
MTVGTLGVVGSAKNLKLQLSFINTRQQYVQITLFERNSFKVAEEEFSTELVEIIKTALPTLKNKKVEFEEDGDQIKQIREKGQAWVGAAEQIAPYVLPSGNITETPRNVNASNFHNPYNFVPALPRDGITGDLGDCAPAGHSYYHGDKYSGRIAVKLTTVTPLLIPDASKEEINNNHKTYPVRIGKDGKPYLPPTSIKGMLRSAYEAVTNSRLAVFEDHDSRLAYRMPATMGLQMVPARIEGDNIVLYPGTSRIGNNGRPANNDPMYAAWLPYYQNRIAYDGSRDYQMAEHGDHVRFWAERYTRGNFCYWRVRQIARHNQNLGNRPERGRNYGQHHSTGVIEQFEGFVYKTNKNIGNKHDERVFIIDRESIEIPLSRDLRRKWRELITSYQEIHKKEVDRGDTGPSAVNGAVWSRQIIADESERNLSDGTLCYAHVKKEDGQYKILNLYPVMITRGLYEIAPVDLLDETLKPATDKKQLSPADRVFGWVNQRGNGCYKGQLRIHSVTCQHDDAIDDFGNQNFSVPLAILGQPKPEQARFYCADDRKGIPLEDGYDRDDGYSDSEQGLRGRKVYPHHKGLPNGYWSNPTEDRSQQAIQGHYQEYRRPKKDGLEQRDDQNRSVKGWVKPLTEFTFEIDVTNLSEVELGALLWLLTLPDLHFHRLGGGKPLGFGSVRLDIDPDKTDLRNGAGWRDYYGSLLETSQPDFTTLISQWINAFQTAVKEEYGSSSFDQVTFIKASGQSLQGFHDNASIHYPRSTPEPKPDGEAFKWFVANEKGRRLALPALEKSQSFPIKPS

SEQ ID NO: 11 - DNA, 1104 bp, Synechocystis sp.
  source 1..1104: mol_type = other DNA; strain = PCC 6803
gtggtggatctaaaatccttagctggggccgaaatggtgggattacgctggcaactgcgcttcgaccgcccctgtcgcctggaaagtcattacgttaaaggactccatgcttggtttttgcatcaagtgcaggccattgatcccgatgtttctgcctggctccatgatggtcaaggggaaaagcccttcaccatttcccgcctgatagggcctaccctctggcaagaaggtcattggcactggcaaataaataagacctaccattggcaattaaatttactatcaggggctttaatcgaagctttacaaccttggctagcccgtttgccaaacaaaattgtcctagctcgccaaacattatgggtagaagccgttgattgttacctagccccccataactatcaacagttatggccccagggtgctttaccccgacggcaagagtttactttcactagccctaccagtttccgtcgccaaggcaatcactatccgttaccagagccccgcaatgttctgcaaagttatctacggcgttggaatgatttttctggtttggcgttcgagccggagccatttttggactattgggtgccccaaaatgtggtgatcgatcgccattggttggagtcggtgaagaccacagcgggaaaacaaggctcagtggtgggatttgtgggagcagtgtccctagtccttacgccccaggcccgtaatgatggggatgattatggccgcttgttccatgccctctgtcgatatggaccctactgtggcactgggcataaaaccacctttggtttggggcaaacaatggcgggctgggctaccccggacctaaaaacttttgcgtgcctccaagaagatttacagactcaggtgttaacgcaacggatagatcaatgcgcctctctcctcctagcccagcgtcaacggacaggagggcagagagcccaggaaatttgccatacgctagccactatttttgtccgccgagaacagggggaatcattgcaagaaatcgccctggatttacagttaccttatgagacagcccgcacctacagcaaacgagctaagcgggccttagccaatgttcaataa

SEQ ID NO: 12 - protein, 367 aa, Synechocystis sp.
  REGION 1..367: note = PCC 6803
VVDLKSLAGAEMVGLRWQLRFDRPCRLESHYVKGLHAWFLHQVQAIDPDVSAWLHDGQGEKPFTISRLIGPTLWQEGHWHWQINKTYHWQLNLLSGALIEALQPWLARLPNKIVLARQTLWVEAVDCYLAPHNYQQLWPQGALPRRQEFTFTSPTSFRRQGNHYPLPEPRNVLQSYLRRWNDFSGLAFEPEPFLDYWVPQNVVIDRHWLESVKTTAGKQGSVVGFVGAVSLVLTPQARNDGDDYGRLFHALCRYGPYCGTGHKTTFGLGQTMAGWATPDLKTFACLQEDLQTQVLTQRIDQCASLLLAQRQRTGGQRAQEICHTLATIFVRREQGESLQEIALDLQLPYETARTYSKRAKRALANVQ

SEQ ID NO: 13 - DNA, 1677 bp, synthetic construct
  source 1..1677: mol_type = other DNA
atgtttctagttctaattgagacttccggtaatcagcattttattttctcgactaataaactaagggaaaatattggtgcatcagagttgacctatcttgctacaacggaaatattgttccagggggtggatagggttttccagactaactactatgaccaatggtctgacacaaactccctaaattttttggcagatagtaagcttaatcccgccattgatgatcctaaaaataacgctgacattgaaattttattggctacctctggaaaggcgatcgccctggtgaaagaagagggcaaggctaaacaattaattaaagaagttaccaagcaggccctaatcaatgccccgggtttagaaattggtggtatttatgtgaattgtaattggcaagataaattaggggttgccaaagcagttaaagaagcccataaacagttcgaagtaaatagggctaaacgggctggggctaatggtcgctttttgcggttaccgatcgccgctgggtgcagtgtaagtgaattgcctgcctctgattttgactataatgccgatggtgacaagattcctgtttctacagtcagtaaagttaaacgggagactgcgaaatctgccaaaaaacgtttgcggagcgttgatggtcggctagttaacgacctagcacaattagaaaagtcctttgacgaattagattggttagcagtggtccatgccgatggtaatggtttggggcaaattttactaagtcttgagaaatatattggtgagcaaacaaaccgcaattatattgataaatatcgtagactttctttagccctggataactgcaccatcaacgcttttaaaatggcgatcgctgtcttcaaagaagattccaaaaaaattgatttacccattgtcccattgattttaggtggagatgacctaacggtaatttgtcggggggactacgccctagaattcaccagggaatttcttgaagcatttgaagggcagacagaaacagcagctgatatcaaagtaatagcccaaaaagcctttggcgttgatcgcctttctgcctgcgctgggatcagtattattaagccccattttcccttctctgttgcctatactttggcggaaagattaattaaatcagctaaggaggtcaaacaaaaagttactgtgacaaatagttcgccaataactccttttccctgctctgccattgattttcatattctctatgacagtagcggcattgattttgaccgtattcgtgaaaaattacggccggaagataataccgagctttacaaccgtccctatgtggtgacagcagcggagaacctcagccaagcccagggttatgaatggtcccaggcccacagtttgcaaacactagcggatcgggttagttatttacgttccgaagatggggaaggaaaatctgcattacccagcagtcaaagccatgccctacgaacggcattgtacctagagaaaaatgaagcagacgctcaatatagcttaattagccaacgctacaaaattctcaaaaactttgcggaggacggagagaataaatcactatttcatctcgaaaatggcaagtacgtcaccagatttttagatgcactggatgccaaagatttttttgctaacgctaaccataaaaaccaaggagaataa

SEQ ID NO: 14 - protein, 558 aa, synthetic construct
MFLVLIETSGNQHFIFSTNKLRENIGASELTYLATTEILFQGVDRVFQTNYYDQWSDTNSLNFLADSKLNPAIDDPKNNADIEILLATSGKAIALVKEEGKAKQLIKEVTKQALINAPGLEIGGIYVNCNWQDKLGVAKAVKEAHKQFEVNRAKRAGANGRFLRLPIAAGCSVSELPASDFDYNADGDKIPVSTVSKVKRETAKSAKKRLRSVDGRLVNDLAQLEKSFDELDWLAVVHADGNGLGQILLSLEKYIGEQTNRNYIDKYRRLSLALDNCTINAFKMAIAVFKEDSKKIDLPIVPLILGGDDLTVICRGDYALEFTREFLEAFEGQTETAADIKVIAQKAFGVDRLSACAGISIIKPHFPFSVAYTLAERLIKSAKEVKQKVTVTNSSPITPFPCSAIDFHILYDSSGIDFDRIREKLRPEDNTELYNRPYVVTAAENLSQAQGYEWSQAHSLQTLADRVSYLRSEDGEGKSALPSSQSHALRTALYLEKNEADAQYSLISQRYKILKNFAEDGENKSLFHLENGKYVTRFLDALDAKDFFANANHKNQGE

SEQ ID NO: 15 - DNA, 1677 bp, synthetic construct
  source 1..1677: mol_type = other DNA
atgtttctagttctaattgagacttccggtaatcagcattttattttctcgactaataaactaagggaaaatattggtgcatcagagttgacctatcttgctacaacggaaatattgttccagggggtggatagggttttccagactaactactatgaccaatggtctgacacaaactccctaaattttttggcagatagtaagcttaatcccgccattgatgatcctaaaaataacgctgacattgaaattttattggctacctctggaaaggcgatcgccctggtgaaagaagagggcaaggctaaacaattaattaaagaagttaccaagcaggccctaatcaatgccccgggtttagaaattggtggtatttatgtgaattgtaattggcaagataaattaggggttgccaaagcagttaaagaagcccataaacagttcgaagtaaatagggctaaacgggctggggctaatggtcgctttttgcggttaccgatcgccgctgggtgcagtgtaagtgaattgcctgcctctgattttgactataatgccgatggtgacaagattcctgtttctacagtcagtaaagttaaacgggagactgcgaaatctgccaaaaaacgtttgcggagcgttgatggtcggctagttaacgacctagcacaattagaaaagtcctttgacgaattagattggttagcagtggtccatgccgatggtaatggtttggggcaaattttactaagtcttgagaaatatattggtgagcaaacaaaccgcaattatattgataaatatcgtagactttctttagccctggataactgcaccatcaacgcttttaaaatggcgatcgctgtcttcaaagaagattccaaaaaaattgatttacccattgtcccattgattttaggtggagctgccctaacggtaatttgtcggggggactacgccctagaattcaccagggaatttcttgaagcatttgaagggcagacagaaacacatgatgatatcaaagtaatagcccaaaaagcctttggcgttgatcgcctttctgcctgcgctgggatcagtattattaagccccattttcccttctctgttgcctatactttggcggaaagattaattaaatcagctaaggaggtcaaacaaaaagttactgtgacaaatagttcgccaataactccttttccctgctctgccattgattttcatattctctatgacagtagcggcattgattttgaccgtattcgtgaaaaattacggccggaagataataccgagctttacaaccgtccctatgtggtgacagcagcggagaacctcagccaagcccagggttatgaatggtcccaggcccacagtttgcaaacactagcggatcgggttagttatttacgttccgaagatggggaaggaaaatctgcattacccagcagtcaaagccatgccctacgaacggcattgtacctagagaaaaatgaagcagacgctcaatatagcttaattagccaacgctacaaaattctcaaaaactttgcggaggacggagagaataaatcactatttcatctcgaaaatggcaagtacgtcaccagatttttagatgcactggatgccaaagatttttttgctaacgctaaccataaaaaccaaggagaataa

SEQ ID NO: 16 - protein, 558 aa, synthetic construct
MFLVLIETSGNQHFIFSTNKLRENIGASELTYLATTEILFQGVDRVFQTNYYDQWSDTNSLNFLADSKLNPAIDDPKNNADIEILLATSGKAIALVKEEGKAKQLIKEVTKQALINAPGLEIGGIYVNCNWQDKLGVAKAVKEAHKQFEVNRAKRAGANGRFLRLPIAAGCSVSELPASDFDYNADGDKIPVSTVSKVKRETAKSAKKRLRSVDGRLVNDLAQLEKSFDELDWLAVVHADGNGLGQILLSLEKYIGEQTNRNYIDKYRRLSLALDNCTINAFKMAIAVFKEDSKKIDLPIVPLILGGAALTVICRGDYALEFTREFLEAFEGQTETHDDIKVIAQKAFGVDRLSACAGISIIKPHFPFSVAYTLAERLIKSAKEVKQKVTVTNSSPITPFPCSAIDFHILYDSSGIDFDRIREKLRPEDNTELYNRPYVVTAAENLSQAQGYEWSQAHSLQTLADRVSYLRSEDGEGKSALPSSQSHALRTALYLEKNEADAQYSLISQRYKILKNFAEDGENKSLFHLENGKYVTRFLDALDAKDFFANANHKNQGE

SEQ ID NO: 17 - DNA, 2375 bp, synthetic construct
  source 1..2375: mol_type = other DNA
atgcgaggaattgagataaccataaccatgcagagtgattggcacgttggcactggcatgggtcggggggaactgccagtgttgtacaacgggatggagataatctgccctatattcccggcaaaaccttaacaggtattctgcgggatagctgtgaacaggttgccctaggtttagataatggtcaaacccgagggctttggcatgggtggattaattttatttttggcgatcaacctgccctagctcaaggagctattgagccagaacctagacctgccctaatcgccattggttctgcacaccttgaccctaagttaaaagcggcttttcagggcaaaaaacaattgcaagaggcgatcgcctttatgaagccaggggtggctatcgatgcaatcacgggcacagctaagaaagattttttacgctttgaagaagtagttcgtttgggagcgaaattaactgcggaagttgagttaaatttacccgataatttgagcgaaaccaataaaaaagttattgctggtattttagccagtggagcaaagttaaccgagagattaggcggtaaacgtcgccggggcaatgggcgctgtgaattaaaatttagtggttattctgatcaacaaattcaatggttgaaagacaattatcaatctgttgatcaaccacctaagtatcaacaaaataaattacaatctgccggagataatccagaacagcaacccccttggcatattattcccttaaccattaaaaccctttctcctgttgttttaccagctcgtacagtcggtaacgttgtcgaatgtttagactatattcccgggcgttatctactgggctatattcacaaaaccctaggggaatatttcgacgttagtcaggcaatcgccgctggggatttaattattaccaatgccacgataaaaattgatggtaaagcaggacgagctaccccattttgtttgtttggggaaaaactagatggaggattaggtaaaggtaaaggagtttataaccgtttccaagaatcggaacctgatggcattcaattaaagggagaacggggcggctatgttggccaatttgaacaggagcaaaggaatctgccaaatacggggaaaattaattcagagttatttacccataacaccattcaagatgatgtccagcggcccaccagtgatgtggggggagtttatagctatgaagctattatagccggacaaacattcgtcgctgagttacgtttaccagatagcttagtcaagcaaattacaagcaaaaataaaaattggcaagctcaactaaaagctacaattcgcattggtcagtctaaaaaagatcagtatggcaaaatcgaagttacgtcgggaaactctgctgatttgcctaagcctacgggcaacaataaaactctttctatttggttcttatccgatatccttctccgaggcgatcgcctaaattttaatgctactccggatgatctcaaaaaatacttagaaaatgctctggatatcaagctcaaagaacgatcagacaatgatttaatttgcattgctctccgttcccagcggacagaatcctggcaagtacggtggggtttaccccggccatctctagtgggttggcaagctggtagttgtctgatttatgacattgaatctggcactgttaatgccgaaaaattgcaagaattaatgatcaccggcattggcgatcggtgtacagagggttacggtcaaatcggttttaacgatccattactttcggcttccctaggaaagttgacagctaagcctaaagcttctaacaatcagtcccaaaacagccaatccaacccattacccactaatcatcctacccaagattatgctcgattaattgaaaaagcggcttggcgggaagcaattcaaaataaagccttagccttggcatctagccgagcgaaacgggaagaaattttaggcattaaaattatgggaaaagatagtcaacccaccatgactcaattaggaggatttcgctccgtattaaaacggctacactcaagaaataatcgagatattgtcacaggttatttaacagctctagagcaggtttctaatcgaaaagaaaaatggagtaataccagccaaggattaactaaaattcgtaatttagtcacccaggaaaatctcatttggaatcatcttgatattgatttttcgccgttaactattacccaaaatggtgttaatcagctaaagtctgaactttgggcggaagcagtgcgaacccttgttgacgctatcattcggggtcataaacgggacttagaaaaagctcaagaaaacgaatctaatcaacagtcacagggagcagcttaa

SEQ ID NO: 18 - protein, 791 aa, synthetic construct
MRGIEITITMQSDWHVGTGMGRGELASVVQRDGDNLPYIPGKTLTGILRDSCEQVALGLDNGQTRGLWHGWINFIFGDQPALAQGAIEPEPRPALIAIGSAHLDPKLKAAFQGKKQLQEAIAFMKPGVAIDAITGTAKKDFLRFEEVVRLGAKLTAEVELNLPDNLSETNKKVIAGILASGAKLTERLGGKRRRGNGRCELKFSGYSDQQIQWLKDNYQSVDQPPKYQQNKLQSAGDNPEQQPPWHIIPLTIKTLSPVVLPARTVGNVVECLDYIPGRYLLGYIHKTLGEYFDVSQAIAAGDLIITNATIKIDGKAGRATPFCLFGEKLDGGLGKGKGVYNRFQESEPDGIQLKGERGGYVGQFEQEQRNLPNTGKINSELFTHNTIQDDVQRPTSDVGGVYSYEAIIAGQTFVAELRLPDSLVKQITSKNKNWQAQLKATIRIGQSKKDQYGKIEVTSGNSADLPKPTGNNKTLSIWFLSDILLRGDRLNFNATPDDLKKYLENALDIKLKERSDNDLICIALRSQRTESWQVRWGLPRPSLVGWQAGSCLIYDIESGTVNAEKLQELMITGIGDRCTEGYGQIGFNDPLLSASLGKLTAKPKASNNQSQNSQSNPLPTNHPTQDYARLIEKAAWREAIQNKALALASSRAKREEILGIKIMGKDSQPTMTQLGGFRSVLKRLHSRNNRDIVTGYLTALEQVSNRKEKWSNTSQGLTKIRNLVTQENLIWNHLDIDFSPLTITQNGVNQLKSELWAEAVRTLVDAIIRGHKRDLEKAQENESNQQSQGAA

SEQ ID NO: 19 - DNA, 1569 bp, synthetic construct
  source 1..1569: mol_type = other DNA
atggctagaaaagttactacacgctggaaaattacaggcacattaattgcagaaacccctttacacattggtggtgtgggtggcgacgctgatacggctttagccctggcggttaatggtgcgggtgaatattatgtgccagggacaagtttagccggtgctctgcggggttggatgacccagttattgaataatgatgagtcccaaattaaagatctttggggtgatcatttagatgcaaaacggggagctagctttgttattgttgacgatgcggttatccatatacccaataatgctgatgttgaaattagggagggtgttggcatcgatcgccattttggaaccgccgccaatgggtttaaatatagccgagcagttattcccaagggttctaaatttaaattgccattaacttttgacagtcaagatgatgggctaccgaatgcgttgattcaattgttgtgtgccttagaagcaggggatattcgccttggggccgcaaaaacccggggtttaggtcgcattaaactagatgatttaaagttaaaatcctttgctttagataaaccagaaggtattttttctgctttattagaccaaggtaaaaaattagattggaatcaattaaaagcaaacgttacctaccagtctcctccctatctaggtattagtattacctggaatcccaaagatcccgtcatggtgaaagctgaaggggatggactggcgatcgatattttgcccctcgttagtcaagtgggaagtgatgttcgatttgtcattcccggcagttccattaaggggattttacgaacccaggctgaacgtattattcgtactatttgccagtctaatggttctgagaaaaacttcctagaacaattacgaatcaatctggttaatgaattatttgggtctgcttctttgagccaaaaacaaaatggcaaggatatagatctgggtaaaatcggagccttggcagtgaatgattgtttttctagtttatccatgaccccagatcaatggaaagcggtagagaatgccacggagatgacggggaatttacagcctgctcttaaacaagctacgggttatcccaataatattagccaagcttacaaagtacttcaaccggccatgcacgtcgctgtagatcggtggacagggggagctgccgaaggaatgctttacagcgtgctcgaacccattggggtcacctgggaaccgatccaagttcacttggacattgcccgtctcaaaaattattaccacggtaaggaagaaaaacttaaaccggcgatcgccctattgcttcttgtattgcgggatttagctaacaaaaaaattcccgtaggctatggcactaaccgcggtatgggaacgattactgtcagtcaaatcaccctcaatggcaaagccctccccactgaacttgaacctttaaacaaaacaatgacttgtcctaatctcaccgatctagatgaggcatttcgtcaggacttaagcactgcttggaaagagtggattgccgatcccattgatctatgccagcaggaggccgcctaa

SEQ ID NO: 20 - protein, 522 aa, synthetic construct
MARKVTTRWKITGTLIAETPLHIGGVGGDADTALALAVNGAGEYYVPGTSLAGALRGWMTQLLNNDESQIKDLWGDHLDAKRGASFVIVDDAVIHIPNNADVEIREGVGIDRHFGTAANGFKYSRAVIPKGSKFKLPLTFDSQDDGLPNALIQLLCALEAGDIRLGAAKTRGLGRIKLDDLKLKSFALDKPEGIFSALLDQGKKLDWNQLKANVTYQSPPYLGISITWNPKDPVMVKAEGDGLAIDILPLVSQVGSDVRFVIPGSSIKGILRTQAERIIRTICQSNGSEKNFLEQLRINLVNELFGSASLSQKQNGKDIDLGKIGALAVNDCFSSLSMTPDQWKAVENATEMTGNLQPALKQATGYPNNISQAYKVLQPAMHVAVDRWTGGAAEGMLYSVLEPIGVTWEPIQVHLDIARLKNYYHGKEEKLKPAIALLLLVLRDLANKKIPVGYGTNRGMGTITVSQITLNGKALPTELEPLNKTMTCPNLTDLDEAFRQDLSTAWKEWIADPIDLCQQEAA

SEQ ID NO: 21 - DNA, 1569 bp, synthetic construct
  source 1..1569: mol_type = other DNA
atggctagaaaagttactacacgctggaaaattacaggcacattaattgcagaaacccctttacacattggtggtgtgggtggcgacgctgatacggatttagccctggcggttaatggtgcgggtgaatattatgtgccagggacaagtttagccggtgctctgcggggttggatgacccagttattgaataatgatgagtcccaaattaaagatctttggggtgatcatttagatgcaaaacggggagctagctttgttattgttgacgatgcggttatccatatacccaataatgctgatgttgaaattagggagggtgttggcatcgatcgccattttggaaccgccgccaatgggtttaaatatagccgagcagttattcccaagggttctaaatttaaattgccattaacttttgacagtcaagatgatgggctaccgaatgcgttgattcaattgttgtgtgccttagaagcaggggatattcgccttggggccgcaaaaacccggggtttaggtcgcattaaactagatgatttaaagttaaaatcctttgctttagataaaccagaaggtattttttctgctttattagaccaaggtaaaaaattagattggaatcaattaaaagcaaacgttacctaccagtctcctccctatctaggtattagtattacctggaatcccaaagatcccgtcatggtgaaagctgaaggggatggactggcgatcgctattttgcccctcgttagtcaagtgggaagtgatgttcgatttgtcattcccggcagttccattaaggggattttacgaacccaggctgaacgtattattcgtactatttgccagtctaatggttctgagaaaaacttcctagaacaattacgaatcaatctggttaatgaattatttgggtctgcttctttgagccaaaaacaaaatggcaaggatatagatctgggtaaaatcggagccttggcagtgaatgattgtttttctagtttatccatgaccccagatcaatggaaagcggtagagaatgccacggagatgacggggaatttacagcctgctcttaaacaagctacgggttatcccaataatattagccaagcttacaaagtacttcaaccggccatgcacgtcgctgtagatcggtggacagggggagctgccgaaggaatgctttacagcgtgctcgaacccattggggtcacctgggaaccgatccaagttcacttggacattgcccgtctcaaaaattattaccacggtaaggaagaaaaacttaaaccggcgatcgccctattgcttcttgtattgcgggatttagctaacaaaaaaattcccgtaggctatggcactaaccgcggtatgggaacgattactgtcagtcaaatcaccctcaatggcaaagccctccccactgaacttgaacctttaaacaaaacaatgacttgtcctaatctcaccgatctagatgaggcatttcgtcaggacttaagcactgcttggaaagagtggattgccgatcccattgatctatgccagcaggaggccgcctaa

SEQ ID NO: 22 - protein, 522 aa, synthetic construct
MARKVTTRWKITGTLIAETPLHIGGVGGDADTDLALAVNGAGEYYVPGTSLAGALRGWMTQLLNNDESQIKDLWGDHLDAKRGASFVIVDDAVIHIPNNADVEIREGVGIDRHFGTAANGFKYSRAVIPKGSKFKLPLTFDSQDDGLPNALIQLLCALEAGDIRLGAAKTRGLGRIKLDDLKLKSFALDKPEGIFSALLDQGKKLDWNQLKANVTYQSPPYLGISITWNPKDPVMVKAEGDGLAIAILPLVSQVGSDVRFVIPGSSIKGILRTQAERIIRTICQSNGSEKNFLEQLRINLVNELFGSASLSQKQNGKDIDLGKIGALAVNDCFSSLSMTPDQWKAVENATEMTGNLQPALKQATGYPNNISQAYKVLQPAMHVAVDRWTGGAAEGMLYSVLEPIGVTWEPIQVHLDIARLKNYYHGKEEKLKPAIALLLLVLRDLANKKIPVGYGTNRGMGTITVSQITLNGKALPTELEPLNKTMTCPNLTDLDEAFRQDLSTAWKEWIADPIDLCQQEAA

SEQ ID NO: 23 - RNA, 73 nt, synthetic construct
  source 1..73: mol_type = other RNA
actgaaactgtagtagaaccaatcggggtcgtcaataactcccggttcaacaccctcttttccccgtcagggg

SEQ ID NO: 24 - RNA, 60 nt, synthetic construct
  source 1..60: mol_type = other RNA
catgacggatcgcgggagttattgacgaccccgattggttctactacaaacgtgatacta

SEQ ID NO: 25 - DNA, 36 bp, synthetic construct
  source 1..36: mol_type = other DNA
tgtagtagaaccaatcggggtcgtcaataactcccg

SEQ ID NO: 26 - DNA, 37 bp, synthetic construct
  source 1..37: mol_type = other DNA
gttcaacaccctcttttccccgtcaggggactgaaac

SEQ ID NO: 27 - DNA, 6204 bp, synthetic construct
  source 1..6204: mol_type = other DNA
atgcgaggaattgagataaccataaccatgcagagtgattggcacgttggcactggcatgggtcggggggaactggacagtgttgtacaacgggatggagataatctgccctatattcccggcaaaaccttaacaggtattctgcgggatagctgtgaacaggttgccctaggtttagataatggtcaaacccgagggctttggcatgggtggattaattttatttttggcgatcaacctgccctagctcaaggagctattgagccagaacctagacctgccctaatcgccattggttctgcacaccttgaccctaagttaaaagcggcttttcagggcaaaaaacaattgcaagaggcgatcgcctttatgaagccaggggtggctatcgatgcaatcacgggcacagctaagaaagattttttacgctttgaagaagtagttcgtttgggagcgaaattaactgcggaagttgagttaaatttacccgataatttgagcgaaaccaataaaaaagttattgctggtattttagccagtggagcaaagttaaccgagagattaggcggtaaacgtcgccggggcaatgggcgctgtgaattaaaatttagtggttattctgatcaacaaattcaatggttgaaagacaattatcaatctgttgatcaaccacctaagtatcaacaaaataaattacaatctgccggagataatccagaacagcaacccccttggcatattattcccttaaccattaaaaccctttctcctgttgttttaccagctcgtacagtcggtaacgttgtcgaatgtttagactatattcccgggcgttatctactgggctatattcacaaaaccctaggggaatatttcgacgttagtcaggcaatcgccgctggggatttaattattaccaatgccacgataaaaattgatggtaaagcaggacgagctaccccattttgtttgtttggggaaaaactagatggaggattaggtaaaggtaaaggagtttataaccgtttccaagaatcggaacctgatggcattcaattaaagggagaacggggcggctatgttggccaatttgaacaggagcaaaggaatctgccaaatacggggaaaattaattcagagttatttacccataacaccattcaagatgatgtccagcggcccaccagtgatgtggggggagtttatagctatgaagctattatagccggacaaacattcgtcgctgagttacgtttaccagatagcttagtcaagcaaattacaagcaaaaataaaaattggcaagctcaactaaaagctacaattcgcattggtcagtctaaaaaagatcagtatggcaaaatcgaagttacgtcgggaaactctgctgatttgcctaagcctacgggcaacaataaaactctttctatttggttcttatccgatatccttctccgaggcgatcgcctaaattttaatgctactccggatgatctcaaaaaatacttagaaaatgctctggatatcaagctcaaagaacgatcagacaatgatttaatttgcattgctctccgttcccagcggacagaatcctggcaagtacggtggggtttaccccggccatctctagtgggttggcaagctggtagttgtctgatttatgacattgaatctggcactgttaatgccgaaaaattgcaagaattaatgatcaccggcattggcgatcggtgtacagagggttacggtcaaatcggttttaacgatccattactttcggcttccctaggaaagttgacagctaagcctaaagcttctaacaatcagtcccaaaacagccaatccaacccattacccactaatcatcctacccaagattatgctcgattaattgaaaaagcggcttggcgggaagcaattcaaaataaagccttagccttggcatctagccgagcgaaacgggaagaaattttaggcattaaaattatgggaaaagatagtcaacccaccatgactcaattaggaggatttcgctccgtattaaaacggctacactcaagaaataatcgagatattgtcacaggttatttaacagctctagagcaggtttctaatcgaaaagaaaaatggagtaataccagccaaggattaactaaaattcgtaatttagtcacccaggaaaatctcatttggaatcatcttgatattgatttttcgccgttaactattacccaaaatggtgttaatcagctaaagtctgaactttgggcggaagcagtgcgaacccttgttgacgctatcattcggggtcataaacgggacttagaaaaagctcaagaaaacgaatctaatcaacagtcacagggagcagctctgaaaattacccgccgcattctgggcgatgcggaatttcatggcaaaccggatcgcctggaaaaaagccgcagcgtgagcattggcagcgtgctgatggctagaaaagttactacacgctggaaaattacaggcacattaattgcagaaacccctttacacattggtggtgtgggtggcgacgctgatacggatttagccctggcggttaatggtgcgggtgaatattatgtgccagggacaagtttagccggtgctctgcggggttggatgacccagttattgaataatgatgagtcccaaattaaagatctttggggtgatcatttagatgcaaaacggggagctagctttgttattgttgacgatgcggttatccatatacccaataatgctgatgttgaaattagggagggtgttggcatcgatcgccattttggaaccgccgccaatgggtttaaatatagccgagcagttattcccaagggttctaaatttaaattgccattaacttttgacagtcaagatgatgggctaccgaatgcgttgattcaattgttgtgtgccttagaagcaggggatattcgccttggggccgcaaaaacccggggtttaggtcgcattaaactagatgatttaaagttaaaatcctttgctttagataaaccagaaggtattttttctgctttattagaccaaggtaaaaaattagattggaatcaattaaaagcaaacgttacctaccagtctcctccctatctaggtattagtattacctggaatcccaaagatcccgtcatggtgaaagctgaaggggatggactggcgatcgatattttgcccctcgttagtcaagtgggaagtgatgttcgatttgtcattcccggcagttccattaaggggattttacgaacccaggctgaacgtattattcgtactatttgccagtctaatggttctgagaaaaacttcctagaacaattacgaatcaatctggttaatgaattatttgggtctgcttctttgagccaaaaacaaaatggcaaggatatagatctgggtaaaatcggagccttggcagtgaatgattgtttttctagtttatccatgaccccagatcaatggaaagcggtagagaatgccacggagatgacggggaatttacagcctgctcttaaacaagctacgggttatcccaataatattagccaagcttacaaagtacttcaaccggccatgcacgtcgctgtagatcggtggacagggggagctgccgaaggaatgctttacagcgtgctcgaacccattggggtcacctgggaaccgatccaagttcacttggacattgcccgtctcaaaaattattaccacggtaaggaagaaaaacttaaaccggcgatcgccctattgcttcttgtattgcgggatttagctaacaaaaaaattcccgtaggctatggcactaaccgcggtatgggaacgattactgtcagtcaaatcaccctcaatggcaaagccctccccactgaacttgaacctttaaacaaaacaatgacttgtcctaatctcaccgatctagatgaggcatttcgtcaggacttaagcactgcttggaaagagtggattgccgatcccattgatctatgccagcaggaggccgccctgggcaacccgaaaggccaggaactgaaactggatccgccgagcgcggatgcgacccaggcgggcgtgccggcgcagcagaacgcggcgaaaacccaggcgcagggcgcgcaggaaaaatttcataacccctacaactttgtcccagccctaccccgcgatggcataaccggagatttaggcgactgtgctcctgctggtcatagctattaccatggcgataaatacagcggcagaattgccgtcaaactaacaaccgttacccctctattgattcctgacgcttcaaaagaagagataaataacaaccataaaacctatccggttcgtatcggcaaagatggcaagccctatctacctcccacttccattaagggaatgttgcgctctgcctatgaagcggtcactaattcccgcttagccgtgtttgaagatcatgactctcgcttggcctatcgaatgcctgccaccatgggattgcaaatggttcctgcccgcattgaaggtgataatattgttctttacccaggaacctcaaggataggcaataatggccgaccagctaacaatgatcctatgtatgcggcatggcttccttactatcaaaatcgtattgcttatgatggtagtcgtgattatcagatggctgagcatggtgatcatgtcagattttgggctgagcgatataccagaggaaacttctgctattggcgtgtcagacaaattgcacgacacaatcaaaatttaggtaatcggcctgaacgaggacgtaattacggtcaacatcattcaacaggagtcattgaacaatttgaaggatttgtttacaaaaccaataaaaatattgggaataaacatgacgaacgagtatttattattgatcgagaaagtatcgaaatacctctatctcgagatttacggcgaaaatggcgagaattaattacaagctatcaggaaatacacaaaaaggaagttgatagaggtgatactggcccttccgctgtaaatggggctgtttggtcacggcaaattattgcagatgaatcagagcggaatttatcggatgggactctttgttatgctcatgttaagaaagaagatggacagtacaaaattctcaatctttatcctgtaatgatcacacggggattatatgaaattgcgccggttgacttattagatgaaaccctaaagcctgcgacggataaaaagcaactatccccagcagaccgcgtatttggctgggtcaatcaacggggcaatggttgctacaaaggacaattacgaattcatagcgtaacttgccaacatgatgatgccattgatgattttggtaatcaaaatttctctgttccccttgctattttgggacaacctaaaccagaacaggctcgtttttattgtgccgatgatcgaaaaggaattcctttagaagatggctatgatcgtgacgacggctatagtgattcagaacaaggcttgcgaggacgcaaagtctatcctcaccacaaggggttaccaaatggctactggagtaatccaacggaagaccgaagtcaacaagctatccaaggtcattaccaagaatatcgtcgtcctaaaaaggatggtcttgaacaaagagatgatcaaaatcgttctgtaaaaggttgggtaaaaccactgaccgagtttacttttgaaattgacgttactaatctttcggaagttgagttaggtgctctattgtggttgttaaccttacctgatttgcatttccaccgtctaggaggaggtaaaccgttaggttttggtagtgttcgtttagatattgaccctgacaagacagacctaagaaatggggcaggatggcgtgattattacggctctttactagaaacaagtcaaccagattttacaactctaattagtcagtggattaatgcttttcaaacggctgttaaagaggagtatggtagcagtagttttgatcaggttactttcatcaaagcttctggtcagagtctccaaggatttcatgataatgcatctatccattatcctcgttctactcctgagcccaagccagatggagaagcttttaagtggtttgttgccaatgaaaaaggtcgacgattagccttgccagcgctggaaaaatcccagagttttccaatcaaacctagttaa

SEQ ID NO: 28 - protein, 2067 aa, synthetic construct
MRGIEITITMQSDWHVGTGMGRGELDSVVQRDGDNLPYIPGKTLTGILRDSCEQVALGLDNGQTRGLWHGWINFIFGDQPALAQGAIEPEPRPALIAIGSAHLDPKLKAAFQGKKQLQEAIAFMKPGVAIDAITGTAKKDFLRFEEVVRLGAKLTAEVELNLPDNLSETNKKVIAGILASGAKLTERLGGKRRRGNGRCELKFSGYSDQQIQWLKDNYQSVDQPPKYQQNKLQSAGDNPEQQPPWHIIPLTIKTLSPVVLPARTVGNVVECLDYIPGRYLLGYIHKTLGEYFDVSQAIAAGDLIITNATIKIDGKAGRATPFCLFGEKLDGGLGKGKGVYNRFQESEPDGIQLKGERGGYVGQFEQEQRNLPNTGKINSELFTHNTIQDDVQRPTSDVGGVYSYEAIIAGQTFVAELRLPDSLVKQITSKNKNWQAQLKATIRIGQSKKDQYGKIEVTSGNSADLPKPTGNNKTLSIWFLSDILLRGDRLNFNATPDDLKKYLENALDIKLKERSDNDLICIALRSQRTESWQVRWGLPRPSLVGWQAGSCLIYDIESGTVNAEKLQELMITGIGDRCTEGYGQIGFNDPLLSASLGKLTAKPKASNNQSQNSQSNPLPTNHPTQDYARLIEKAAWREAIQNKALALASSRAKREEILGIKIMGKDSQPTMTQLGGFRSVLKRLHSRNNRDIVTGYLTALEQVSNRKEKWSNTSQGLTKIRNLVTQENLIWNHLDIDFSPLTITQNGVNQLKSELWAEAVRTLVDAIIRGHKRDLEKAQENESNQQSQGAALKITRRILGDAEFHGKPDRLEKSRSVSIGSVLMARKVTTRWKITGTLIAETPLHIGGVGGDADTDLALAVNGAGEYYVPGTSLAGALRGWMTQLLNNDESQIKDLWGDHLDAKRGASFVIVDDAVIHIPNNADVEIREGVGIDRHFGTAANGFKYSRAVIPKGSKFKLPLTFDSQDDGLPNALIQLLCALEAGDIRLGAAKTRGLGRIKLDDLKLKSFALDKPEGIFSALLDQGKKLDWNQLKANVTYQSPPYLGISITWNPKDPVMVKAEGDGLAIDILPLVSQVGSDVRFVIPGSSIKGILRTQAERIIRTICQSNGSEKNFLEQLRINLVNELFGSASLSQKQNGKDIDLGKIGALAVNDCFSSLSMTPDQWKAVENATEMTGNLQPALKQATGYPNNISQAYKVLQPAMHVAVDRWTGGAAEGMLYSVLEPIGVTWEPIQVHLDIARLKNYYHGKEEKLKPAIALLLLVLRDLANKKIPVGYGTNRGMGTITVSQITLNGKALPTELEPLNKTMTCPNLTDLDEAFRQDLSTAWKEWIADPIDLCQQEAALGNPKGQELKLDPPSADATQAGVPAQQNAAKTQAQGAQEKFHNPYNFVPALPRDGITGDLGDCAPAGHSYYHGDKYSGRIAVKLTTVTPLLIPDASKEEINNNHKTYPVRIGKDGKPYLPPTSIKGMLRSAYEAVTNSRLAVFEDHDSRLAYRMPATMGLQMVPARIEGDNIVLYPGTSRIGNNGRPANNDPMYAAWLPYYQNRIAYDGSRDYQMAEHGDHVRFWAERYTRGNFCYWRVRQIARHNQNLGNRPERGRNYGQHHSTGVIEQFEGFVYKTNKNIGNKHDERVFIIDRESIEIPLSRDLRRKWRELITSYQEIHKKEVDRGDTGPSAVNGAVWSRQIIADESERNLSDGTLCYAHVKKEDGQYKILNLYPVMITRGLYEIAPVDLLDETLKPATDKKQLSPADRVFGWVNQRGNGCYKGQLRIHSVTCQHDDAIDDFGNQNFSVPLAILGQPKPEQARFYCADDRKGIPLEDGYDRDDGYSDSEQGLRGRKVYPHHKGLPNGYWSNPTEDRSQQAIQGHYQEYRRPKKDGLEQRDDQNRSVKGWVKPLTEFTFEIDVTNLSEVELGALLWLLTLPDLHFHRLGGGKPLGFGSVRLDIDPDKTDLRNGAGWRDYYGSLLETSQPDFTTLISQWINAFQTAVKEEYGSSSFDQVTFIKASGQSLQGFHDNASIHYPRSTPEPKPDGEAFKWFVANEKGRRLALPALEKSQSFPIKPS

SEQ ID NO: 29 - DNA, 753 bp, Serratia sp.
  source 1..753: mol_type = other DNA; strain = ATCC 39006
atgactaatcaggcaaaaaagttatctagaattaatggtagggagtttttaaaacagtcctttaatttacaacaacaactattggcctctcaattaaatttatcccgaacgattacgcatgatggaacgatgggggaggttaatgaaagttattttttgagtattatccgccagtatttgcctgaacgttactcggttgaccggggagttgtggtggattcagaaggccagaccagcgaccagatagatgcagtgatttttgaccggcattacacaccgacattattagaccaacaagggcacaggtttattccggcagaggcggtgtacgcggtactggaggttaaaccaaccattaataaaacctaccttgaatatgcagccgataaagctgcatctgtccgaaaattatatcgaaccagtacggtaataaaaaatatttacggtacggccaaaccggtcgaacatttcccgatcgtagcaggtattgtggcgattgatgttgagtggcaagacggactcggaaaggcatttactgaaaatttgcaggctgtttccagcgatgaaaaccgaaaactggattgcggtctggcggtgtcgggcgcatgttttgatagttatgatgaggaaataaaaatcagaagcggtgaaaatgcattaatcttttttctgttccgtttgctcggtaaattgcaatcattaggtacggtgcccgcaattgactggcgggtgtatatagatagtctggaataa

SEQ ID NO: 30 - protein, 250 aa, Serratia sp.
  REGION 1..250: note = ATCC 39006
MTNQAKKLSRINGREFLKQSFNLQQQLLASQLNLSRTITHDGTMGEVNESYFLSIIRQYLPERYSVDRGVVVDSEGQTSDQIDAVIFDRHYTPTLLDQQGHRFIPAEAVYAVLEVKPTINKTYLEYAADKAASVRKLYRTSTVIKNIYGTAKPVEHFPIVAGIVAIDVEWQDGLGKAFTENLQAVSSDENRKLDCGLAVSGACFDSYDEEIKIRSGENALIFFLFRLLGKLQSLGTVPAIDWRVYIDSLE

SEQ ID NO: 31 - DNA, 200 bp, Serratia sp.
  source 1..200: mol_type = other DNA; strain = ATCC 39006
ccctacgctccctccagcgctgtcggggatatagtcactcggcaagggcgcccttgaggattgattactgaactctagtatggtaaactgtgaaaactcataaagctgacgaagtaaaagaatcaaactaataactcaatccagtctaaagagtagaaagttggtgaaagattgtgagtcagtcacttaatggtcttaga

SEQ ID NO: 32 - DNA, 200 bp, Serratia sp.
  source 1..200: mol_type = other DNA; strain = ATCC 39006
ccctacgctccctccagcgctgtcggggatatagtcactcggagttagagagttttaggattgattactgaactctagtatggtaaactgtgaaaactcataaagctgacgaagtaaaagaatcaaactaataactcaatccagtctaaagagtagaaagttggtgaaagattgtgagtcagtcacttaatggtcttaga

SEQ ID NO: 33 - DNA, 200 bp, Serratia sp.
  source 1..200: mol_type = other DNA; strain = ATCC 39006
ccctacgctccctccagcgctgtcggggatatagtcactcggagttggcgccttttaggattgattactgaactctagtatggtaaactgtgaaaactcataaagctgacgaagtaaaagaatcaaactaataactcaatccagtctaaagagtagaaagttggtgaaagattgtgagtcagtcacttaatggtcttaga

SEQ ID NO: 34 - skipped

SEQ ID NO: 35 - RNA, 37 nt, synthetic construct
  source 1..37: mol_type = other RNA
actgaaactgtagtagaaccaatcggggtcgtcaata

SEQ ID NO: 36-37 - skipped (2 entries)

SEQ ID NO: 38 - DNA, 14 bp, synthetic construct
  source 1..14: mol_type = other DNA
caagggcgcccttg

SEQ ID NO: 39 - DNA, 38 bp, synthetic construct
  source 1..38: mol_type = other DNA
tacttccaatccaatgcaatgactaatcaggcaaaaaa

SEQ ID NO: 40 - DNA, 39 bp, synthetic construct
  source 1..39: mol_type = other DNA
ttatccacttccaatgttattattccagactatctatat

SEQ ID NO: 41 - DNA, 46 bp, synthetic construct
  source 1..46: mol_type = other DNA
gcgaattcgagctcggtaccaaagaggagaaattaactatggtgag

SEQ ID NO: 42 - DNA, 37 bp, synthetic construct
  source 1..37: mol_type = other DNA
cttttttgcctgattagtcatggatccgcctccaccg

SEQ ID NO: 43 - DNA, 46 bp, synthetic construct
  source 1..46: mol_type = other DNA
agggcggtggaggcggatccatgactaatcaggcaaaaaagttatc

SEQ ID NO: 44 - DNA, 46 bp, synthetic construct
  source 1..46: mol_type = other DNA
caaaaggtcatccactgcagttattccagactatctatatacaccc

SEQ ID NO: 45 - DNA, 798 bp, synthetic construct
  source 1..798: mol_type = other DNA
tcgtcttcacctcgagaaatcaaagaggagaaattaactatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagggcggtggaggcggatcccctgttgatagatccagtaatgac

SEQ ID NO: 46 - DNA, 50 bp, synthetic construct
  source 1..50: mol_type = other DNA
tatagaattcaaagaggagaaattaactatgactaatcaggcaaaaaagt

SEQ ID NO: 47 - DNA, 39 bp, synthetic construct
  source 1..39: mol_type = other DNA
tataaagcttttattccagactatctatatacacccgcc

SEQ ID NO: 48 - DNA, 29 bp, synthetic construct
  source 1..29: mol_type = other DNA
gactctagacacgtggagaaaccaaagcc

SEQ ID NO: 49 - DNA, 27 bp, synthetic construct
  source 1..27: mol_type = other DNA
gatcccgggtcagttccttgccgtagc

SEQ ID NO: 50 - DNA, 23 bp, synthetic construct
  source 1..23: mol_type = other DNA
ccagataaatgcagtgatttttg

SEQ ID NO: 51 - DNA, 19 bp, synthetic construct
  source 1..19: mol_type = other DNA
tgcatttatctggtcgctg

SEQ ID NO: 52 - DNA, 23 bp, synthetic construct
  source 1..23: mol_type = other DNA
gtactgaatgttaaaccaaccat

SEQ ID NO: 53 - DNA, 21 bp, synthetic construct
  source 1..21: mol_type = other DNA
ttaacattcagtaccgcgtac

SEQ ID NO: 54 - DNA, 25 bp, synthetic construct
  source 1..25: mol_type = other DNA
ggttcttccaaccattaataaaacc

SEQ ID NO: 55 - DNA, 19 bp, synthetic construct
  source 1..19: mol_type = other DNA
gttggaagaacctccagta

SEQ ID NO: 56 - DNA, 200 bp, synthetic construct; identical to SEQ ID NO: 32
  source 1..200: mol_type = other DNA

SEQ ID NO: 57 - DNA, 200 bp, synthetic construct; identical to SEQ ID NO: 31
  source 1..200: mol_type = other DNA

SEQ ID NO: 58 - DNA, 200 bp, synthetic construct; identical to SEQ ID NO: 33
  source 1..200: mol_type = other DNA

SEQ ID NO: 59 - DNA, 28 bp, synthetic construct
  source 1..28: mol_type = other DNA
tattgcatgcggctgacgatctggcgtc

SEQ ID NO: 60 - DNA, 31 bp, synthetic construct
  source 1..31: mol_type = other DNA
tcttggatccgctagcggcctgccggagaac

SEQ ID NO: 61 - DNA, 20 bp, synthetic construct
  source 1..20: mol_type = other DNA
cacactttgctatgccatag

SEQ ID NO: 62 - DNA, 56 bp, synthetic construct
  source 1..56: mol_type = other DNA
cgaagacgaaagggcctcgtgatacgcaagctttatggcttgtaaaccgttttgtg

SEQ ID NO: 63 - DNA, 39 bp, synthetic construct
  source 1..39: mol_type = other DNA
aaagaaatcataaaaaatttatttgctttgtgagcggat

SEQ ID NO: 64 - DNA, 27 bp, synthetic construct
  source 1..27: mol_type = other DNA
tttatgcatcttcagtcagggagcgtc

SEQ ID NO: 65 - DNA, 30 bp, synthetic construct
  source 1..30: mol_type = other DNA
ttttactagtagacgttcaacaacgtcatg

SEQ ID NO: 66 - DNA, 30 bp, synthetic construct
  source 1..30: mol_type = other DNA
ttttggtaccgaagttatattcgcgcggtg

SEQ ID NO: 67 - DNA, 20 bp, synthetic construct
  source 1..20: mol_type = other DNA
cacactttgctatgccatag

SEQ ID NO: 68 - DNA, 24 bp, synthetic construct
  source 1..24: mol_type = other DNA
gtcattactggatctatcaacagg

SEQ ID NO: 69 - DNA, 14 bp, synthetic construct
  source 1..14: mol_type = other DNA
cannggcgccnntg

SEQ ID NO: 70 - DNA, 24 bp, synthetic construct
  source 1..24: mol_type = other DNA
ctcggcaagggcgcccttgaggat

SEQ ID NO: 71 - DNA, 24 bp, synthetic construct
  source 1..24: mol_type = other DNA
atcctcaagggcgcccttgccgag

SEQ ID NO: 72 - DNA, 38 bp, synthetic construct
  source 1..38: mol_type = other DNA
tatacatatggcatacagactgtttttcagtgtgatag

SEQ ID NO: 73 - DNA, 29 bp, synthetic construct
  source 1..29: mol_type = other DNA
cgagggtaccgggactccaaccccccaag

SEQ ID NO: 74 - DNA, 31 bp, synthetic construct
  source 1..31: mol_type = other DNA
tataccatggtggatctaaaatccttagctg

SEQ ID NO: 75 - DNA, 31 bp, synthetic construct
  source 1..31: mol_type = other DNA
attcggatccttattgaacattggctaaggc

SEQ ID NO: 76 - DNA, 60 bp, synthetic construct
  source 1..60: mol_type = other DNA
tgggtaccgaaaacctgtattttcagggctttctagttctaattgagacttccggtaatc

SEQ ID NO: 77 - DNA, 53 bp, synthetic construct
  source 1..53: mol_type = other DNA
cggccgcaagcttgtcgacctgcagttaactaggtttgattggaaaactctgg

SEQ ID NO: 78 - RNA, 60 nt, synthetic construct; identical to SEQ ID NO: 24
  source 1..60: mol_type = other RNA

SEQ ID NO: 79 - DNA, 40 bp, synthetic construct
  source 1..40: mol_type = other DNA
ggcgccgctgcgacggctttagccctggcggttaatggtg

SEQ ID NO: 80 - DNA, 36 bp, synthetic construct
  source 1..36: mol_type = other DNA
aaagccgtcgcagcggcgccacccacaccaccaatg

SEQ ID NO: 81 - DNA, 43 bp, synthetic construct
  source 1..43: mol_type = other DNA
ggctggactggcgatcgctattttgcccctcgttagtcaagtg

SEQ ID NO: 82 - DNA, 40 bp, synthetic construct
  source 1..40: mol_type = other DNA
tagcgatcgccagtccagccccttcagctttcaccatgac

SEQ ID NO: 83 - DNA, 47 bp, synthetic construct
  source 1..47: mol_type = other DNA
gcctaagttagtaactttaccactaccaccaatatgaaattaccctc

SEQ ID NO: 84 - DNA, 32 bp, synthetic construct
  source 1..32: mol_type = other DNA
gtaaagttactaacttaggcggcctcctgctg

SEQ ID NO: 85 - DNA, 31 bp, synthetic construct
  source 1..31: mol_type = other DNA
gaactagccagtgttgtacaacgggatggag

SEQ ID NO: 86 - DNA, 33 bp, synthetic construct
  source 1..33: mol_type = other DNA
tgtacaacactggctagttccccccgacccatg

SEQ ID NO: 87 - DNA, 37 bp, synthetic construct
  source 1..37: mol_type = other DNA
aagtaaaatgacacccagaaatgttaacgctagcaac

SEQ ID NO: 88 - DNA, 44 bp, synthetic construct
  source 1..44: mol_type = other DNA
ttctgggtgtcattttacttaacctccaatttaattaaacgttc

SEQ ID NO: 89 - RNA, 60 nt, synthetic construct; identical to SEQ ID NO: 24
  source 1..60: mol_type = other RNA

SEQ ID NO: 90 - RNA, 60 nt, synthetic construct; identical to SEQ ID NO: 24
  source 1..60: mol_type = other RNA

SEQ ID NO: 91 - RNA, 60 nt, synthetic construct; identical to SEQ ID NO: 24
  source 1..60: mol_type = other RNA

SEQ ID NO: 92 - RNA, 43 nt, synthetic construct
  source 1..43: mol_type = other RNA
catgacggatcgcgggagttattgacgaccccgattggttcta

SEQ ID NO: 93 - RNA, 37 nt, synthetic construct
  source 1..37: mol_type = other RNA
catgacggatcgcgggagttattgacgaccccgattg

SEQ ID NO: 94 - RNA, 31 nt, synthetic construct
  source 1..31: mol_type = other RNA
catgacggatcgcgggagttattgacgaccc

SEQ ID NO: 95 - RNA, 27 nt, synthetic construct
  source 1..27: mol_type = other RNA
catgacggatcgcgggagttattgacg

SEQ ID NO: 96 - RNA, 60 nt, synthetic construct
  source 1..60: mol_type = other RNA
catgacggatcgcgggagttattgacgaccccgattggttctactacagtttcagtcccc

SEQ ID NO: 97 - RNA, 37 nt, synthetic construct; identical to SEQ ID NO: 93
  source 1..37: mol_type = other RNA

SEQ ID NO: 98 - DNA, 24 bp, synthetic construct
  source 1..24: mol_type = other DNA
atcctcaagggcgcccttgccgag

SEQ ID NO: 99 - DNA, 22 bp, synthetic construct
  source 1..22: mol_type = other DNA
ctcgccctggttcttgtgattg

SEQ ID NO: 100 - DNA, 51 bp, synthetic construct
  source 1..51: mol_type = other DNA
gagagcaaccagcagtctcaaggagccgctgaaggcagaggaagcctactg

SEQ ID NO: 101 - DNA, 19 bp, synthetic construct
  source 1..19: mol_type = other DNA
agcggctccttgagactgc

SEQ ID NO: 102 - DNA, 51 bp, synthetic construct
  source 1..51: mol_type = other DNA
cctatcgacctgtgccaacaggaagctgctgaaggcagaggaagcctactg

SEQ ID NO: 103 - DNA, 19 bp, synthetic construct
  source 1..19: mol_type = other DNA
agcagcttcctgttggcac

SEQ ID NO: 104 - DNA, 51 bp, synthetic construct
  source 1..51: mol_type = other DNA
gacgagcggctgatcaagctggaagtgaaggaaggcagaggaagcctactg

SEQ ID NO: 105 - DNA, 23 bp, synthetic construct
  source 1..23: mol_type = other DNA
cttcacttccagcttgatcagcc

SEQ ID NO: 106 - DNA, 55 bp, synthetic construct
  source 1..55: mol_type = other DNA
tggtatggctgattatgatcctctagactaactaggcttgatagggaaagactgg

SEQ ID NO: 107 - DNA, 52 bp, synthetic construct
  source 1..52: mol_type = other DNA
agatccgctagggatccgccgccaccatggctcaccatcaccatcatcacag

SEQ ID NO: 108 - DNA, 55 bp, synthetic construct
  source 1..55: mol_type = other DNA
ctggagaattcaccggtgccgccaccatggccaatctggataagatgctgaacac

SEQ ID NO: 109 - DNA, 56 bp, synthetic construct
  source 1..56: mol_type = other DNA
tatcccctgattctgtggataaccgtattacggcagtgaaaaaaatgctttatttg

SEQ ID NO: 110 - DNA, 53 bp, synthetic construct
  source 1..53: mol_type = other DNA
tggtatggctgattatgatcctctagactacttcacttccagcttgatcagcc

SEQ ID NO: 111 - DNA, 49 bp, synthetic construct
  source 1..49: mol_type = other DNA
cgggcaaagagagccctggctaacgtgcaggaaggcagaggaagcctac

SEQ ID NO: 112 - DNA, 60 bp, synthetic construct
  source 1..60: mol_type = other DNA
agatccgctagggatccgccgccaccatggctgattacaaggatgacgatgacaagatgg

SEQ ID NO: 113 - DNA, 73 bp, synthetic construct
  source 1..73: mol_type = other DNA
aaaccttctcctttagacaccatggtggcgaccggtagcggttcaacaccctcttttccccgtcaggggactg

SEQ ID NO: 114 - DNA, 73 bp, synthetic construct
  source 1..73: mol_type = other DNA
gtttcagtcccctgacggggaaaagagggtgttgaaccgctaccggtcgccaccatggtgtctaaaggagaag

SEQ ID NO: 115 - DNA, 73 bp, synthetic construct
  source 1..73: mol_type = other DNA
aaacgcttcatatggtctggatatctggcaaaacactggagttcaacaccctcttttccccgtcaggggactg

SEQ ID NO: 116 - DNA, 73 bp, synthetic construct
  source 1..73: mol_type = other DNA
gtttcagtcccctgacggggaaaagagggtgttgaactccagtgttttgccagatatccagaccatatgaagc

SEQ ID NO: 117 - DNA, 73 bp, synthetic construct
  source 1..73: mol_type = other DNA
aaacgttgtaccccaccatcttcaatgttatggcgtatttgttcaacaccctcttttccccgtcaggggactg

SEQ ID NO: 118 - DNA, 73 bp, synthetic construct
  source 1..73: mol_type = other DNA
gtttcagtcccctgacggggaaaagagggtgttgaacaaatacgccataacattgaagatggtggggtacaac

SEQ ID NO: 119 - DNA, 73 bp, synthetic construct
  source 1..73: mol_type = other DNA
aaacgcatatcagcttcaacgtgagtttgccataggtggcgttcaacaccctcttttccccgtcaggggactg

SEQ ID NO: 120 - DNA, 73 bp, synthetic construct
  source 1..73: mol_type = other DNA
gtttcagtcccctgacggggaaaagagggtgttgaacgccacctatggcaaactcacgttgaagctgatatgc

SEQ ID NO: 121 - DNA, 73 bp, synthetic construct
  source 1..73: mol_type = other DNA
aaacgaggattacacggcataggtcagcctaagtcatcgagttcaacaccctcttttccccgtcaggggactg

SEQ ID NO: 122 - DNA, 73 bp, synthetic construct
  source 1..73: mol_type = other DNA
gtttcagtcccctgacggggaaaagagggtgttgaactcgatgacttaggctgacctatgccgtgtaatcctc

SEQ ID NO: 123 - DNA, 73 bp, synthetic construct
  source 1..73: mol_type = other DNA
aaacgctaggatataatgctgaggacctgaactcgtactggttcaacaccctcttttccccgtcaggggactg

SEQ ID NO: 124 - DNA, 73 bp, synthetic construct
  source 1..73: mol_type = other DNA
gtttcagtcccctgacggggaaaagagggtgttgaaccagtacgagttcaggtcctcagcattatatcctagc

SEQ ID NO: 125 - DNA, 257 bp, synthetic construct
  source 1..257: mol_type = other DNA
ccatggtggcggcaccggtgaattctccaggcgatctgacggttcactaaacgagctctgcttatataggcctcccaccgtacacgccacctcgacatactcgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaa

SEQ ID NO: 126 - DNA, 125 bp, synthetic construct
  source 1..125: mol_type = other DNA
gacgtatgttcccatagtaacgccaatagggactttccattgacgtcaatgggtggagtatttacggtaaactgcccacttggcagtacatcaagtgtatcatatgccaagtacgccccctattg

SEQ ID NO: 127 - DNA, 377 bp, synthetic construct
  source 1..377: mol_type = other DNA
gccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagggatccgccgccaccatgg

SEQ ID NO: 128 - DNA, 2484 bp, synthetic construct
  source 1..2484: mol_type = other DNA
gaaggcagaggaagcctacttacatgcggtgatgttgaggaaaatccgggtccagattacaaggatgacgatgacaagatggcaccgaagaaaaaacgtaaggtgcgtggtatgcggggcatcgagatcacaatcaccatgcagtctgattggcacgtgggcacaggcatgggcagaggagagctggacagcgtggttcagcgggatggcgacaatctgccatacatccctggaaagaccctgactggcatcctgcgggacagctgtgaacaggtggccctgggcctggacaacggccaaacaagaggactgtggcacggctggatcaacttcatcttcggcgaccagcctgccctggctcagggtgccatcgaaccagagccacggcctgctctgattgcaatcggatccgctcacctggatcctaagctgaaggccgccttccagggcaagaagcagctgcaggaggccatcgcttttatgaagcccggcgtggccatcgatgccattacaggcaccgccaagaaggatttcctgagattcgaggaagtcgtcagactgggtgctaagctgaccgccgaggtggaacttaacctgccagacaatctgtctgaaaccaacaaaaaagtgatagctggcatcctggctagcggtgccaagctgaccgagcggctgggcggaaagcggagaagaggcaacggcagatgcgagctgaagttcagcggctacagcgatcaacaaatccagtggctgaaagacaactaccaaagcgtggaccagccccctaagtaccagcagaacaagctgcagagtgcgggcgacaatcctgagcagcagccaccttggcatatcatccctctgaccatcaaaaccctgagccctgtggtgctgcctgctagaacagtgggcaacgtggtggaatgcctggactacatccctggcagatacctgctgggctacatccacaaaacactgggagaatacttcgacgtgtcacaggccattgcggccggagatctgatcattaccaatgccacaatcaagatcgacggcaaagccggaagggccacccctttctgcctgtttggcgagaagctcgatggcggccttggcaaaggcaagggcgtgtacaaccggtttcaggaaagcgagcctgacggcatccagctgaagggcgaacggggaggctatgtgggccagttcgagcaagagcagagaaatctgcccaacaccggcaagatcaacagcgaactgttcacccacaacacaatccaagatgatgtgcagaggcctacctccgacgtgggaggcgtctacagctacgaggccatcattgccggacagacattcgtggctgagctgagactgcccgattctctggtgaagcagatcaccagcaagaacaagaactggcaggcccagctgaaggcaaccatcagaatcggccagtccaagaaggaccagtacggcaaaatcgaagtgacctctggcaacagcgctgatctgcctaagcctaccggcaacaacaagaccctgagcatctggttcctgtccgacattctgctgagaggcgacagactcaacttcaatgccacaccagacgacctgaagaaatacctggagaacgccctggatatcaagctgaaggaacggtccgacaacgacctgatctgcatcgctctgcggagccagcggacagagagctggcaggtgagatggggcctgcctagacccagcctggtgggatggcaagccggctcttgtctgatctacgacatcgagtccggcaccgtgaacgccgagaaactccaggagctgatgatcaccggcatcggggatagatgcaccgagggctatggccagatcggcttcaacgaccccctcctgagcgccagcctgggcaagctgaccgccaagcctaaggccagcaacaaccagtcccagaattctcagtctaaccccctgcccacgaaccaccctacacaggactacgccagactgatcgagaaggccgcctggcgggaagctattcagaacaaggctctggccctggcctctagccgcgccaaaagagaggaaatcctgggcatcaagatcatgggcaaggacagccaacctaccatgacccagctgggcggatttagatctgtgctgaaaaggctgcacagcagaaacaacagagatatcgtgacaggctatctgacagcacttgagcaggtcagcaatagaaaggaaaagtggtccaataccagccagggcctgaccaagatccgcaacctggtgacccaggagaaccttatctggaaccacctggacatcgacttctctcctctgacaatcacgcagaacggcgttaaccagctgaagagcgagctgtgggccgaagccgtgcggaccctggtcgacgccatcatccggggccacaagcgggacctggaaaaggcccaggagaacgagagcaaccagcagtctcaaggagccgct

SEQ ID NO: 129 - DNA, 1677 bp, synthetic construct
  source 1..1677: mol_type = other DNA
gaaggcagaggaagcctactgacctgtggcgacgtcgaggaaaatcctggtccagactataaggacgacgacgacaagatggctccgaaaaagaagcgtaaggtccgtggcatggccagaaaggtgacaaccagatggaagatcaccggaacactgatcgccgagacacctctgcacatcggaggagttggtggtgatgccgataccgacctggcactggctgttaacggtgctggtgagtactacgttcctggtaccagcctggccggagctctgagggggtggatgacccagctgctgaacaatgacgagagccagatcaaggacctgtggggcgaccacctggacgctaaaagaggcgccagctttgtgatcgtggacgacgccgtgatccacatcccaaacaacgcggacgtggaaatccgggaaggagtgggcatcgatagacatttcggcaccgccgccaacggcttcaagtacagtagagccgtgatccctaagggcagcaagttcaagctgcctctgaccttcgactcccaagatgacggactgcctaatgctctgattcagctgctctgtgctctggaagccggagacattcgcctgggagctgcaaagacacggggtcttggaagaatcaagctggatgacctgaagctgaagagctttgccctggataagcccgagggcattttctccgccctgctggatcaaggtaagaaactggattggaaccagcttaaggccaatgtgacttaccagagccctccttacctgggcatcagcatcacatggaatcctaaggatcctgtgatggtgaaggccgagggcgatggcctggccatcgacatcctgcccctggtgtctcaggttggctctgatgtgcggttcgtcatccccggcagcagcatcaagggaattctgcggacccaggccgagcggattatcagaaccatctgccagagcaacggcagcgagaagaacttcctggaacagctaagaatcaacctggttaacgagctgttcggctccgcctctctgagccaaaagcagaacggcaaggacatcgacctgggaaaaatcggcgccctggccgtgaacgactgcttcagcagcctgtctatgacacccgaccagtggaaagccgtggaaaacgccacagagatgaccggaaatctgcaaccagccctgaagcaggccaccggatatcctaataacatcagccaagcttataaggtgctgcagcctgccatgcacgtggccgtcgacagatggaccggtggagccgctgagggcatgctgtacagcgtgctggaacccatcggcgtgacatgggagcccatccaggtgcacctggacatcgctagactgaaaaactactaccacggcaaagaggaaaagctgaaacctgctatcgccctgctgctgctggtgctcagagatctggctaacaagaagatccccgtgggctacggcaccaaccggggcatgggcaccatcaccgtgtcccagatcaccctgaacggcaaggctctgcctacagagctggaaccactgaacaaaaccatgacctgtcctaacctgacagacctggatgaggcctttagacaggacctgtctacagcctggaaggaatggatcgccgatcctatcgacctgtgccaacaggaagctgct

SEQ ID NO: 130 - DNA, 1822 bp, synthetic construct
  source 1..1822: mol_type = other DNA
agcagagccagggagccgctctgaagatcacaaggcgcatcctgggcgacgcagagttccacggcaagcccgacagactggaaaagagccgcagcgtgtctatcggctctgtgctgatggccagaaaggtgacaaccagatggaagatcaccggaacactgatcgccgagacacctctgcacatcggaggagttggtggtgatgccgataccgacctggcactggctgttaacggtgctggtgagtactacgttcctggtaccagcctggccggagctctgagggggtggatgacccagctgctgaacaatgacgagagccagatcaaggacctgtggggcgaccacctggacgctaaaagaggcgccagctttgtgatcgtggacgacgccgtgatccacatcccaaacaacgcggacgtggaaatccgggaaggagtgggcatcgatagacatttcggcaccgccgccaacggcttcaagtacagtagagccgtgatccctaagggcagcaagttcaagctgcctctgaccttcgactcccaagatgacggactgcctaatgctctgattcagctgctctgtgctctggaagccggagacattcgcctgggagctgcaaagacacggggtcttggaagaatcaagctggatgacctgaagctgaagagctttgccctggataagcccgagggcattttctccgccctgctggatcaaggtaagaaactggattggaaccagcttaaggccaatgtgacttaccagagccctccttacctgggcatcagcatcacatggaatcctaaggatcctgtgatggtgaaggccgagggcgatggcctggccatcgacatcctgcccctggtgtctcaggttggctctgatgtgcggttcgtcatccccggcagcagcatcaagggaattctgcggacccaggccgagcggattatcagaaccatctgccagagcaacggcagcgagaagaacttcctggaacagctaagaatcaacctggttaacgagctgttcggctccgcctctctgagccaaaagcagaacggcaaggacatcgacctgggaaaaatcggcgccctggccgtgaacgactgcttcagcagcctgtctatgacacccgaccagtggaaagccgtggaaaacgccacagagatgaccggaaatctgcaaccagccctgaagcaggccaccggatatcctaataacatcagccaagcttataaggtgctgcagcctgccatgcacgtggccgtcgacagatggaccggtggagccgctgagggcatgctgtacagcgtgctggaacccatcggcgtgacatgggagcccatccaggtgcacctggacatcgctagactgaaaaactactaccacggcaaagaggaaaagctgaaacctgctatcgccctgctgctgctggtgctcagagatctggctaacaagaagatccccgtgggctacggcaccaaccggggcatgggcaccatcaccgtgtcccagatcaccctgaacggcaaggctctgcctacagagctggaaccactgaacaaaaccatgacctgtcctaacctgacagacctggatgaggcctttagacaggacctgtctacagcctggaaggaatggatcgccgatcctatcgacctgtgccagcaggaggctgctctcggcaaccccaaaggccaagagcttaaactggatcctccatccgctgacgccacccaggctggcgtgcccgcgcaacagaatgccgccaagacacaggctcagggagcccaggagaagatgaccgtgggaacgctggg

SEQ ID NO: 131 - DNA, 2745 bp, synthetic construct
  source 1..2745: mol_type = other DNA
gaaggcagaggaagcctactgacatgcggagatgtggaagagaaccccggacctgactacaaggacgacgacgacaagatggcccctaagaagaaacggaaggtgcggggcatgaccgtgggaacgctgggagtcgtgggcagcgccaagaacctgaaactgcagctgagcttcattaacaccagacagcagtacgtgcagatcactctgttcgagagaaacagctttaaggtggccgaagaagaattcagcacagagctggtggaaataatcaaaaccgccctgcctacacttaagaacaagaaagtggaattcgaggaggacggcgaccagatcaagcagatcagagagaagggccaggcctgggtgggcgccgctgagcagatcgccccttatgtgctgcccagcggaaatatcacagaaacccctaggaatgtgaacgccagcaacttccacaatccttacaacttcgtgcccgctctgcccagagatggcatcaccggcgatctgggcgattgcgcccctgctggccacagctactatcacggcgacaagtacagcggcaggattgccgtgaaactgacaaccgtgacacctctgctgatccccgacgctagcaaggaagagatcaacaataatcacaagacctaccccgtgcggatcggcaaagatggcaagccctacctgccaccaacatctattaagggcatgctgagaagcgcctacgaggccgttaccaacagccggctggccgtgttcgaggaccacgacagccgcctggcttatagaatgcctgccaccatgggactgcagatggtgcctgccagaatcgagggcgataatatcgtgctgtaccccggcacctctcggatcggcaacaacggccggcctgctaataacgaccctatgtacgccgcctggctgccttactaccagaacagaatcgcctacgacggctctagagattaccagatggccgagcacggcgaccatgtgcggttctgggccgaaagatacacccgaggcaacttttgttactggagagtgcgccagatcgcaagacataaccagaacctgggtaacagacctgagagaggccggaactacggccaacaccacagcaccggcgtgatcgagcagttcgaaggcttcgtgtacaagacaaacaaaaacatcggcaacaagcacgacgagagagttttcatcatcgaccgggagtccatcgaaatccctctcagccgggatctccggcggaagtggcgggaactgatcaccagctaccaggagatccacaagaaggaagtggatagaggagatacaggcccttccgccgtgaacggcgccgtgtggagccgacagatcatcgctgatgagagcgagcggaacctgagcgacggcaccctgtgctacgcccacgtgaagaaagaggacggccagtacaagatcctgaacctgtaccccgtgatgatcaccagaggcctgtacgagatcgcccctgtggacctgctggacgagacactgaagcctgcaaccgacaagaagcaactgagccctgccgacagagtgtttggatgggttaaccagagaggaaacggatgttataaaggccagctgagaatccactctgtgacctgccagcacgatgatgccattgatgacttcggcaatcagaatttcagcgtgccactggccatcctgggccagcccaagccagaacaggccagattctactgcgccgacgaccggaagggaatccccctggaagacggctacgacagagacgacggctactctgatagcgagcagggcctgcgaggcaggaaggtctacccccaccacaaaggactgccaaacggctactggtccaaccccacagaagatagatctcagcaggcgatccagggccactaccaagagtacagaagacccaagaaggacggcctggaacaaagagacgaccagaaccggagcgtgaagggctgggtcaaacctctcacagagttcaccttcgagatcgacgtgacaaacctgtccgaggtggaactgggcgctctgctctggctgctgaccctgccagatctgcacttccaccggctgggcggcggaaagcctctgggtttcggcagcgtgcggctggacattgaccccgataagaccgacctgagaaatggcgccggctggcgagattactacggctcgctgctcgagacaagccagcctgactttaccaccctgatcagccagtggatcaacgccttccagaccgccgtgaaggaagagtacggatccagcagcttcgaccaagtgacctttatcaaggccagcggccaaagcctgcagggcttccacgacaatgcttctatccattatcctagatccacccctgagcctaagcctgacggcgaggcttttaagtggtttgtggccaacgagaaggggagaagactggccctgccggccctggaaaagagccagtctttccctatcaagcctagttagtctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgccgtaatacggttatccacagaatcaggggataacgcaggaaagaaactagt

SEQ ID NO: 132 - DNA, 1776 bp, synthetic construct
  source 1..1776: mol_type = other DNA
gaaggcagaggaagcctacttacatgcggcgatgtggaagaaaaccccggccctcaccatcaccatcatcacagcggagactacaaggatgacgatgataagatgttcctggtgctgatcgaaaccagcggcaaccagcacttcatcttcagcaccaacaaactgcgggaaaacatcggcgccagcgagctgacctacctggcaaccaccgagatcctcttccagggcgtggaccgggtctttcagaccaattactacgaccagtggagcgacaccaacagcctgaacttcctggcagatagcaagctgaaccccgccatcgacgaccccaagaacaacgccgatatcgagatcctgctggccacaagcggaaaggccatcgccctggtgaaggaggaaggcaaggccaagcagctgatcaaagaggtgactaagcaggctctgattaacgctcctggactggaaattggcggcatctacgtgaactgcaactggcaggacaagctgggcgtcgcgaaggccgtgaaagaagctcacaagcaatttgaggtgaaccgggccaaaagagccggcgctaatggcaggttcctgcggctgccaatcgctgctggctgctctgtgtccgagctgcctgcttctgattttgactacaacgctgacggcgacaagatccctgtctccaccgtgtctaaagtgaagagagagacagccaaaagcgctaaaaagcggctgagaagcgtggatggcagacttgttaatgacctggctcagctggaaaaatcattcgacgaactggattggctggccgtggtgcacgccgacggcaacggcctgggccagatcctcctgagcctggaaaaatacatcggagagcagaccaaccggaactacatcgataagtaccggagactgtctcttgctctggacaactgcaccatcaacgcctttaagatggccatcgctgtgttcaaggaagatagcaagaagatcgacctgcctatcgtgcctctgatcctgggaggagatgacctgacagtgatctgtaggggcgattacgccctggagttcaccagagagttcctggaggccttcgagggccagacagagacacacgacgacatcaaggtgatcgcccagaaagccttcggtgtggacagactgtccgcctgcgccggcatcagcatcatcaagcctcacttccccttcagcgtggcctatacactggccgaaagactgatcaagagcgccaaggaggtgaagcagaaggtgaccgttaccaattctagccctatcaccccttttccatgtagcgccattgatttccacatcctgtacgacagcagcggcatcgactttgatagaatcagagagaagctgcggcctgaggataacacagaactgtacaacagaccctacgtggtcaccgccgccgaaaacctgagccaggcccaaggctacgagtggtcccaagcccactccctgcagaccctggcggacagagtgtcctacctgcgcagcgaggacggcgaaggcaagtctgccctgcccagcagccagagccacgccctgagaacagccctgtatctggaaaagaatgaagccgacgcccagtacagcctgatctctcaaagatacaagatcttgaagaacttcgccgaggacggcgagaacaagtctctgttccatctggaaaatggaaagtacgtgacccggttcctcgatgccctcgacgccaaggacttcttcgccaacgccaatcacaagaaccagggcgag

SEQ ID NO: 133 - DNA, 1776 bp, synthetic construct
  source 1..1776: mol_type = other DNA
gaaggcagaggaagcctacttacatgcggcgatgtggaagaaaaccccggccctcaccatcaccatcatcacagcggagactacaaggatgacgatgataagatgttcctggtgctgatcgaaaccagcggcaaccagcacttcatcttcagcaccaacaaactgcgggaaaacatcggcgccagcgagctgacctacctggcaaccaccgagatcctcttccagggcgtggaccgggtctttcagaccaattactacgaccagtggagcgacaccaacagcctgaacttcctggcagatagcaagctgaaccccgccatcgacgaccccaagaacaacgccgatatcgagatcctgctggccacaagcggaaaggccatcgccctggtgaaggaggaaggcaaggccaagcagctgatcaaagaggtgactaagcaggctctgattaacgctcctggactggaaattggcggcatctacgtgaactgcaactggcaggacaagctgggcgtcgcgaaggccgtgaaagaagctcacaagcaatttgaggtgaaccgggccaaaagagccggcgctaatggcaggttcctgcggctgccaatcgctgctggctgctctgtgtccgagctgcctgcttctgattttgactacaacgctgacggcgacaagatccctgtctccaccgtgtctaaagtgaagagagagacagccaaaagcgctaaaaagcggctgagaagcgtggatggcagacttgttaatgacctggctcagctggaaaaatcattcgacgaactggattggctggccgtggtgcacgccgacggcaacggcctgggccagatcctcctgagcctggaaaaatacatcggagagcagaccaaccggaactacatcgataagtaccggagactgtctcttgctctggacaactgcaccatcaacgcctttaagatggccatcgctgtgttcaaggaagatagcaagaagatcgacctgcctatcgtgcctctgatcctgggtggagctgccctgacagtgatctgtaggggcgattacgccctggagttcaccagagagttcctggaggccttcgagggccagacagagacagccgctgacatcaaggtgatcgcccagaaagccttcggtgtggacagactgtccgcctgcgccggcatcagcatcatcaagcctcacttccccttcagcgtggcctatacactggccgaaagactgatcaagagcgccaaggaggtgaagcagaaggtgaccgttaccaattctagccctatcaccccttttccatgtagcgccattgatttccacatcctgtacgacagcagcggcatcgactttgatagaatcagagagaagctgcggcctgaggataacacagaactgtacaacagaccctacgtggtcaccgccgccgaaaacctgagccaggcccaaggctacgagtggtcccaagcccactccctgcagaccctggcggacagagtgtcctacctgcgcagcgaggacggcgaaggcaagtctgccctgcccagcagccagagccacgccctgagaacagccctgtatctggaaaagaatgaagccgacgcccagtacagcctgatctctcaaagatacaagatcttgaagaacttcgccgaggacggcgagaacaagtctctgttccatctggaaaatggaaagtacgtgacccggttcctcgatgccctcgacgccaaggacttcttcgccaacgccaatcacaagaaccagggcgag

SEQ ID NO: 134 - DNA, 2402 bp, synthetic construct
  source 1..2402: mol_type = other DNA
taatacggttatccacagaatcaggggataacgcaggaaagaaactagtgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtttcacacactcgagatctgttcaacaccctcttttccccgtcaggggactgaaactgagacctttcacacaggaaacagtttttttacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgtgacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagacta

SEQ ID NO: 135 - DNA, 1902 bp, synthetic construct
  source 1..1902: mol_type = other DNA
atggccaatctggataagatgctgaacaccaccgtgaccgaggtgcggcagttcctgcaagtggacagagtgtgtgtgttccagttcgaggaagattactctggcgtggtggtcgtcgaggccgttgacgaccggtggatcagcatcctgaagacccaggtgcgcgacagatacttcatggaaacaagaggcgaagagtactcccacggaagatatcaggccatcgccgacatctacaccgccaacctgaccgagtgctacagagatctgctgacacagtttcaggtgcgggccatcctggccgtgcccatcctgcagggcaagaagctgtggggcctgctcgtggcccaccagctggctgctcctagacagtggcagacatgggagatcgacttcctgaaacagcaagctgtggtggtgggcatcgccattcagcagagcgaaggcagaggaagcctactgacgtgtggagacgtggaagagaacccaggccctgactacaaggatgatgatgataagatggcccctaagaagaagagaaaggtgcgcggcatggtcgacctgaagagcctggctggcgccgaaatggtgggcctcagatggcagctgagattcgaccggccttgccgcctggagagccactacgtgaaaggtctgcatgcctggttcctgcatcaggtgcaggccattgaccccgacgtgtctgcctggctgcacgacggccaaggcgagaagcctttcaccatcagcagattgatcggccctacactgtggcaggagggccactggcactggcaaatcaacaaaacctaccactggcagctgaacctgctgagcggcgccctgatcgaggccctgcagccttggctggctagactgccaaacaagatcgttctggccagacagacactgtgggtggaagctgtggactgctacctggcccctcacaactaccagcagctgtggcctcaaggagccctgcctagacggcaagaatttacctttacaagccccaccagcttcagaagacagggcaaccactatcctctgccggaacctaggaacgtgctccagtcctacctgcggagatggaatgacttcagcggcctggccttcgagccagagcctttcctggactactgggtgccccagaatgtggtcatcgaccggcactggctggaaagcgtgaagaccaccgccggaaagcaggggagcgtggtgggcttcgtgggcgccgtgtctcttgtgctgacaccccaggccagaaacgacggcgatgactacggaagactgtttcacgcgctgtgtagatacggcccctattgcggcaccggccacaagacaaccttcggcctgggccagaccatggccggctgggccacacctgatctgaaaaccttcgcctgtctgcaagaagatctgcagacccaggtgctgacacagagaatcgatcagtgcgcctctctactgctggctcagagacagcgcaccggaggacagcgggctcaggagatctgccacaccctggccaccatcttcgtgagacgggaacagggcgagtccctgcaggagatcgccctggatctgcagctgccctacgagacagcccggacctacagcaagcgggcaaagagagccctggctaacgtgcagtagtctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgccgtaatacggttatccacagaatcaggggataacgcaggaaagaaactagt

SEQ ID NO: 136 - DNA, 690 bp, synthetic construct
  source 1..690: mol_type = other DNA
gaaggcagaggaagcctactgacctgcggcgatgtggaagagaaccccggccctgactacaaggacgatgatgacaagatggcccctaagaagaaacggaaagtgcggggaatgcctgctggcggaagactgatgaagaacctttatcactaccatcagtacgagatcacactggaatccgccgtggatagctgtaaaaaccacctgcaggccgctatcggcctgctgtacagccctcagaagtgcgagctggtgaaactggacaacagcggcaagctggtcgacagctacaaccggctgaagttcaacaacctgggcgtgtttgaggccagattcttcaacctcaactgcgaactgagatgggttaacgagtctaatggcaacggaacagccgtgctgctgagcgaatctgatatcaccctgaccggcttcgagaagggcctgcaagagttcatcaccgccattgatcagcagtacctgctgtggggcgagcctgccaagcacccccccaacgccgacggctggcagcggctggccgaagctagaatcggaaagctggacatccctctggataatcctctgaaaccaaaggacagagtgtttctgaccagcgaggaatacatcgccgaggtggacgacttcggcaattgcgccgtgatcgacgagcggctgatcaagctggaagtgaag

SEQ ID NO: 137 - DNA, 2402 bp, synthetic construct; identical to SEQ ID NO: 134
  source 1..2402: mol_type = other DNA

SEQ ID NO: 138 - DNA, 73 bp, synthetic construct
  source 1..73: mol_type = other DNA
aaaccttactcttgtcagctatatcctcttcaaaaagtccgttcaacaccctcttttccccgtcaggggactg

SEQ ID NO: 139 - DNA, 73 bp, synthetic construct
  source 1..73: mol_type = other DNA
gtttcagtcccctgacggggaaaagagggtgttgaacggactttttgaagaggatatagctgacaagagtaag

SEQ ID NO: 140 - DNA, 73 bp, synthetic construct
  source 1..73: mol_type = other DNA
aaacttccgctagtgttggttagaatatcagaagccagaggttcaacaccctcttttccccgtcaggggactg

SEQ ID NO: 141 - DNA, 73 bp, synthetic construct
  source 1..73: mol_type = other DNA
gtttcagtcccctgacggggaaaagagggtgttgaacctctggcttctgatattctaaccaacactagcggaa

SEQ ID NO: 142 - DNA, 73 bp, synthetic construct
  source 1..73: mol_type = other DNA
aaaccgtagtaatcactgcccaacccagtgcttctggtcagttcaacaccctcttttccccgtcaggggactg

SEQ ID NO: 143 - DNA, 73 bp, synthetic construct
  source 1..73: mol_type = other DNA
gtttcagtcccctgacggggaaaagagggtgttgaactgaccagaagcactgggttgggcagtgattactacg

SEQ ID NO: 144 - DNA, 73 bp, synthetic construct
  source 1..73: mol_type = other DNA
aaacaattagtgaaaggtcagtggccaaatctctacggacgttcaacaccctcttttccccgtcaggggactg

SEQ ID NO: 145 - DNA, 73 bp, synthetic construct
  source 1..73: mol_type = other DNA
gtttcagtcccctgacggggaaaagagggtgttgaacgtccgtagagatttggccactgacctttcactaatt

SEQ ID NO: 146 - DNA, 73 bp, synthetic construct
  source 1..73: mol_type = other DNA
aaacggatatagctgacaagagtaagctcgaaggcgctgggttcaacaccctcttttccccgtcaggggactg

SEQ ID NO: 147 - DNA, 73 bp, synthetic construct
  source 1..73: mol_type = other DNA
gtttcagtcccctgacggggaaaagagggtgttgaacccagcgccttcgagcttactcttgtcagctatatcc

SEQ ID NO: 148 - DNA, 28 bp, synthetic construct
  source 1..28: mol_type = other DNA
tagtctagaggatcataatcagccatac

SEQ ID NO: 149 - DNA, 25 bp, synthetic construct
  source 1..25: mol_type = other DNA
taatacggttatccacagaatcagg

SEQ ID NO: 150 - DNA, 24 bp, synthetic construct
  source 1..24: mol_type = other DNA
ctcggcaagggcgcccttgaggat

SEQ ID NO: 151 - DNA, 24 bp, synthetic construct
  source 1..24: mol_type = other DNA
atcctcaagggcgcccttgccgag

SEQ ID NO: 152 - DNA, 1569 bp, synthetic construct
  source 1..1569: mol_type = other DNA
atggctagaaaagttactacacgctggaaaattacaggcacattaattgcagaaacccctttacacattggtggtgtgggtggcgacgctgatacggctttagccctggcggttaatggtgcgggtgaatattatgtgccagggacaagtttagccggtgctctgcggggttggatgacccagttattgaataatgatgagtcccaaattaaagatctttggggtgatcatttagatgcaaaacggggagctagctttgttattgttgacgatgcggttatccatatacccaataatgctgatgttgaaattagggagggtgttggcatcgatcgccattttggaaccgccgccaatgggtttaaatatagccgagcagttattcccaagggttctaaatttaaattgccattaacttttgacagtcaagatgatgggctaccgaatgcgttgattcaattgttgtgtgccttagaagcaggggatattcgccttggggccgcaaaaacccggggtttaggtcgcattaaactagatgatttaaagttaaaatcctttgctttagataaaccagaaggtattttttctgctttattagaccaaggtaaaaaattagattggaatcaattaaaagcaaacgttacctaccagtctcctccctatctaggtattagtattacctggaatcccaaagatcccgtcatggtgaaagctgaaggggatggactggcgatcgctattttgcccctcgttagtcaagtgggaagtgatgttcgatttgtcattcccggcagttccattaaggggattttacgaacccaggctgaacgtattattcgtactatttgccagtctaatggttctgagaaaaacttcctagaacaattacgaatcaatctggttaatgaattatttgggtctgcttctttgagccaaaaacaaaatggcaaggatatagatctgggtaaaatcggagccttggcagtgaatgattgtttttctagtttatccatgaccccagatcaatggaaagcggtagagaatgccacggagatgacggggaatttacagcctgctcttaaacaagctacgggttatcccaataatattagccaagcttacaaagtacttcaaccggccatgcacgtcgctgtagatcggtggacagggggagctgccgaaggaatgctttacagcgtgctcgaacccattggggtcacctgggaaccgatccaagttcacttggacattgcccgtctcaaaaattattaccacggtaaggaagaaaaacttaaaccggcgatcgccctattgcttcttgtattgcgggatttagctaacaaaaaaattcccgtaggctatggcactaaccgcggtatgggaacgattactgtcagtcaaatcaccctcaatggcaaagccctccccactgaacttgaacctttaaacaaaacaatgacttgtcctaatctcaccgatctagatgaggcatttcgtcaggacttaagcactgcttggaaagagtggattgccgatcccattgatctatgccagcaggaggccgcctaa

SEQ ID NO: 153 - protein, 522 aa, synthetic construct
MARKVTTRWKITGTLIAETPLHIGGVGGDADTALALAVNGAGEYYVPGTSLAGALRGWMTQLLNNDESQIKDLWGDHLDAKRGASFVIVDDAVIHIPNNADVEIREGVGIDRHFGTAANGFKYSRAVIPKGSKFKLPLTFDSQDDGLPNALIQLLCALEAGDIRLGAAKTRGLGRIKLDDLKLKSFALDKPEGIFSALLDQGKKLDWNQLKANVTYQSPPYLGISITWNPKDPVMVKAEGDGLAIAILPLVSQVGSDVRFVIPGSSIKGILRTQAERIIRTICQSNGSEKNFLEQLRINLVNELFGSASLSQKQNGKDIDLGKIGALAVNDCFSSLSMTPDQWKAVENATEMTGNLQPALKQATGYPNNISQAYKVLQPAMHVAVDRWTGGAAEGMLYSVLEPIGVTWEPIQVHLDIARLKNYYHGKEEKLKPAIALLLLVLRDLANKKIPVGYGTNRGMGTITVSQITLNGKALPTELEPLNKTMTCPNLTDLDEAFRQDLSTAWKEWIADPIDLCQQEAA

SEQ ID NO: 154 - DNA, 36 bp, synthetic construct
  source 1..36: mol_type = other DNA
agcggctccctggctctgctggttgctctcgttctc

SEQ ID NO: 155 - DNA, 28 bp, synthetic construct
  source 1..28: mol_type = other DNA
gagagcaaccagcagagccagggagccg

SEQ ID NO: 156 - DNA, 23 bp, synthetic construct
  source 1..23: mol_type = other DNA
cgactcccagcgttcccacggtc

SEQ ID NO: 157 - DNA, 23 bp, synthetic construct
  source 1..23: mol_type = other DNA
agaagatgaccgtgggaacgctg